(19) United States
(12) Patent Application Publication
(10) Pub. No.: US 2023/0270875 A1
(43) Pub. Date: Aug. 31, 2023

(54) METHODS OF USING ANTI-CD79B IMMUNOCONJUGATES

(71) Applicant: Genentech, Inc., South San Francisco, CA (US)

(72) Inventors: Jamie Harue HIRATA, South San Francisco, CA (US); Lisa MUSICK, South San Francisco, CA (US)

(73) Assignee: Genentech, Inc., South San Francisco, CA (US)

(21) Appl. No.: 18/048,986

(22) Filed: Oct. 24, 2022

Related U.S. Application Data
(63) Continuation of application No. PCT/US21/28921, filed on Apr. 23, 2021.
(60) Provisional application No. 63/015,447, filed on Apr. 24, 2020.
(60) Provisional application No. 63/024,322, filed on May 13, 2020.
(60) Provisional application No. 63/037,591, filed on Jun. 10, 2020.
(60) Provisional application No. 63/108,806, filed on Nov. 2, 2020.
(60) Provisional application No. 63/120,684, filed on Dec. 2, 2020.

(51) Int. Cl. A61K 47/68; A61K 31/635; A61K 39/395; C07K 16/28; A61K 45/06; A61P 35/00

(52) U.S. Cl. CPC A61K 47/6811; A61K 47/6849; A61K 31/635; A61K 39/39558; C07K 16/2803; C07K 16/2887; A61K 45/06; A61P 35/00; A61K 2039/545

(57) ABSTRACT

Provided herein are methods of treating B-cell proliferative disorders (such as Follicular Lymphoma and diffuse large B cell lymphoma) using immunoconjugates comprising anti-CD79b antibodies in combination with a Bcl-2 inhibitor (such as venetoclax) and an anti-CD20 antibody (such as obinutuzumab or rituximab).

### CROSS-REFERENCE TO RELATED APPLICATIONS

This application claims the benefit of U.S. Provisional Application 63/015,447, filed Apr. 24, 2020, U.S. Provisional Application 63/024,322, filed May 13, 2020, U.S. Provisional Application 63/037,591, filed Jun. 10, 2020, U.S. Provisional Application 63/108,806, filed Nov. 2, 2020, and U.S. Provisional Application 63/120,684, filed Dec. 2, 2020, each of which is hereby incorporated by reference in its entirety.

### REFERENCE TO AN ELECTRONIC SEQUENCE LISTING

The contents of the electronic sequence listing (146392050801seglist.xml; Size: 76,498 bytes; and Date of Creation: Apr. 5, 2023) are herein incorporated by reference in their entirety.

### FIELD OF THE INVENTION

The present disclosure relates to methods of treating B-cell proliferative disorders, e.g., follicular lymphoma (FL) and diffuse large B-cell lymphoma (DLBCL), by administering an immunoconjugate comprising anti-CD79b antibody in combination with a Bcl-2 inhibitor (e.g., venetoclax) and an anti-CD20 antibody (e.g., obinutuzumab or rituximab).

### BACKGROUND OF THE INVENTION

Non-Hodgkin's lymphoma (NHL) is the most common hematologic malignancy in adults. NHL is most often of B-cell origin. This includes a range of different subtypes of B-cell lymphoma, which are broadly divided into indolent and aggressive lymphomas, each with unique characteristics.

Follicular lymphoma (FL) is the most common subtype of indolent B-cell lymphoma, and FL accounts for about 22% of all newly diagnosed cases of B-cell lymphoma (Armitage et al. (1998) “New approach to classifying non-Hodgkin's lymphomas: clinical features of the major histologic subtypes. Non-Hodgkin's Lymphoma Classification Project.” J Clin Oncol. 16:2780-95). Approximately 90% of the cases have a t(14:18) translocation, which juxtaposes BCL2 with the IgH locus and results in deregulated expression of Bcl-2. FL remains an incurable disease with the currently available therapies. The addition of rituximab, an anti-CD20 monoclonal antibody, to commonly used induction chemotherapy, including CHOP (cyclophosphamide, doxorubicin, vincristine, and prednisolone or prednisone), CVP (cyclophosphamide, vincristine, and prednisone), fludarabine, or bendamustine (Zelenetz et al. (2014) “Non-Hodgkin's lymphoma, Version 2.2014.” J Natl Compr Canc Netw. 12:916-46; Dreyling et al. (2014). “Newly diagnosed and relapsed follicular lymphoma: ESMO clinical recommendations for diagnosis, treatment and follow-up.” Ann Oncol. 25: iii76-82), followed by rituximab maintenance therapy led to prolonged remission and improved patient outcomes (Salles et al. (2013) “Updated 6 year follow-up of the PRIMA study confirms the benefit of 2-year rituximab maintenance in follicular lymphoma patients responding to frontline immunochemotherapy.” Blood. Abstract 509). However, despite significant therapeutic progress with the use of chemoimmunotherapy as first-line treatment, most patients eventually relapse. Relapses are characterized by increasing refractoriness and decreasing duration of response to subsequent lines of therapy. Therefore, FL remains a disease with a high unmet medical need.

Diffuse large B-cell lymphoma (DLBCL) is the most common aggressive NHL; it accounts for approximately 30% of all NHLs diagnosed annually (Armitage and Weisenburger 1998). The use of immunochemotherapy, most commonly rituximab plus CHOP (R-CHOP) for newly diagnosed DLBCL, led to a significant improvement in survival in patients of all age groups (Pfreundschuh et al. 2011; Coiffier et al. 2010). However, nearly 40% of patients with DLBCL will eventually die of relapsed disease or disease that is refractory to first-line therapy. Patients with a high-risk International Prognostic Index (IPI) have a 5-year PFS rate of 40% following treatment with R-CHOP (Zhou et al. 2014). Second-line therapies include high-dose chemotherapy regimens such as rituximab plus ifosfamide, carboplatin, and etoposide or rituximab plus cisplatin, cytosine arabinoside, and dexamethasone followed by autologous stem-cell transplantation (SCT). Approximately half of patients do not achieve a complete remission after salvage treatment (Gisselbrecht et al. 2010). Moreover, elderly patients or patients with comorbidities are often deemed ineligible for this aggressive therapy. Therefore, DLBCL remains a disease with a high unmet medical need.

Accordingly, there is a need in the art for new treatments to provide additional therapeutic options and improve outcomes for Non-Hodgkin's lymphoma (NHL) patients, such as FL and DLBCL patients.

All references cited herein, including patent applications and publications, are hereby incorporated by reference in their entirety.

### SUMMARY

In some aspects, provided herein is a method for treating follicular lymphoma (FL) in a human in need thereof comprising administering to the human an effective amount of: (a) an immunoconjugate comprising the formula

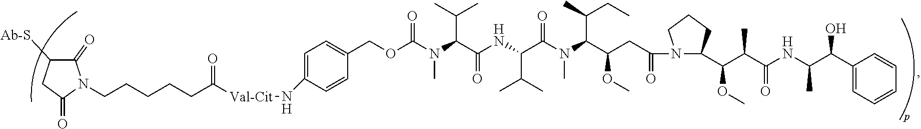

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) a selective Bcl-2 inhibitor, or a pharmaceutically acceptable salt thereof, and (c) an anti-CD20 antibody, wherein the human achieves a complete response (CR) during or after administration of the immunoconjugate, the selective Bcl-2 inhibitor, or a pharmaceutically acceptable salt thereof, and the anti-CD20 antibody. In some embodiments, p is between 3 and 4 or between 2 and 5. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin-piiq. In some embodiments, the selective Bcl-2 inhibitor is venetoclax, or a pharmaceutically acceptable salt thereof. In some embodiments, the polatuzumab vedotin-piiq is administered at a dose of about 1.8 mg/kg and the venetoclax or a pharmaceutically acceptable salt thereof is administered at a dose of about 800 mg. In some embodiments, the anti-CD20 antibody is rituximab. In some embodiments, the anti-CD20 antibody is obinutuzumab. In some embodiments, the polatuzumab vedotin-piiq is administered at a dose of about 1.8 mg/kg, the venetoclax or a pharmaceutically acceptable salt thereof is administered at a dose of about 800 mg, and the obinutuzumab is administered at a dose of about 1000 mg. In some embodiments, administration of the polatuzumab vedotin-piiq, the venetoclax or a pharmaceutically acceptable salt thereof, and the obinutuzumab to a plurality of humans results in a complete response in at least about 55%, at least about 57%, at least about 60%, at least about 65%, at least about 70%, at least about 75%, at least about 80%, at least about 85%, at least about 90%, at least about 95%, or 100% of the humans during or after administration of the polatuzumab vedotin-piiq, the venetoclax or a pharmaceutically acceptable salt thereof, and the obinutuzumab. In some embodiments, the duration of the complete response is at least about 1 month, at least about 2 months, at least about 3 months, or more. In some embodiments, administration of the polatuzumab vedotin-piiq, the venetoclax or a pharmaceutically acceptable salt thereof, and the obinutuzumab to a plurality of humans results in an objective response in at least about 87%, at least about 90%, at least about 95%, or 100% of the humans during or after administration of the polatuzumab vedotin-piiq, the venetoclax or a pharmaceutically acceptable salt thereof, and the obinutuzumab. In some embodiments, administration of the polatuzumab vedotin-piiq, the venetoclax or a pharmaceutically acceptable salt thereof, and the obinutuzumab to a plurality of humans results in an objective response in at least about 70%, at least about 75%, at least about 78%, at least about 80%, at least about 85%, at least about 90%, at least about 95%, or 100% of the humans during or after administration of the polatuzumab vedotin-piiq, the venetoclax or a pharmaceutically acceptable salt thereof, and the obinutuzumab. In some embodiments, administration of the polatuzumab vedotin-piiq, the venetoclax or a pharmaceutically acceptable salt thereof, and the obinutuzumab does not result in peripheral neuropathy of Grade 3 or greater in the human. In some embodiments, administration of the polatuzumab vedotin-piiq, the venetoclax or a pharmaceutically acceptable salt thereof, and the obinutuzumab does not result in tumor lysis syndrome in the human. In some embodiments, administration of the polatuzumab vedotin-piiq, the venetoclax or a pharmaceutically acceptable salt thereof, and the obinutuzumab to a plurality of humans results in a Grade 3 or Grade 4 adverse event in about 64% or less of the humans. In some embodiments, administration of the polatuzumab vedotin-piiq, the venetoclax or a pharmaceutically acceptable salt thereof, and the obinutuzumab to a plurality of humans results in a Grade 3 or Grade 4 adverse event in about 59% or less of the humans. In some embodiments, administration of the polatuzumab vedotin-piiq, the venetoclax or a pharmaceutically acceptable salt thereof, and the obinutuzumab to a plurality of humans results in a Grade 3 or Grade 4 adverse event in about 73% or less of the humans. In some embodiments, the polatuzumab vedotin-piiq, the venetoclax or a pharmaceutically acceptable salt thereof, and the obinutuzumab are administered during an induction phase, optionally, wherein the induction phase comprises at least six 21-day cycles. In some embodiments, (i) the polatuzumab vedotin-piiq is administered intravenously at a dose of about 1.8 mg/kg on Day 1 of the first 21-day cycle, the venetoclax or a pharmaceutically acceptable salt thereof is administered orally at a dose of about 800 mg on each of Days 1-21 of the first 21-day cycle, and the obinutuzumab is administered intravenously at a dose of about 1000 mg on each of Days 1, 8, and 15 of the first 21-day cycle, and (ii) the polatuzumab vedotin-piiq is administered intravenously at a dose of about 1.8 mg/kg on Day 1 of each of the second, third, fourth, fifth, and sixth 21-day cycles, the venetoclax or a pharmaceutically acceptable salt thereof is administered orally at a dose of about 800 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles. In some embodiments, the polatuzumab vedotin-piiq, the venetoclax or a pharmaceutically acceptable salt thereof, and the obinutuzumab are administered sequentially during the induction phase. In some embodiments, (i) the venetoclax or a pharmaceutically acceptable salt thereof is administered prior to the obinutuzumab and the obinutuzumab is administered prior to the polatuzumab vedotin-piiq on Day 1 of the first 21-day cycle, and the venetoclax or a pharmaceutically acceptable salt thereof is administered prior to the obinutuzumab on days 8 and 15 of the first 21-day cycle; and (ii) the venetoclax or a pharmaceutically acceptable salt thereof is administered prior to the obinutuzumab and the obinutuzumab is administered prior to the polatuzumab vedotin-piiq on Day 1 of each of the second, third, fourth, fifth, and sixth 21-day cycles. In some embodiments, administration of the polatuzumab vedotin-piiq, the venetoclax or a pharmaceutically acceptable salt thereof, and the obinutuzumab results in a complete response in the human after the six 21-day cycles. In some embodiments, administration of the polatuzumab vedotin-piiq, the venetoclax or a pharmaceutically acceptable salt thereof, and the obinutuzumab to a plurality of humans results in a complete response in at least about 55%, at least about 57%, at least about 60%, at least about 65%, at least about 70%, at least about 75%, at least about 80%, at least about 85%, at least about 90%, at least about 95%, or 100% of the humans after the six 21-day cycles. In some embodiments, the duration of the complete response is at least about 1 month, at least about 2 months, at least about 3 months, or more. In some embodiments, administration of the polatuzumab vedotin-piiq, the venetoclax or a pharmaceutically acceptable salt thereof, and the obinutuzumab to a plurality of humans results in an objective response in at least about 87%, at least about 90%, at least about 95%, or 100% of the humans after the six 21-day cycles. In some embodiments, administration of the polatuzumab vedotin-piiq, the venetoclax or a pharmaceutically acceptable salt thereof, and the obinutuzumab to a plurality of humans results in an objective response in at least about 70%, at least about 75%, at least about 78%, at least about 80%, at least about 85%, at least about 90%, at least about 95%, or 100% of the humans after the six 21-day cycles. In some embodiments, the venetoclax or a pharmaceutically acceptable salt thereof and the obinutuzumab are further administered during a maintenance phase after the sixth 21-day cycle of the induction phase, wherein the venetoclax or a pharmaceutically acceptable salt thereof is administered orally at a dose of about 800 mg once per day during the maintenance phase, and wherein the obinutuzumab is administered intravenously at a dose of about 1000 mg once every two months during the maintenance phase. In some embodiments, the venetoclax or a pharmaceutically acceptable salt thereof is administered for a maximum of 8 months during the maintenance phase. In some embodiments, the obinutuzumab is administered during the maintenance phase starting on Day 1 of the second month after the sixth 21-day cycle of the induction phase. In some embodiments, the obinutuzumab is administered for a maximum of 24 months during the maintenance phase. In some embodiments, the venetoclax or a pharmaceutically acceptable salt thereof and the obinutuzumab are administered sequentially during the maintenance phase. In some embodiments, the venetoclax or a pharmaceutically acceptable salt thereof is administered prior to the obinutuzumab on Day 1 of each of month 2, 4, 6, 8, 10, 12, 14, 16, 18, 20, 22, and 24 during the maintenance phase. In some embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin.

In another aspect, provided herein a method for treating follicular lymphoma (FL) in a human in need thereof comprising administering to the human an effective amount of: (a) an immunoconjugate at a dose of about 1.8 mg/kg, wherein the immunoconjugate comprises the formula

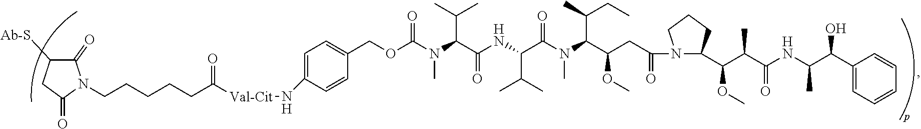

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) venetoclax or a pharmaceutically acceptable salt thereof at a dose of about 800 mg, and (c) obinutuzumab at a dose of about 1000 mg. In some embodiments, administration of the immunoconjugate, the venetoclax or a pharmaceutically acceptable salt thereof, and the obinutuzumab to a plurality of humans results in a complete response in at least about 55%, at least about 57%, at least about 60%, at least about 65%, at least about 70%, at least about 75%, at least about 80%, at least about 85%, at least about 90%, at least about 95%, or 100% of the humans during or after administration of the immunoconjugate, the venetoclax or a pharmaceutically acceptable salt thereof, and the obinutuzumab. In some embodiments, the duration of the complete response is at least about 1 month, at least about 2 months, at least about 3 months, or more. In some embodiments, administration of the immunoconjugate, the venetoclax or a pharmaceutically acceptable salt thereof, and the obinutuzumab to a plurality of humans results in an objective response in at least about 87%, at least about 90%, at least about 95%, or 100% of the humans during or after administration of the immunoconjugate, the venetoclax or a pharmaceutically acceptable salt thereof, and the obinutuzumab. In some embodiments, administration of the immunoconjugate, the venetoclax or a pharmaceutically acceptable salt thereof, and the obinutuzumab to a plurality of humans results in an objective response in at least about 70%, at least about 75%, at least about 78%, at least about 80%, at least about 85%, at least about 90%, at least about 95%, or 100% of the humans during or after administration of the immunoconjugate, the venetoclax or a pharmaceutically acceptable salt thereof, and the obinutuzumab. In some embodiments, administration of the immunoconjugate, the venetoclax or a pharmaceutically acceptable salt thereof, and the obinutuzumab does not result in a peripheral neuropathy of Grade 3 or greater in the human. In some embodiments, administration of the immunoconjugate, the venetoclax or a pharmaceutically acceptable salt thereof, and the obinutuzumab does not result in tumor lysis syndrome in the human. In some embodiments, administration of the immunoconjugate, the venetoclax or a pharmaceutically acceptable salt thereof, and the obinutuzumab to a plurality of humans results in a Grade 3 or Grade 4 adverse event in about 64% or less of the humans. In some embodiments, administration of the immunoconjugate, the venetoclax or a pharmaceutically acceptable salt thereof, and the obinutuzumab to a plurality of humans results in a Grade 3 or Grade 4 adverse event in about 59% or less of the humans. In some embodiments, administration of the immunoconjugate, the venetoclax or a pharmaceutically acceptable salt thereof, and the obinutuzumab to a plurality of humans results in a Grade 3 or Grade 4 adverse event in about 73% or less of the humans. In some embodiments, p is between 3 and 4 or between 2 and 5. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin-piiq. In some embodiments, the polatuzumab vedotin-piiq, the venetoclax or a pharmaceutically acceptable salt thereof, and the obinutuzumab are administered during an induction phase, optionally, wherein the induction phase comprises at least six 21-day cycles. In some embodiments, (i) the polatuzumab vedotin-piiq is administered intravenously at a dose of about 1.8 mg/kg on Day 1 of the first 21-day cycle, the venetoclax or a pharmaceutically acceptable salt thereof is administered orally at a dose of about 800 mg on each of Days 1-21 of the first 21-day cycle, and the obinutuzumab is administered intravenously at a dose of about 1000 mg on each of Days 1, 8, and 15 of the first 21-day cycle, and (ii) the polatuzumab vedotin-piiq is administered intravenously at a dose of about 1.8 mg/kg on Day 1 of each of the second, third, fourth, fifth, and sixth 21-day cycles, the venetoclax or a pharmaceutically acceptable salt thereof is administered orally at a dose of about 800 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles. In some embodiments, the polatuzumab vedotin-piiq, the venetoclax or a pharmaceutically acceptable salt thereof, and the obinutuzumab are administered sequentially during the induction phase. In some embodiments, (i) the venetoclax or a pharmaceutically acceptable salt thereof is administered prior to the obinutuzumab and the obinutuzumab is administered prior to the polatuzumab vedotin-piiq on Day 1 of the first 21-day cycle, and the venetoclax or a pharmaceutically acceptable salt thereof is administered prior to the obinutuzumab on days 8 and 15 of the first 21-day cycle; and (ii) the venetoclax or a pharmaceutically acceptable salt thereof is administered prior to the obinutuzumab and the obinutuzumab is administered prior to the polatuzumab vedotin-piiq on Day 1 of each of the second, third, fourth, fifth, and sixth 21-day cycles. In some embodiments, administration of the polatuzumab vedotin-piiq, the venetoclax or a pharmaceutically acceptable salt thereof, and the obinutuzumab results in a complete response in the human after the six 21-day cycles. In some embodiments, administration of the polatuzumab vedotin-piiq, the venetoclax or a pharmaceutically acceptable salt thereof, and the obinutuzumab to a plurality of humans results in a complete response in at least about 55%, at least about 60%, at least about 65%, at least about 70%, at least about 75%, at least about 80%, at least about 85%, at least about 90%, at least about 95%, or 100% of the humans after the six 21-day cycles. In some embodiments, the duration of the complete response is at least about 1 month, at least about 2 months, at least about 3 months, or more. In some embodiments, administration of the polatuzumab vedotin-piiq, the venetoclax or a pharmaceutically acceptable salt thereof, and the obinutuzumab to a plurality of humans results in an objective response in at least about 87%, at least about 90%, at least about 95%, or 100% of the humans after the six 21-day cycles. In some embodiments, administration of the polatuzumab vedotin-piiq, the venetoclax or a pharmaceutically acceptable salt thereof, and the obinutuzumab to a plurality of humans results in an objective response in at least about 70%, at least about 75%, at least about 78%, at least about 80%, at least about 85%, at least about 90%, at least about 95%, or 100% of the humans after the six 21-day cycles. In some embodiments, the venetoclax or a pharmaceutically acceptable salt thereof and the obinutuzumab are further administered during a maintenance phase after the sixth 21-day cycle of the induction phase, wherein the venetoclax or a pharmaceutically acceptable salt thereof is administered orally at a dose of about 800 mg once per day during the maintenance phase, and wherein the obinutuzumab is administered intravenously at a dose of about 1000 mg once every two months during the maintenance phase. In some embodiments, the venetoclax or a pharmaceutically acceptable salt thereof is administered for a maximum of 8 months during the maintenance phase. In some embodiments, the obinutuzumab is administered during the maintenance phase starting on Day 1 of the second month after the sixth 21-day cycle of the induction phase. In some embodiments, the obinutuzumab is administered for a maximum of 24 months during the maintenance phase. In some embodiments, the venetoclax or a pharmaceutically acceptable salt thereof and the obinutuzumab are administered sequentially during the maintenance phase. In some embodiments, the venetoclax or a pharmaceutically acceptable salt thereof is administered prior to the obinutuzumab on Day 1 of each of month 2, 4, 6, 8, 10, 12, 14, 16, 18, 20, 22, and 24 during the maintenance phase. In some embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin.

In another aspect, provided herein is a method for treating follicular lymphoma (FL) in a human in need thereof comprising administering to the human an effective amount of: (a) an immunoconjugate at dose of about 1.8 mg/kg, wherein the immunoconjugate comprises the formula

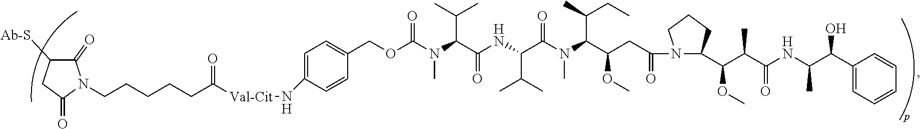

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) venetoclax or a pharmaceutically acceptable salt thereof at a dose of about 800 mg, and (c) obinutuzumab at a dose of about 1000 mg, wherein the human achieves a complete response (CR) during or after administration of the immunoconjugate, the venetoclax, and the obinutuzumab. In some embodiments, p is between 3 and 4 or between 2 and 5. In some embodiments, administration of the immunoconjugate, the venetoclax or a pharmaceutically acceptable salt thereof, and the obinutuzumab to a plurality of humans results in a complete response in at least about 55%, at least about 57%, at least about 60%, at least about 65%, at least about 70%, at least about 75%, at least about 80%, at least about 85%, at least about 90%, at least about 95%, or 100% of the humans during or after administration of the immunoconjugate, the venetoclax or a pharmaceutically acceptable salt thereof, and the obinutuzumab. In some embodiments, the duration of the complete response is at least about 1 month, at least about 2 months, at least about 3 months, or more. In some embodiments, administration of the immunoconjugate, the venetoclax or a pharmaceutically acceptable salt thereof, and the obinutuzumab to a plurality of humans results in an objective response in at least about 87%, at least about 90%, at least about 95%, or 100% of the humans during or after administration of the immunoconjugate, the venetoclax or a pharmaceutically acceptable salt thereof, and the obinutuzumab. In some embodiments, administration of the immunoconjugate, the venetoclax or a pharmaceutically acceptable salt thereof, and the obinutuzumab to a plurality of humans results in an objective response in at least about 70%, at least about 75%, at least about 78%, at least about 80%, at least about 85%, at least about 90%, at least about 95%, or 100% of the humans during or after administration of the immunoconjugate, the venetoclax or a pharmaceutically acceptable salt thereof, and the obinutuzumab. In some embodiments, administration of the immunoconjugate, the venetoclax or a pharmaceutically acceptable salt thereof, and the obinutuzumab does not result in peripheral neuropathy of Grade 3 or greater in the human. In some embodiments, administration of the immunoconjugate, the venetoclax or a pharmaceutically acceptable salt thereof, and the obinutuzumab does not result in tumor lysis syndrome in the human. In some embodiments, administration of the immunoconjugate, the venetoclax or a pharmaceutically acceptable salt thereof, and the obinutuzumab to a plurality of humans results in a Grade 3 or Grade 4 adverse event in about 64% or less of the humans. In some embodiments, administration of the immunoconjugate, the venetoclax or a pharmaceutically acceptable salt thereof, and the obinutuzumab to a plurality of humans results in a Grade 3 or Grade 4 adverse event in about 59% or less of the humans. In some embodiments, administration of the immunoconjugate, the venetoclax or a pharmaceutically acceptable salt thereof, and the obinutuzumab to a plurality of humans results in a Grade 3 or Grade 4 adverse event in about 73% or less of the humans. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin-piiq. In some embodiments, the polatuzumab vedotin-piiq, the venetoclax or a pharmaceutically acceptable salt thereof, and the obinutuzumab are administered during an induction phase, optionally, wherein the induction phase comprises at least six 21-day cycles. In some embodiments, (i) the polatuzumab vedotin-piiq is administered intravenously at a dose of about 1.8 mg/kg on Day 1 of the first 21-day cycle, the venetoclax or a pharmaceutically acceptable salt thereof is administered orally at a dose of about 800 mg on each of Days 1-21 of the first 21-day cycle, and the obinutuzumab is administered intravenously at a dose of about 1000 mg on each of Days 1, 8, and 15 of the first 21-day cycle, and (ii) the polatuzumab vedotin-piiq is administered intravenously at a dose of about 1.8 mg/kg on Day 1 of each of the second, third, fourth, fifth, and sixth 21-day cycles, the venetoclax or a pharmaceutically acceptable salt thereof is administered orally at a dose of about 800 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles. In some embodiments, the polatuzumab vedotin-piiq, the venetoclax or a pharmaceutically acceptable salt thereof, and the obinutuzumab are administered sequentially during the induction phase. In some embodiments, (i) the venetoclax or a pharmaceutically acceptable salt thereof is administered prior to the obinutuzumab and the obinutuzumab is administered prior to the polatuzumab vedotin-piiq on Day 1 of the first 21-day cycle, and the venetoclax or a pharmaceutically acceptable salt thereof is administered prior to the obinutuzumab on days 8 and 15 of the first 21-day cycle; and (ii) the venetoclax or a pharmaceutically acceptable salt thereof is administered prior to the obinutuzumab and the obinutuzumab is administered prior to the polatuzumab vedotin-piiq on Day 1 of each of the second, third, fourth, fifth, and sixth 21-day cycles. In some embodiments, administration of the polatuzumab vedotin-piiq, the venetoclax or a pharmaceutically acceptable salt thereof, and the obinutuzumab results in a complete response in the human after the six 21-day cycles. In some embodiments, administration of the polatuzumab vedotin-piiq, the venetoclax or a pharmaceutically acceptable salt thereof, and the obinutuzumab to a plurality of humans results in a complete response in at least about 55%, at least about 57%, at least about 60%, at least about 65%, at least about 70%, at least about 75%, at least about 80%, at least about 85%, at least about 90%, at least about 95%, or 100% of the humans after the six 21-day cycles. In some embodiments, the duration of the complete response is at least about 1 month, at least about 2 months, at least about 3 months, or more. In some embodiments, administration of the polatuzumab vedotin-piiq, the venetoclax or a pharmaceutically acceptable salt thereof, and the obinutuzumab to a plurality of humans results in an objective response in at least about 87%, at least about 90%, at least about 95%, or 100% of the humans after the six 21-day cycles. In some embodiments, administration of the polatuzumab vedotin-piiq, the venetoclax or a pharmaceutically acceptable salt thereof, and the obinutuzumab to a plurality of humans results in an objective response in at least about 70%, at least about 75%, at least about 78%, at least about 80%, at least about 85%, at least about 90%, at least about 95%, or 100% of the humans after the six 21-day cycles. In some embodiments, the venetoclax or a pharmaceutically acceptable salt thereof and the obinutuzumab are further administered during a maintenance phase after the sixth 21-day cycle of the induction phase, wherein the venetoclax or a pharmaceutically acceptable salt thereof is administered orally at a dose of about 800 mg once per day during the maintenance phase, and wherein the obinutuzumab is administered intravenously at a dose of about 1000 mg once every two months during the maintenance phase. In some embodiments, the venetoclax or a pharmaceutically acceptable salt thereof is administered for a maximum of 8 months during the maintenance phase. In some embodiments, the obinutuzumab is administered during the maintenance phase starting on Day 1 of the second month after the sixth 21-day cycle of the induction phase. In some embodiments, the obinutuzumab is administered for a maximum of 24 months during the maintenance phase. In some embodiments, the venetoclax or a pharmaceutically acceptable salt thereof and the obinutuzumab are administered sequentially during the maintenance phase. In some embodiments, the venetoclax or a pharmaceutically acceptable salt thereof is administered prior to the obinutuzumab on Day 1 of each of months 2, 4, 6, 8, 10, 12, 14, 16, 18, 20, 22, and 24 during the maintenance phase. In some embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin.

In another aspect, provided herein is a method of treating follicular lymphoma (FL) in a human in need thereof, comprising administering to the human, during an induction phase, an effective amount of: (a) polatuzumab vedotin-piiq at a dose of about 1.8 mg/kg, (b) venetoclax or a pharmaceutically acceptable salt thereof at a dose of about 800 mg, and (c) obinutuzumab at a dose of about 1000 mg, wherein the human achieves a complete response during or after the induction phase. In some embodiments, administration of the polatuzumab vedotin-piiq, the venetoclax or a pharmaceutically acceptable salt thereof, and the obinutuzumab to a plurality of humans results in a complete response in at least about 55%, at least about 57%, at least about 60%, at least about 65%, at least about 70%, at least about 75%, at least about 80%, at least about 85%, at least about 90%, at least about 95%, or 100% of the humans during or after the induction phase. In some embodiments, the duration of the complete response is at least about 1 month, at least about 2 months, at least about 3 months, or more. In some embodiments, administration of the polatuzumab vedotin-piiq, the venetoclax or a pharmaceutically acceptable salt thereof, and the obinutuzumab to a plurality of humans results in an objective response in at least about 87%, at least about 90%, at least about 95%, or 100% of the humans during or after the induction phase. In some embodiments, administration of the polatuzumab vedotin-piiq, the venetoclax or a pharmaceutically acceptable salt thereof, and the obinutuzumab to a plurality of humans results in an objective response in at least about 70%, at least about 75%, at least about 78%, at least about 80%, at least about 85%, at least about 90%, at least about 95%, or 100% of the humans during or after the induction phase. In some embodiments, administration of the polatuzumab vedotin-piiq, the venetoclax or a pharmaceutically acceptable salt thereof, and the obinutuzumab does not result in peripheral neuropathy of Grade 3 or greater in the human. In some embodiments, administration of the polatuzumab vedotin-piiq, the venetoclax or a pharmaceutically acceptable salt thereof, and the obinutuzumab does not result in tumor lysis syndrome in the human. In some embodiments, administration of the polatuzumab vedotin-piiq, the venetoclax or a pharmaceutically acceptable salt thereof, and the obinutuzumab to a plurality of humans results in a Grade 3 or Grade 4 adverse event in about 64% or less of the humans. In some embodiments, administration of the polatuzumab vedotin-piiq, the venetoclax or a pharmaceutically acceptable salt thereof, and the obinutuzumab to a plurality of humans results in a Grade 3 or Grade 4 adverse event in about 59% or less of the humans. In some embodiments, administration of the polatuzumab vedotin-piiq, the venetoclax or a pharmaceutically acceptable salt thereof, and the obinutuzumab to a plurality of humans results in a Grade 3 or Grade 4 adverse event in about 73% or less of the humans. In some embodiments, the induction phase comprises at least six 21-day cycles. In some embodiments, (i) the polatuzumab vedotin-piiq is administered intravenously at a dose of about 1.8 mg/kg on Day 1 of the first 21-day cycle, the venetoclax or a pharmaceutically acceptable salt thereof is administered orally at a dose of about 800 mg on each of Days 1-21 of the first 21-day cycle, and the obinutuzumab is administered intravenously at a dose of about 1000 mg on each of Days 1, 8, and 15 of the first 21-day cycle, and (ii) the polatuzumab vedotin-piiq is administered intravenously at a dose of about 1.8 mg/kg on Day 1 of each of the second, third, fourth, fifth, and sixth 21-day cycles, the venetoclax or a pharmaceutically acceptable salt thereof is administered orally at a dose of about 800 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles. In some embodiments, the polatuzumab vedotin-piiq, the venetoclax or a pharmaceutically acceptable salt thereof, and the obinutuzumab are administered sequentially during the induction phase. In some embodiments, (i) the venetoclax or a pharmaceutically acceptable salt thereof is administered prior to the obinutuzumab and the obinutuzumab is administered prior to the polatuzumab vedotin-piiq on Day 1 of the first 21-day cycle, and the venetoclax or a pharmaceutically acceptable salt thereof is administered prior to the obinutuzumab on days 8 and 15 of the first 21-day cycle; and (ii) the venetoclax or a pharmaceutically acceptable salt thereof is administered prior to the obinutuzumab and the obinutuzumab is administered prior to the polatuzumab vedotin-piiq on Day 1 of each of the second, third, fourth, fifth, and sixth 21-day cycles. In some embodiments, the venetoclax or a pharmaceutically acceptable salt thereof and the obinutuzumab are further administered during a maintenance phase after the sixth 21-day cycle of the induction phase, wherein the venetoclax or a pharmaceutically acceptable salt thereof is administered orally at a dose of about 800 mg once per day during the maintenance phase, and wherein the obinutuzumab is administered intravenously at a dose of about 1000 mg once every two months during the maintenance phase. In some embodiments, the venetoclax or a pharmaceutically acceptable salt thereof is administered for a maximum of 8 months during the maintenance phase. In some embodiments, the obinutuzumab is administered during the maintenance phase starting on Day 1 of the second month after the sixth 21-day cycle of the induction phase. In some embodiments, the obinutuzumab is administered for a maximum of 24 months during the maintenance phase. In some embodiments, the venetoclax or a pharmaceutically acceptable salt thereof and the obinutuzumab are administered sequentially during the maintenance phase. In some embodiments, the venetoclax or a pharmaceutically acceptable salt thereof is administered prior to the obinutuzumab on Day 1 of each of months 2, 4, 6, 8, 10, 12, 14, 16, 18, 20, 22, and 24 during the maintenance phase.

In some embodiments which may be combined with any of the preceding aspects or embodiments, the method further comprises administering a prophylactic treatment for tumor lysis syndrome (TLS), wherein the prophylactic treatment for tumor lysis syndrome (TLS) comprises a uric acid-reducing agent and/or a hydration regimen prior to the start of treatment. In some embodiments, the hydration regimen comprises administering about 2 to about 3 liters per day of fluids, wherein the fluids are administered starting at about 24 hours to about 48 hours prior to the start of treatment. In some embodiments, the fluids are administered orally or intravenously. In some embodiments, the uric acid-reducing agent is allopurinol. In some embodiments, the allopurinol is administered orally at a dose of about 300 mg/day starting at about 72 hours prior to the first dose of venetoclax or a pharmaceutically acceptable salt thereof, and wherein administration of allopurinol continues for between about 3 to about 7 days after administration of the first dose of venetoclax or a pharmaceutically acceptable salt thereof. In some embodiments which may be combined with any of the preceding aspects or embodiments, the method further comprises administering granulocyte colony stimulating factor (G-CSF) if a Grade 3 or Grade 4 adverse event of neutropenia occurs.

In some embodiments which may be combined with any of the preceding aspects or embodiments, the human has an Eastern Cooperative Oncology Group (ECOG) Performance Status score of 0, 1, or 2 prior to the start of treatment. In some embodiments which may be combined with any of the preceding aspects or embodiments, the FL is relapsed or refractory to a prior treatment for FL. In some embodiments, the prior treatment for FL comprises a chemoimmunotherapy regimen comprising an anti-CD20 monoclonal antibody. In some embodiments which may be combined with any of the preceding aspects or embodiments, the FL is histologically documented as being CD20-positive. In some embodiments which may be combined with any of the preceding aspects or embodiments, the FL is fluorodeoxyglucose (FDG)-avid FL. In some embodiments which may be combined with any of the preceding aspects or embodiments, the FL is positron emission tomography (PET)-positive FL. In some embodiments which may be combined with any of the preceding aspects or embodiments, the human has at least one bi-dimensionally measurable lesion prior to treatment, wherein the lesion is at least 1.5 centimeters in its largest dimension as measured by computed tomography (CT) scan or magnetic resonance imaging (MRI). In some embodiments which may be combined with any of the preceding aspects or embodiments, the FL is not grade 3b FL. In some embodiments which may be combined with any of the preceding aspects or embodiments, the human does not have peripheral neuropathy of grade greater than 1 prior to treatment. In some embodiments which may be combined with any of the preceding aspects or embodiments, the FL has a histologic grade of 1, 2, or 3a prior to treatment. In some embodiments which may be combined with any of the preceding aspects or embodiments, the human has FL with bone marrow involvement prior to treatment. In some embodiments which may be combined with any of the preceding aspects or embodiments, the human has FL with an Ann Arbor stage of 1, 2, 3, or 4 prior to treatment. In some embodiments which may be combined with any of the preceding aspects or embodiments, the human has FL with a Follicular Lymphoma International Prognostic Index (FLIPI) score of 0, 1, 2, 3, 4, or 5 prior to treatment. In some embodiments which may be combined with any of the preceding aspects or embodiments, the human has received at least one prior treatment for FL. In some embodiments which may be combined with any of the preceding aspects or embodiments, the human has bulky disease of greater than 7 centimeters prior to treatment. In some embodiments which may be combined with any of the preceding aspects or embodiments, the FL is refractory to a prior treatment comprising an anti-CD20 agent. In some embodiments which may be combined with any of the preceding aspects or embodiments, the FL progressed within 24 months of completing a first treatment for FL. In some embodiments which may be combined with any of the preceding aspects or embodiments, the human has FL that progressed within about 24 months of initiation of the first anti-lymphoma treatment with chemoimmunotherapy administered to the human prior to treatment according to the methods provided herein. In some embodiments which may be combined with any of the preceding aspects or embodiments, the human has FL that did not progress within about 24 months of initiation of the first anti-lymphoma treatment with chemoimmunotherapy administered to the human prior to treatment according to the methods provided herein. In some embodiments which may be combined with any of the preceding aspects or embodiments, the human has received 1 or 2 lines of anti-lymphoma therapy prior to treatment according to the methods provided herein. In some embodiments which may be combined with any of the preceding aspects or embodiments, the human has received 3 or more lines of anti-lymphoma therapy prior to treatment according to the methods provided herein. In some embodiments which may be combined with any of the preceding aspects or embodiments, the human has FL with a Follicular Lymphoma International Prognostic Index (FLIPI) score of 0-2 prior to treatment according to the methods provided herein. In some embodiments which may be combined with any of the preceding aspects or embodiments, the human has FL with a Follicular Lymphoma International Prognostic Index (FLIPI) score of 3-5 prior to treatment according to the methods provided herein. In some embodiments which may be combined with any of the preceding aspects or embodiments, the human has refractory FL prior to treatment according to the methods provided herein. In some embodiments which may be combined with any of the preceding aspects or embodiments, the human has non-refractory FL prior to treatment according to the methods provided herein.

In another aspect, provided herein is a kit comprising an immunoconjugate comprising the formula

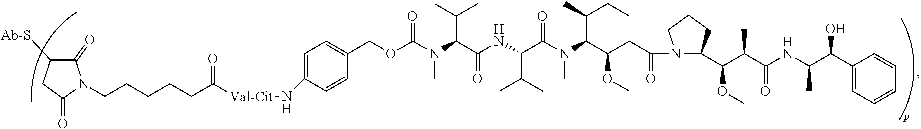

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with a selective Bcl-2 inhibitor or a pharmaceutically acceptable salt thereof and an anti-CD20 antibody for treating a human in need thereof having follicular lymphoma (FL) according to any of the methods provided herein.

In another aspect, provided herein is a kit comprising an immunoconjugate comprising the formula

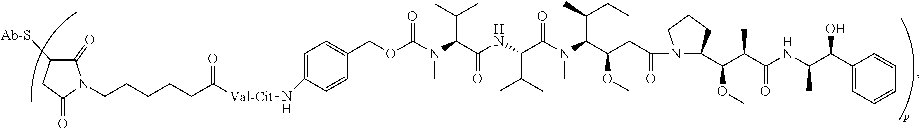

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with venetoclax or a pharmaceutically acceptable salt thereof and obinutuzumab for treating a human in need thereof having follicular lymphoma (FL) according to any of the methods provided herein.

In another aspect, provided herein is an immunoconjugate comprising the formula

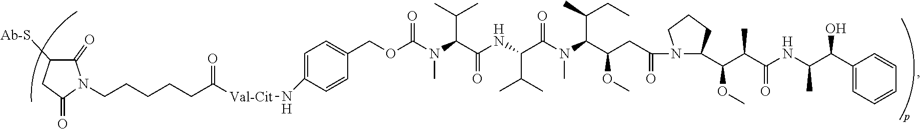

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with venetoclax or a pharmaceutically acceptable salt thereof and obinutuzumab for treating a human in need thereof having follicular lymphoma (FL) according to any of the methods provided herein.

In another aspect, provided herein is an immunoconjugate comprising the formula

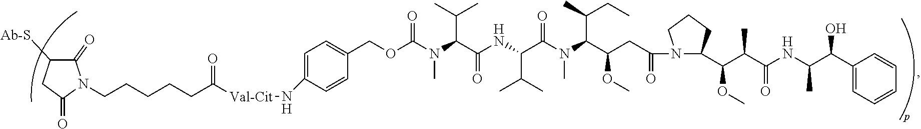

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in the manufacture of a medicament in combination with venetoclax or a pharmaceutically acceptable salt thereof and obinutuzumab for treating a human in need thereof having follicular lymphoma (FL) according to any of the methods provided herein.

In some embodiments which may be combined with any of the preceding aspects or embodiments, p is between 3 and 4 or between 2 and 5. In some embodiments which may be combined with any of the preceding aspects or embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments which may be combined with any of the preceding aspects or embodiments, the anti-CD79b antibody comprises (i) a heavy chain comprising the amino acid sequence of SEQ ID NO: 36 and (ii) a light chain comprising the amino acid sequence of SEQ ID NO: 35.

In another aspect, provided herein is a kit comprising polatuzumab vedotin-piiq for use in combination with venetoclax or a pharmaceutically acceptable salt thereof and obinutuzumab for treating a human in need thereof having follicular lymphoma (FL) according to any of the methods provided herein.

In another aspect, provided herein is polatuzumab vedotin-piiq for use in combination with venetoclax or a pharmaceutically acceptable salt thereof and obinutuzumab for treating a human in need thereof having follicular lymphoma (FL) according to any of the methods provided herein.

In another aspect, provided herein is polatuzumab vedotin-piiq for use in the manufacture of a medicament in combination with venetoclax or a pharmaceutically acceptable salt thereof and obinutuzumab for treating a human in need thereof having follicular lymphoma (FL) according to any of the methods provided herein.

In another aspect, provided herein is method for treating diffuse large B-cell lymphoma (DLBCL) in a human in need thereof comprising administering to the human an effective amount of: (a) an immunoconjugate comprising the formula

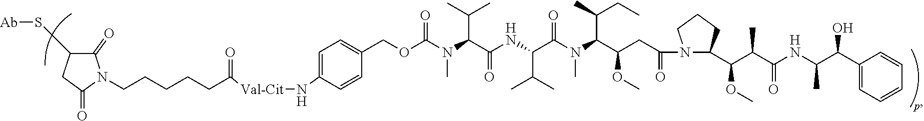

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) a selective Bcl-2 inhibitor, or a pharmaceutically acceptable salt thereof, and (c) an anti-CD20 antibody, wherein the human achieves a complete response (CR) during or after administration of the immunoconjugate, the selective Bcl-2 inhibitor, or a pharmaceutically acceptable salt thereof, and the anti-CD20 antibody. In some embodiments, p is between 3 and 4 or between 2 and 5. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin-piiq. In some embodiments, the selective Bcl-2 inhibitor is venetoclax, or a pharmaceutically acceptable salt thereof. In some embodiments, the polatuzumab vedotin-piiq is administered at a dose of about 1.8 mg/kg and the venetoclax or a pharmaceutically acceptable salt thereof is administered at a dose of about 800 mg. In some embodiments, the anti-CD20 antibody is rituximab. In some embodiments, the polatuzumab vedotin-piiq is administered at a dose of about 1.8 mg/kg, the venetoclax or a pharmaceutically acceptable salt thereof is administered at a dose of about 800 mg, and the rituximab is administered at a dose of about 375 mg/m². In some embodiments, administration of the polatuzumab vedotin-piiq, the venetoclax or a pharmaceutically acceptable salt thereof, and the rituximab to a plurality of humans results in a complete response in at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans during or after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to a plurality of humans results in a best complete response rate of at least about 35%, at least about 38%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the polatuzumab vedotin-piiq, the venetoclax or a pharmaceutically acceptable salt thereof, and the rituximab to a plurality of humans results in an objective response in at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100% of the humans during or after administration of the polatuzumab vedotin-piiq, the venetoclax or a pharmaceutically acceptable salt thereof, and the rituximab. In some embodiments, the duration of the complete response or the objective response is at least about 3 months, at least about 4 months, at least about 5 months, at least about 6 months, at least about 7 months, or more. In some embodiments, administration of the polatuzumab vedotin-piiq, the venetoclax or a pharmaceutically acceptable salt thereof, and the rituximab to a plurality of humans results in a best overall response in at least about 60%, at least about 65%, at least about 70%, at least about 75%, at least about 80%, at least about 85%, at least about 90%, at least about 95%, or 100% of the humans during or after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to a plurality of humans results in a six-month progression-free survival rate of at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the polatuzumab vedotin-piiq, the venetoclax or a pharmaceutically acceptable salt thereof, and the rituximab to the human results in progression-free survival of the human for at least about 3 months, at least about 4 months, at least about 5 months, at least about 6 months, at least about 7 months, or more after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to the human results in survival of the human for at least about 6 months, at least about 7 months, at least about 8 months, at least about 9 months, at least about 10 months, at least about 11 months, or more after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to the human results in a reduction in the sum of the product of diameters (SPD) of at least about 50%, at least about 60%, at least about 70%, at least about 80%, at least about 90%, at least about 95%, at least about 99%, or 100% compared to the SPD prior to administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to a plurality of humans results in a serious adverse event in about 40% or less, about 37% or less, about 35% or less, or about 30% or less of the humans. In some embodiments, the polatuzumab vedotin-piiq, the venetoclax or a pharmaceutically acceptable salt thereof, and the rituximab are administered during an induction phase, optionally, wherein the induction phase comprises at least six 21-day cycles. In some embodiments, the polatuzumab vedotin-piiq is administered intravenously at a dose of about 1.8 mg/kg on Day 1 of each of the first, second, third, fourth, fifth, and sixth 21-day cycles, the venetoclax or a pharmaceutically acceptable salt thereof is administered orally at a dose of about 800 mg on each of Days 1-21 of each of the first, second, third, fourth, fifth, and sixth 21-day cycles, and the rituximab is administered intravenously at a dose of about 375 mg/m² on Day 1 of each of the first, second, third, fourth, fifth, and sixth 21-day cycles. In some embodiments, the polatuzumab vedotin-piiq, the venetoclax or a pharmaceutically acceptable salt thereof, and the rituximab are administered sequentially during the induction phase. In some embodiments, the venetoclax or a pharmaceutically acceptable salt thereof is administered prior to the rituximab and the rituximab is administered prior to the polatuzumab vedotin-piiq on Day 1 of each of the first, second, third, fourth, fifth, and sixth 21-day cycles. In some embodiments, administration of the polatuzumab vedotin-piiq, the venetoclax or a pharmaceutically acceptable salt thereof, and the rituximab results in a complete response in the human after the six 21-day cycles. In some embodiments, administration of the polatuzumab vedotin-piiq, the venetoclax or a pharmaceutically acceptable salt thereof, and the rituximab to a plurality of humans results in a complete response in at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans after the six 21-day cycles. In some embodiments, administration of the polatuzumab vedotin-piiq, the venetoclax or a pharmaceutically acceptable salt thereof, and the rituximab to a plurality of humans results in a best complete response rate of at least about 35%, at least about 38%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the polatuzumab vedotin-piiq, the venetoclax or a pharmaceutically acceptable salt thereof, and the rituximab to a plurality of humans results in an objective response in at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100% of the humans after the six 21-day cycles. In some embodiments, the duration of the complete response or the objective response is at least about 3 months, at least about 4 months, at least about 5 months, at least about 6 months, at least about 7 months, or more. In some embodiments, administration of the polatuzumab vedotin-piiq, the venetoclax or a pharmaceutically acceptable salt thereof, and the rituximab to a plurality of humans results in a best overall response in at least about 60%, at least about 65%, at least about 70%, at least about 75%, at least about 80%, at least about 85%, at least about 90%, at least about 95%, or 100% of the humans after the six 21-day cycles. In some embodiments, administration of the polatuzumab vedotin-piiq, the venetoclax or a pharmaceutically acceptable salt thereof, and the rituximab to a plurality of humans results in a six-month progression-free survival rate of at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the polatuzumab vedotin-piiq, the venetoclax or a pharmaceutically acceptable salt thereof, and the rituximab to the human results in progression-free survival of the human for at least about 3 months, at least about 4 months, at least about 5 months, at least about 6 months, at least about 7 months, or more after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to the human results in survival of the human for at least about 6 months, at least about 7 months, at least about 8 months, at least about 9 months, at least about 10 months, at least about 11 months, or more after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to the human results in a reduction in the sum of the product of diameters (SPD) of at least about 50%, at least about 60%, at least about 70%, at least about 80%, at least about 90%, at least about 95%, at least about 99%, or 100% after the six 21-day cycles compared to the SPD prior to administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to a plurality of humans results in a serious adverse event in about 40% or less, about 37% or less, about 35% or less, or about 30% or less of the humans after the six 21-day cycles. In some embodiments, the venetoclax or a pharmaceutically acceptable salt thereof and the rituximab are further administered during a consolidation phase after the sixth 21-day cycle of the induction phase, wherein the venetoclax or a pharmaceutically acceptable salt thereof is administered orally at a dose of about 800 mg per day during the consolidation phase, and wherein the rituximab is administered intravenously at a dose of about 375 mg/m² once every two months during the consolidation phase. In some embodiments, the venetoclax or a pharmaceutically acceptable salt thereof and the rituximab are administered for a maximum of 8 months during the consolidation phase. In some embodiments, the rituximab is administered during the consolidation phase starting on Day 1 of the second month after the sixth 21-day cycle of the induction phase. In some embodiments, the venetoclax or a pharmaceutically acceptable salt thereof and the rituximab are administered sequentially during the consolidation phase. In some embodiments, the venetoclax or a pharmaceutically acceptable salt thereof is administered prior to the rituximab on Day 1 of each of months 2, 4, 6, and 8 during the consolidation phase. In some embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin.

In another aspect, provided herein is a method for treating diffuse large B-cell lymphoma (DLBCL) in a human in need thereof comprising administering to the human an effective amount of: (a) an immunoconjugate at a dose of about 1.8 mg/kg, wherein the immunoconjugate comprises the formula

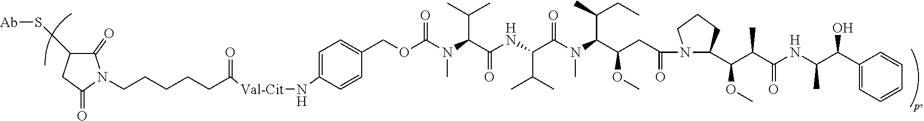

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) venetoclax or a pharmaceutically acceptable salt thereof at a dose of about 800 mg, and (c) rituximab at a dose of about 375 mg/m². In some embodiments, administration of the immunoconjugate, the venetoclax or a pharmaceutically acceptable salt thereof, and the rituximab to a plurality of humans results in a complete response in at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans during or after administration of the immunoconjugate, the venetoclax or a pharmaceutically acceptable salt thereof, and the rituximab. In some embodiments, administration of the immunoconjugate, the venetoclax or a pharmaceutically acceptable salt thereof, and the rituximab to a plurality of humans results in a best complete response rate of at least about 35%, at least about 38%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the immunoconjugate, the venetoclax or a pharmaceutically acceptable salt thereof, and the rituximab to a plurality of humans results in an objective response in at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100% of the humans during or after administration of the immunoconjugate, the venetoclax or a pharmaceutically acceptable salt thereof, and the rituximab. In some embodiments, the duration of the complete response or the objective response is at least about 3 months, at least about 4 months, at least about 5 months, at least about 6 months, at least about 7 months, or more. In some embodiments, administration of the immunoconjugate, the venetoclax or a pharmaceutically acceptable salt thereof, and the rituximab to a plurality of humans results in a best overall response in at least about 60%, at least about 65%, at least about 70%, at least about 75%, at least about 80%, at least about 85%, at least about 90%, at least about 95%, or 100% of the humans during or after administration of the immunoconjugate, the venetoclax or a pharmaceutically acceptable salt thereof, and the rituximab. In some embodiments, administration of the immunoconjugate, the venetoclax or a pharmaceutically acceptable salt thereof, and the rituximab to a plurality of humans results in a six-month progression-free survival rate of at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the immunoconjugate, the venetoclax or a pharmaceutically acceptable salt thereof, and the rituximab to the human results in progression-free survival of the human for at least about 3 months, at least about 4 months, at least about 5 months, at least about 6 months, at least about 7 months, or more after administration of the immunoconjugate, the venetoclax or a pharmaceutically acceptable salt thereof, and the rituximab. In some embodiments, administration of the immunoconjugate, the venetoclax or a pharmaceutically acceptable salt thereof, and the rituximab to the human results in survival of the human for at least about 6 months, at least about 7 months, at least about 8 months, at least about 9 months, at least about 10 months, at least about 11 months, or more after administration of the immunoconjugate, the venetoclax or a pharmaceutically acceptable salt thereof, and the rituximab. In some embodiments, administration of the immunoconjugate, the venetoclax or a pharmaceutically acceptable salt thereof, and the rituximab to the human results in a reduction in the sum of the product of diameters (SPD) of at least about 50%, at least about 60%, at least about 70%, at least about 80%, at least about 90%, at least about 95%, at least about 99%, or 100% compared to the SPD prior to administration of the immunoconjugate, the venetoclax or a pharmaceutically acceptable salt thereof, and the rituximab. In some embodiments, administration of the immunoconjugate, the venetoclax or a pharmaceutically acceptable salt thereof, and the rituximab to a plurality of humans results in a serious adverse event in about 40% or less, about 37% or less, about 35% or less, or about 30% or less of the humans. In some embodiments, p is between 3 and 4 or between 2 and 5. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin-piiq. In some embodiments, the polatuzumab vedotin-piiq, the venetoclax or a pharmaceutically acceptable salt thereof, and the rituximab are administered during an induction phase, optionally, wherein the induction phase comprises at least six 21-day cycles. In some embodiments, the polatuzumab vedotin-piiq is administered intravenously at a dose of about 1.8 mg/kg on Day 1 of each of the first, second, third, fourth, fifth, and sixth 21-day cycles, the venetoclax or a pharmaceutically acceptable salt thereof is administered orally at a dose of about 800 mg on each of Days 1-21 of each of the first, second, third, fourth, fifth, and sixth 21-day cycles, and the rituximab is administered intravenously at a dose of about 375 mg/m² on Day 1 of each of the first, second, third, fourth, fifth, and sixth 21-day cycles. In some embodiments, the polatuzumab vedotin-piiq, the venetoclax or a pharmaceutically acceptable salt thereof, and the rituximab are administered sequentially during the induction phase. In some embodiments, the venetoclax or a pharmaceutically acceptable salt thereof is administered prior to the rituximab and the rituximab is administered prior to the polatuzumab vedotin-piiq on Day 1 of each of the first, second, third, fourth, fifth, and sixth 21-day cycles. In some embodiments, administration of the polatuzumab vedotin-piiq, the venetoclax or a pharmaceutically acceptable salt thereof, and the rituximab results in a complete response in the human after the six 21-day cycles. In some embodiments, administration of the polatuzumab vedotin-piiq, the venetoclax or a pharmaceutically acceptable salt thereof, and the rituximab to a plurality of humans results in a complete response in at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans after the six 21-day cycles. In some embodiments, administration of the polatuzumab vedotin-piiq, the venetoclax or a pharmaceutically acceptable salt thereof, and the rituximab to a plurality of humans results in a best complete response rate of at least about 35%, at least about 38%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the polatuzumab vedotin-piiq, the venetoclax or a pharmaceutically acceptable salt thereof, and the rituximab to a plurality of humans results in an objective response in at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100% of the humans after the six 21-day cycles. In some embodiments, the duration of the complete response or the objective response is at least about 3 months, at least about 4 months, at least about 5 months, at least about 6 months, at least about 7 months, or more. In some embodiments, administration of the polatuzumab vedotin-piiq, the venetoclax or a pharmaceutically acceptable salt thereof, and the rituximab to a plurality of humans results in a best overall response in at least about 60%, at least about 65%, at least about 70%, at least about 75%, at least about 80%, at least about 85%, at least about 90%, at least about 95%, or 100% of the humans after the six 21-day cycles. In some embodiments, administration of the polatuzumab vedotin-piiq, the venetoclax or a pharmaceutically acceptable salt thereof, and the rituximab to a plurality of humans results in a six-month progression-free survival rate of at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the polatuzumab vedotin-piiq, the venetoclax or a pharmaceutically acceptable salt thereof, and the rituximab to the human results in progression-free survival of the human for at least about 3 months, at least about 4 months, at least about 5 months, at least about 6 months, at least about 7 months, or more after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to the human results in survival of the human for at least about 6 months, at least about 7 months, at least about 8 months, at least about 9 months, at least about 10 months, at least about 11 months, or more after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to the human results in a reduction in the sum of the product of diameters (SPD) of at least about 50%, at least about 60%, at least about 70%, at least about 80%, at least about 90%, at least about 95%, at least about 99%, or 100% after the six 21-day cycles compared to the SPD prior to administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to a plurality of humans results in a serious adverse event in about 40% or less, about 37% or less, about 35% or less, or about 30% or less of the humans after the six 21-day cycles. In some embodiments, the venetoclax or a pharmaceutically acceptable salt thereof and the rituximab are further administered during a consolidation phase after the sixth 21-day cycle of the induction phase, wherein the venetoclax or a pharmaceutically acceptable salt thereof is administered orally at a dose of about 800 mg per day during the consolidation phase, and wherein the rituximab is administered intravenously at a dose of about 375 mg/m² once every two months during the consolidation phase. In some embodiments, the venetoclax or a pharmaceutically acceptable salt thereof and the rituximab are administered for a maximum of 8 months during the consolidation phase. In some embodiments, the rituximab is administered during the consolidation phase starting on Day 1 of the second month after the sixth 21-day cycle of the induction phase. In some embodiments, the venetoclax or a pharmaceutically acceptable salt thereof and the rituximab are administered sequentially during the consolidation phase. In some embodiments, the venetoclax or a pharmaceutically acceptable salt thereof is administered prior to the rituximab on Day 1 of each of months 2, 4, 6, and 8 during the consolidation phase. In some embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin.

In another aspect, provided herein is a method for treating diffuse large B-cell lymphoma (DLBCL) in a human in need thereof comprising administering to the human an effective amount of: (a) an immunoconjugate at a dose of about 1.8 mg/kg, wherein the immunoconjugate comprises the formula

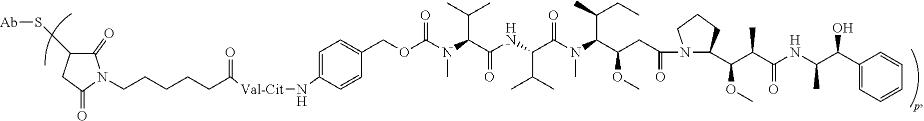

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) venetoclax or a pharmaceutically acceptable salt thereof at a dose of about 800 mg, and (c) rituximab at a dose of about 375 mg/m², wherein the human achieves a complete response (CR) during or after administration of the immunoconjugate, the venetoclax, and the rituximab. In some embodiments, p is between 3 and 4 or between 2 and 5. In some embodiments, administration of the immunoconjugate, the venetoclax or a pharmaceutically acceptable salt thereof, and the rituximab to a plurality of humans results in a complete response in at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans during or after administration of the immunoconjugate, the venetoclax or a pharmaceutically acceptable salt thereof, and the rituximab. In some embodiments, administration of the immunoconjugate, the venetoclax or a pharmaceutically acceptable salt thereof, and the rituximab to a plurality of humans results in a best complete response rate of at least about 35%, at least about 38%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the immunoconjugate, the venetoclax or a pharmaceutically acceptable salt thereof, and the rituximab to a plurality of humans results in an objective response in at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100% of the humans during or after administration of the immunoconjugate, the venetoclax or a pharmaceutically acceptable salt thereof, and the rituximab. In some embodiments, the duration of the complete response or the objective response is at least about 3 months, at least about 4 months, at least about 5 months, at least about 6 months, at least about 7 months, or more. In some embodiments, administration of the immunoconjugate, the venetoclax or a pharmaceutically acceptable salt thereof, and the rituximab to a plurality of humans results in a best overall response in at least about 60%, at least about 65%, at least about 70%, at least about 75%, at least about 80%, at least about 85%, at least about 90%, at least about 95%, or 100% of the humans during or after administration of the immunoconjugate, the venetoclax or a pharmaceutically acceptable salt thereof, and the rituximab. In some embodiments, administration of the immunoconjugate, the venetoclax or a pharmaceutically acceptable salt thereof, and the rituximab to a plurality of humans results in a six-month progression-free survival rate of at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the immunoconjugate, the venetoclax or a pharmaceutically acceptable salt thereof, and the rituximab to the human results in progression-free survival of the human for at least about 3 months, at least about 4 months, at least about 5 months, at least about 6 months, at least about 7 months, or more after administration of the immunoconjugate, the venetoclax or a pharmaceutically acceptable salt thereof, and the rituximab. In some embodiments, administration of the immunoconjugate, the venetoclax or a pharmaceutically acceptable salt thereof, and the rituximab to the human results in survival of the human for at least about 6 months, at least about 7 months, at least about 8 months, at least about 9 months, at least about 10 months, at least about 11 months, or more after administration of the immunoconjugate, the venetoclax or a pharmaceutically acceptable salt thereof, and the rituximab. In some embodiments, administration of the immunoconjugate, the venetoclax or a pharmaceutically acceptable salt thereof, and the rituximab to the human results in a reduction in the sum of the product of diameters (SPD) of at least about 50%, at least about 60%, at least about 70%, at least about 80%, at least about 90%, at least about 95%, at least about 99%, or 100% compared to the SPD prior to administration of the immunoconjugate, the venetoclax or a pharmaceutically acceptable salt thereof, and the rituximab. In some embodiments, administration of the immunoconjugate, the venetoclax or a pharmaceutically acceptable salt thereof, and the rituximab to a plurality of humans results in a serious adverse event in about 40% or less, about 37% or less, about 35% or less, or about 30% or less of the humans. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35. In some embodiments, the immunoconjugate is polatuzumab vedotin-piiq. In some embodiments, the polatuzumab vedotin-piiq, the venetoclax or a pharmaceutically acceptable salt thereof, and the rituximab are administered during an induction phase, optionally, wherein the induction phase comprises at least six 21-day cycles. In some embodiments, the polatuzumab vedotin-piiq is administered intravenously at a dose of about 1.8 mg/kg on Day 1 of each of the first, second, third, fourth, fifth, and sixth 21-day cycles, the venetoclax or a pharmaceutically acceptable salt thereof is administered orally at a dose of about 800 mg on each of Days 1-21 of each of the first, second, third, fourth, fifth, and sixth 21-day cycles, and the rituximab is administered intravenously at a dose of about 375 mg/m² on Day 1 of each of the first, second, third, fourth, fifth, and sixth 21-day cycles. In some embodiments, the polatuzumab vedotin-piiq, the venetoclax or a pharmaceutically acceptable salt thereof, and the rituximab are administered sequentially during the induction phase. In some embodiments, the venetoclax or a pharmaceutically acceptable salt thereof is administered prior to the rituximab and the rituximab is administered prior to the polatuzumab vedotin-piiq on Day 1 of each of the first, second, third, fourth, fifth, and sixth 21-day cycles. In some embodiments, administration of the polatuzumab vedotin-piiq, the venetoclax or a pharmaceutically acceptable salt thereof, and the rituximab results in a complete response in the human after the six 21-day cycles. In some embodiments, administration of the polatuzumab vedotin-piiq, the venetoclax or a pharmaceutically acceptable salt thereof, and the rituximab to a plurality of humans results in a complete response in at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans after the six 21-day cycles. In some embodiments, administration of the polatuzumab vedotin-piiq, the venetoclax or a pharmaceutically acceptable salt thereof, and the rituximab to a plurality of humans results in a best complete response rate of at least about 35%, at least about 38%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the polatuzumab vedotin-piiq, the venetoclax or a pharmaceutically acceptable salt thereof, and the rituximab to a plurality of humans results in an objective response in at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100% of the humans after the six 21-day cycles. In some embodiments, the duration of the complete response or the objective response is at least about 3 months, at least about 4 months, at least about 5 months, at least about 6 months, at least about 7 months, or more. In some embodiments, administration of the polatuzumab vedotin-piiq, the venetoclax or a pharmaceutically acceptable salt thereof, and the rituximab to a plurality of humans results in a best overall response in at least about 60%, at least about 65%, at least about 70%, at least about 75%, at least about 80%, at least about 85%, at least about 90%, at least about 95%, or 100% of the humans after the six 21-day cycles. In some embodiments, administration of the polatuzumab vedotin-piiq, the venetoclax or a pharmaceutically acceptable salt thereof, and the rituximab to a plurality of humans results in a six-month progression-free survival rate of at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the polatuzumab vedotin-piiq, the venetoclax or a pharmaceutically acceptable salt thereof, and the rituximab to the human results in progression-free survival of the human for at least about 3 months, at least about 4 months, at least about 5 months, at least about 6 months, at least about 7 months, or more after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to the human results in survival of the human for at least about 6 months, at least about 7 months, at least about 8 months, at least about 9 months, at least about 10 months, at least about 11 months, or more after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to the human results in a reduction in the sum of the product of diameters (SPD) of at least about 50%, at least about 60%, at least about 70%, at least about 80%, at least about 90%, at least about 95%, at least about 99%, or 100% after the six 21-day cycles compared to the SPD prior to administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to a plurality of humans results in a serious adverse event in about 40% or less, about 37% or less, about 35% or less, or about 30% or less of the humans after the six 21-day cycles. In some embodiments, the venetoclax or a pharmaceutically acceptable salt thereof and the rituximab are further administered during a consolidation phase after the sixth 21-day cycle of the induction phase, wherein the venetoclax or a pharmaceutically acceptable salt thereof is administered orally at a dose of about 800 mg per day during the consolidation phase, and wherein the rituximab is administered intravenously at a dose of about 375 mg/m² once every two months during the consolidation phase. In some embodiments, the venetoclax or a pharmaceutically acceptable salt thereof and the rituximab are administered for a maximum of 8 months during the consolidation phase. In some embodiments, the rituximab is administered during the consolidation phase starting on Day 1 of the second month after the sixth 21-day cycle of the induction phase. In some embodiments, the venetoclax or a pharmaceutically acceptable salt thereof and the rituximab are administered sequentially during the consolidation phase. In some embodiments, the venetoclax or a pharmaceutically acceptable salt thereof is administered prior to the rituximab on Day 1 of each of months 2, 4, 6, and 8 during the consolidation phase. In some embodiments, the anti-CD79b antibody comprises a heavy chain comprising the amino acid sequence of SEQ ID NO: 37 and a light chain comprising the amino acid sequence of SEQ ID NO: 35. In some embodiments, the anti-CD79b antibody comprises a heavy chain comprising the amino acid sequence of SEQ ID NO: 36 and a light chain comprising the amino acid sequence of SEQ ID NO: 38. In some embodiments, the immunoconjugate is iladatuzumab vedotin.

In another aspect, provided herein is a method of treating diffuse large B-cell lymphoma (DLBCL) in a human in need thereof, comprising administering to the human, during an induction phase, an effective amount of: (a) polatuzumab vedotin-piiq at a dose of about 1.8 mg/kg, (b) venetoclax or a pharmaceutically acceptable salt thereof at a dose of about 800 mg, and (c) rituximab at a dose of about 375 mg/m², wherein the human achieves a complete response during or after the induction phase. In some embodiments, administration of the polatuzumab vedotin-piiq, the venetoclax or a pharmaceutically acceptable salt thereof, and the rituximab to a plurality of humans results in a complete response in at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans during or after the induction phase. In some embodiments, administration of the polatuzumab vedotin-piiq, the venetoclax or a pharmaceutically acceptable salt thereof, and the rituximab to a plurality of humans results in a best complete response rate of at least about 35%, at least about 38%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the polatuzumab vedotin-piiq, the venetoclax or a pharmaceutically acceptable salt thereof, and the rituximab to a plurality of humans results in an objective response in at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100% of the humans during or after the induction phase. In some embodiments, the duration of the complete response or the objective response is at least about 3 months, at least about 4 months, at least about 5 months, at least about 6 months, at least about 7 months, or more. In some embodiments, administration of the polatuzumab vedotin-piiq, the venetoclax or a pharmaceutically acceptable salt thereof, and the rituximab to a plurality of humans results in a best overall response in at least about 60%, at least about 65%, at least about 70%, at least about 75%, at least about 80%, at least about 85%, at least about 90%, at least about 95%, or 100% of the humans during or after the induction phase. In some embodiments, administration of the polatuzumab vedotin-piiq, the venetoclax or a pharmaceutically acceptable salt thereof, and the rituximab to a plurality of humans results in a six-month progression-free survival rate of at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, administration of the polatuzumab vedotin-piiq, the venetoclax or a pharmaceutically acceptable salt thereof, and the rituximab to the human results in progression-free survival of the human for at least about 3 months, at least about 4 months, at least about 5 months, at least about 6 months, at least about 7 months, or more after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to the human results in survival of the human for at least about 6 months, at least about 7 months, at least about 8 months, at least about 9 months, at least about 10 months, at least about 11 months, or more after administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to the human results in a reduction in the sum of the product of diameters (SPD) of at least about 50%, at least about 60%, at least about 70%, at least about 80%, at least about 90%, at least about 95%, at least about 99%, or 100% compared to the SPD prior to administration of the polatuzumab vedotin-piiq, the venetoclax or a pharmaceutically acceptable salt thereof, and the rituximab. In some embodiments, administration of the polatuzumab vedotin-piiq, the venetoclax or a pharmaceutically acceptable salt thereof, and the rituximab to a plurality of humans results in a serious adverse event in about 40% or less, about 37% or less, about 35% or less, or about 30% or less of the humans. In some embodiments, the induction phase comprises at least six 21-day cycles. In some embodiments, the polatuzumab vedotin-piiq is administered intravenously at a dose of about 1.8 mg/kg on Day 1 of each of the first, second, third, fourth, fifth, and sixth 21-day cycles, the venetoclax or a pharmaceutically acceptable salt thereof is administered orally at a dose of about 800 mg on each of Days 1-21 of each of the first, second, third, fourth, fifth, and sixth 21-day cycles, and the rituximab is administered intravenously at a dose of about 375 mg/m² on Day 1 of each of the first, second, third, fourth, fifth, and sixth 21-day cycles. In some embodiments, the polatuzumab vedotin-piiq, the venetoclax or a pharmaceutically acceptable salt thereof, and the rituximab are administered sequentially during the induction phase. In some embodiments, the venetoclax or a pharmaceutically acceptable salt thereof is administered prior to the rituximab and the rituximab is administered prior to the polatuzumab vedotin-piiq on Day 1 of each of the first, second, third, fourth, fifth, and sixth 21-day cycles. In some embodiments, the venetoclax or a pharmaceutically acceptable salt thereof and the rituximab are further administered during a consolidation phase after the sixth 21-day cycle of the induction phase, wherein the venetoclax or a pharmaceutically acceptable salt thereof is administered orally at a dose of about 800 mg per day during the consolidation phase, and wherein the rituximab is administered intravenously at a dose of about 375 mg/m² once every two months during the consolidation phase. In some embodiments, the venetoclax or a pharmaceutically acceptable salt thereof and the rituximab are administered for a maximum of 8 months during the consolidation phase. In some embodiments, the rituximab is administered during the consolidation phase starting on Day 1 of the second month after the sixth 21-day cycle of the induction phase. In some embodiments, the venetoclax or a pharmaceutically acceptable salt thereof and the rituximab are administered sequentially during the consolidation phase. In some embodiments, the venetoclax or a pharmaceutically acceptable salt thereof is administered prior to the rituximab on Day 1 of each of months 2, 4, 6, and 8 during the consolidation phase.

In some embodiments which may be combined with any of the preceding aspects or embodiments, the method further comprises administering a prophylactic treatment for tumor lysis syndrome (TLS), wherein the prophylactic treatment for tumor lysis syndrome (TLS) comprises a uric acid-reducing agent and/or a hydration regimen prior to the start of treatment. In some embodiments, the hydration regimen comprises administering about 2 to about 3 liters per day of fluids, wherein the fluids are administered starting at about 24 hours to about 48 hours prior to the start of treatment. In some embodiments, the fluids are administered orally or intravenously. In some embodiments, the uric acid-reducing agent is allopurinol. In some embodiments, the allopurinol is administered orally at a dose of about 300 mg/day starting at about 72 hours prior to the first dose of venetoclax or a pharmaceutically acceptable salt thereof, and wherein administration of allopurinol continues for between about 3 and about 7 days after administration of the first dose of venetoclax or a pharmaceutically acceptable salt thereof.

In some embodiments which may be combined with any of the preceding aspects or embodiments, the method further comprises administering granulocyte colony stimulating factor (G-CSF) if a Grade 3 or Grade 4 adverse event of neutropenia occurs.

In some embodiments which may be combined with any of the preceding aspects or embodiments, the method further comprises administering a platelet transfusion if a Grade 3 or Grade 4 adverse event of thrombocytopenia occurs.

In some embodiments which may be combined with any of the preceding aspects or embodiments, the human has an Eastern Cooperative Oncology Group (ECOG) Performance Status score of 0, 1, or 2 prior to the start of treatment. In some embodiments which may be combined with any of the preceding aspects or embodiments, the DLBCL is relapsed or refractory to a prior treatment for DLBCL. In some embodiments, the prior treatment for DLBCL comprises a chemoimmunotherapy regimen comprising an anti-CD20 monoclonal antibody. In some embodiments which may be combined with any of the preceding aspects or embodiments, the DLBCL is histologically documented as being CD20-positive. In some embodiments which may be combined with any of the preceding aspects or embodiments, the DLBCL is fluorodeoxyglucose (FDG)-avid DLBCL. In some embodiments which may be combined with any of the preceding aspects or embodiments, the DLBCL is positron emission tomography (PET)-positive DLBCL. In some embodiments which may be combined with any of the preceding aspects or embodiments, the human has at least one bi-dimensionally measurable lesion prior to treatment, wherein the lesion is at least 1.5 centimeters in its largest dimension as measured by computed tomography (CT) scan or magnetic resonance imaging (MRI). In some embodiments which may be combined with any of the preceding aspects or embodiments, the human does not have a history of transformation of indolent disease to DLBCL. In some embodiments which may be combined with any of the preceding aspects or embodiments, the human does not have peripheral neuropathy of grade greater than 1 prior to treatment. In some embodiments which may be combined with any of the preceding aspects or embodiments, the human has DLBCL with an Ann Arbor stage of 1, 2, 3, or 4 prior to treatment. In some embodiments which may be combined with any of the preceding aspects or embodiments, the human has DLBCL with an International Prognostic Index (IPI) score of 0, 1, 2, 3, 4, or 5 prior to treatment. In some embodiments which may be combined with any of the preceding aspects or embodiments, the human has received at least one prior treatment for DLBCL. In some embodiments, the prior treatment for DLBCL comprises a chimeric antigen receptor (CAR) T-cell treatment for DLBCL. In some embodiments which may be combined with any of the preceding aspects or embodiments, the human has bulky disease of 7 centimeters or greater prior to treatment. In some embodiments which may be combined with any of the preceding aspects or embodiments, the human has extranodal disease. In some embodiments which may be combined with any of the preceding aspects or embodiments, the DLBCL is refractory to a prior treatment comprising an anti-CD20 agent. In some embodiments which may be combined with any of the preceding aspects or embodiments, the DLBCL did not respond, progressed, or relapsed within about 6 months after the last prior treatment for DLBCL administered to the human. In some embodiments which may be combined with any of the preceding aspects or embodiments, the DLBCL did not respond, progressed, or relapsed within about 6 months after the first prior treatment for DLBCL administered to the human. In some embodiments which may be combined with any of the preceding aspects or embodiments, the human has DLBCL with a cell of origin of activated B cell (ABC). In some embodiments which may be combined with any of the preceding aspects or embodiments, the human has DLBCL with a cell of origin of germinal center B cell (GCB). In some embodiments which may be combined with any of the preceding aspects or embodiments, the DLBCL is BCL2-positive DLBCL. In some embodiments which may be combined with any of the preceding aspects or embodiments, the DLBCL is BCL2-negative DLBCL. In some embodiments which may be combined with any of the preceding aspects or embodiments, the DLBCL is double expresser DLBCL. In some embodiments which may be combined with any of the preceding aspects or embodiments, the DLBCL is not double expresser DLBCL.

In another aspect, provided herein is a kit comprising an immunoconjugate comprising the formula

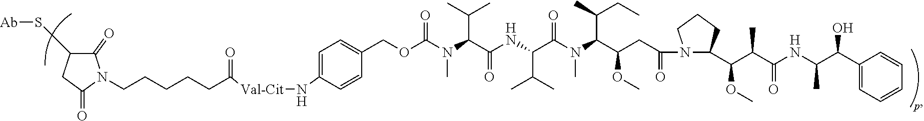

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with a selective Bcl-2 inhibitor or a pharmaceutically acceptable salt thereof and an anti-CD20 antibody for treating a human in need thereof having diffuse large B-cell lymphoma (DLBCL) according to any of the methods provided herein.

In another aspect, provided herein is a kit comprising an immunoconjugate comprising the formula

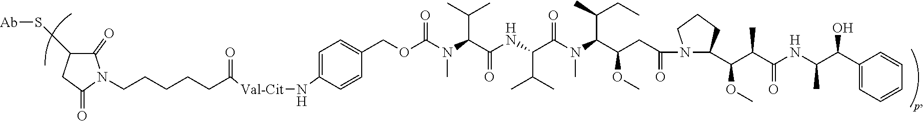

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with venetoclax or a pharmaceutically acceptable salt thereof and rituximab for treating a human in need thereof having diffuse large B-cell lymphoma (DLBCL) according to any of the methods provided herein.

In another aspect, provided herein is an immunoconjugate comprising the formula

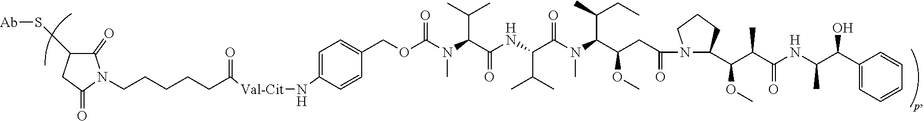

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with venetoclax or a pharmaceutically acceptable salt thereof and rituximab for treating a human in need thereof having diffuse large B-cell lymphoma (DLBCL) according to any of the methods provided herein.

In another aspect, provided herein is an immunoconjugate comprising the formula

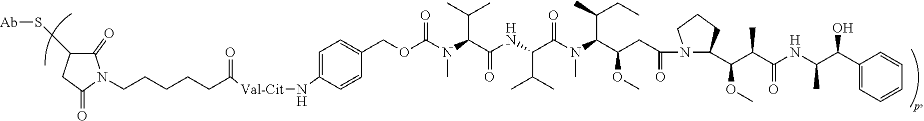

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in the manufacture of a medicament in combination with venetoclax or a pharmaceutically acceptable salt thereof and rituximab for treating a human in need thereof having diffuse large B-cell lymphoma (DLBCL) according to any of the methods provided herein.

In some embodiments which may be combined with any of the preceding aspects or embodiments, p is between 3 and 4 or between 2 and 5. In some embodiments which may be combined with any of the preceding aspects or embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments which may be combined with any of the preceding aspects or embodiments, the anti-CD79b antibody comprises (i) a heavy chain comprising the amino acid sequence of SEQ ID NO: 36 and (ii) a light chain comprising the amino acid sequence of SEQ ID NO: 35.

In another aspect, provided herein is a kit comprising polatuzumab vedotin-piiq for use in combination with venetoclax or a pharmaceutically acceptable salt thereof and rituximab for treating a human in need thereof having diffuse large B-cell lymphoma (DLBCL) according to any of the methods provided herein.

In another aspect, provided herein is polatuzumab vedotin-piiq for use in combination with venetoclax or a pharmaceutically acceptable salt thereof and rituximab for treating a human in need thereof having diffuse large B-cell lymphoma (DLBCL) according to any of the methods provided herein.

In another aspect, provided herein is polatuzumab vedotin-piiq for use in the manufacture of a medicament in combination with venetoclax or a pharmaceutically acceptable salt thereof and rituximab for treating a human in need thereof having diffuse large B-cell lymphoma (DLBCL) according to any of the methods provided herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B provide diagrams of the design of the study described in Example 1. FIG. 1A shows a diagram of the dose escalation phase (Phase Ib) of the study described in Example 1. FIG. 1B shows a diagram of the expansion phase (Phase II) of the study described in Example 1. In FIGS. 1A-1B, C=cycle; CR=complete response; D=day; D1C6=Day 1 of Cycle 6; DLBCL=diffuse large B-cell lymphoma; EOI=end of induction; FL=follicular lymphoma; G=obinutuzumab; IV=intravenous; M=month; PO=by mouth; Pola=polatuzumab vedotin; PR=partial response; QD=once a day; R=rituximab; RP2D=recommended Phase II dose; SD=stable disease; V=venetoclax. ^{a} After completion of induction treatment, all patients continue to receive daily venetoclax treatment (during Month 1) until response is assessed at EOI. Venetoclax is discontinued if response assessments at EOI indicate that a patient is not eligible for post-induction treatment.

FIGS. 2A-2B provide diagrams of the dosing regimens used in the study described in Example 1. FIG. 2A shows the induction dosing regimens for patients with FL and DLBCL. FIG. 2B shows the post-induction dosing regimens for patients with FL and DLBCL, which are termed “maintenance” and “consolidation,” respectively.

FIGS. 3A-3B show diagrams of the dose escalation schemes used in the dose escalation phase of the study described in Example 1. FIG. 3A is a diagram of the dose escalation scheme used in the FL dose escalation phase of the study. Dose escalation of polatuzumab vedotin and venetoclax administered in combination with a fixed dose of obinutuzumab was carried out using a 3+3 design. FIG. 3B is a diagram of the dose escalation scheme used in the DLBCL dose escalation phase of the study. The dose of venetoclax was escalated as indicated.

FIGS. 4A-4B provide diagrams outlining guidelines for administering obinutuzumab infusions to patients with FL in the study described in Example 1. FIG. 4A provides guidelines for administering the first infusion of obinutuzumab. ^{a}All patients receive full premedication with an oral corticosteroid, antihistamine, and oral analgesic/anti-pyretic prior to the first obinutuzumab infusion. ^{b}Supportive treatment includes acetaminophen/paracetamol and an antihistamine such as diphenhydramine, if not administered within the previous 4 hours. IV saline may be indicated. For bronchospasm, urticaria, or dyspnea, patients may require antihistamines, oxygen, corticosteroids (e.g., 100 mg oral prednisone or equivalent), and/or bronchodilators. IRR=infusion-related reaction; IV=intravenous. FIG. 4B provides guidelines for administering the second and subsequent infusions of obinutuzumab. ^{a}Patients receive full premedication with an oral corticosteroid, antihistamine, and oral analgesic/anti-pyretic prior to the obinutuzumab infusion. In the case of a recurrent Grade 3 IRR, obinutuzumab may be discontinued. ^{b}Patients who experience wheezing, urticaria, or other symptoms of anaphylaxis receive full premedication prior to all subsequent doses.

FIGS. 5A-5B provide efficacy results for the FL dose escalation phase of the study described in Example 1. FIG. 5A shows PET/CT images of a patient with FL enrolled in cohort 1 at initial screening (left panel) and at the end of induction treatment (right panel), which resulted in a significant signal decrease in cervical, axillary, mediastinal, and abdominal lymph nodes. FIG. 5B is a waterfall plot summarizing the percent change in the sum of perpendicular distances (SPD) by CT scan at end of induction compared to baseline, separated by cohort. P=polatuzumab vedotin (associated number indicates dose in mg/kg), V=venetoclax (associated number indicates dose in mg), O=obinutuzumab (associated number indicates dose in mg).

FIG. 6 provides a diagram depicting the design of the study described in Examples 1-2. The numbers of patients in the safety and efficacy evaluable populations included in an interim analysis described in Example 2 are provided. RP2D=recommended Phase II dose; Pola=polatuzumab vedotin; Ven=venetoclax; G=obinutuzumab; IV=intravenous; PO=oral; CR=complete response; PR=partial response; SD=stable disease.

FIG. 7 provides a Swimlane plot showing the time to response and the duration of responses for each patient in the efficacy evaluable population in the interim analysis described in Example 2. The responses were assessed by the investigator according to the Modified Lugano 2014 criteria. The time after initiation of study treatment is shown on the x-axis (months). Each horizontal bar represents an individual patient. The symbols shown in the legend indicate the last day of obinutuzumab (G) treatment, the last day of polatuzumab vedotin (Pola) treatment, the last day of venetoclax (Ven) treatment, the time of occurrence of a partial response (PR), and the time of occurrence of a complete response (CR). The stars indicate patients that remain on treatment.

FIG. 8 provides an overview of the study populations of the Phase Ib/II study described in Example 1 at the time of the primary analysis described in Example 3. RP2D=Recommended Phase II Dose.

FIG. 9 provides a summary of Grade 3-4 adverse events that occurred in ≥5% of patients in the Phase Ib/II study described in Examples 1 and 3. The adverse events are reported by preferred term. The number and percent of patients that were administered G-CSF or platelet transfusions during the induction or consolidation phases of the study are provided in the graph. SOC=system organ class.

FIG. 10 provides a Swimlane plot showing the time to response and the duration of INV-assessed responses observed in the efficacy-evaluable population of the Phase Ib/II study described in Examples 1 and 3. Each bar represents an individual patient. The number of months from the start of treatment is provided on the x-axis.

FIG. 11 shows an analysis of progression-free survival (PFS) of patients in the Phase Ib/II study described in Examples 1 and 3. The median PFS time (months) and the 6-month PFS rate are provided in the graph. The number of patients at risk at each indicated time on the x-axis is provided below the graph. CI=confidence interval. PFS was assessed by the investigator.

FIG. 12 shows the percent change in the sum of the product of diameters (SPD; assessed by the investigator) at end of induction (EOI) for patients in the Phase Ib/II study described in Examples 1 and 3. The corresponding responses assessed using the Modified Lugano criteria are shown for each patient.

FIG. 13 provides a diagram depicting the design of the study described in Examples 1, 2 and 4. *21-day cycles. CR, complete response; G, obinutuzumab; IV, intravenous; PO, oral administration; Pola, polatuzumab vedotin; PR, partial response; RP2D, recommended Phase 2 dose; SD, stable disease; Ven, venetoclax.

FIG. 14 provides a summary of the primary analysis populations described in Example 4.

### DETAILED DESCRIPTION

As used herein, the term “polatuzumab vedotin” refers to an anti-CD79b immunoconjugate having the IUPHAR/BPS Number 8404, the KEGG Number D10761, or the CAS Registry Number 1313206-42-6. Polatuzumab vedotin-piiq is also interchangeably referred to as “polatuzumab vedotin-piiq”, “huMA79bv28-MC-vc-PAB-MMAE”, “DCDS4501A”, or “RG7596.”

Provided herein are methods for treating follicular lymphoma (FL) in a human in need thereof comprising administering to the human an effective amount of: (a) an immunoconjugate comprising the formula

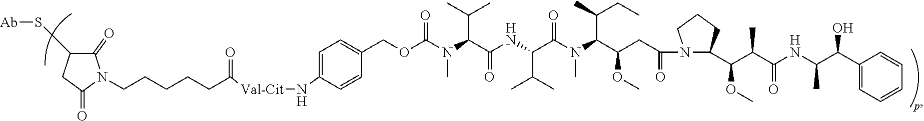

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) a Bcl-2 inhibitor, and (c) an anti-CD20 antibody, wherein the human achieves a complete response (CR) during or after treatment. In some embodiments, the anti-CD79b immunoconjugate is huMA79bv28-MC-vc-PAB-MMAE. In some embodiments, the immunoconjugate is polatuzumab vedotin-piiq (CAS Registry Number 1313206-42-6). In some embodiments, the anti-CD79b immunoconjugate is huMA79bv28-MC-vc-PAB-MMAE. In some embodiments, the immunoconjugate is polatuzumab vedotin-piiq (CAS Registry Number 1313206-42-6). In some embodiments, the immunoconjugate is polatuzumab vedotin-piiq. In some embodiments, the Bcl-2 inhibitor is venetoclax. In some embodiments, the anti-CD20 antibody is a humanized B-Ly1 antibody. In some embodiments, the humanized B-Ly1 antibody is obinutuzumab. In some embodiments, the anti-CD20 antibody is rituximab. In some embodiments, the anti-CD20 antibody is ofatumumab, ublituximab, and/or ibritumomab tiuxetan.

Also provided herein are methods for treating diffuse large B-cell lymphoma (DLBCL) in a human in need thereof comprising administering to the human an effective amount of: (a) an immunoconjugate comprising the formula

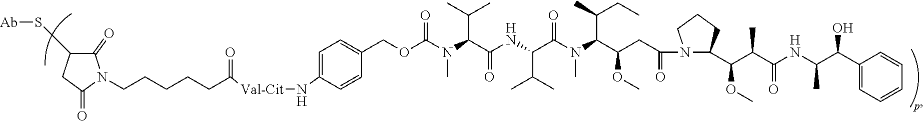

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) a Bcl-2 inhibitor, and (c) an anti-CD20 antibody, wherein the human achieves a complete response (CR) during or after treatment. In some embodiments, the anti-CD79b immunoconjugate is huMA79bv28-MC-vc-PAB-MMAE. In some embodiments, the immunoconjugate is polatuzumab vedotin-piiq (CAS Registry Number 1313206-42-6). In some embodiments, the anti-CD79b immunoconjugate is huMA79bv28-MC-vc-PAB-MMAE. In some embodiments, the immunoconjugate is polatuzumab vedotin-piiq (CAS Registry Number 1313206-42-6). In some embodiments, the immunoconjugate is polatuzumab vedotin-piiq. In some embodiments, the Bcl-2 inhibitor is venetoclax. In some embodiments, the anti-CD20 antibody is rituximab. In some embodiments, the anti-CD20 antibody is ofatumumab, ublituximab, and/or ibritumomab tiuxetan.

### I. General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

### II. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The term “CD79b,” as used herein, refers to any native CD79b from any vertebrate source, including mammals such as primates (e.g., humans, cynomologus monkey (“cyno”)) and rodents (e.g., mice and rats), unless otherwise indicated. Human CD79b is also referred herein to as “Igβ,” “B29,” “DNA225786,” or “PR036249.” An exemplary CD79b sequence including the signal sequence is shown in SEQ ID NO: 1. An exemplary CD79b sequence without the signal sequence is shown in SEQ ID NO: 2. The term “CD79b” encompasses “full-length,” unprocessed CD79b as well as any form of CD79b that results from processing in the cell. The term also encompasses naturally occurring variants of CD79b, e.g., splice variants, allelic variants, and isoforms. The CD79b polypeptides described herein may be isolated from a variety of sources, such as from human tissue types or from another source, or prepared by recombinant or synthetic methods. A “native sequence CD79b polypeptide” comprises a polypeptide having the same amino acid sequence as the corresponding CD79b polypeptide derived from nature. Such native sequence CD79b polypeptides can be isolated from nature or can be produced by recombinant or synthetic means. The term “native sequence CD79b polypeptide” specifically encompasses naturally-occurring truncated or secreted forms of the specific CD79b polypeptide (e.g., an extracellular domain sequence), naturally-occurring variant forms (e.g., alternatively spliced forms) and naturally-occurring allelic variants of the polypeptide.

“CD20” as used herein refers to the human B-lymphocyte antigen CD20 (also known as CD20, B-lymphocyte surface antigen B1, Leu-16, Bp35, BM5, and LF5; the sequence is characterized by the SwissProt database entry P11836) is a hydrophobic transmembrane protein with a molecular weight of approximately 35 kD located on pre-B and mature B lymphocytes. (Valentine, M. A., et al., J. Biol. Chem. 264(19) (1989 11282-11287; Tedder, T. F., et al, Proc. Natl. Acad. Sci. U.S.A. 85 (1988) 208-12; Stamenkovic, I., et al., J. Exp. Med. 167 (1988) 1975-80; Einfeld, D. A. et al., EMBO J. 7 (1988) 711-7; Tedder, T. F., et al., J. Immunol. 142 (1989) 2560-8). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and non-lymphoid tissues. This gene encodes the B-lymphocyte surface molecule, which plays a role in the development and differentiation of B-cells into plasma cells. This family member is localized to 11q12, among a cluster of family members. Alternative splicing of this gene results in two transcript variants, which encode the same protein.

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediate the killing of cells expressing CD20 (e.g., a tumor cell) by inactivating CD20. The killing of the cells expressing CD20 may occur by one or more of the following mechanisms: Cell death/apoptosis induction, ADCC and CDC. Synonyms of CD20, as recognized in the art, include B-lymphocyte antigen CD20, B-lymphocyte surface antigen B1, Leu-16, Bp35, BM5, and LF5.

The term “expression of the CD20” antigen is intended to indicate a significant level of expression of the CD20 antigen in a cell, e.g., a T- or B-Cell. In one embodiment, patients to be treated according to the methods of this invention express significant levels of CD20 on a B-cell tumor or cancer. Patients having a “CD20 expressing cancer” can be determined by standard assays known in the art. E.g., CD20 antigen expression is measured using immunohistochemical (IHC) detection, FACS or via PCR-based detection of the corresponding mRNA.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “epitope” refers to the particular site on an antigen molecule to which an antibody binds.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “anti-CD79b antibody” or “an antibody that binds to CD79b” refers to an antibody that is capable of binding CD79b with sufficient affinity such that the antibody is useful as a diagnostic and/or therapeutic agent in targeting CD79b. Preferably, the extent of binding of an anti-CD79b antibody to an unrelated, non-CD79b protein is less than about 10% of the binding of the antibody to CD79b as measured, e.g., by a radioimmunoassay (RIA). In certain embodiments, an antibody that binds to CD79b has a dissociation constant (Kd) of ≤1 μM, ≤100 nM, ≤10 nM, ≤1 nM, or ≤0.1 nM. In certain embodiments, anti-CD79b antibody binds to an epitope of CD79b that is conserved among CD79b from different species.

The term “anti-CD20 antibody” according to the invention refers to an antibody that is capable of binding CD20 with sufficient affinity such that the antibody is useful as a diagnostic and/or therapeutic agent in targeting CD20. Preferably, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of ≤1 μM, ≤100 nM, ≤10 nM, ≤1 nM, or ≤0.1 nM. In certain embodiments, anti-CD20 antibody binds to an epitope of CD20 that is conserved among CD20 from different species.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007). The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

“Isolated nucleic acid encoding an anti-CD79b antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B-cell receptor); and B-cell activation.

“CD79b polypeptide variant” means a CD79b polypeptide, preferably an active CD79b polypeptide, as defined herein having at least about 80% amino acid sequence identity with a full-length native sequence CD79b polypeptide sequence as disclosed herein, a CD79b polypeptide sequence lacking the signal peptide as disclosed herein, an extracellular domain of a CD79b polypeptide, with or without the signal peptide, as disclosed herein or any other fragment of a full-length CD79b polypeptide sequence as disclosed herein (such as those encoded by a nucleic acid that represents only a portion of the complete coding sequence for a full-length CD79b polypeptide). Such CD79b polypeptide variants include, for instance, CD79b polypeptides wherein one or more amino acid residues are added, or deleted, at the N- or C-terminus of the full-length native amino acid sequence. Ordinarily, a CD79b polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence CD79b polypeptide sequence as disclosed herein, a CD79b polypeptide sequence lacking the signal peptide as disclosed herein, an extracellular domain of a CD79b polypeptide, with or without the signal peptide, as disclosed herein or any other specifically defined fragment of a full-length CD79b polypeptide sequence as disclosed herein. Ordinarily, CD79b variant polypeptides are at least about 10 amino acids in length, alternatively at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 amino acids in length, or more. Optionally, CD79b variant polypeptides will have no more than one conservative amino acid substitution as compared to the native CD79b polypeptide sequence, alternatively no more than 2, 3, 4, 5, 6, 7, 8, 9, or 10 conservative amino acid substitution as compared to the native CD79b polypeptide sequence.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

wherein X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including but not limited to a cytotoxic agent.

In the context of the formulas provided herein, “p” refers to the average number of drug moieties per antibody, which can range, e.g., from about 1 to about 20 drug moieties per antibody, and in certain embodiments, from 1 to about 8 drug moieties per antibody. The invention includes a composition comprising a mixture of antibody-drug compounds of Formula I where the average drug loading per antibody is about 2 to about 5, or about 3 to about 4, (e.g., about 3.5).

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The terms “cancer” and “cancerous” refer to or describe the physiological condition in mammals that is typically characterized by unregulated cell growth. Examples of cancer include, but are not limited to, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), follicular lymphoma (e.g., relapsed/refractory follicular lymphoma) intermediate grade diffuse NHL, diffuse large B-cell lymphoma (DLBCL), aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “effective amount” of an agent, e.g., a pharmaceutical formulation, refers to an amount effective, at dosages and for periods of time necessary, to achieve the desired therapeutic or prophylactic result.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, reduction of free light chain, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, the antibodies described herein are used to delay development of a disease or to slow the progression of a disease.

The term “CD79b-positive cancer” refers to a cancer comprising cells that express CD79b on their surface. In some embodiments, expression of CD79b on the cell surface is determined, for example, using antibodies to CD79b in a method such as immunohistochemistry, FACS, etc. Alternatively, CD79b mRNA expression is considered to correlate to CD79b expression on the cell surface and can be determined by a method selected from in situ hybridization and RT-PCR (including quantitative RT-PCR).

As used herein, “in conjunction with” refers to administration of one treatment modality in addition to another treatment modality. As such, “in conjunction with” refers to administration of one treatment modality before, during, or after administration of the other treatment modality to the individual.

A “chemotherapeutic agent” is a chemical compound useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, everolimus, sotrataurin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR© (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin. Additional examples include of chemotherapeutic agents include bendamustine (or bendamustine-HCl) (TREANDA®), ibrutinib, lenalidomide, and/or idelalisib (GS-1101).

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine.

In some embodiments, the chemotherapeutic agent includes topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

Chemotherapetuic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ublituximab, ofatumumab, ibritumomab tiuxetan, pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1λ antibody genetically modified to recognize interleukin-12 p40 protein.

The term “package insert” is used to refer to instructions customarily included in commercial packages of therapeutic products, that contain information about the indications, usage, dosage, administration, combination therapy, contraindications and/or warnings concerning the use of such therapeutic products.

“Alkyl” is C₁-C₁₈ hydrocarbon containing normal, secondary, tertiary or cyclic carbon atoms. Examples are methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃) 3), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃.

The term “C₁-C₈ alkyl,” as used herein refers to a straight chain or branched, saturated or unsaturated hydrocarbon having from 1 to 8 carbon atoms. Representative “C₁-C₈ alkyl” groups include, but are not limited to, -methyl, -ethyl, -n-propyl, -n-butyl, -n-pentyl, -n-hexyl, -n-heptyl, -n-octyl, -n-nonyl and -n-decyl; while branched C₁-C₈ alkyls include, but are not limited to, -isopropyl, -sec-butyl, -isobutyl, -tert-butyl, -isopentyl, 2-methylbutyl, unsaturated C₁-C₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, -3-methyl-1 butynyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂ —NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

The term “C₁-C₁₂ alkyl,” as used herein refers to a straight chain or branched, saturated or unsaturated hydrocarbon having from 1 to 12 carbon atoms. A C₁-C₁₂ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂ —NHC(O)R′, —SO₃R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

The term “C₁-C₆ alkyl,” as used herein refers to a straight chain or branched, saturated or unsaturated hydrocarbon having from 1 to 6 carbon atoms. Representative “C₁-C₆ alkyl” groups include, but are not limited to, -methyl, -ethyl, -n-propyl, -n-butyl, -n-pentyl, -and n-hexyl; while branched C₁-C₆ alkyls include, but are not limited to, -isopropyl, -sec-butyl, -isobutyl, -tert-butyl, -isopentyl, and 2-methylbutyl; unsaturated C₁-C₆ alkyls include, but are not limited to, -vinyl, -allyl, -1-butenyl, -2-butenyl, and -isobutylenyl, -1-pentenyl, -2-pentenyl, -3-methyl-1-butenyl, -2-methyl-2-butenyl, -2,3-dimethyl-2-butenyl, 1-hexyl, 2-hexyl, and 3-hexyl. A C₁-C₆ alkyl group can be unsubstituted or substituted with one or more groups, as described above for C₁-C₈ alkyl group.

The term “C₁-C₄ alkyl,” as used herein refers to a straight chain or branched, saturated or unsaturated hydrocarbon having from 1 to 4 carbon atoms. Representative “C₁-C₄ alkyl” groups include, but are not limited to, -methyl, -ethyl, -n-propyl, -n-butyl; while branched C₁-C₄ alkyls include, but are not limited to, -isopropyl, -sec-butyl, -isobutyl, -tert-butyl; unsaturated C₁-C₄ alkyls include, but are not limited to, -vinyl, -allyl, -1-butenyl, -2-butenyl, and -isobutylenyl. A C₁-C₄ alkyl group can be unsubstituted or substituted with one or more groups, as described above for C₁-C₈ alkyl group.

“Alkoxy” is an alkyl group singly bonded to an oxygen. Exemplary alkoxy groups include, but are not limited to, methoxy (—OCH₃) and ethoxy (—OCH₂CH₃). A “C₁-C₅ alkoxy” is an alkoxy group with 1 to 5 carbon atoms. Alkoxy groups may can be unsubstituted or substituted with one or more groups, as described above for alkyl groups.

“Alkenyl” is C₂-C₁₈ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H7), and 5-hexenyl (—CH₂CH₂CH₂CH₂CH═CH₂). A “C₂-C₈ alkenyl” is a hydrocarbon containing 2 to 8 normal, secondary, tertiary or cyclic carbon atoms with at least one site of unsaturation, i.e. a carbon-carbon, sp² double bond.

“Alkynyl” is C₂-C₁₈ hydrocarbon containing normal, secondary, tertiary or cyclic carbon atoms with at least one site of unsaturation, i.e. a carbon-carbon, sp triple bond. Examples include, but are not limited to: acetylenic (—C≡CH) and propargyl (—CH₂C≡CH). A “C₂-C₈ alkynyl” is a hydrocarbon containing 2 to 8 normal, secondary, tertiary or cyclic carbon atoms with at least one site of unsaturation, i.e. a carbon-carbon, sp triple bond.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

A “C₁-C₁₀ alkylene” is a straight chain, saturated hydrocarbon group of the formula —(CH₂)₁₋₁₀—. Examples of a C₁-C₁₀ alkylene include methylene, ethylene, propylene, butylene, pentylene, hexylene, heptylene, ocytylene, nonylene and decalene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene (—C≡C—), propargyl (—CH₂C≡C—), and 4-pentynyl (—CH₂CH₂CH₂C≡C—).

“Aryl” refers to a carbocyclic aromatic group. Examples of aryl groups include, but are not limited to, phenyl, naphthyl and anthracenyl. A carbocyclic aromatic group or a heterocyclic aromatic group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂ —NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

A “C₅-C₂₀ aryl” is an aryl group with 5 to 20 carbon atoms in the carbocyclic aromatic rings. Examples of C₅-C₂₀ aryl groups include, but are not limited to, phenyl, naphthyl and anthracenyl. A C₅-C₂₀ aryl group can be substituted or unsubstituted as described above for aryl groups. A “C₅-C₁₄ aryl” is an aryl group with 5 to 14 carbon atoms in the carbocyclic aromatic rings. Examples of C₅-C₁₄ aryl groups include, but are not limited to, phenyl, naphthyl and anthracenyl. A C₅-C₁₄ aryl group can be substituted or unsubstituted as described above for aryl groups.

An “arylene” is an aryl group which has two covalent bonds and can be in the ortho, meta, or para configurations as shown in the following structures:

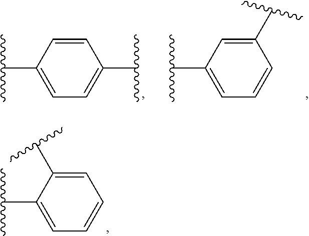

in which the phenyl group can be unsubstituted or substituted with up to four groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂ —NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

“Heteroarylalkyl” refers to an acyclic alkyl radical in which one of the hydrogen atoms bonded to a carbon atom, typically a terminal or sp³ carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo [4,5], [5,5], [5,6], or [6,6] system.

“Substituted alkyl,” “substituted aryl,” and “substituted arylalkyl” mean alkyl, aryl, and arylalkyl respectively, in which one or more hydrogen atoms are each independently replaced with a substituent. Typical substituents include, but are not limited to, —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NR₂, —SO₃⁻, —SO₃H, —S(═O)₂R, —OS(═O)₂OR, —S(═O)₂NR, —S(═O)R, —OP(═O)(OR)₂, —P(═O)(OR)₂, —PO⁻₃, —PO₃H2, —C(═O)R, —C(═O)X, —C(═S)R, —CO₂R, —CO₂⁻, —C(═S)OR, —C(═O)SR, —C(═S)SR, —C(═O)NR₂, —C(═S)NR₂, —C(═NR)NR₂, where each X is independently a halogen: F, Cl, Br, or I; and each R is independently —H, C₂-C₁₈ alkyl, C₆-C₂₀ aryl, C₃-C₁₄ heterocycle, protecting group or prodrug moiety. Alkylene, alkenylene, and alkynylene groups as described above may also be similarly substituted.

“Heteroaryl” and “heterocycle” refer to a ring system in which one or more ring atoms is a heteroatom, e.g., nitrogen, oxygen, and sulfur. The heterocycle radical comprises 3 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system.

Exemplary heterocycles are described, e.g., in Paquette, Leo A., “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, $-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or $-carboline. Still more typically, nitrogen bonded heterocycles include 1-aziridyl, 1-azetedyl, 1-pyrrolyl, 1-imidazolyl, 1-pyrazolyl, and 1-piperidinyl.

A “C₃-C₈ heterocycle” refers to an aromatic or non-aromatic C₃-C₈ carbocycle in which one to four of the ring carbon atoms are independently replaced with a heteroatom from the group consisting of O, S and N. Representative examples of a C₃-C₈ heterocycle include, but are not limited to, benzofuranyl, benzothiophene, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl. A C₃-C₈ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₈ heterocyclo” refers to a C₃-C₈ heterocycle group defined above wherein one of the heterocycle group's hydrogen atoms is replaced with a bond. A C₃-C₈ heterocyclo can be unsubstituted or substituted with up to six groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

A “C₃-C₂₀ heterocycle” refers to an aromatic or non-aromatic C₃-C₈ carbocycle in which one to four of the ring carbon atoms are independently replaced with a heteroatom from the group consisting of O, S and N. A C₃-C₂₀ heterocycle can be unsubstituted or substituted with up to seven groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; wherein each R′ is independently selected from H, —C₁-C₈ alkyl and aryl.

“C₃-C₂₀ heterocyclo” refers to a C₃-C₂₀ heterocycle group defined above wherein one of the heterocycle group's hydrogen atoms is replaced with a bond.

“Carbocycle” means a saturated or unsaturated ring having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cycloheptyl, and cyclooctyl.

A “C₃-C₈ carbocycle” is a 3-, 4-, 5-, 6-, 7- or 8-membered saturated or unsaturated non-aromatic carbocyclic ring. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂—NHC(O)R′, —S(O)₂R′, —S(O)R′, —OH, -halogen, —N₃, —NH₂, —NH(R′), —N(R′)₂ and —CN; where each R′ is independently selected from H, —C₁-C₈ alkyl and aryl.

A “C₃-C₈ carbocyclo” refers to a C₃-C₈ carbocycle group defined above wherein one of the carbocycle groups' hydrogen atoms is replaced with a bond.

“Linker” refers to a chemical moiety comprising a covalent bond or a chain of atoms that covalently attaches an antibody to a drug moiety. In various embodiments, linkers include a divalent radical such as an alkyldiyl, an aryldiyl, a heteroaryldiyl, moieties such as: —(CR₂)ₙO(CR₂)ₙ—, repeating units of alkyloxy (e.g., polyethylenoxy, PEG, polymethyleneoxy) and alkylamino (e.g., polyethyleneamino, Jeffamine™); and diacid ester and amides including succinate, succinamide, diglycolate, malonate, and caproamide. In various embodiments, linkers can comprise one or more amino acid residues, such as valine, phenylalanine, lysine, and homolysine.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Leaving group” refers to a functional group that can be substituted by another functional group. Certain leaving groups are well known in the art, and examples include, but are not limited to, a halide (e.g., chloride, bromide, iodide), methanesulfonyl (mesyl), p-toluenesulfonyl (tosyl), trifluoromethylsulfonyl (triflate), and trifluoromethylsulfonate.

The term “protecting group” refers to a substituent that is commonly employed to block or protect a particular functionality while reacting other functional groups on the compound. For example, an “amino-protecting group” is a substituent attached to an amino group that blocks or protects the amino functionality in the compound. Suitable amino-protecting groups include, but are not limited to, acetyl, trifluoroacetyl, t-butoxycarbonyl (BOC), benzyloxycarbonyl (CBZ) and 9-fluorenylmethylenoxycarbonyl (Fmoc). For a general description of protecting groups and their use, see T. W. Greene, Protective Groups in Organic Synthesis, John Wiley & Sons, New York, 1991, or a later edition.

### III Methods for Treating Follicular Lymphoma

Provided herein are methods for treating follicular lymphoma (FL) in a human in need thereof comprising administering to the human an effective amount of: (a) an immunoconjugate comprising the formula

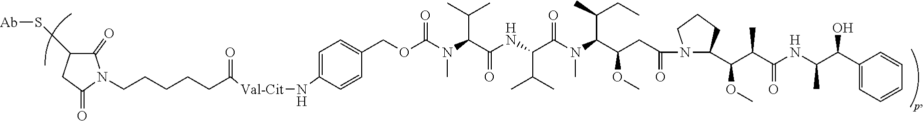

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) a Bcl-2 inhibitor, and (c) an anti-CD20 antibody, wherein the human achieves a complete response (CR) during or after treatment. In some embodiments, the anti-CD79b immunoconjugate is huMA79bv28-MC-vc-PAB-MMAE. In some embodiments, the immunoconjugate is polatuzumab vedotin-piiq (CAS Registry Number 1313206-42-6). In some embodiments, the anti-CD79b immunoconjugate is huMA79bv28-MC-vc-PAB-MMAE. In some embodiments, the immunoconjugate is polatuzumab vedotin-piiq (CAS Registry Number 1313206-42-6). In some embodiments, the immunoconjugate is polatuzumab vedotin-piiq. In some embodiments, the Bcl-2 inhibitor is venetoclax. In some embodiments, the anti-CD20 antibody is a humanized B-Ly1 antibody. In some embodiments, the humanized B-Ly1 antibody is obinutuzumab. In some embodiments, the anti-CD20 antibody is rituximab. In some embodiments, the anti-CD20 antibody is ofatumumab, ublituximab, and/or ibritumomab tiuxetan.

The terms “co-administration” or “co-administering” refer to the administration of the anti-CD79b immunoconjugate, the Bcl-2 inhibitor, and the anti-CD20 antibody as two (or more) separate formulations (or as one single formulation comprising the anti-CD79b immunoconjugate, the Bcl-2 inhibitor, and the anti-CD20 antibody). Where separate formulations are used, the co-administration can be simultaneous or sequential in either order, wherein preferably there is a time period while all active agents simultaneously exert their biological activities. The anti-CD79b immunoconjugate, the Bcl-2 inhibitor, and the anti-CD20 antibody are co-administered either simultaneously or sequentially.

Anti-CD79b immunoconjugates and additional therapeutic agents (e.g., a Bcl-2 inhibitor, and an anti-CD20 antibody) provided herein for use in any of the therapeutic methods described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immunoconjugate need not be, but is optionally formulated with one or more agents currently used to prevent or treat the disorder in question.

The amount of co-administration of the anti-CD79b immunoconjugate and the additional therapeutic agent and the timing of co-administration will depend on the type (species, gender, age, weight, etc.) and condition of the patient being treated and the severity of the disease or condition being treated. The anti-CD79b immunoconjugate, the Bcl-2 inhibitor and the anti-CD20 antibody are suitably co-administered to the patient at one time or over a series of treatments e.g., on the same day or on the day after.

In some embodiments, the dosage of anti-CD79b immunoconjugate (such as huMA79bv28-MC-vc-PAB-MMAE or polatuzumab vedotin-piiq) is between about any of 1.4-5 mg/kg, 1.4-4 mg/kg, 1.4-3.2 mg/kg, 1.4-2.4 mg/kg, or 1.4-1.8 mg/kg. In some embodiments of any of the methods, the dosage of anti-CD79 immunoconjugate is about any of 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, and/or 4.8 mg/kg. In some embodiments, the dosage of anti-CD79b immunoconjugate is about 1.4 mg/kg. In some embodiments, the dosage of anti-CD79b immunoconjugate is about 1.8 mg/kg. In some embodiments, the dosage of anti-CD79b immunoconjugate is about 2.4 mg/kg. In some embodiments, the dosage of anti-CD79b immunoconjugate is about 3.2 mg/kg. In some embodiments, the dosage of anti-CD79b immunoconjugate is about 3.6 mg/kg. In some embodiments of any of the methods, the anti-CD79b immunoconjugate is administered q3wk (e.g., on day 1 of each 21-day cycle). In some embodiments, the anti-CD79b immunoconjugate is administered via intravenous infusion. In some embodiments, the dosage administered via infusion is in the range of about 1 mg to about 1,500 mg per dose. Alternatively, the dosage range is of about 1 mg to about 1,500 mg, about 1 mg to about 1,000 mg, about 400 mg to about 1200 mg, about 600 mg to about 1000 mg, about 10 mg to about 500 mg, about 10 mg to about 300 mg, about 10 mg to about 200 mg, and about 1 mg to about 200 mg. In some embodiments, the dosage administered via infusion is in the range of about 1 μg/m² to about 10,000 μg/m² per dose. Alternatively, the dosage range is of about 1 μg/m² to about 1000 μg/m2, about 1 μg/m² to about 800 μg/m2, about 1 μg/m² to about 600 μg/m2, about 1 μg/m² to about 400 μg/m2, about 10 μg/m² to about 500 μg/m2, about 10 μg/m² to about 300 μg/m2, about 10 μg/m² to about 200 μg/m2, and about 1 μg/m² to about 200 μg/m2. The dose may be administered once per day, once per week, multiple times per week, but less than once per day, multiple times per month but less than once per day, multiple times per month but less than once per week, once per month, once every 21 days, or intermittently to relieve or alleviate symptoms of the disease. Administration may continue at any of the intervals and doses described herein until remission of the tumor or symptoms of the B-cell proliferative disorder being treated. Administration may continue after remission or relief of symptoms is achieved where such remission or relief is prolonged by such continued administration.

In some embodiments, the dosage of the anti-CD20 antibody is between about 300-1600 mg/m² and/or 300-2000 mg. In some embodiments, the dosage of the anti-CD20 antibody is about any of 300 mg/m², 375 mg/m², 600 mg/m², 1000 mg/m², or 1250 mg/m² and/or 300 mg, 1000 mg, or 2000 mg. In some embodiments, the anti-CD20 antibody is rituximab and the dosage administered is 375 mg/m². In some embodiments, the anti-CD20 antibody is obinutuzumab and the dosage administered is 1000 mg. In some embodiments, the anti-CD20 antibody is administered q1w (i.e., once per week). In some embodiments, the anti-CD20 antibody is administered on days 1, 8 and 15 of a 21-day cycle. In some embodiments, the anti-CD20 antibody is administered q3w (i.e., every 3 weeks or once every 21 days). In some embodiments, the anti-CD20 antibody is administered on day 1 of a 21-day cycle. In some embodiments, the anti-CD20 antibody is administered on days 1, 8, and 15 of the first 21-day cycle, and on day 1 of the subsequent 21-day cycles (e.g., on day 1 of cycles 2, 3, 4, 5, and 6). In some embodiments, the anti-CD20 antibody is administered once every two months. In some embodiments, the dosage of an afucosylated anti-CD20 antibody (preferably the afucosylated humanized B-Ly1 antibody) may be 800 to 1600 mg (in one embodiment 800 to 1200 mg, such as 1000 mg) or 400 to 1200 mg (in one embodiment 800 to 1200 mg). In some embodiments, the dose is a flat dose 1000 mg in a three-weeks-dosage schedule (e.g., once every 21 days).

In some embodiments, the dose of the Bcl-2 inhibitor (e.g., venetoclax) is between about 100 mg to about 800 mg (e.g., any of about 100 mg, 200 mg, 300 mg, 400 mg, 500 mg, 600 mg, 700 mg, or 800 mg). In some embodiments, venetoclax is administered at a dose of between about 100 mg to about 800 mg. In some embodiments, venetoclax is administered at a dose of about 100 mg. In some embodiments, venetoclax is administered at a dose of about 200 mg. In some embodiments, venetoclax is administered at a dose of about 400 mg. In some embodiments, venetoclax is administered at a dose of about 600 mg. In some embodiments, venetoclax is administered at a dose of about 800 mg.

An immunoconjugate provided herein (and any additional therapeutic agents, e.g., a Bcl-2 inhibitor and an anti-CD20 antibody) for use in any of the therapeutic methods described herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The anti-CD79b immunoconjugate (e.g., huMA79bv28-MC-vc-PAB-MMAE or polatuzumab vedotin-piiq), the Bcl-2 inhibitor (such as venetoclax) and the anti-CD20 antibody (such as obinutuzumab or rituximab) may be administered by the same route of administration or by different routes of administration. In some embodiments, the anti-CD79b immunoconjugate (e.g., polatuzumab vedotin-piiq) is administered intravenously, intramuscularly, subcutaneously, topically, orally, transdermally, intraperitoneally, intraorbitally, by implantation, by inhalation, intrathecally, intraventricularly, or intranasally. In some embodiments, the immunoconjugate (e.g., polatuzumab vedotin-piiq) is administered by intravenous infusion. In some embodiments, the anti-CD20 antibody (such as obinutuzumab or rituximab) is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD20 antibody (e.g., rituximab or obinutuzumab) is administered by intravenous infusion. In some embodiments, the Bcl-2 inhibitor (such as venetoclax) is administered intravenously, intramuscularly, subcutaneously, topically, orally, transdermally, intraperitoneally, intraorbitally, by implantation, by inhalation, intrathecally, intraventricularly, or intranasally. In some embodiments, the Bcl-2 inhibitor (e.g., venetoclax) is administered orally, e.g., in a tablet, capsule, or any other suitable means for oral administration known in the art or described herein. In some embodiments, the anti-CD79b immunoconjugate and the anti-CD20 antibody (such as obinutuzumab or rituximab) are each administered via intravenous infusion, and the Bcl-2 inhibitor (such as venetoclax) is administered orally. An effective amount of the anti-CD79b immunoconjugate, the Bcl-2 inhibitor (such as venetoclax) and the anti-CD20 antibody (such as obinutuzumab or rituximab) may be administered for prevention or treatment of disease.

In some embodiments, the anti-CD79b immunoconjugate (e.g., huMA79bv28-MC-vc-PAB-MMAE or polatuzumab vedotin-piiq) is administered at a dose between about 1.4 mg/kg to about 1.8 mg/kg. In some embodiments, the anti-CD79b immunoconjugate (e.g., huMA79bv28-MC-vc-PAB-MMAE or polatuzumab vedotin-piiq) is administered at a dose of about 1.4 mg/kg. In some embodiments, the anti-CD79b immunoconjugate (e.g., huMA79bv28-MC-vc-PAB-MMAE or polatuzumab vedotin-piiq) is administered at a dose of about 1.8 mg/kg. Alternatively or additionally, in some embodiments, the Bcl-2 inhibitor (such as venetoclax) is administered at a dose between about 200 mg and about 800 mg. In some embodiments, the Bcl-2 inhibitor (such as venetoclax) is administered at a dose of about 200 mg. In some embodiments, the Bcl-2 inhibitor (such as venetoclax) is administered at a dose of about 400 mg. In some embodiments, the Bcl-2 inhibitor (such as venetoclax) is administered at a dose of about 600 mg. In some embodiments, the Bcl-2 inhibitor (such as venetoclax) is administered at a dose of about 800 mg. Alternatively or additionally, in some embodiments, the anti-CD20 antibody is obinutuzumab. In some embodiments, the obinutuzumab is administered at a dose of about 1000 mg. Alternatively or additionally, in some embodiments, the anti-CD20 antibody is rituximab. In some embodiments, the rituximab is administered at a dose of about 375 mg/m².

In some embodiments, the anti-CD79b immunoconjugate (e.g., polatuzumab vedotin-piiq), the Bcl-2 inhibitor (e.g., venetoclax), and the anti-CD20 antibody (e.g., obinutuzumab or rituximab) are administered during an induction phase. An induction phase refers to a phase of treatment wherein the anti-CD79b immunoconjugate, the Bcl-2 inhibitor, and the anti-CD20 antibody are administered to a human. In some embodiments, the induction phase comprises less than one complete 21-day cycle. In some embodiments, the induction phase comprises between one and six (e.g., any of 1, 2, 3, 4, 5, or 6) 21-day cycles. In some embodiments, the induction phase comprises at least six 21-day cycles. In some embodiments, the anti-CD79b immunoconjugate, the Bcl-2 inhibitor (e.g., venetoclax) and the anti-CD20 antibody (e.g., obinutuzumab or rituximab) are administered for at least six 21-day cycles.

In some embodiments, during the induction phase, the immunoconjugate is administered intravenously at a dose of about 1.4 mg/kg on Day 1 of the first 21-day cycle, the Bcl-2 inhibitor is venetoclax, and the venetoclax is administered orally at a dose of about 200 mg on each of Days 1-21 of the first 21-day cycle, and the anti-CD20 antibody is obinutuzumab, and the obinutuzumab is administered intravenously at a dose of about 1000 mg on each of Days 1, 8, and 15 of the first 21-day cycle, and the immunoconjugate is administered intravenously at a dose of about 1.4 mg/kg on Day 1 of each of the second, third, fourth, fifth, and sixth 21-day cycles, the venetoclax is administered orally at a dose of about 200 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles.

In some embodiments, during the induction phase, the immunoconjugate is administered intravenously at a dose of about 1.4 mg/kg on Day 1 of the first 21-day cycle, the Bcl-2 inhibitor is venetoclax, and the venetoclax is administered orally at a dose of about 200 mg on each of Days 1-21 of the first 21-day cycle, and the anti-CD20 antibody is rituximab, and the rituximab is administered intravenously at a dose of about 375 mg/m² on each of Days 1, 8, and 15 of the first 21-day cycle, and the immunoconjugate is administered intravenously at a dose of about 1.4 mg/kg on Day 1 of each of the second, third, fourth, fifth, and sixth 21-day cycles, the venetoclax is administered orally at a dose of about 200 mg on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles.

In some embodiments, during the induction phase, the immunoconjugate is administered intravenously at a dose of about 1.4 mg/kg on Day 1 of the first 21-day cycle, the Bcl-2 inhibitor is venetoclax, and the venetoclax is administered orally at a dose of about 400 mg on each of Days 1-21 of the first 21-day cycle, and the anti-CD20 antibody is obinutuzumab, and the obinutuzumab is administered intravenously at a dose of about 1000 mg on each of Days 1, 8, and 15 of the first 21-day cycle, and the immunoconjugate is administered intravenously at a dose of about 1.4 mg/kg on Day 1 of each of the second, third, fourth, fifth, and sixth 21-day cycles, the venetoclax is administered orally at a dose of about 400 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles.

In some embodiments, during the induction phase, the immunoconjugate is administered intravenously at a dose of about 1.4 mg/kg on Day 1 of the first 21-day cycle, the Bcl-2 inhibitor is venetoclax, and the venetoclax is administered orally at a dose of about 400 mg on each of Days 1-21 of the first 21-day cycle, and the anti-CD20 antibody is rituximab, and the rituximab is administered intravenously at a dose of about 375 mg/m² on each of Days 1, 8, and 15 of the first 21-day cycle, and the immunoconjugate is administered intravenously at a dose of about 1.4 mg/kg on Day 1 of each of the second, third, fourth, fifth, and sixth 21-day cycles, the venetoclax is administered orally at a dose of about 400 mg on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles.

In some embodiments, during the induction phase, the immunoconjugate is administered intravenously at a dose of about 1.4 mg/kg on Day 1 of the first 21-day cycle, the Bcl-2 inhibitor is venetoclax, and the venetoclax is administered orally at a dose of about 600 mg on each of Days 1-21 of the first 21-day cycle, and the anti-CD20 antibody is obinutuzumab, and the obinutuzumab is administered intravenously at a dose of about 1000 mg on each of Days 1, 8, and 15 of the first 21-day cycle, and the immunoconjugate is administered intravenously at a dose of about 1.4 mg/kg on Day 1 of each of the second, third, fourth, fifth, and sixth 21-day cycles, the venetoclax is administered orally at a dose of about 600 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles.

In some embodiments, during the induction phase, the immunoconjugate is administered intravenously at a dose of about 1.4 mg/kg on Day 1 of the first 21-day cycle, the Bcl-2 inhibitor is venetoclax, and the venetoclax is administered orally at a dose of about 600 mg on each of Days 1-21 of the first 21-day cycle, and the anti-CD20 antibody is rituximab, and the rituximab is administered intravenously at a dose of about 375 mg/m² on each of Days 1, 8, and 15 of the first 21-day cycle, and the immunoconjugate is administered intravenously at a dose of about 1.4 mg/kg on Day 1 of each of the second, third, fourth, fifth, and sixth 21-day cycles, the venetoclax is administered orally at a dose of about 600 mg on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles.

In some embodiments, during the induction phase, the immunoconjugate is administered intravenously at a dose of about 1.4 mg/kg on Day 1 of the first 21-day cycle, the Bcl-2 inhibitor is venetoclax, and the venetoclax is administered orally at a dose of about 800 mg on each of Days 1-21 of the first 21-day cycle, and the anti-CD20 antibody is obinutuzumab, and the obinutuzumab is administered intravenously at a dose of about 1000 mg on each of Days 1, 8, and 15 of the first 21-day cycle, and the immunoconjugate is administered intravenously at a dose of about 1.4 mg/kg on Day 1 of each of the second, third, fourth, fifth, and sixth 21-day cycles, the venetoclax is administered orally at a dose of about 800 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles.

In some embodiments, during the induction phase, the immunoconjugate is administered intravenously at a dose of about 1.4 mg/kg on Day 1 of the first 21-day cycle, the Bcl-2 inhibitor is venetoclax, and the venetoclax is administered orally at a dose of about 800 mg on each of Days 1-21 of the first 21-day cycle, and the anti-CD20 antibody is rituximab, and the rituximab is administered intravenously at a dose of about 375 mg/m² on each of Days 1, 8, and 15 of the first 21-day cycle, and the immunoconjugate is administered intravenously at a dose of about 1.4 mg/kg on Day 1 of each of the second, third, fourth, fifth, and sixth 21-day cycles, the venetoclax is administered orally at a dose of about 800 mg on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles.

In some embodiments, during the induction phase, the immunoconjugate is administered intravenously at a dose of about 1.8 mg/kg on Day 1 of the first 21-day cycle, the Bcl-2 inhibitor is venetoclax, and the venetoclax is administered orally at a dose of about 200 mg on each of Days 1-21 of the first 21-day cycle, and the anti-CD20 antibody is obinutuzumab, and the obinutuzumab is administered intravenously at a dose of about 1000 mg on each of Days 1, 8, and 15 of the first 21-day cycle, and the immunoconjugate is administered intravenously at a dose of about 1.8 mg/kg on Day 1 of each of the second, third, fourth, fifth, and sixth 21-day cycles, the venetoclax is administered orally at a dose of about 200 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles.

In some embodiments, during the induction phase, the immunoconjugate is administered intravenously at a dose of about 1.8 mg/kg on Day 1 of the first 21-day cycle, the Bcl-2 inhibitor is venetoclax, and the venetoclax is administered orally at a dose of about 200 mg on each of Days 1-21 of the first 21-day cycle, and the anti-CD20 antibody is rituximab, and the rituximab is administered intravenously at a dose of about 375 mg/m² on each of Days 1, 8, and 15 of the first 21-day cycle, and the immunoconjugate is administered intravenously at a dose of about 1.8 mg/kg on Day 1 of each of the second, third, fourth, fifth, and sixth 21-day cycles, the venetoclax is administered orally at a dose of about 200 mg on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles.

In some embodiments, during the induction phase, the immunoconjugate is administered intravenously at a dose of about 1.8 mg/kg on Day 1 of the first 21-day cycle, the Bcl-2 inhibitor is venetoclax, and the venetoclax is administered orally at a dose of about 400 mg on each of Days 1-21 of the first 21-day cycle, and the anti-CD20 antibody is obinutuzumab, and the obinutuzumab is administered intravenously at a dose of about 1000 mg on each of Days 1, 8, and 15 of the first 21-day cycle, and the immunoconjugate is administered intravenously at a dose of about 1.8 mg/kg on Day 1 of each of the second, third, fourth, fifth, and sixth 21-day cycles, the venetoclax is administered orally at a dose of about 400 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles.

In some embodiments, during the induction phase, the immunoconjugate is administered intravenously at a dose of about 1.8 mg/kg on Day 1 of the first 21-day cycle, the Bcl-2 inhibitor is venetoclax, and the venetoclax is administered orally at a dose of about 400 mg on each of Days 1-21 of the first 21-day cycle, and the anti-CD20 antibody is rituximab, and the rituximab is administered intravenously at a dose of about 375 mg/m² on each of Days 1, 8, and 15 of the first 21-day cycle, and the immunoconjugate is administered intravenously at a dose of about 1.8 mg/kg on Day 1 of each of the second, third, fourth, fifth, and sixth 21-day cycles, the venetoclax is administered orally at a dose of about 400 mg on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles.

In some embodiments, during the induction phase, the immunoconjugate is administered intravenously at a dose of about 1.8 mg/kg on Day 1 of the first 21-day cycle, the Bcl-2 inhibitor is venetoclax, and the venetoclax is administered orally at a dose of about 600 mg on each of Days 1-21 of the first 21-day cycle, and the anti-CD20 antibody is obinutuzumab, and the obinutuzumab is administered intravenously at a dose of about 1000 mg on each of Days 1, 8, and 15 of the first 21-day cycle, and the immunoconjugate is administered intravenously at a dose of about 1.8 mg/kg on Day 1 of each of the second, third, fourth, fifth, and sixth 21-day cycles, the venetoclax is administered orally at a dose of about 600 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles.

In some embodiments, during the induction phase, the immunoconjugate is administered intravenously at a dose of about 1.8 mg/kg on Day 1 of the first 21-day cycle, the Bcl-2 inhibitor is venetoclax, and the venetoclax is administered orally at a dose of about 600 mg on each of Days 1-21 of the first 21-day cycle, and the anti-CD20 antibody is rituximab, and the rituximab is administered intravenously at a dose of about 375 mg/m² on each of Days 1, 8, and 15 of the first 21-day cycle, and the immunoconjugate is administered intravenously at a dose of about 1.8 mg/kg on Day 1 of each of the second, third, fourth, fifth, and sixth 21-day cycles, the venetoclax is administered orally at a dose of about 600 mg on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles.

In some embodiments, during the induction phase, the immunoconjugate is administered intravenously at a dose of about 1.8 mg/kg on Day 1 of the first 21-day cycle, the Bcl-2 inhibitor is venetoclax, and the venetoclax is administered orally at a dose of about 800 mg on each of Days 1-21 of the first 21-day cycle, and the anti-CD20 antibody is obinutuzumab, and the obinutuzumab is administered intravenously at a dose of about 1000 mg on each of Days 1, 8, and 15 of the first 21-day cycle, and the immunoconjugate is administered intravenously at a dose of about 1.8 mg/kg on Day 1 of each of the second, third, fourth, fifth, and sixth 21-day cycles, the venetoclax is administered orally at a dose of about 800 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles.

In some embodiments, during the induction phase, the immunoconjugate is administered intravenously at a dose of about 1.8 mg/kg on Day 1 of the first 21-day cycle, the Bcl-2 inhibitor is venetoclax, and the venetoclax is administered orally at a dose of about 800 mg on each of Days 1-21 of the first 21-day cycle, and the anti-CD2 antibody is rituximab, and the rituximab is administered intravenously at a dose of about 375 mg/m² on each of Days 1, 8, and 15 of the first 21-day cycle, and the immunoconjugate is administered intravenously at a dose of about 1.8 mg/kg on Day 1 of each of the second, third, fourth, fifth, and sixth 21-day cycles, the venetoclax is administered orally at a dose of about 800 mg on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles.

The dosing and administration schedules for exemplary induction phases are provided in Tables A-L below:

### Tables A-L: Dosing and Administration Schedules for Exemplary Induction Phases

TABLE A

| Drugs | Cycle 1 (21 days) | Cycles 2-6 (21 days each) |
|---|---|---|
| Anti-CD79b | 1.4 mg/kg on Day 1 | 1.4 mg/kg on Day 1 |
| immunoconjugate | | |
| (polatuzumab | | |
| vedotin-piiq) | | |
| Bcl-2 inhibitor | 200 mg on each of | 200 mg on each of |
| (venetoclax) | Days 1-21 | Days 1-21 |
| Anti-CD20 Antibody | 1000 mg on each of | 1000 mg on Day 1 |
| (obinutuzumab) | Days 1, 8, and 15 | |

TABLE B

| Drugs | Cycle 1 (21 days) | Cycles 2-6 (21 days each) |
|---|---|---|
| Anti-CD79b | 1.4 mg/kg on Day 1 | 1.4 mg/kg on Day 1 |
| immunoconjugate | | |
| (polatuzumab | | |
| vedotin-piiq) | | |
| Bcl-2 inhibitor | 400 mg on each of | 400 mg on each of |
| (venetoclax) | Days 1-21 | Days 1-21 |
| Anti-CD20 Antibody | 1000 mg on each of | 1000 mg on Day 1 |
| (obinutuzumab) | Days 1, 8, and 15 | |

TABLE C

| Drugs | Cycle 1 (21 days) | Cycles 2-6 (21 days each) |
|---|---|---|
| Anti-CD79b | 1.4 mg/kg on Day 1 | 1.4 mg/kg on Day 1 |
| immunoconjugate | | |
| (polatuzumab | | |
| vedotin-piiq) | | |
| Bcl-2 inhibitor | 600 mg on each of | 600 mg on each of |
| (venetoclax) | Days 1-21 | Days 1-21 |
| Anti-CD20 Antibody | 1000 mg on each of | 1000 mg on Day 1 |
| (obinutuzumab) | Days 1, 8, and 15 | |

TABLE D

| Drugs | Cycle 1 (21 days) | Cycles 2-6 (21 days each) |
|---|---|---|
| Anti-CD79b | 1.4 mg/kg on Day 1 | 1.4 mg/kg on Day 1 |
| immunoconjugate | | |
| (polatuzumab | | |
| vedotin-piiq) | | |
| Bcl-2 inhibitor | 800 mg on each of | 800 mg on each of |
| (venetoclax) | Days 1-21 | Days 1-21 |
| Anti-CD20 Antibody | 1000 mg on each of | 1000 mg on Day 1 |
| (obinutuzumab) | Days 1, 8, and 15 | |

TABLE E

| Drugs | Cycle 1 (21 days) | Cycles 2-6 (21 days each) |
|---|---|---|
| Anti-CD79b | 1.8 mg/kg on Day 1 | 1.8 mg/kg on Day 1 |
| immunoconjugate | | |
| (polatuzumab | | |
| vedotin-piiq) | | |
| Bcl-2 inhibitor | 200 mg on each of | 200 mg on each of |
| (venetoclax) | Days 1-21 | Days 1-21 |
| Anti-CD20 Antibody | 1000 mg on each of | 1000 mg on Day 1 |
| (obinutuzumab) | Days 1, 8, and 15 | |

TABLE F

| Drugs | Cycle 1 (21 days) | Cycles 2-6 (21 days each) |
|---|---|---|
| Anti-CD79b | 1.8 mg/kg on Day 1 | 1.8 mg/kg on Day 1 |
| immunoconjugate | | |
| (polatuzumab | | |
| vedotin-piiq) | | |
| Bcl-2 inhibitor | 400 mg on each of | 400 mg on each of |
| (venetoclax) | Days 1-21 | Days 1-21 |
| Anti-CD20 Antibody | 1000 mg on each of | 1000 mg on Day 1 |
| (obinutuzumab) | Days 1, 8, and 15 | |

TABLE G

| Drugs | Cycle 1 (21 days) | Cycles 2-6 (21 days each) |
|---|---|---|
| Anti-CD79b | 1.8 mg/kg on Day 1 | 1.8 mg/kg on Day 1 |
| immunoconjugate | | |
| (polatuzumab | | |
| vedotin-piiq) | | |
| Bcl-2 inhibitor | 600 mg on each of | 600 mg on each of |
| (venetoclax) | Days 1-21 | Days 1-21 |
| Anti-CD20 Antibody | 1000 mg on each of | 1000 mg on Day 1 |
| (obinutuzumab) | Days 1, 8, and 15 | |

TABLE H

| Drugs | Cycle 1 (21 days) | Cycles 2-6 (21 days each) |
|---|---|---|
| Anti-CD79b | 1.8 mg/kg on Day 1 | 1.8 mg/kg on Day 1 |
| immunoconjugate | | |
| (polatuzumab | | |
| vedotin-piiq) | | |
| Bcl-2 inhibitor | 800 mg on each of | 800 mg on each of |
| (venetoclax) | Days 1-21 | Days 1-21 |
| Anti-CD20 Antibody | 1000 mg on each of | 1000 mg on Day 1 |
| (obinutuzumab) | Days 1, 8, and 15 | |

In some embodiments, the anti-CD79b immunoconjugate, the Bcl-2 inhibitor, and the anti-CD20 antibody are administered sequentially during an induction phase. In some embodiments, the Bcl-2 inhibitor is administered prior to the anti-CD20 antibody and the anti-CD20 antibody is administered prior to the immunoconjugate on Day 1 of the first 21-day cycle, and the Bcl-2 inhibitor is administered prior to the anti-CD20 antibody on days 8 and 15 of the first 21-day cycle; and the Bcl-2 inhibitor is administered prior to the anti-CD20 antibody and the anti-CD20 antibody is administered prior to the immunoconjugate on Day 1 of each of the second, third, fourth, fifth, and sixth 21-day cycles.

In some embodiments, the immunoconjugate is polatuzumab vedotin-piiq, the anti-CD20 antibody is obinutuzumab, and the Bcl-2 inhibitor is venetoclax. In some embodiments, the polatuzumab vedotin-piiq, the venetoclax, and the obinutuzumab are administered sequentially during the induction phase. In some embodiments, the venetoclax is administered prior to the obinutuzumab and the obinutuzumab is administered prior to the polatuzumab vedotin-piiq on Day 1 of the first 21-day cycle, and the venetoclax is administered prior to the obinutuzumab on days 8 and 15 of the first 21-day cycle; and the venetoclax is administered prior to the obinutuzumab and the obinutuzumab is administered prior to the polatuzumab vedotin-piiq on Day 1 of each of the second, third, fourth, fifth, and sixth 21-day cycles.

In some embodiments, the immunoconjugate is polatuzumab vedotin-piiq, the anti-CD20 antibody is rituximab, and the Bcl-2 inhibitor is venetoclax. In some embodiments, the polatuzumab vedotin-piiq, the venetoclax, and the rituximab are administered sequentially during the induction phase. In some embodiments, the venetoclax is administered prior to the rituximab and the rituximab is administered prior to the polatuzumab vedotin-piiq on Day 1 of the first 21-day cycle, and the venetoclax is administered prior to the rituximab on days 8 and 15 of the first 21-day cycle; and the venetoclax is administered prior to the rituximab and the rituximab is administered prior to the polatuzumab vedotin-piiq on Day 1 of each of the second, third, fourth, fifth, and sixth 21-day cycles.

In some embodiments, the anti-CD20 antibody is rituximab. In some embodiments, the rituximab is administered at a dose of about 375 mg/m² in combination with an anti-CD79b immunoconjugate and a Bcl-2 inhibitor according to any induction phase provided herein.

In some embodiments, the anti-CD20 antibody is obinutuzumab. In some embodiments, the obinutuzumab is administered at a dose of about 1000 mg in combination with an anti-CD79b immunoconjugate and a Bcl-2 inhibitor according to any induction phase provided herein.

In some embodiments, during the induction phase, the immunoconjugate (e.g., polatuzumab vedotin-piiq) is administered intravenously at a dose of between about 1.4 mg/kg and about 1.8 mg/kg (e.g., 1.4 mg/kg or 1.8 mg/kg) on Day 1 of the first 21-day cycle, the Bcl-2 inhibitor is venetoclax, and the venetoclax is administered orally at a dose of between about 200 mg to about 800 mg (e.g., any of 200 mg, 400 mg, 600 mg, or 800 mg) on each of Days 1-21 of the first 21-day cycle, and the anti-CD20 antibody is rituximab, and the rituximab is administered intravenously at a dose of about 375 mg/m² on each of Days 1, 8, and 15 of the first 21-day cycle, and the immunoconjugate is administered intravenously at a dose of between about 1.4 mg/kg and about 1.8 mg/kg (e.g., 1.4 mg/kg or 1.8 mg/kg) on Day 1 of each of the second, third, fourth, fifth, and sixth 21-day cycles, the venetoclax is administered orally at a dose of between about 200 mg to about 800 mg (e.g., any of 200 mg, 400 mg, 600 mg, or 800 mg) on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles.

In some embodiments, during the induction phase, the immunoconjugate (e.g., polatuzumab vedotin-piiq) is administered intravenously at a dose of between about 1.4 mg/kg and about 1.8 mg/kg (e.g., 1.4 mg/kg or 1.8 mg/kg) on Day 1 of the first 21-day cycle, the Bcl-2 inhibitor is venetoclax, and the venetoclax is administered orally at a dose of between about 200 mg to about 800 mg (e.g., any of 200 mg, 400 mg, 600 mg, or 800 mg) on each of Days 1-21 of the first 21-day cycle, and the anti-CD20 antibody is rituximab, and the rituximab is administered intravenously at a dose of about 375 mg/m² on Day 1 of the first 21-day cycle, and the immunoconjugate is administered intravenously at a dose of between about 1.4 mg/kg and about 1.8 mg/kg (e.g., 1.4 mg/kg or 1.8 mg/kg) on Day 1 of each of the second, third, fourth, fifth, and sixth 21-day cycles, the venetoclax is administered orally at a dose of between about 200 mg to about 800 mg (e.g., any of 200 mg, 400 mg, 600 mg, or 800 mg) on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles.

In some embodiments, a human treated according to the methods provided herein achieves a complete response (CR) during or after treatment. In some embodiments, the human achieves a complete response during induction treatment. In some embodiments, the human achieves a complete response at the end of induction treatment (e.g., after six 21-day cycles). In some embodiments, the human achieves a complete response after six 21-day cycles. In some embodiments, the human achieves a complete response after one, two, three, four, five, or six 21-day cycles.

In some embodiments, among a plurality of humans treated, at least about 55%, at least about 57%, at least about 60%, at least about 65%, at least about 70%, at least about 75%, at least about 80%, at least about 85%, at least about 90%, at least about 95%, or 100% of the humans achieve a complete response during or after treatment. In some embodiments, among a plurality of humans treated, at least about 55%, at least about 57%, at least about 60%, at least about 65%, at least about 70%, at least about 75%, at least about 80%, at least about 85%, at least about 90%, at least about 95%, or 100% of the humans achieve a complete response during induction treatment. In some embodiments, among a plurality of humans treated, at least about 55%, at least about 57%, at least about 60%, at least about 65%, at least about 70%, at least about 75%, at least about 80%, at least about 85%, at least about 90%, at least about 95%, or 100% of the humans achieve a complete response at the end of induction treatment (e.g., after six 21-day cycles). In some embodiments, among a plurality of humans treated, at least about 55%, at least about 57%, at least about 60%, at least about 65%, at least about 70%, at least about 75%, at least about 80%, at least about 85%, at least about 90%, at least about 95%, or 100% of the humans achieve a complete response after six 21-day cycles. In some embodiments, among a plurality of humans treated, at least about 55%, at least about 57%, at least about 60%, at least about 65%, at least about 70%, at least about 75%, at least about 80%, at least about 85%, at least about 90%, at least about 95%, or 100% of the humans achieve a complete response after one, two, three, four, five, or six 21-day cycles.

In some embodiments, the duration of the complete response is at least about 1 month, at least about 2 months, at least about 3 months, or more. In some embodiments, the duration of a complete response is measured from the first occurrence of a complete response (e.g., as described below) to disease progression (e.g., according to the modified Lugano 2014 criteria). In some embodiments, the duration of a complete response is measured from the first occurrence of a complete response to relapse (e.g., according to the modified Lugano 2014 criteria). In some embodiments, the duration of a complete response is measured from the first occurrence of a complete response to disease progression or relapse (e.g., according to the modified Lugano 2014 criteria). In some embodiments, the duration of a complete response is measured from the first occurrence of a complete response to disease progression or relapse (e.g., according to the modified Lugano 2014 criteria), or death from any cause.

In some embodiments, among a plurality of humans treated, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% of the humans achieve an objective response during or after treatment. In some embodiments, among a plurality of humans treated, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% of the humans achieve an objective response during induction treatment. In some embodiments, among a plurality of humans treated, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% of the humans achieve an objective response at the end of induction treatment (e.g., after six 21-day cycles). In some embodiments, among a plurality of humans treated, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% of the humans achieve an objective response after six 21-day cycles. In some embodiments, among a plurality of humans treated, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% of the humans achieve an objective response after one, two, three, four, five, or six 21-day cycles.

Complete responses are evaluated according to the modified Lugano 2014 criteria, as described in Example 1 herein. In some embodiments, complete responses are assessed on the basis of PET-CT scans according to the modified Lugano 2014 criteria, as described in Example 1 herein. In some embodiments, complete responses are assessed on the basis of CT scans alone according to the modified Lugano 2014 criteria, as described in Example 1 herein.

As used herein, an objective response refers to a complete response or a partial response, evaluated according to the modified Lugano 2014 criteria, as described in Example 1 herein. Thus, a human treated according to the methods provided herein that achieves an objective response achieves a complete response or a partial response, assessed according to the modified Lugano 2014 criteria, as described in Example 1 herein.

In some embodiments, objective responses are assessed on the basis of PET-CT scans according to the modified Lugano 2014 criteria, as described in Example 1 herein. In some embodiments, objective responses are assessed on the basis of CT scans alone according to the modified Lugano 2014 criteria, as described in Example 1 herein.

Further details regarding clinical staging of and response criteria for lymphomas such as FL are provided in, e.g., Van Heertum et al. (2017) Drug Des. Devel. Ther. 11: 1719-1728; Cheson et al. (2016) Blood. 128: 2489-2496; Cheson et al. (2014) J. Clin. Oncol. 32(27): 3059-3067; Barrington et al. (2017) J. Clin. Oncol. 32(27): 3048-3058; Gallamini et al. (2014) Haematologica. 99(6): 1107-1113; Barrinton et al. (2010) Eur. J. Nucl. Med. Mol. Imaging. 37(10): 1824-33; Moskwitz (2012) Hematology Am Soc. Hematol. Educ. Program 2012: 397-401; and Follows et al. (2014) Br. J. Haematology 166: 34-49. The progress of any one of the methods of treatment provided herein can be monitored by techniques known in the art.

In some embodiments, the Bcl-2 inhibitor (e.g., venetoclax) and the anti-CD20 antibody (e.g., obinutuzumab or rituximab) are further administered post-induction, e.g., during a maintenance phase following the sixth 21-day cycle. The maintenance phase or “post-induction treatment” refers to a treatment phase following an induction phase. In some embodiments, the maintenance phase begins immediately after the end of the induction phase. In some embodiments, the induction phase and the maintenance phase are separated by an interval of time. In some embodiments, the maintenance phase begins at least about 1, 2, 3, 4, 5, 6, 7, 8, 9, or 10 weeks after the end of the induction phase.

In some embodiments, during the maintenance phase, the Bcl-2 inhibitor (e.g., venetoclax) is administered orally at a dose between about 200 mg to about 800 mg once daily following the sixth 21-day cycle of the induction phase, and the anti-CD20 antibody (e.g., obinutuzumab or rituximab) is administered intravenously at a dose of about 1000 mg or about 375 mg/m² on Day 1 of every other month starting at 2 months after the sixth 21-day cycle of the induction phase. In some embodiments, the Bcl-2 inhibitor (e.g., venetoclax) is administered during the maintenance phase for a maximum of 8 months. In some embodiments, the anti-CD20 antibody (e.g., obinutuzumab or rituximab) is administered during the maintenance phase for a maximum of 24 months.

In some embodiments, during the maintenance phase, the Bcl-2 inhibitor is venetoclax, and the venetoclax is administered orally at a dose of about 200 mg once daily following the sixth 21-day cycle of the induction phase for up to 8 months, and the anti-CD20 antibody is obinutuzumab, and the obinutuzumab is administered intravenously at a dose of about 1000 mg on Day 1 of every other month starting at 2 months after the sixth 21-day cycle of the induction phase for up to 24 months (e.g., on Day 1 of each of months 2, 4, 6, 8, 10, 12, 14, 16, 18, 20, 22, and 24).

In some embodiments, during the maintenance phase, the Bcl-2 inhibitor is venetoclax, and the venetoclax is administered orally at a dose of about 200 mg once daily following the sixth 21-day cycle of the induction phase for up to 8 months, and the anti-CD20 antibody is rituximab, and the rituximab is administered intravenously at a dose of about 375 mg/m² on Day 1 of every other month starting at 2 months after the sixth 21-day cycle of the induction phase for up to 24 months (e.g., on Day 1 of each of months 2, 4, 6, 8, 10, 12, 14, 16, 18, 20, 22, and 24).

In some embodiments, during the maintenance phase, the Bcl-2 inhibitor is venetoclax, and the venetoclax is administered orally at a dose of about 400 mg once daily following the sixth 21-day cycle of the induction phase for up to 8 months, and the anti-CD20 antibody is obinutuzumab, and the obinutuzumab is administered intravenously at a dose of about 1000 mg on Day 1 of every other month starting at 2 months after the sixth 21-day cycle of the induction phase for up to 24 months (e.g., on Day 1 of each of months 2, 4, 6, 8, 10, 12, 14, 16, 18, 20, 22, and 24).

In some embodiments, during the maintenance phase, the Bcl-2 inhibitor is venetoclax, and the venetoclax is administered orally at a dose of about 400 mg once daily following the sixth 21-day cycle of the induction phase for up to 8 months, and the anti-CD20 antibody is rituximab, and the rituximab is administered intravenously at a dose of about 375 mg/m² on Day 1 of every other month starting at 2 months after the sixth 21-day cycle of the induction phase for up to 24 months (e.g., on Day 1 of each of months 2, 4, 6, 8, 10, 12, 14, 16, 18, 20, 22, and 24).

In some embodiments, during the maintenance phase, the Bcl-2 inhibitor is venetoclax, and the venetoclax is administered orally at a dose of about 600 mg once daily following the sixth 21-day cycle of the induction phase for up to 8 months, and the anti-CD20 antibody is obinutuzumab, and the obinutuzumab is administered intravenously at a dose of about 1000 mg on Day 1 of every other month starting at 2 months after the sixth 21-day cycle of the induction phase for up to 24 months (e.g., on Day 1 of each of months 2, 4, 6, 8, 10, 12, 14, 16, 18, 20, 22, and 24).

In some embodiments, during the maintenance phase, the Bcl-2 inhibitor is venetoclax, and the venetoclax is administered orally at a dose of about 600 mg once daily following the sixth 21-day cycle of the induction phase for up to 8 months, and the anti-CD20 antibody is rituximab, and the rituximab is administered intravenously at a dose of about 375 mg/m² on Day 1 of every other month starting at 2 months after the sixth 21-day cycle of the induction phase for up to 24 months (e.g., on Day 1 of each of months 2, 4, 6, 8, 10, 12, 14, 16, 18, 20, 22, and 24).

In some embodiments, during the maintenance phase, the Bcl-2 inhibitor is venetoclax, and the venetoclax is administered orally at a dose of about 800 mg once daily following the sixth 21-day cycle of the induction phase for up to 8 months, and the anti-CD20 antibody is obinutuzumab, and the obinutuzumab is administered intravenously at a dose of about 1000 mg on Day 1 of every other month starting at 2 months after the sixth 21-day cycle of the induction phase for up to 24 months (e.g., on Day 1 of each of months 2, 4, 6, 8, 10, 12, 14, 16, 18, 20, 22, and 24).

In some embodiments, during the maintenance phase, the Bcl-2 inhibitor is venetoclax, and the venetoclax is administered orally at a dose of about 800 mg once daily following the sixth 21-day cycle of the induction phase for up to 8 months, and the anti-CD20 antibody is rituximab, and the rituximab is administered intravenously at a dose of about 375 mg/m² on Day 1 of every other month starting at 2 months after the sixth 21-day cycle of the induction phase for up to 24 months (e.g., on Day 1 of each of months 2, 4, 6, 8, 10, 12, 14, 16, 18, 20, 22, and 24).

The dosing and administration schedules for exemplary maintenance phases are provided in Tables I-L below:

### Tables I-L: Dosing and Administration Schedules for Exemplary Maintenance Phases (a Month Refers to 28 Days)

TABLE I

| Drugs | Dose and Frequency of Administration |
|---|---|
| Bcl-2 inhibitor | 200 mg once daily for 8 months (Months 1-8) |
| (venetoclax) | |
| Anti-CD20 Antibody | 1000 mg on Day 1 of every other month (i.e., |
| (obinutuzumab) | every 2 months) for 24 months, starting |
| | with Month 2 (e.g., Months 2, 4, 6, 8, etc.) |

TABLE J

| Drugs | Dose and Frequency of Administration |
|---|---|
| Bcl-2 inhibitor | 400 mg once daily for 8 months (Months 1-8) |
| (venetoclax) | |
| Anti-CD20 Antibody | 1000 mg on Day 1 of every other month (i.e., |
| (obinutuzumab) | every 2 months) for 24 months, starting |
| | with Month 2 (e.g., Months 2, 4, 6, 8, etc.) |

TABLE K

| | Dose and Frequency of |
|---|---|
| Drugs | Administration |
| Bcl-2 inhibitor | 600 mg once daily for 8 months |
| (venetoclax) | (Months 1-8) |
| Anti-CD20 Antibody | 1000 mg on Day 1 of every other |
| (obinutuzumab) | month (i.e., every 2 months) for 24 |
| | months, starting with Month 2 (e.g., |
| | Months 2, 4, 6, 8, etc.) |

TABLE L

| | Dose and Frequency of |
|---|---|
| Drugs | Administration |
| Bcl-2 inhibitor | 800 mg once daily for 8 months |
| (venetoclax) | (Months 1-8) |
| Anti-CD20 Antibody | 1000 mg on Day 1 of every other |
| (obinutuzumab) | month (i.e., every 2 months) for 24 |
| | months, starting with Month 2 (e.g., |
| | Months 2, 4, 6, 8, etc.) |

In some embodiments, the anti-CD20 antibody is rituximab. In some embodiments, the rituximab is administered at a dose of about 375 mg/m². In some embodiments, the rituximab is administered in combination with a Bcl-2 inhibitor (e.g., venetoclax) according to any maintenance phase provided herein. In some embodiments, during the maintenance phase, the Bcl-2 inhibitor is venetoclax, and the venetoclax is administered orally at a dose of between about 200 mg to about 800 mg (e.g., any of about 200 mg, about 400 mg, about 600 mg, or about 800 mg) once daily following the sixth 21-day cycle of the induction phase for up to 8 months, and the anti-CD20 antibody is rituximab, and the rituximab is administered intravenously at a dose of about 375 mg/m² on Day 1 of every other month starting at 2 months after the sixth 21-day cycle of the induction phase for up to 8 months (e.g., on Day 1 of each of months 2, 4, 6, and 8).

In some embodiments, a month comprises 28 days.

Any one of the exemplary induction phases provided herein (e.g., shown in Tables A-H) may be followed by any one of the exemplary maintenance phases provided herein (e.g., shown in Tables I-L).

In some embodiments, during the induction phase, the immunoconjugate is administered intravenously at a dose of about 1.8 mg/kg on Day 1 of the first 21-day cycle, the venetoclax is administered orally at a dose of about 800 mg on each of Days 1-21 of the first 21-day cycle, and the obinutuzumab is administered intravenously at a dose of about 1000 mg on each of Days 1, 8, and 15 of the first 21-day cycle, and the immunoconjugate is administered intravenously at a dose of about 1.8 mg/kg on Day 1 of each of the second, third, fourth, fifth, and sixth 21-day cycles, the venetoclax is administered orally at a dose of about 800 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles. In some embodiments, the induction phase is followed by a maintenance phase, wherein the venetoclax is administered at a dose of about 800 mg and the obinutuzumab is administered at a dose of about 1000 mg during the maintenance phase. In some embodiments, during the maintenance phase, the venetoclax is administered orally at a dose of about 800 mg once daily for 8 months following the sixth 21-day cycle of the induction phase, and the obinutuzumab is administered intravenously at a dose of about 1000 mg on Day 1 of every other month (i.e., every two months) for 24 months starting at two months after the sixth 21-day cycle of the induction phase.

In some embodiments, during the induction phase, the immunoconjugate is administered intravenously at a dose of about 1.8 mg/kg on Day 1 of the first 21-day cycle, the venetoclax is administered orally at a dose of about 800 mg on each of Days 1-21 of the first 21-day cycle, and the rituximab is administered intravenously at a dose of about 375 mg/m² on each of Days 1, 8, and 15 of the first 21-day cycle, and the immunoconjugate is administered intravenously at a dose of about 1.8 mg/kg on Day 1 of each of the second, third, fourth, fifth, and sixth 21-day cycles, the venetoclax is administered orally at a dose of about 800 mg on each of Days 1-21 of each of the second, third, fourth, fifth, and sixth 21-day cycles, and the rituximab is administered intravenously at a dose of about 375 mg/m² on Day 1 of each of the second, third, fourth, fifth, and sixth 21-day cycles. In some embodiments, the induction phase is followed by a maintenance phase, wherein the venetoclax is administered at a dose of about 800 mg and the rituximab is administered at a dose of about 375 mg/m² during the maintenance phase. In some embodiments, during the maintenance phase, the venetoclax is administered orally at a dose of about 800 mg once daily for 8 months following the sixth 21-day cycle of the induction phase, and the rituximab is administered intravenously at a dose of about 375 mg/m² on Day 1 of every other month (i.e., every two months) for 24 months starting at two months after the sixth 21-day cycle of the induction phase.

In some embodiments, the Bcl-2 inhibitor (e.g., venetoclax) and the anti-CD20 antibody (e.g., rituximab or obinutuzumab) are administered sequentially during the maintenance phase. In some embodiments, the Bcl-2 inhibitor (e.g., venetoclax) is administered prior to the anti-CD20 antibody (e.g., rituximab or obinutuzumab) on Day 1 of every other month during the maintenance phase (e.g., on Day 1 of each of months 2, 4, 6, 8, 10, 12, 14, 16, 18, 20, 22, and 24). In some embodiments, the venetoclax is administered prior to the obinutuzumab on Day 1 of each of months 2, 4, 6, 8, 10, 12, 14, 16, 18, 20, 22, and 24 during the maintenance phase. In some embodiments, the venetoclax is administered prior to the rituximab on Day 1 of each of months 2, 4, 6, 8, 10, 12, 14, 16, 18, 20, 22, and 24 during the maintenance phase.

In some embodiments, the Bcl-2 inhibitor, e.g., venetoclax, is administered for a maximum of 8 months during the maintenance phase (e.g., for a maximum of about 1 month, for a maximum of about 2 months, for a maximum of about 3 months, for a maximum of about 4 months, for a maximum of about 5 months, for a maximum of about 6 months, for a maximum of about 7 months, or for a maximum of about 8 months).

In some embodiments, the anti-CD20 antibody, e.g., obinutuzumab or rituximab, is administered during the maintenance phase starting on Day 1 of the second month after the sixth 21-day cycle of the induction phase. In some embodiments, the anti-CD20 antibody, e.g., obinutuzumab or rituximab, is administered during the maintenance phase for a maximum of 24 months (e.g., for a maximum of about 1 month, for a maximum of about 2 months, for a maximum of about 3 months, for a maximum of about 4 months, for a maximum of about 5 months, for a maximum of about 6 months, for a maximum of about 7 months, for a maximum of about 8 months, for a maximum of about 9 months, for a maximum of about 10 months, for a maximum of about 11 months, for a maximum of about 12 months, for a maximum of about 13 months, for a maximum of about 14 months, for a maximum of about 15 months, for a maximum of about 16 months, for a maximum of about 17 months, for a maximum of about 18 months, for a maximum of about 19 months, for a maximum of about 20 months, for a maximum of about 21 months, for a maximum of about 22 months, for a maximum of about 23 months, or for a maximum of about 24 months). In some embodiments, the anti-CD20 antibody is obinutuzumab, and obinutuzumab is administered during the maintenance phase for a maximum of 24 months (e.g., for a maximum of about 1 month, for a maximum of about 2 months, for a maximum of about 3 months, for a maximum of about 4 months, for a maximum of about 5 months, for a maximum of about 6 months, for a maximum of about 7 months, for a maximum of about 8 months, for a maximum of about 9 months, for a maximum of about 10 months, for a maximum of about 11 months, for a maximum of about 12 months, for a maximum of about 13 months, for a maximum of about 14 months, for a maximum of about 15 months, for a maximum of about 16 months, for a maximum of about 17 months, for a maximum of about 18 months, for a maximum of about 19 months, for a maximum of about 20 months, for a maximum of about 21 months, for a maximum of about 22 months, for a maximum of about 23 months, or for a maximum of about 24 months). In some embodiments, the anti-CD20 antibody is rituximab, and rituximab is administered during the maintenance phase for a maximum of 8 months (e.g., for a maximum of about 1 month, for a maximum of about 2 months, for a maximum of about 3 months, for a maximum of about 4 months, for a maximum of about 5 months, for a maximum of about 6 months, for a maximum of about 7 months, or for a maximum of about 8 months).

In some embodiments, the individual has an Eastern Cooperative Oncology Group (ECOG) Performance Status of 0, 1, or 2. In some embodiments, the individual has relapsed or refractory (R/R) FL after treatment with at least 1 prior chemoimmunotherapy regimen that included an anti-CD20 monoclonal antibody. In some embodiments, the individual has histologically documented CD20-positive Non-Hodgkin lymphoma (e.g., FL). In some embodiments, the individual has fluorodeoxyglucose (FDG)-avid lymphoma (i.e., PET-positive lymphoma). In some embodiments, the individual has at least one bi-dimensionally measurable lesion (>1.5 cm in its largest dimension by CT scan or magnetic resonance imaging [MRI]). In some embodiments, the individual does not have a known CD20-negative status at disease relapse or progression. In some embodiments, the individual has not undergone a prior allogeneic stem cell transplant (SCT). In some embodiments, the individual has not completed an autologous SCT within 100 days prior to initiation of treatment according to a method provided herein. In some embodiments, the individual does not have Grade 3b FL. In some embodiments, the individual does not have history of transformation of indolent disease to diffuse large B cell lymphoma (DLBCL). In some embodiments, the individual does not have Grade 1 or greater peripheral neuropathy. In some embodiments, the individual does not have CNS lymphoma or leptomeningeal infiltration. In some embodiments, the individual is not receiving greater than 20 mg per day of a corticosteroid, e.g., prednisone. In some embodiments, the individual is administered up to 100 mg per day of a corticosteroid, e.g., prednisone, for up to 5 days. In some embodiments, the individual is not taking a warfarin treatment. In some embodiments, the individual is not taking a strong or moderate CYP3A inhibitor such as fluconazole, ketoconazole, and clarithromycin, or a strong or moderate CYP3A inducer such as rifampin and carbamazepine within 7 days prior to initiation of treatment according to any method provided herein. In some embodiments, the individual has not consumed grapefruit, grapefruit products, Seville oranges, Seville orange products (e.g., marmalade that contains Seville oranges), star fruit, or star fruit products within 3 days prior to initiation of treatment according to any method provided herein. In some embodiments, the individual does not have a history of progression multifocal Leukoencephalopathy (PML). In some embodiments, the individual has received at least one prior treatment for FL, e.g., any of 1, 2, 3, 4, 5, 6, 7, or more prior treatments for FL. In some embodiments, the individual has FL with a histological grade of 1, 2, or 3a. In some embodiments, the individual has FL with bone marrow involvement. In some embodiments, the individual has FL with an Ann Arbor state of between 1 to 2, or between 3 to 4. In some embodiments, the individual has a Follicular Lymphoma International Prognostic Index (FLIPI) score of between about 0 to about 5, e.g., any of 0, 1, 2, 3, 4, or 5. In some embodiments, the individual has bulky disease (e.g., greater than 7 cm). In some embodiments, the individual is refractory to a treatment with an anti-CD20 agent (e.g., no response or progression or relapse of FL within 6 months of completing an FL treatment with an anti-CD20 agent). In some embodiments, the individual is refractory to the last prior treatment for FL (e.g., no response or progression or relapse within 6 months of completion of the last prior FL treatment). In some embodiments, the individual had progression of disease within 24 months of completing an initial FL treatment. In some embodiments, the individual had progression of disease within 24 months of completing the last prior FL treatment. In some embodiments, the individual had progression of disease within 24 months of completing an FL treatment.

In some embodiments, a human treated according to the methods provided herein does not experience peripheral neuropathy of grade 3 or greater. In some embodiments, among a plurality of humans treated according to the methods provided herein, about 64% or less of the humans experience a Grade 3 or Grade 4 adverse event. In some embodiments, after administration of the immunoconjugate, the Bcl-2 inhibitor, and the anti-CD20 antibody, the human does not experience peripheral neuropathy of grade 3 or greater. In some embodiments, among a plurality of humans treated according to the methods provided herein, about 64% or less (e.g., any of 64% or less, 60% or less, 55% or less, 50% or less, 45% or less, 40% or less, 35% or less, 30% or less, 25% or less, 20% or less, 15% or less, 10% or less, 5% or less, 2.5% or less, 2% or less, or 1% or less) of the humans experience a Grade 3 or Grade 4 adverse event.

In some embodiments, among a plurality of humans treated according to the methods provided herein, about 59% or less (e.g., any of 59% or less, 55% or less, 50% or less, 45% or less, 40% or less, 35% or less, 30% or less, 25% or less, 20% or less, 15% or less, 10% or less, 5% or less, 2.5% or less, 2% or less, or 1% or less) of the humans experience a Grade 3 or Grade 4 adverse event.

In some embodiments, among a plurality of humans treated according to the methods provided herein, about 75% or less (e.g., any of 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, 2.5% or less, 2% or less, or 1% or less) of the humans experience a Grade 3 or Grade 4 adverse event. In some embodiments, among a plurality of humans treated according to the methods provided herein, about 73% or less of the humans experience a Grade 3 or Grade 4 adverse event.

In some embodiments, after administration of the polatuzumab vedotin-piiq, the venetoclax, and the obinutuzumab, the human does not experience peripheral neuropathy of grade 3 or greater. In some embodiments, after administration of the polatuzumab vedotin-piiq, the venetoclax, and the obinutuzumab, the human does not develop tumor lysis syndrome. In some embodiments, among a plurality of humans treated with the polatuzumab vedotin-piiq, the venetoclax, and the obinutuzumab according to the methods provided herein, about 64% or less of the humans experience a Grade 3 or Grade 4 adverse event.

In some embodiments, the methods of treating FL provided herein further include administering a prophylactic treatment for tumor lysis syndrome (TLS), for example, as described in Example 1 herein. In some embodiments, the prophylactic treatment for tumor lysis syndrome (TLS) comprises a uric acid-reducing agent and/or a hydration regimen prior to the start of treatment. In some embodiments, the hydration regimen comprises administering about 2 to about 3 liters per day of fluids (e.g., water, saline, or other suitable fluids), wherein the fluids are administered starting at about 24 hours to about 48 hours prior to the start of treatment. In some embodiments, the fluids are administered orally or intravenously. In some embodiments, the fluids are administered orally. In some embodiments, the fluids are administered intravenously. In some embodiments, the uric acid-reducing agent is allopurinol. In some embodiments, the allopurinol is administered orally at a dose of about 300 mg/day starting at about 72 hours prior to the first dose of venetoclax, and wherein administration of allopurinol continues for between about 3 to about 7 days after administration of the first dose of venetoclax. In some embodiments, the prophylactic treatment for tumor lysis syndrome (TLS) comprises administering rasburicase intravenously to humans with elevated uric acid levels before the start of treatment, wherein the rasburicase is administered until normalization of serum uric acid and other evidence (e.g., laboratory test results) of TLS.

In some embodiments, the methods of treating FL provided herein further include treating or preventing adverse events as described in Example 1 herein. In some embodiments, the methods of treating FL provided herein further include treating an occurrence of a hematologic adverse event, e.g., neutropenia, as described in Example 1 herein. In some embodiments, the methods of treating FL provided herein further include administering granulocyte colony stimulating factor (G-CSF) if a Grade 3 or Grade 4 adverse event of neutropenia occurs.

### IV. Methods for Treating Diffuse Large B-Cell Lymphoma

Also provided herein are methods for treating diffuse large B-cell lymphoma (DLBCL) in a human in need thereof comprising administering to the human an effective amount of: (a) an immunoconjugate comprising the formula

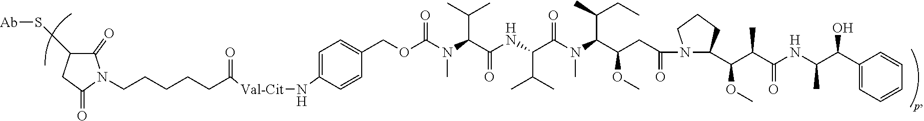

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and wherein p is between 1 and 8, (b) a Bcl-2 inhibitor, and (c) an anti-CD20 antibody, wherein the human achieves a complete response (CR) during or after treatment. In some embodiments, the anti-CD79b immunoconjugate is huMA79bv28-MC-vc-PAB-MMAE. In some embodiments, the immunoconjugate is polatuzumab vedotin-piiq (CAS Registry Number 1313206-42-6). In some embodiments, the anti-CD79b immunoconjugate is huMA79bv28-MC-vc-PAB-MMAE. In some embodiments, the immunoconjugate is polatuzumab vedotin-piiq (CAS Registry Number 1313206-42-6). In some embodiments, the immunoconjugate is polatuzumab vedotin-piiq. In some embodiments, the Bcl-2 inhibitor is venetoclax. In some embodiments, the anti-CD20 antibody is rituximab. In some embodiments, the anti-CD20 antibody is obinutuzumab. In some embodiments, the anti-CD20 antibody is ofatumumab, ublituximab, and/or ibritumomab tiuxetan.

The terms “co-administration” or “co-administering” refer to the administration of the anti-CD79b immunoconjugate, the Bcl-2 inhibitor, and the anti-CD20 antibody as two (or more) separate formulations (or as one single formulation comprising the anti-CD79b immunoconjugate, the Bcl-2 inhibitor, and the anti-CD20 antibody). Where separate formulations are used, the co-administration can be simultaneous or sequential in either order, wherein preferably there is a time period while all active agents simultaneously exert their biological activities. The anti-CD79b immunoconjugate, the Bcl-2 inhibitor, and the anti-CD20 antibody are co-administered either simultaneously or sequentially.

Anti-CD79b immunoconjugates and additional therapeutic agents (e.g., a Bcl-2 inhibitor, and an anti-CD20 antibody) provided herein for use in any of the therapeutic methods described herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The immunoconjugate need not be, but is optionally formulated with one or more agents currently used to prevent or treat the disorder in question.

The amount of co-administration of the anti-CD79b immunoconjugate and the additional therapeutic agent and the timing of co-administration will depend on the type (species, gender, age, weight, etc.) and condition of the patient being treated and the severity of the disease or condition being treated. The anti-CD79b immunoconjugate, the Bcl-2 inhibitor and the anti-CD20 antibody are suitably co-administered to the patient at one time or over a series of treatments e.g., on the same day or on the day after.

In some embodiments, the dosage of anti-CD79b immunoconjugate (such as huMA79bv28-MC-vc-PAB-MMAE or polatuzumab vedotin-piiq) is between about any of 1.4-5 mg/kg, 1.4-4 mg/kg, 1.4-3.2 mg/kg, 1.4-2.4 mg/kg, or 1.4-1.8 mg/kg. In some embodiments of any of the methods, the dosage of anti-CD79 immunoconjugate is about any of 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, and/or 4.8 mg/kg. In some embodiments, the dosage of anti-CD79b immunoconjugate is about 1.4 mg/kg. In some embodiments, the dosage of anti-CD79b immunoconjugate is about 1.8 mg/kg. In some embodiments, the dosage of anti-CD79b immunoconjugate is about 2.4 mg/kg. In some embodiments, the dosage of anti-CD79b immunoconjugate is about 3.2 mg/kg. In some embodiments, the dosage of anti-CD79b immunoconjugate is about 3.6 mg/kg. In some embodiments of any of the methods, the anti-CD79b immunoconjugate is administered q3wk or q3w (e.g., on day 1 of each 21-day cycle, once every 3 weeks, or once every 21 days). In some embodiments, the anti-CD79b immunoconjugate is administered via intravenous infusion. In some embodiments, the dosage administered via infusion is in the range of about 1 mg to about 1,500 mg per dose. Alternatively, the dosage range is of about 1 mg to about 1,500 mg, about 1 mg to about 1,000 mg, about 400 mg to about 1200 mg, about 600 mg to about 1000 mg, about 10 mg to about 500 mg, about 10 mg to about 300 mg, about 10 mg to about 200 mg, and about 1 mg to about 200 mg. In some embodiments, the dosage administered via infusion is in the range of about 1 μg/m² to about 10,000 μg/m² per dose. Alternatively, the dosage range is of about 1 μg/m² to about 1000 μg/m², about 1 μg/m² to about 800 μg/m², about 1 μg/m² to about 600 μg/m², about 1 μg/m² to about 400 μg/m², about 10 μg/m² to about 500 μg/m², about 10 μg/m² to about 300 μg/m², about 10 μg/m² to about 200 μg/m², and about 1 μg/m² to about 200 μg/m². The dose may be administered once per day, once per week, multiple times per week, but less than once per day, multiple times per month but less than once per day, multiple times per month but less than once per week, once per month, once every 21 days, or intermittently to relieve or alleviate symptoms of the disease. Administration may continue at any of the intervals and doses described herein until remission of the tumor or symptoms of the B-cell proliferative disorder being treated. Administration may continue after remission or relief of symptoms is achieved where such remission or relief is prolonged by such continued administration.

In some embodiments, the dosage of the anti-CD20 antibody is between about 300-1600 mg/m² and/or 300-2000 mg. In some embodiments, the dosage of the anti-CD20 antibody is about any of 300 mg/m², 375 mg/m², 600 mg/m², 1000 mg/m², or 1250 mg/m² and/or 300 mg, 1000 mg, or 2000 mg. In some embodiments, the anti-CD20 antibody is rituximab and the dosage administered is 375 mg/m². In some embodiments, the anti-CD20 antibody is obinutuzumab and the dosage administered is 1000 mg. In some embodiments, the anti-CD20 antibody is administered q1w (i.e., once per week). In some embodiments, the anti-CD20 antibody is administered on days 1, 8, and 15 of a 21-day cycle. In some embodiments, the anti-CD20 antibody is administered q3w (i.e., every 3 weeks or once every 21 days). In some embodiments, the anti-CD20 antibody is administered on day lof a 21-day cycle. In some embodiments, the anti-CD20 antibody is administered on days 1, 8, and 15 of the first 21-day cycle, and on day 1 of the subsequent 21-day cycles (e.g., on day 1 of cycles 2, 3, 4, 5, and 6). In some embodiments, the anti-CD20 antibody is administered once every two months. In some embodiments, the dosage of an afucosylated anti-CD20 antibody (preferably the afucosylated humanized B-Ly1 antibody) may be 800 to 1600 mg (in one embodiment 800 to 1200 mg, such as 1000 mg) or 400 to 1200 mg (in one embodiment 800 to 1200 mg). In some embodiments, the dose is a flat dose of 1000 mg in a three-weeks-dosage schedule (e.g., once every 21 days).

In some embodiments, the dose of the Bcl-2 inhibitor (e.g., venetoclax) is between about 100 mg to about 800 mg (e.g., any of about 100 mg, 200 mg, 300 mg, 400 mg, 500 mg, 600 mg, 700 mg, or 800 mg). In some embodiments, venetoclax is administered at a dose of between about 100 mg to about 800 mg. In some embodiments, venetoclax is administered at a dose of about 100 mg. In some embodiments, venetoclax is administered at a dose of about 200 mg. In some embodiments, venetoclax is administered at a dose of about 400 mg. In some embodiments, venetoclax is administered at a dose of about 600 mg. In some embodiments, venetoclax is administered at a dose of about 800 mg.

An immunoconjugate provided herein (and any additional therapeutic agents, e.g., a Bcl-2 inhibitor and an anti-CD20 antibody) for use in any of the therapeutic methods described herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The anti-CD79b immunoconjugate (e.g., huMA79bv28-MC-vc-PAB-MMAE or polatuzumab vedotin-piiq), the Bcl-2 inhibitor (such as venetoclax) and the anti-CD20 antibody (such as rituximab or obinutuzumab) may be administered by the same route of administration or by different routes of administration. In some embodiments, the anti-CD79b immunoconjugate (e.g., polatuzumab vedotin-piiq) is administered intravenously, intramuscularly, subcutaneously, topically, orally, transdermally, intraperitoneally, intraorbitally, by implantation, by inhalation, intrathecally, intraventricularly, or intranasally. In some embodiments, the immunoconjugate (e.g., polatuzumab vedotin-piiq) is administered by intravenous infusion. In some embodiments, the anti-CD20 antibody (such as rituximab or obinutuzumab) is administered intravenously, intramuscularly, subcutaneously, topically, orally, transdermally, intraperitoneally, intraorbitally, by implantation, by inhalation, intrathecally, intraventricularly, or intranasally. In some embodiments, the anti-CD20 antibody (e.g., rituximab or obinutuzumab) is administered by intravenous infusion. In some embodiments, the Bcl-2 inhibitor (such as venetoclax) is administered intravenously, intramuscularly, subcutaneously, topically, orally, transdermally, intraperitoneally, intraorbitally, by implantation, by inhalation, intrathecally, intraventricularly, or intranasally. In some embodiments, the Bcl-2 inhibitor (e.g., venetoclax) is administered orally, e.g., in a tablet, capsule, or any other suitable means for oral administration known in the art or described herein. In some embodiments, the anti-CD79b immunoconjugate and the anti-CD20 antibody (such as rituximab or obinutuzumab) are each administered via intravenous infusion, and the Bcl-2 inhibitor (such as venetoclax) is administered orally. An effective amount of the anti-CD79b immunoconjugate, the Bcl-2 inhibitor (such as venetoclax) and the anti-CD20 antibody (such as rituximab or obinutuzumab) may be administered for prevention or treatment of disease.

In some embodiments, the anti-CD79b immunoconjugate (e.g., huMA79bv28-MC-vc-PAB-MMAE or polatuzumab vedotin-piiq) is administered at a dose between about 1.4 mg/kg to about 1.8 mg/kg. In some embodiments, the anti-CD79b immunoconjugate (e.g., huMA79bv28-MC-vc-PAB-MMAE or polatuzumab vedotin-piiq) is administered at a dose of about 1.4 mg/kg. In some embodiments, the anti-CD79b immunoconjugate (e.g., huMA79bv28-MC-vc-PAB-MMAE or polatuzumab vedotin-piiq) is administered at a dose of about 1.8 mg/kg. Alternatively or additionally, in some embodiments, the Bcl-2 inhibitor (such as venetoclax) is administered at a dose between about 200 mg and about 800 mg. In some embodiments, the Bcl-2 inhibitor (such as venetoclax) is administered at a dose of about 200 mg. In some embodiments, the Bcl-2 inhibitor (such as venetoclax) is administered at a dose of about 400 mg. In some embodiments, the Bcl-2 inhibitor (such as venetoclax) is administered at a dose of about 600 mg. In some embodiments, the Bcl-2 inhibitor (such as venetoclax) is administered at a dose of about 800 mg. Alternatively or additionally, in some embodiments, the anti-CD20 antibody is rituximab. In some embodiments, the rituximab is administered at a dose of about 375 mg/m². Alternatively or additionally, in some embodiments, the anti-CD20 antibody is obinutuzumab. In some embodiments, the obinutuzumab is administered at a dose of about 1000 mg. In some embodiments, the polatuzumab vedotin-piiq is administered at a dose of about 1.8 mg/kg, the venetoclax is administered at a dose of about 400 mg, about 600 mg, or about 800 mg, and the rituximab is administered at a dose of about 375 mg/m². In some embodiments, the polatuzumab vedotin-piiq is administered at a dose of about 1.8 mg/kg, the venetoclax is administered at a dose of about 400 mg, about 600 mg, or about 800 mg, and the obinutuzumab is administered at a dose of about 1000 mg. In some embodiments, the polatuzumab vedotin-piiq is administered at a dose of about 1.8 mg/kg, the venetoclax is administered at a dose of about 400 mg, and the rituximab is administered at a dose of about 375 mg/m². In some embodiments, the polatuzumab vedotin-piiq is administered at a dose of about 1.8 mg/kg, the venetoclax is administered at a dose of about 400 mg, and the obinutuzumab is administered at a dose of about 1000 mg. In some embodiments, the polatuzumab vedotin-piiq is administered at a dose of about 1.8 mg/kg, the venetoclax is administered at a dose of about 600 mg, and the rituximab is administered at a dose of about 375 mg/m². In some embodiments, the polatuzumab vedotin-piiq is administered at a dose of about 1.8 mg/kg, the venetoclax is administered at a dose of about 600 mg, and the obinutuzumab is administered at a dose of about 1000 mg. In some embodiments, the polatuzumab vedotin-piiq is administered at a dose of about 1.8 mg/kg, the venetoclax is administered at a dose of about 800 mg, and the rituximab is administered at a dose of about 375 mg/m². In some embodiments, the polatuzumab vedotin-piiq is administered at a dose of about 1.8 mg/kg, the venetoclax is administered at a dose of about 800 mg, and the obinutuzumab is administered at a dose of about 1000 mg.

In some embodiments, the anti-CD79b immunoconjugate (e.g., polatuzumab vedotin-piiq), the Bcl-2 inhibitor (e.g., venetoclax), and the anti-CD20 antibody (e.g., obinutuzumab or rituximab) are administered during an induction phase. An induction phase refers to a phase of treatment wherein the anti-CD79b immunoconjugate, the Bcl-2 inhibitor, and the anti-CD20 antibody are administered to a human. In some embodiments, the induction phase comprises less than one complete 21-day cycle. In some embodiments, the induction phase comprises between one and six (e.g., any of 1, 2, 3, 4, 5, or 6) 21-day cycles. In some embodiments, the induction phase comprises at least six 21-day cycles.

In some embodiments, during the induction phase, the immunoconjugate is administered intravenously at a dose of about 1.8 mg/kg on Day 1 of each of the first, second, third, fourth, fifth, and sixth 21-day cycles, the anti-CD20 antibody is administered intravenously at a dose of about 375 mg/m² or about 1000 mg on Day 1 of each of the first, second, third, fourth, fifth, and sixth 21-day cycles, and the Bcl-2 inhibitor is administered orally at a dose of about 400 mg, about 600 mg, or about 800 mg on each of Days 1-21 of each of the first, second, third, fourth, fifth, and sixth 21-day cycles.

In some embodiments, during the induction phase, the polatuzumab vedotin-piiq is administered intravenously at a dose of about 1.8 mg/kg on Day 1 of each of the first, second, third, fourth, fifth, and sixth 21-day cycles, the rituximab is administered intravenously at a dose of about 375 mg/m² on Day 1 of each of the first, second, third, fourth, fifth, and sixth 21-day cycles, and the venetoclax is administered orally at a dose of about 400 mg, about 600 mg, or about 800 mg on each of Days 1-21 of each of the first, second, third, fourth, fifth, and sixth 21-day cycles.

In some embodiments, during the induction phase, the polatuzumab vedotin-piiq is administered intravenously at a dose of about 1.8 mg/kg on Day 1 of each of the first, second, third, fourth, fifth, and sixth 21-day cycles, the obinutuzumab is administered intravenously at a dose of about 1000 mg on Day 1 of each of the first, second, third, fourth, fifth, and sixth 21-day cycles, and the venetoclax is administered orally at a dose of about 400 mg, about 600 mg, or about 800 mg on each of Days 1-21 of each of the first, second, third, fourth, fifth, and sixth 21-day cycles.

In some embodiments, during the induction phase, the polatuzumab vedotin-piiq is administered intravenously at a dose of about 1.8 mg/kg on Day 1 of each of the first, second, third, fourth, fifth, and sixth 21-day cycles, the rituximab is administered intravenously at a dose of about 375 mg/m² on Day 1 of each of the first, second, third, fourth, fifth, and sixth 21-day cycles, and the venetoclax is administered orally at a dose of about 400 mg on each of Days 1-21 of each of the first, second, third, fourth, fifth, and sixth 21-day cycles.

In some embodiments, during the induction phase, the polatuzumab vedotin-piiq is administered intravenously at a dose of about 1.8 mg/kg on Day 1 of each of the first, second, third, fourth, fifth, and sixth 21-day cycles, the obinutuzumab is administered intravenously at a dose of about 1000 mg on Day 1 of each of the first, second, third, fourth, fifth, and sixth 21-day cycles, and the venetoclax is administered orally at a dose of about 400 mg on each of Days 1-21 of each of the first, second, third, fourth, fifth, and sixth 21-day cycles.

In some embodiments, during the induction phase, the polatuzumab vedotin-piiq is administered intravenously at a dose of about 1.8 mg/kg on Day 1 of each of the first, second, third, fourth, fifth, and sixth 21-day cycles, the rituximab is administered intravenously at a dose of about 375 mg/m² on Day 1 of each of the first, second, third, fourth, fifth, and sixth 21-day cycles, and the venetoclax is administered orally at a dose of about 600 mg on each of Days 1-21 of each of the first, second, third, fourth, fifth, and sixth 21-day cycles.

In some embodiments, during the induction phase, the polatuzumab vedotin-piiq is administered intravenously at a dose of about 1.8 mg/kg on Day 1 of each of the first, second, third, fourth, fifth, and sixth 21-day cycles, the obinutuzumab is administered intravenously at a dose of about 1000 mg on Day 1 of each of the first, second, third, fourth, fifth, and sixth 21-day cycles, and the venetoclax is administered orally at a dose of about 600 mg on each of Days 1-21 of each of the first, second, third, fourth, fifth, and sixth 21-day cycles.

In some embodiments, during the induction phase, the polatuzumab vedotin-piiq is administered intravenously at a dose of about 1.8 mg/kg on Day 1 of each of the first, second, third, fourth, fifth, and sixth 21-day cycles, the rituximab is administered intravenously at a dose of about 375 mg/m² on Day 1 of each of the first, second, third, fourth, fifth, and sixth 21-day cycles, and the venetoclax is administered orally at a dose of about 800 mg on each of Days 1-21 of each of the first, second, third, fourth, fifth, and sixth 21-day cycles.

In some embodiments, during the induction phase, the polatuzumab vedotin-piiq is administered intravenously at a dose of about 1.8 mg/kg on Day 1 of each of the first, second, third, fourth, fifth, and sixth 21-day cycles, the obinutuzumab is administered intravenously at a dose of about 1000 mg on Day 1 of each of the first, second, third, fourth, fifth, and sixth 21-day cycles, and the venetoclax is administered orally at a dose of about 800 mg on each of Days 1-21 of each of the first, second, third, fourth, fifth, and sixth 21-day cycles.

In some embodiments, the immunoconjugate, the Bcl-2 inhibitor, and the anti-CD20 antibody are administered sequentially during the induction phase. In some embodiments, the Bcl-2 inhibitor is administered prior to the anti-CD20 antibody and the anti-CD20 antibody is administered prior to the immunoconjugate on Day 1 of each of the first, second, third, fourth, fifth, and sixth 21-day cycles.

In some embodiments, the anti-CD20 antibody is rituximab. In some embodiments, the rituximab is administered at a dose of about 375 mg/m² in combination with an anti-CD79b immunoconjugate and a Bcl-2 inhibitor according to any induction phase provided herein.

In some embodiments, the anti-CD20 antibody is obinutuzumab. In some embodiments, the obinutuzumab is administered at a dose of about 1000 mg in combination with an anti-CD79b immunoconjugate and a Bcl-2 inhibitor according to any induction phase provided herein.

In some embodiments, the polatuzumab vedotin-piiq, the venetoclax, and the rituximab are administered sequentially during the induction phase. In some embodiments, the venetoclax is administered prior to the rituximab and the rituximab is administered prior to the polatuzumab vedotin-piiq on Day 1 of each of the first, second, third, fourth, fifth, and sixth 21-day cycles. In some embodiments, the polatuzumab vedotin-piiq, the venetoclax, and the obinutuzumab are administered sequentially during the induction phase. In some embodiments, the venetoclax is administered prior to the obinutuzumab and the obinutuzumab is administered prior to the polatuzumab vedotin-piiq on Day 1 of each of the first, second, third, fourth, fifth, and sixth 21-day cycles.

In some embodiments, a human treated according to the methods provided herein achieves a complete response (CR) during or after treatment. In some embodiments, the human achieves a complete response during induction treatment. In some embodiments, the human achieves a complete response at the end of induction treatment (e.g., after six 21-day cycles). In some embodiments, the human achieves a complete response after six 21-day cycles. In some embodiments, the human achieves a complete response after one, two, three, four, five, or six 21-day cycles.

In some embodiments, among a plurality of humans treated, at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans achieve a complete response during or after treatment. In some embodiments, among a plurality of humans treated, at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans achieve a complete response during induction treatment. In some embodiments, among a plurality of humans treated, at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans achieve a complete response at the end of induction treatment (e.g., after six 21-day cycles). In some embodiments, among a plurality of humans treated, at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans achieve a complete response after six 21-day cycles. In some embodiments, among a plurality of humans treated, at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans achieve a complete response after one, two, three, four, five, or six 21-day cycles. In some embodiments, among a plurality of humans treated, at least about 29% of the humans achieve a complete response during or after treatment. In some embodiments, among a plurality of humans treated, at least about 29% of the humans achieve a complete response during induction treatment. In some embodiments, among a plurality of humans treated, at least about 29% of the humans achieve a complete response at the end of induction treatment (e.g., after six 21-day cycles). In some embodiments, among a plurality of humans treated, at least about 29% of the humans achieve a complete response after six 21-day cycles. In some embodiments, among a plurality of humans treated, at least about 29% of the humans achieve a complete response after one, two, three, four, five, or six 21-day cycles. In some embodiments, among a plurality of humans treated, at least about 31% of the humans achieve a complete response during or after treatment. In some embodiments, among a plurality of humans treated, at least about 31% of the humans achieve a complete response during induction treatment. In some embodiments, among a plurality of humans treated, at least about 31% of the humans achieve a complete response at the end of induction treatment (e.g., after six 21-day cycles). In some embodiments, among a plurality of humans treated, at least about 31% of the humans achieve a complete response after six 21-day cycles. In some embodiments, among a plurality of humans treated, at least about 31% of the humans achieve a complete response after one, two, three, four, five, or six 21-day cycles.

In some embodiments, treatment of a plurality of humans according to the methods provided herein results in a best complete response rate of at least about 35%, at least about 38%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%. In some embodiments, treatment of a plurality of humans according to the methods provided herein results in a best complete response rate of at least about 38%. In some embodiments, the best complete response rate refers to the proportion of humans in a plurality of humans treated according to the methods provided herein who achieved a complete response at any time after initiation of treatment.

In some embodiments, the duration of the complete response is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more. In some embodiments, the duration of the complete response is at least about 1 month, at least about 2 months, at least about 3 months, at least about 4 months, at least about 5 months, at least about 6 months, at least about 7 months, or more. In some embodiments, the duration of the complete response is at least about 3 months or more. In some embodiments, the duration of the complete response is at least about 4 months or more. In some embodiments, the duration of the complete response is at least about 5 months or more. In some embodiments, the duration of the complete response is at least about 6 months or more. In some embodiments, the duration of the complete response is at least about 7 months or more. In some embodiments, the duration of a complete response is measured from the first occurrence of a complete response (e.g., as described below) to disease progression (e.g., according to the modified Lugano 2014 criteria). In some embodiments, the duration of a complete response is measured from the first occurrence of a complete response to relapse (e.g., according to the modified Lugano 2014 criteria). In some embodiments, the duration of a complete response is measured from the first occurrence of a complete response to disease progression or relapse (e.g., according to the modified Lugano 2014 criteria). In some embodiments, the duration of a complete response is measured from the first occurrence of a complete response to disease progression or relapse (e.g., according to the modified Lugano 2014 criteria), or death from any cause.

In some embodiments, among a plurality of humans treated, at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans achieve an objective response during or after treatment. In some embodiments, among a plurality of humans treated, at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans achieve an objective response during induction treatment. In some embodiments, among a plurality of humans treated, at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans achieve an objective response at the end of induction treatment (e.g., after six 21-day cycles). In some embodiments, among a plurality of humans treated, at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans achieve an objective response after six 21-day cycles. In some embodiments, among a plurality of humans treated, at least about 25%, at least about 27%, at least about 29%, at least about 31%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% of the humans achieve an objective response after one, two, three, four, five, or six 21-day cycles. In some embodiments, among a plurality of humans treated, at least about 29% of the humans achieve an objective response during or after treatment. In some embodiments, among a plurality of humans treated, at least about 29% of the humans achieve an objective response during induction treatment. In some embodiments, among a plurality of humans treated, at least about 29% of the humans achieve an objective response at the end of induction treatment (e.g., after six 21-day cycles). In some embodiments, among a plurality of humans treated, at least about 29% of the humans achieve an objective response after six 21-day cycles. In some embodiments, among a plurality of humans treated, at least about 29% of the humans achieve an objective response after one, two, three, four, five, or six 21-day cycles. In some embodiments, among a plurality of humans treated, at least about 42% of the humans achieve an objective response during or after treatment. In some embodiments, among a plurality of humans treated, at least about 42% of the humans achieve an objective response during induction treatment. In some embodiments, among a plurality of humans treated, at least about 42% of the humans achieve an objective response at the end of induction treatment (e.g., after six 21-day cycles). In some embodiments, among a plurality of humans treated, at least about 42% of the humans achieve an objective response after six 21-day cycles. In some embodiments, among a plurality of humans treated, at least about 42% of the humans achieve an objective response after one, two, three, four, five, or six 21-day cycles.

As used herein, an objective response refers to a complete response or a partial response, evaluated according to the modified Lugano 2014 criteria, as described in Example 1 herein. Thus, a human treated according to the methods provided herein that achieves an objective response achieves a complete response or a partial response, assessed according to the modified Lugano 2014 criteria, as described in Example 1 herein.

In some embodiments, objective responses are assessed on the basis of PET-CT scans according to the modified Lugano 2014 criteria, as described in Example 1 herein. In some embodiments, objective responses are assessed on the basis of CT scans alone according to the modified Lugano 2014 criteria, as described in Example 1 herein.

In some embodiments, the duration of the objective response is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more. In some embodiments, the duration of the objective response is at least about 1 month, at least about 2 months, at least about 3 months, at least about 4 months, at least about 5 months, at least about 6 months, at least about 7 months, or more. In some embodiments, the duration of the objective response is at least about 3 months or more. In some embodiments, the duration of the objective response is at least about 4 months or more. In some embodiments, the duration of the objective response is at least about 5 months or more. In some embodiments, the duration of the objective response is at least about 6 months or more. In some embodiments, the duration of the objective response is at least about 7 months or more. In some embodiments, the duration of an objective response is measured from the first occurrence of an objective response (e.g., as described herein) to disease progression (e.g., according to the modified Lugano 2014 criteria). In some embodiments, the duration of an objective response is measured from the first occurrence of an objective response to relapse (e.g., according to the modified Lugano 2014 criteria). In some embodiments, the duration of an objective response is measured from the first occurrence of an objective response to disease progression or relapse (e.g., according to the modified Lugano 2014 criteria). In some embodiments, the duration of an objective response is measured from the first occurrence of an objective response to disease progression or relapse (e.g., according to the modified Lugano 2014 criteria), or death from any cause.

In some embodiments, among a plurality of humans treated, at least about 60%, at least about 65%, at least about 70%, at least about 75%, at least about 80%, at least about 85%, at least about 90%, at least about 95%, or 100% of the humans achieve a best overall response (BOR) during or after treatment. In some embodiments, among a plurality of humans treated, at least about 60%, at least about 65%, at least about 70%, at least about 75%, at least about 80%, at least about 85%, at least about 90%, at least about 95%, or 100% of the humans achieve a best overall response (BOR) during induction treatment. In some embodiments, among a plurality of humans treated, at least about 60%, at least about 65%, at least about 70%, at least about 75%, at least about 80%, at least about 85%, at least about 90%, at least about 95%, or 100% of the humans achieve a best overall response (BOR) at the end of induction treatment (e.g., after six 21-day cycles). In some embodiments, among a plurality of humans treated, at least about 60%, at least about 65%, at least about 70%, at least about 75%, at least about 80%, at least about 85%, at least about 90%, at least about 95%, or 100% of the humans achieve a best overall response (BOR) after six 21-day cycles. In some embodiments, among a plurality of humans treated, at least about 60%, at least about 65%, at least about 70%, at least about 75%, at least about 80%, at least about 85%, at least about 90%, at least about 95%, or 100% of the humans achieve a best overall response (BOR) after one, two, three, four, five, or six 21-day cycles. In some embodiments, among a plurality of humans treated, at least about 65% of the humans achieve a best overall response (BOR) during or after treatment. In some embodiments, among a plurality of humans treated, at least about 65% of the humans achieve a best overall response (BOR) during induction treatment. In some embodiments, among a plurality of humans treated, at least about 65% of the humans achieve a best overall response (BOR) at the end of induction treatment (e.g., after six 21-day cycles). In some embodiments, among a plurality of humans treated, at least about 65% of the humans achieve a best overall response (BOR) after six 21-day cycles. In some embodiments, among a plurality of humans treated, at least about 65% of the humans achieve a best overall response (BOR) after one, two, three, four, five, or six 21-day cycles. In some embodiments, the duration of the BOR is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more. In some embodiments, the duration of the BOR is at least about 1 month, at least about 2 months, at least about 3 months, at least about 4 months, at least about 5 months, at least about 6 months, at least about 7 months, or more. In some embodiments, the duration of the BOR is at least about 3 months or more. In some embodiments, the duration of the BOR is at least about 4 months or more. In some embodiments, the duration of the BOR is at least about 5 months or more. In some embodiments, the duration of the BOR is at least about 6 months or more. In some embodiments, the duration of the BOR is at least about 7 months or more. In some embodiments, the duration of a BOR is measured from the first occurrence of a complete response or partial response (e.g., as described below) to disease progression (e.g., according to the modified Lugano 2014 criteria). In some embodiments, the duration of a BOR is measured from the first occurrence of a complete response or a partial response to relapse (e.g., according to the modified Lugano 2014 criteria). In some embodiments, the duration of a BOR is measured from the first occurrence of a complete response or a partial response to disease progression or relapse (e.g., according to the modified Lugano 2014 criteria). In some embodiments, the duration of a BOR is measured from the first occurrence of a complete response or a partial response to disease progression or relapse (e.g., according to the modified Lugano 2014 criteria), or death from any cause.

In some embodiments, a best overall response (BOR) refers to the best response of a complete response or a partial response (i.e., to the occurrence of a complete response or a partial response), evaluated according to the modified Lugano 2014 criteria, as described in Example 1 herein. In some embodiments, a human treated according to the methods provided herein that achieves a best overall response (BOR) achieves a complete response or a partial response, assessed according to the modified Lugano 2014 criteria, as described in Example 1 herein. In some embodiments, responses are assessed on the basis of PET-CT scans according to the modified Lugano 2014 criteria, as described in Example 1 herein. In some embodiments, responses are assessed on the basis of CT scans alone according to the modified Lugano 2014 criteria, as described in Example 1 herein.

Complete responses are evaluated according to the modified Lugano 2014 criteria, as described in Example 1 herein.

Further details regarding clinical staging of and response criteria for lymphomas such as DLBCL are provided in, e.g., Van Heertum et al. (2017) Drug Des. Devel. Ther. 11: 1719-1728; Cheson et al. (2016) Blood. 128: 2489-2496; Cheson et al. (2014) J. Clin. Oncol. 32(27): 3059-3067; Barrington et al. (2017) J. Clin. Oncol. 32(27): 3048-3058; Gallamini et al. (2014) Haematologica. 99(6): 1107-1113; Barrinton et al. (2010) Eur. J. Nucl. Med. Mol. Imaging. 37(10): 1824-33; Moskwitz (2012) Hematology Am Soc. Hematol. Educ. Program 2012: 397-401; and Follows et al. (2014) Br. J. Haematology 166: 34-49. The progress of any one of the methods of treatment provided herein can be monitored by techniques known in the art.

In some embodiments, treatment of a plurality of humans according to the methods provided herein results in a six-month progression-free survival rate of at least about 25%, at least about 27%, at least about 29%, at least about 31%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, or 100% of the humans in the plurality. In some embodiments, treatment of a plurality of humans according to the methods provided herein results in a six-month progression-free survival rate of at least about 27% of the humans in the plurality. In some embodiments, treatment of a plurality of humans according to the methods provided herein results in a six-month progression-free survival rate of at least about 42% of the humans in the plurality. In some embodiments, treatment of a plurality of humans according to the methods provided herein results in a six-month progression-free survival rate of at least about 57% of the humans in the plurality. In some embodiments, treatment of a plurality of humans according to the methods provided herein results in a six-month progression-free survival rate of at least about 60% of the humans in the plurality.

In some embodiments, the progression-free survival rate refers to the proportion of humans in a plurality of humans treated according to the methods provided herein that exhibit progression-free survival at six months after initiation of treatment, e.g., with the immunoconjugate (e.g., polatuzumab vedotin-piiq), the Bcl-2 inhibitor (e.g., venetoclax), and the anti-CD20 antibody (e.g., rituximab or obinutuzumab). In some embodiments, progression-free survival refers to the time from initiation of treatment with the immunoconjugate (e.g., polatuzumab vedotin-piiq), the Bcl-2 inhibitor (e.g., venetoclax), and the anti-CD20 antibody (e.g., rituximab or obinutuzumab) to the time of the first occurrence of disease progression or relapse, or death from any cause.

In some embodiments, treatment of a human according to the methods provided herein results in progression-free survival of the human for at least about 1 month, at least about 2 months, at least about 3 months, at least about 4 months, at least about 5 months, at least about 6 months, at least about 7 months, at least about 8 months, at least about 9 months, at least about 12 months, or more. In some embodiments, treatment of a human according to the methods provided herein results in progression-free survival of the human for at least about 3 months or more. In some embodiments, treatment of a human according to the methods provided herein results in progression-free survival of the human for at least about 4 months or more. In some embodiments, treatment of a human according to the methods provided herein results in progression-free survival of the human for at least about 7 months or more.

In some embodiments, treatment of a plurality of humans according to the methods provided herein results in a median progression-free survival of the humans in the plurality of at least about 3 months or more. In some embodiments, treatment of a plurality of humans according to the methods provided herein results in a median progression-free survival of the humans in the plurality of at least about 4 months or more. In some embodiments, treatment of a plurality of humans according to the methods provided herein results in a median progression-free survival of the humans in the plurality of at least about 7 months or more. In some embodiments, treatment of a plurality of humans according to the methods provided herein results in a median progression-free survival of the humans in the plurality of between about 3 months and about 7 months or more.

In some embodiments, progression-free survival of a human treated according to the methods provided herein refers to the time from initiation of treatment with the immunoconjugate (e.g., polatuzumab vedotin-piiq), the Bcl-2 inhibitor (e.g., venetoclax), and the anti-CD20 antibody (e.g., rituximab or obinutuzumab) to the time of the first occurrence of disease progression or relapse, or death from any cause.

In some embodiments, treatment of a human according to the methods provided herein results in survival of the human for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more. In some embodiments, treatment of a human according to the methods provided herein results in survival of the human for at least about 6 months or more. In some embodiments, treatment of a human according to the methods provided herein results in survival of the human for at least about 7 months or more. In some embodiments, treatment of a human according to the methods provided herein results in survival of the human for at least about 11 months or more.

In some embodiments, survival of a human treated according to the methods provided herein is defined as the time from initiation of treatment with the immunoconjugate (e.g., polatuzumab vedotin-piiq), the Bcl-2 inhibitor (e.g., venetoclax), and the anti-CD20 antibody (e.g., rituximab or obinutuzumab) to the time of death from any cause.

In some embodiments, treatment of a plurality of humans according to the methods provided herein results in a median overall survival of the humans of at least about 3 months or more. In some embodiments, treatment of a plurality of humans according to the methods provided herein results in a median overall survival of the humans of at least about 6 months or more. In some embodiments, treatment of a plurality of humans according to the methods provided herein results in a median overall survival of the humans of at least about 7 months or more. In some embodiments, treatment of a plurality of humans according to the methods provided herein results in a median overall survival of the humans of at least about 11 months or more. In some embodiments, treatment of a plurality of humans according to the methods provided herein results in a median overall survival of the humans of at least about 12 months or more.

In some embodiments, overall survival is defined as the time from initiation of treatment with the immunoconjugate (e.g., polatuzumab vedotin-piiq), the Bcl-2 inhibitor (e.g., venetoclax), and the anti-CD20 antibody (e.g., rituximab or obinutuzumab) to the time of death from any cause.

In some embodiments, treatment of a human according to the methods provided herein results in a decrease in the sum of the product of diameters (SPD), e.g., as compared to prior to administration of the immunoconjugate (e.g., polatuzumab vedotin-piiq), the Bcl-2 inhibitor (e.g., venetoclax), and the anti-CD20 antibody (e.g., rituximab or obinutuzumab). In some embodiments, the decrease in SPD is a decrease of at least about 5%, at least about 10%, at least about 20%, at least about 30%, at least about 40%, at least about 50%, at least about 60%, at least about 70%, at least about 80%, at least about 90%, at least about 95%, at least about 99%, or 100%, e.g., as compared to prior to administration of the immunoconjugate (e.g., polatuzumab vedotin-piiq), the Bcl-2 inhibitor (e.g., venetoclax), and the anti-CD20 antibody (e.g., rituximab or obinutuzumab). In some embodiments, the decrease in SPD is a decrease of at least about 50%, e.g., as compared to prior to administration of the immunoconjugate (e.g., polatuzumab vedotin-piiq), the Bcl-2 inhibitor (e.g., venetoclax), and the anti-CD20 antibody (e.g., rituximab or obinutuzumab).

In some embodiments, among a plurality of humans treated according to the methods provided herein, about 40% or less (e.g., any of about 40% or less, about 37% or less, about 35% or less, about 30% or less, about 25% or less, about 20% or less, about 15% or less, about 10% or less, about 5% or less, about 2.5% or less, or about 1% or less) of the humans in the plurality experience a serious adverse event. In some embodiments, adverse events are assessed according to the adverse event severity grading scale for the NCI CTCAE (v4.0). In some embodiments, a serious adverse event is any adverse event that causes or leads to death, is life threatening, requires or prolongs inpatient hospitalization, results in persistent or significant disability or incapacity, is a congenital anomaly or birth defect in a neonate or infant born to a mother treated according to the methods provided herein, and/or is otherwise a significant medical event. In some embodiments, a significant medical event is one that jeopardizes the individual, or requires medical or surgical intervention to prevent death, a life threatening condition, hospitalization, prolonged hospitalization, persistent or significant disability or incapacity, or a congenital anomaly or birth defect in a neonate or infant born to a mother treated according to the methods provided herein.

In some embodiments, among a plurality of humans treated according to the methods provided herein, about 79% or less (e.g., any of about 79% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2.5% or less, or about 1% or less) of the humans in the plurality experience a Grade 3 or Grade 4 adverse event. In some embodiments, adverse events are assessed according to the adverse event severity grading scale for the NCI CTCAE (v4.0).

In some embodiments, the Bcl-2 inhibitor (e.g., venetoclax) and the anti-CD20 antibody (e.g., rituximab or obinutuzumab) are further administered post-induction, e.g., during a consolidation phase following the sixth 21-day cycle. The consolidation phase or “post-induction treatment” refers to a treatment phase following an induction phase. In some embodiments, the consolidation phase begins immediately after the end of the induction phase. In some embodiments, the induction phase and the consolidation phase are separated by an interval of time. In some embodiments, the consolidation phase begins at least about 1, 2, 3, 4, 5, 6, 7, 8, 9, or 10 weeks after the end of the induction phase.

In some embodiments, the Bcl-2 inhibitor and the anti-CD20 antibody are further administered during a consolidation phase after the sixth 21-day cycle of the induction phase, wherein the Bcl-2 inhibitor is administered orally at a dose of about 400 mg, about 600 mg, or about 800 mg once per day during the consolidation phase, and wherein the anti-CD20 antibody is administered intravenously at a dose of about 375 mg/m² once every two months during the consolidation phase.

In some embodiments, the venetoclax and the rituximab are further administered during a consolidation phase after the sixth 21-day cycle of the induction phase, wherein the venetoclax is administered orally at a dose of about 400 mg, about 600 mg, or about 800 mg once per day during the consolidation phase, and wherein the rituximab is administered intravenously at a dose of about 375 mg/m² once every two months during the consolidation phase. In some embodiments, the venetoclax and the obinutuzumab are further administered during a consolidation phase after the sixth 21-day cycle of the induction phase, wherein the venetoclax is administered orally at a dose of about 400 mg, about 600 mg, or about 800 mg once per day during the consolidation phase, and wherein the obinutuzumab is administered intravenously at a dose of about 1000 mg once every two months during the consolidation phase.

In some embodiments, the venetoclax and the rituximab are further administered during a consolidation phase after the sixth 21-day cycle of the induction phase, wherein the venetoclax is administered orally at a dose of about 400 mg once per day during the consolidation phase, and wherein the rituximab is administered intravenously at a dose of about 375 mg/m² once every two months during the consolidation phase.

In some embodiments, the venetoclax and the obinutuzumab are further administered during a consolidation phase after the sixth 21-day cycle of the induction phase, wherein the venetoclax is administered orally at a dose of about 400 mg once per day during the consolidation phase, and wherein the obinutuzumab is administered intravenously at a dose of about 1000 mg once every two months during the consolidation phase.

In some embodiments, the venetoclax and the rituximab are further administered during a consolidation phase after the sixth 21-day cycle of the induction phase, wherein the venetoclax is administered orally at a dose of about 600 mg once per day during the consolidation phase, and wherein the rituximab is administered intravenously at a dose of about 375 mg/m² once every two months during the consolidation phase.

In some embodiments, the venetoclax and the obinutuzumab are further administered during a consolidation phase after the sixth 21-day cycle of the induction phase, wherein the venetoclax is administered orally at a dose of about 600 mg once per day during the consolidation phase, and wherein the obinutuzumab is administered intravenously at a dose of about 1000 mg once every two months during the consolidation phase.

In some embodiments, the venetoclax and the rituximab are further administered during a consolidation phase after the sixth 21-day cycle of the induction phase, wherein the venetoclax is administered orally at a dose of about 800 mg once per day during the consolidation phase, and wherein the rituximab is administered intravenously at a dose of about 375 mg/m² once every two months during the consolidation phase.

In some embodiments, the venetoclax and the obinutuzumab are further administered during a consolidation phase after the sixth 21-day cycle of the induction phase, wherein the venetoclax is administered orally at a dose of about 800 mg once per day during the consolidation phase, and wherein the obinutuzumab is administered intravenously at a dose of about 1000 mg once every two months during the consolidation phase.

In some embodiments, the Bcl-2 inhibitor is administered for a maximum of 8 months (e.g., for a maximum of about 1 month, for a maximum of about 2 months, for a maximum of about 3 months, for a maximum of about 4 months, for a maximum of about 5 months, for a maximum of about 6 months, for a maximum of about 7 months, or for a maximum of about 8 months) during the consolidation phase. In some embodiments, the venetoclax is administered for a maximum of 8 months (e.g., for a maximum of about 1 month, for a maximum of about 2 months, for a maximum of about 3 months, for a maximum of about 4 months, for a maximum of about 5 months, for a maximum of about 6 months, for a maximum of about 7 months, or for a maximum of about 8 months) during the consolidation phase.

In some embodiments, the anti-CD20 antibody is administered during the consolidation phase starting on Day 1 of the second month after the sixth 21-day cycle of the induction phase. In some embodiments, the anti-CD20 antibody is administered for a maximum of 8 months (e.g., for a maximum of about 1 month, for a maximum of about 2 months, for a maximum of about 3 months, for a maximum of about 4 months, for a maximum of about 5 months, for a maximum of about 6 months, for a maximum of about 7 months, or for a maximum of about 8 months) during the consolidation phase.

In some embodiments, the rituximab is administered during the consolidation phase starting on Day 1 of the second month after the sixth 21-day cycle of the induction phase. In some embodiments, the rituximab is administered for a maximum of 8 months (e.g., for a maximum of about 1 month, for a maximum of about 2 months, for a maximum of about 3 months, for a maximum of about 4 months, for a maximum of about 5 months, for a maximum of about 6 months, for a maximum of about 7 months, or for a maximum of about 8 months) during the consolidation phase. In some embodiments, the obinutuzumab is administered during the consolidation phase starting on Day 1 of the second month after the sixth 21-day cycle of the induction phase. In some embodiments, the obinutuzumab is administered for a maximum of 8 months (e.g., for a maximum of about 1 month, for a maximum of about 2 months, for a maximum of about 3 months, for a maximum of about 4 months, for a maximum of about 5 months, for a maximum of about 6 months, for a maximum of about 7 months, or for a maximum of about 8 months) during the consolidation phase.

In some embodiments, the venetoclax and the rituximab are administered for a maximum of 8 months (e.g., for a maximum of about 1 month, for a maximum of about 2 months, for a maximum of about 3 months, for a maximum of about 4 months, for a maximum of about 5 months, for a maximum of about 6 months, for a maximum of about 7 months, or for a maximum of about 8 months) during the consolidation phase. In some embodiments, the venetoclax and the obinutuzumab are administered for a maximum of 8 months (e.g., for a maximum of about 1 month, for a maximum of about 2 months, for a maximum of about 3 months, for a maximum of about 4 months, for a maximum of about 5 months, for a maximum of about 6 months, for a maximum of about 7 months, or for a maximum of about 8 months) during the consolidation phase.

In some embodiments, the anti-CD20 antibody and the Bcl-2 inhibitor are administered sequentially during the consolidation phase. In some embodiments, the Bcl-2 inhibitor is administered prior to the anti-CD20 antibody on Day 1 of each of months 2, 4, 6, and 8 during the consolidation phase.

In some embodiments, the venetoclax and the rituximab are administered sequentially during the consolidation phase. In some embodiments, the venetoclax is administered prior to the rituximab on Day 1 of each of months 2, 4, 6, and 8 during the consolidation phase. In some embodiments, the venetoclax and the obinutuzumab are administered sequentially during the consolidation phase. In some embodiments, the venetoclax is administered prior to the obinutuzumab on Day 1 of each of months 2, 4, 6, and 8 during the consolidation phase.

In some embodiments, a month comprises 28 days.

In some embodiments, the human has an Eastern Cooperative Oncology Group Performance Status of 0, 1, or 2. In some embodiments, the human has DLBCL that relapsed or is refractory to a prior treatment with at least 1 prior chemoimmunotherapy regimen that included an anti-CD20 monoclonal antibody. In some embodiments, the DLBCL is histologically documented CD20-positive. In some embodiments, the DLBCL is Fluorodeoxyglucose-avid lymphoma (i.e., PET-positive lymphoma). In some embodiments, the human has at least one bi-dimensionally measurable lesion (>1.5 cm in its largest dimension by CT scan or magnetic resonance imaging). In some embodiments, the human does not have known CD20-negative status at relapse or progression. In some embodiments, the individual has not undergone a prior allogeneic stem cell transplant (SCT). In some embodiments, the individual has not completed an autologous SCT within 100 days prior to initiation of treatment according to a method provided herein. In some embodiments, the individual does not have Grade 3b FL. In some embodiments, the individual does not have history of transformation of indolent disease to DLBCL. In some embodiments, the individual does not have Grade 1 or greater peripheral neuropathy. In some embodiments, the individual does not have CNS lymphoma or leptomeningeal infiltration. In some embodiments, the individual is not receiving greater than 20 mg per day of a corticosteroid, e.g., prednisone. In some embodiments, the individual is receiving a stable dose of 20 mg/day or less of corticosteroids for at least about 4 weeks prior to initiation of treatment according to the methods provided herein (e.g., prior to Day 1 of Cycle 1). In some embodiments, the individual is administered up to 100 mg per day of a corticosteroid, e.g., prednisone, for up to 5 days prior to initiation of treatment according to the methods provided herein. In some embodiments, the individual is not taking or does not require a warfarin treatment. In some embodiments, the individual is not taking a strong or moderate CYP3A inhibitor such as fluconazole, ketoconazole, and clarithromycin, or a strong or moderate CYP3A inducer such as rifampin and carbamazepine within 7 days prior to initiation of treatment according to the methods provided herein. In some embodiments, the individual has not consumed grapefruit, grapefruit products, Seville oranges, Seville orange products (e.g., marmalade that contains Seville oranges), star fruit, or star fruit products within 3 days prior to initiation of treatment according to the methods provided herein. In some embodiments, the individual does not have a history of progression multifocal Leukoencephalopathy (PML). In some embodiments, the individual does not have significant cardiovascular or liver disease prior to initiation of treatment according to the methods provided herein. In some embodiments, the individual does not have inadequate renal or liver function, unless due to DLBCL, prior to initiation of treatment according to the methods provided herein. In some embodiments, the individual does not have inadequate hematologic function, unless due to DBLCL, prior to initiation of treatment according to the methods provided herein. In some embodiments, inadequate hematologic function is defined as hemoglobin <9 g/dL, absolute neutrophil count (ANC)<1.5×10⁹/L, platelet count <75×10⁹/L. In some embodiments, the individual does not have any of the following, unless due to underlying DLBCL: calculated creatinine clearance <50 mL/min with the use of 24-hour creatinine clearance or modified Cockcroft-Gault equation (eCCr; with use of the ideal body mass [IBM] instead of mass): eCCR=((140−Age)·IBM (kg)·[0.85 if female])/(72·serum creatinine (mg/dL)), or if serum creatinine is in gmol/L: eCCR=((140−Age)·IBM (kg)·[1.23 if male, 1.04 if female])/(serum creatinine (gmol/L)); aspartate aminotransferase (AST) or alanine aminotransferase (ALT) >2.5×by upper limit of normal (ULN); serum total bilirubin >1.5×ULN (or >3×ULN for patients with Gilbert syndrome); International Normalized Ratio (INR) or prothrombin time (PT) >1.5×ULN in the absence of therapeutic anticoagulation; or partial thromboplastin time (PTT) or activated PTT (aPTT) >1.5×ULN in the absence of a lupus anticoagulant. In some embodiments, the individual has DLBCL with an Ann Arbor stage of 1, 2, 3, or 4 prior to treatment according to the methods provided herein. In some embodiments, the individual has an International Prognostic Index (IPI) score of 0, 1, 2, 3, 4, or 5 prior to treatment according to the methods provided herein. In some embodiments, the individual has received at least one prior treatment (e.g., any of at least 1, at least 2, at least 3, at least 4, at least 5, at least 6, at least 7, or more) for DLBCL. In some embodiments, the individual has received a prior treatment for DLBCL comprising a chimeric antigen receptor (CAR) T cell therapy. In some embodiments, the individual has extranodal disease prior to treatment according to the methods provided herein. In some embodiments, the individual has bulky disease of 7 centimeters or greater prior to treatment according to the methods provided herein. In some embodiments, the individual has DLBCL that is refractory to a prior treatment comprising an anti-CD20 agent. In some embodiments, the individual has DLBCL that did not respond or progressed or relapsed within about 6 months from the end date of the last prior anti-lymphoma therapy administered to the individual prior to treatment according to the methods provided herein. In some embodiments, the individual has DLBCL that did not respond or progressed or relapsed within about 6 months from the end date of the first anti-lymphoma therapy administered to the individual prior to treatment according to the methods provided herein. In some embodiments, the individual has received an autologous bone marrow transplant prior to treatment according to the methods provided herein. In some embodiments, the individual has DLBCL with a cell of origin of activated B cell (ABC). In some embodiments, the individual has DLBCL with a cell of origin of germinal center B cell (GCB). The cell of origin may be assessed using any method known in the art, such as microarray-based methods (e.g., Lymphochip), immunohistochemistry (IHC), quantitative nuclease protection-based assays (e.g., the HTG EdgeSeq DLBCL COO assay), a NanoString assay (e.g., using the NanoString nCounter System), or a Lymph2Cx 20-gene assay. In some embodiments, the cell of origin is assessed using a NanoString assay. In some embodiments, the individual has BCL2-positive DLBCL. In some embodiments, the individual has BCL2-negative DLBCL. In some embodiments, the individual has MYC-positive DLBCL. In some embodiments, the individual has MYC-negative DLBCL. In some embodiments, the individual has double expresser DLBCL, i.e., DLBCL that is BCL2-positive and MYC-positive. In some embodiments, the individual has DLBCL that is not double expresser DLBCL. In some embodiments, expression of BCL2 and/or MYC is assessed using any method known in the art, such as Western blotting, enzyme-linked immunosorbent assay (ELISA), mass spectrometry, microarray-based methods, RNA sequencing, or immunohistochemistry. In some embodiments, expression of BCL2 and/or MYC is assessed using immunohistochemistry (IHC). In some embodiments, the DLBCL is determined to be BCL2-positive (BCL2+) as described in Morschhauser F, et al., Blood 2020, e.g., based on the percentage of tumor cells positively stained for BCL2 (e.g., ≥50% of tumor cells) and on the intensity of tumor cell staining using an IHC assay. In some embodiments, the DLBCL is determined to be MYC-positive (MYC+) if ≥40% of cells show MYC nuclear staining above background intensity using an IHC assay.

In some embodiments, the methods of treating DLBCL provided herein further include administering a prophylactic treatment for tumor lysis syndrome (TLS), for example, as described in Example 1 herein. In some embodiments, the prophylactic treatment for tumor lysis syndrome (TLS) comprises a uric acid-reducing agent and/or a hydration regimen prior to the start of treatment. In some embodiments, the hydration regimen comprises administering about 2 to about 3 liters per day of fluids (e.g., water, saline, or other suitable fluids), wherein the fluids are administered starting at about 24 hours to about 48 hours prior to the start of treatment. In some embodiments, the fluids are administered orally or intravenously. In some embodiments, the fluids are administered orally. In some embodiments, the fluids are administered intravenously. In some embodiments, the uric acid-reducing agent is allopurinol. In some embodiments, the allopurinol is administered orally at a dose of about 300 mg/day starting at about 72 hours prior to the first dose of venetoclax, and wherein administration of allopurinol continues for between about 3 to about 7 days after administration of the first dose of venetoclax. In some embodiments, the prophylactic treatment for tumor lysis syndrome (TLS) comprises administering rasburicase intravenously to humans with elevated uric acid levels before the start of treatment, wherein the rasburicase is administered until normalization of serum uric acid and other evidence (e.g., laboratory test results) of TLS.

In some embodiments, the methods of treating DLBCL provided herein further include treating or preventing adverse events as described in Example 1 herein. In some embodiments, the methods of treating DLBCL provided herein further include treating an occurrence of a hematologic adverse event, e.g., neutropenia and/or thrombocytopenia, as described in Example 1 herein. In some embodiments, the methods of treating DLBCL provided herein further include administering granulocyte colony stimulating factor (G-CSF) if a Grade 3 or Grade 4 adverse event of neutropenia occurs. In some embodiments, the methods of treating DLBCL provided herein further include administering one or more platelet transfusions if a Grade 3 or Grade 4 adverse event of thrombocytopenia occurs. Treatments for hematologic adverse events, such as administration of G-CSF and/or platelet transfusions may be administered to the patient according to methods known in the art and in a fashion consistent with good medical practice.

### V. Immunoconjugates Comprising an Anti-CD79b Antibody and a Drug/Cytotoxic Agent (“Anti-CD79b Immunoconjugates”)

In some embodiments, the anti-CD79b immunoconjugate comprises an anti-CD79b antibody (Ab) which targets a cancer cell (such as a follicular lymphoma (FL) cell), a drug moiety (D), and a linker moiety (L) that attaches Ab to D. In some embodiments, the anti-CD79b antibody is attached to the linker moiety (L) through one or more amino acid residues, such as lysine and/or cysteine. In some formula Ab-(L-D)p, wherein: (a) Ab is the anti-CD79b antibody which binds CD79b on the surface of a cancer cell (e.g., an FL cell); (b) L is a linker; (c) D is a cytotoxic agent; and (d) p ranges from 1-8.

An exemplary anti-CD79b immunoconjugate comprises Formula I:

Ab-(L-D)ₚ (I)

wherein p is 1 to about 20 (e.g., 1 to 15, 1 to 10, 1 to 8, 2 to 5, or 3 to 4). In some embodiments, the number of drug moieties that can be conjugated to the anti-CD79b antibody is limited by the number of free cysteine residues. In some embodiments, free cysteine residues are introduced into the antibody amino acid sequence by the methods described elsewhere herein. Exemplary anti-CD79b immunoconjugates of Formula I comprise, but are not limited to, anti-CD79b antibodies that comprise 1, 2, 3, or 4 engineered cysteine amino acids (Lyon, R. et al (2012) Methods in Enzym. 502:123-138). In some embodiments, one or more free cysteine residues are already present in the anti-CD79b antibody, without the use of engineering, in which case the existing free cysteine residues may be used to conjugate the anti-CD79b antibody to the drug/cytotoxic agent. In some embodiments, the anti-CD79b antibody is exposed to reducing conditions prior to conjugation of the antibody to the drug/cytotoxic agent in order to generate one or more free cysteine residues.

A. Exemplary Linkers

A “linker” (L) is a bifunctional or multifunctional moiety that can be used to link one or more drug moieties (D) to the anti-CD79b antibody (Ab) to form an anti-CD79b immunoconjugate of Formula I. In some embodiments, anti-CD79b immunoconjugate can be prepared using a linker having reactive functionalities for covalently attaching to the drug and to the anti-CD79b antibody. For example, in some embodiments, a cysteine thiol of the anti-CD79b antibody (Ab) can form a bond with a reactive functional group of a linker or a drug-linker intermediate to make the anti-CD79b immunoconjugate.

In one aspect, a linker has a functionality that is capable of reacting with a free cysteine present on the anti-CD79b antibody to form a covalent bond. Exemplary reactive functionalities include, without limitation, e.g., maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. See, e.g., the conjugation method at page 766 of Klussman, et al (2004), Bioconjugate Chemistry 15(4):765-773, and the Examples herein.

In some embodiments, a linker has a functionality that is capable of reacting with an electrophilic group present on the anti-CD79b antibody. Exemplary electrophilic groups include, without limitation, e.g., aldehyde and ketone carbonyl groups. In some embodiments, a heteroatom of the reactive functionality of the linker can react with an electrophilic group on an antibody and form a covalent bond to an antibody unit. Exemplary reactive functionalities include, but are not limited to, e.g., hydrazide, oxime, amino, hydrazine, thiosemicarbazone, hydrazine carboxylate, and arylhydrazide.

In some embodiments, the linker comprises one or more linker components. Exemplary linker components include, e.g., 6-maleimidocaproyl (“MC”), maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl) cyclohexane-1 carboxylate (“MCC”). Various linker components are known in the art, some of which are described below.

In some embodiments, the linker is a “cleavable linker,” facilitating release of a drug. Nonlimiting exemplary cleavable linkers include acid-labile linkers (e.g., comprising hydrazone), protease-sensitive (e.g., peptidase-sensitive) linkers, photolabile linkers, or disulfide-containing linkers (Chari et al., Cancer Research 52:127-131 (1992); U.S. Pat. No. 5,208,020).

In certain embodiments, a linker (L) has the following Formula II:

-Aₐ-W_{w}-Y_{y}- (II)

wherein A is a “stretcher unit,” and a is an integer from 0 to 1; W is an “amino acid unit,” and w is an integer from 0 to 12; Y is a “spacer unit,” and y is 0, 1, or 2; and Ab, D, and p are defined as above for Formula I. Exemplary embodiments of such linkers are described in U.S. Pat. No. 7,498,298, which is expressly incorporated herein by reference.

In some embodiments, a linker component comprises a “stretcher unit” that links an antibody to another linker component or to a drug moiety. Nonlimiting exemplary stretcher units are shown below (wherein the wavy line indicates sites of covalent attachment to an antibody, drug, or additional linker components):

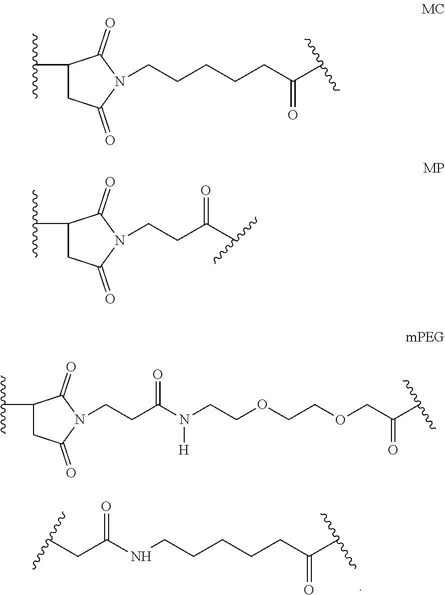

In some embodiments, a linker component comprises an “amino acid unit.” In some such embodiments, the amino acid unit allows for cleavage of the linker by a protease, thereby facilitating release of the drug/cytotoxic agent from the anti-CD79b immunoconjugate upon exposure to intracellular proteases, such as lysosomal enzymes (Doronina et al. (2003) Nat. Biotechnol. 21:778-784). Exemplary amino acid units include, but are not limited to, dipeptides, tripeptides, tetrapeptides, and pentapeptides. Exemplary dipeptides include, but are not limited to, valine-citrulline (vc or val-cit), alanine-phenylalanine (af or ala-phe); phenylalanine-lysine (fk or phe-lys); phenylalanine-homolysine (phe-homolys); and N-methyl-valine-citrulline (Me-val-cit). Exemplary tripeptides include, but are not limited to, glycine-valine-citrulline (gly-val-cit) and glycine-glycine-glycine (gly-gly-gly). An amino acid unit may comprise amino acid residues that occur naturally and/or minor amino acids and/or non-naturally occurring amino acid analogs, such as citrulline. Amino acid units can be designed and optimized for enzymatic cleavage by a particular enzyme, for example, a tumor-associated protease, cathepsin B, C and D, or a plasmin protease.

In some embodiments, a linker component comprises a “spacer” unit that links the antibody to a drug moiety, either directly or through a stretcher unit and/or an amino acid unit. A spacer unit may be “self-immolative” or a “non-self-immolative.” A “non-self-immolative” spacer unit is one in which part or all of the spacer unit remains bound to the drug moiety upon cleavage of the ADC. Examples of non-self-immolative spacer units include, but are not limited to, a glycine spacer unit and a glycine-glycine spacer unit. In some embodiments, enzymatic cleavage of an ADC containing a glycine-glycine spacer unit by a tumor-cell associated protease results in release of a glycine-glycine-drug moiety from the remainder of the ADC. In some such embodiments, the glycine-glycine-drug moiety is subjected to a hydrolysis step in the tumor cell, thus cleaving the glycine-glycine spacer unit from the drug moiety.

A “self-immolative” spacer unit allows for release of the drug moiety. In certain embodiments, a spacer unit of a linker comprises a p-aminobenzyl unit. In some such embodiments, a p-aminobenzyl alcohol is attached to an amino acid unit via an amide bond, and a carbamate, methylcarbamate, or carbonate is made between the benzyl alcohol and the drug (Hamann et al. (2005) Expert Opin. Ther. Patents (2005) 15:1087-1103). In some embodiments, the spacer unit is p-aminobenzyloxycarbonyl (PAB). In some embodiments, an anti-CD79b immunoconjugate comprises a self-immolative linker that comprises the structure:

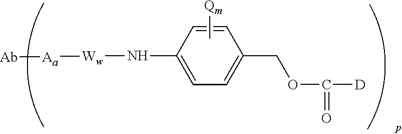

wherein Q is —C₁-C₈ alkyl, —O—(C₁-C₈ alkyl), -halogen, -nitro, or -cyno; m is an integer ranging from 0 to 4; and p ranges from 1 to about 20. In some embodiments, p ranges from 1 to 10, 1 to 7, 1 to 5, or 1 to 4.

Other examples of self-immolative spacers include, but are not limited to, aromatic compounds that are electronically similar to the PAB group, such as 2-aminoimidazol-5-methanol derivatives (U.S. Pat. No. 7,375,078; Hay et al. (1999) Bioorg. Med. Chem. Lett. 9:2237) and ortho- or para-aminobenzylacetals. In some embodiments, spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Linkage of a drug to the α-carbon of a glycine residue is another example of a self-immolative spacer that may be useful in ADC (Kingsbury et al (1984) J. Med. Chem. 27:1447).

In some embodiments, linker L may be a dendritic type linker for covalent attachment of more than one drug moiety to an antibody through a branching, multifunctional linker moiety (Sun et al (2002) Bioorganic & Medicinal Chemistry Letters 12:2213-2215; Sun et al (2003) Bioorganic & Medicinal Chemistry 11:1761-1768). Dendritic linkers can increase the molar ratio of drug to antibody, i.e. loading, which is related to the potency of the ADC. Thus, where an antibody bears only one reactive cysteine thiol group, a multitude of drug moieties may be attached through a dendritic linker.

Non-limiting exemplary linkers are shown below in the context of anti-CD79 immunoconjugates of Formulas III, IV, V:

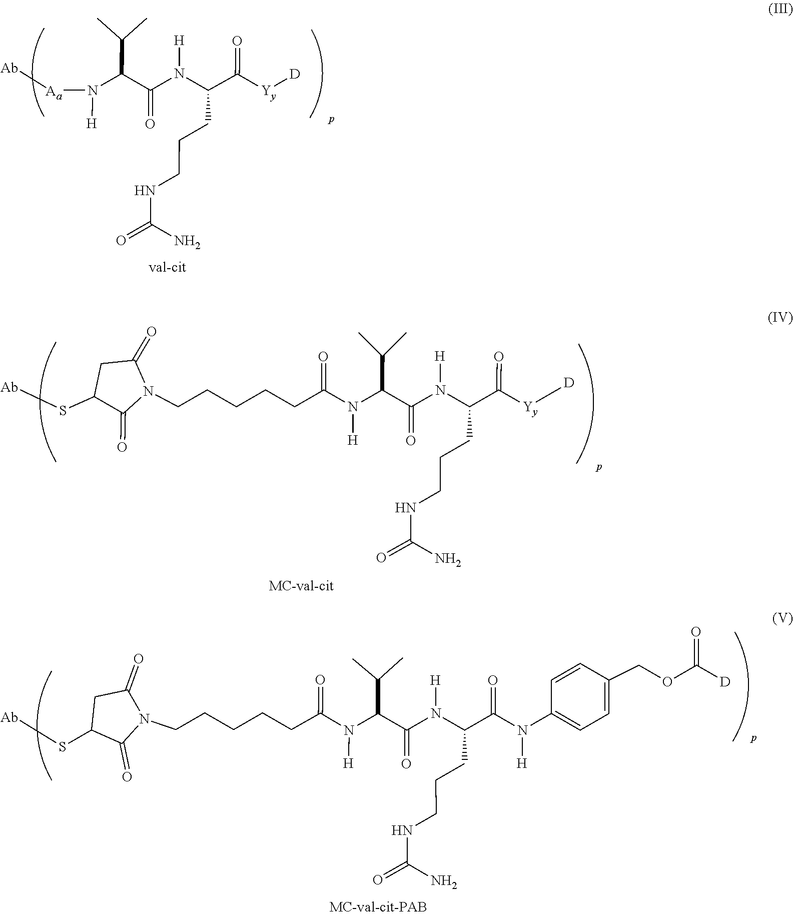

wherein (Ab) is an anti-CD79b antibody, (D) is a drug/cytotoxic agent, “Val-Cit” is a valine-citrulline dipeptide, MC is 6-maleimidocaproyl, PAB is p-aminobenzyloxycarbonyl, and p is 1 to about 20 (e.g., 1 to 15, 1 to 10, 1 to 8, 2 to 5, or 3 to 4).

In some embodiments, the anti-CD79b immunoconjugate comprises a structure of any one of formulas VI-X below:

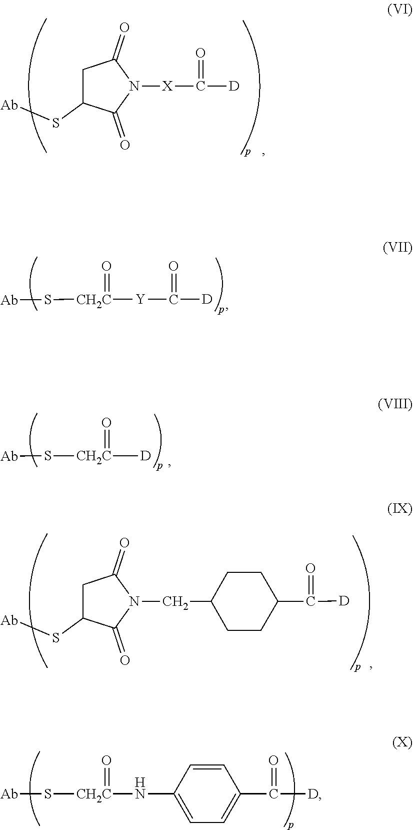

where X is:

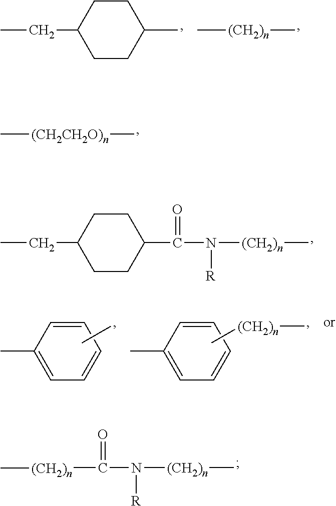

Y is:

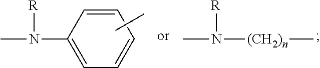

each R is independently H or C₁-C₆ alkyl; and n is 1 to 12.

Typically, peptide-type linkers can be prepared by forming a peptide bond between two or more amino acids and/or peptide fragments. Such peptide bonds can be prepared, for example, according to a liquid phase synthesis method (e.g., E. Schröder and K. Lübke (1965) “The Peptides”, volume 1, pp 76-136, Academic Press).

In some embodiments, a linker is substituted with groups that modulate solubility and/or reactivity. As a nonlimiting example, a charged substituent such as sulfonate (—SO₃⁻) or ammonium may increase water solubility of the linker reagent and facilitate the coupling reaction of the linker reagent with the antibody and/or the drug moiety, or facilitate the coupling reaction of Ab-L (anti-CD79b antibody-linker intermediate) with D, or D-L (drug/cytotoxic agent-linker intermediate) with Ab, depending on the synthetic route employed to prepare the anti-CD79b immunoconjugate. In some embodiments, a portion of the linker is coupled to the antibody and a portion of the linker is coupled to the drug, and then the anti-CD79 Ab-(linker portion)^{a} is coupled to drug/cytotoxic agent-(linker portion)^{b} to form the anti-CD79b immunoconjugate of Formula I. In some such embodiments, the anti-CD79b antibody comprises more than one (linker portion)^{a} substituents, such that more than one drug/cytotoxic agent is coupled to the anti-CD79b antibody in the anti-CD79b immunoconjugate of Formula I.

The anti-CD79b immunoconjugates provided herein expressly contemplate, but are not limited to, anti-CD79b immunoconjugates prepared with the following linker reagents: bis-maleimido-trioxyethylene glycol (BMPEO), N-(β-maleimidopropyloxy)-N-hydroxy succinimide ester (BMPS), N-(ε-maleimidocaproyloxy) succinimide ester (EMCS), N-[γ-maleimidobutyryloxy]succinimide ester (GMBS), 1,6-hexane-bis-vinylsulfone (HBVS), succinimidyl 4-(N-maleimidomethyl)cyclohexane-1-carboxy-(6-amidocaproate) (LC-SMCC), m-maleimidobenzoyl-N-hydroxysuccinimide ester (MBS), 4-(4-N-Maleimidophenyl)butyric acid hydrazide (MPBH), succinimidyl 3-(bromoacetamido)propionate (SBAP), succinimidyl iodoacetate (SIA), succinimidyl (4-iodoacetyl)aminobenzoate (SIAB), N-succinimidyl-3-(2-pyridyldithio) propionate (SPDP), N-succinimidyl-4-(2-pyridylthio)pentanoate (SPP), succinimidyl 4-(N-maleimidomethyl)cyclohexane-1-carboxylate (SMCC), succinimidyl 4-(p-maleimidophenyl)butyrate (SMPB), succinimidyl 6-[(beta-maleimidopropionamido)hexanoate] (SMPH), iminothiolane (IT), sulfo-EMCS, sulfo-GMBS, sulfo-KMUS, sulfo-MBS, sulfo-SIAB, sulfo-SMCC, and sulfo-SMPB, and succinimidyl-(4-vinylsulfone)benzoate (SVSB), and including bis-maleimide reagents: dithiobismaleimidoethane (DTME), 1,4-Bismaleimidobutane (BMB), 1,4 Bismaleimidyl-2,3-dihydroxybutane (BMDB), bismaleimidohexane (BMH), bismaleimidoethane (BMOE), BM(PEG)₂ (shown below), and BM(PEG)₃ (shown below); bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). In some embodiments, bis-maleimide reagents allow the attachment of the thiol group of a cysteine in the antibody to a thiol-containing drug moiety, linker, or linker-drug intermediate. Other functional groups that are reactive with thiol groups include, but are not limited to, iodoacetamide, bromoacetamide, vinyl pyridine, disulfide, pyridyl disulfide, isocyanate, and isothiocyanate.

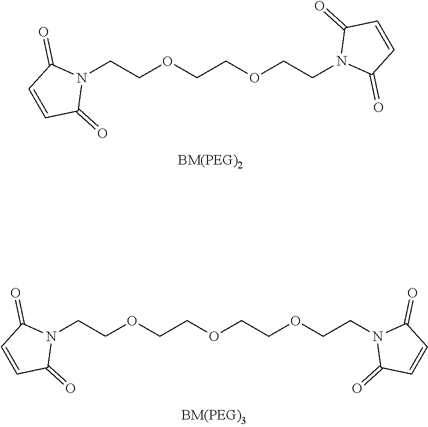 [SMILES: O=C1C=CC(=O)N1CCOCCOCCN1C(=O)C=CC1=O ; O=C1C=CC(=O)N1CCOCCOCCOCCN1C(=O)C=CC1=O]

Certain useful linker reagents can be obtained from various commercial sources, such as Pierce Biotechnology, Inc. (Rockford, Ill.), Molecular Biosciences Inc. (Boulder, Colo.), or synthesized in accordance with procedures described in the art; for example, in Toki et al (2002) J. Org. Chem. 67:1866-1872; Dubowchik, et al. (1997) Tetrahedron Letters, 38:5257-60; Walker, M. A. (1995) J. Org. Chem. 60:5352-5355; Frisch et al (1996) Bioconjugate Chem. 7:180-186; U.S. Pat. No. 6,214,345; WO 02/088172; US 2003130189; US2003096743; WO 03/026577; WO 03/043583; and WO 04/032828.

Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026.

B. Anti-CD79b Antibodies

In some embodiments, the immunoconjugate (e.g., anti-CD79b immunoconjugate) comprises an anti-CD79b antibody that comprises at least one, two, three, four, five, or six HVRs selected from (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some such embodiments, the immunoconjugate comprises an anti-CD79 antibody comprising at least one of: (i) HVR-H3 comprising the amino acid sequence of SEQ ID NO: 23, and/or (ii) HVR-L1 comprising an amino acid sequence of SEQ ID NO: 24. In some embodiments, the immunoconjugate comprises an anti-CD79 antibody comprising at least one of: (i) HVR-H3 comprising the amino acid sequence of SEQ ID NO: 23, and/or (ii) HVR-L1 comprising the amino acid sequence of SEQ ID NO: 24. In some embodiments, the immunoconjugate comprises an anti-CD79b antibody comprising at least one, at least two, or all three VH HVR sequences selected from (a) HVR-H1 comprising the amino acid sequence of SEQ ID NO: 21; (b) HVR-H2 comprising the amino acid sequence of SEQ ID NO: 22; and (c) HVR-H3 comprising the amino acid sequence of SEQ ID NO: 23. In some embodiments, the immunoconjugate comprises an anti-CD79b antibody that comprises an HVR-H3 comprising the amino acid sequence of SEQ ID NO: 23. In some embodiments, the immunoconjugate comprises an anti-CD79b antibody that comprises an HVR-H3 comprising the amino acid sequence of SEQ ID NO: 23 and an HVR-L3 comprising the amino acid sequence of SEQ ID NO: 26. In some embodiments, the immunoconjugate comprises an anti-CD79b antibody that comprises an HVR-H3 comprising the amino acid sequence of SEQ ID NO: 23, an HVR-L3 comprising the amino acid sequence of SEQ ID NO: 26, and an HVR-H2 comprising the amino acid sequence of SEQ ID NO: 22. In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; and (c) HVR-H3 comprising the amino acid sequence of SEQ ID NO: 23.

In some embodiments, the immunoconjugate comprises an anti-CD79b antibody comprising at least one, at least two, or all three VL HVR sequences selected from (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises an anti-CD79b antibody that comprises at least one, at least two, or all three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the immunoconjugate comprises an anti-CD79b antibody that comprises an HVR-L1 comprising the amino acid sequence of SEQ ID NO: 24 In some embodiments, the immunoconjugate comprises an anti-CD79b antibody that comprises (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, the immunoconjugate comprises an anti-CD79b antibody comprising (a) a VH domain comprising at least one, at least two, or all three VH HVR sequences selected from (i) HVR-H1 comprising the amino acid sequence of SEQ ID NO: 21, (ii) HVR-H2 comprising the amino acid sequence of SEQ ID NO: 22, and (iii) HVR-H3 comprising an amino acid sequence selected from SEQ ID NO:23; and (b) a VL domain comprising at least one, at least two, or all three VL HVR sequences selected from (i) HVR-L1 comprising an amino acid sequence of SEQ ID NO: 24, (ii) HVR-L2 comprising the amino acid sequence of SEQ ID NO: 25, and (iii) HVR-L3 comprising the amino acid sequence of SEQ ID NO: 26. In some embodiments, the immunoconjugate comprises an anti-CD79b antibody that comprises at least one of: (i) HVR-H3 comprising the amino acid sequence of SEQ ID NO: 23, and/or (ii) HVR-L1 comprising the amino acid sequence of SEQ ID NO: 24.

In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some embodiments, the immunoconjugate comprises at least one of: HVR-H3 comprising the amino acid sequence of SEQ ID NO: 23 and/or HVR-L1 comprising an amino acid sequence of SEQ ID NO: 24. In some embodiments, the immunoconjugate comprises an anti-CD79b antibody that comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, the anti-CD79b immunoconjugates comprises a humanized anti-CD79b antibody. In some embodiments, an anti-CD79b antibody comprises HVRs as in any of the embodiments provided herein, and further comprises a human acceptor framework, e.g., a human immunoglobulin framework or a human consensus framework. In some embodiments, the human acceptor framework is the human VL kappa 1 (VL_{KI}) framework and/or the VH framework VH_{III}. In some embodiments, a humanized anti-CD79b antibody comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising an amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some embodiments, a humanized anti-CD79b antibody comprises (a) HVR-H1 comprising the amino acid sequence of SEQ ID NO: 21; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, the immunoconjugate (e.g., the anti-CD79b immunoconjugate) comprises an anti-CD79 antibody comprising a heavy chain variable domain (VH) sequence having at least 90%, 91%, 92%, 93%, 94%, 95%, 96%, 97%, 98%, 99%, or 100% sequence identity to the amino acid sequence of SEQ ID NO: 19. In some embodiments, a VH sequence having at least 90%, 91%, 92%, 93%, 94%, 95%, 96%, 97%, 98%, or 99% identity to the amino acid sequence of SEQ ID NO: 19 contains substitutions (e.g., conservative substitutions), insertions, or deletions relative to the reference sequence, but an anti-CD79b immunoconjugate comprising that sequence retains the ability to bind to CD79b. In some embodiments, a total of 1 to 10 amino acids have been substituted, inserted and/or deleted in SEQ ID NO: 19. In some embodiments, a total of 1 to 5 amino acids have been substituted, inserted and/or deleted in SEQ ID NO: 19. In some embodiments, substitutions, insertions, or deletions occur in regions outside the HVRs (i.e., in the FRs). In some embodiments, the immunoconjugate (e.g., the anti-CD79b immunoconjugate) comprises the VH sequence of SEQ ID NO: 19, including post-translational modifications of that sequence. In some embodiments, the VH comprises one, two or three HVRs selected from: (a) HVR-H1 comprising the amino acid sequence of SEQ ID NO: 21, (b) HVR-H2 comprising the amino acid sequence of SEQ ID NO: 22, and (c) HVR-H3 comprising the amino acid sequence of SEQ ID NO: 17 or SEQ ID NO: 23.

In some embodiments, the immunoconjugate (e.g., the anti-CD79b immunoconjugate) comprises an anti-CD79b antibody that comprises a light chain variable domain (VL) having at least 90%, 91%, 92%, 93%, 94%, 95%, 96%, 97%, 98%, 99%, or 100% sequence identity to the amino acid sequence of SEQ ID NO: 20. In certain embodiments, a VL sequence having at least 90%, 91%, 92%, 93%, 94%, 95%, 96%, 97%, 98%, or 99% identity to the amino acid sequence of SEQ ID NO: 20 contains substitutions (e.g., conservative substitutions), insertions, or deletions relative to the reference sequence, but an anti-CD79b immunoconjugate comprising that sequence retains the ability to bind to CD79b. In certain embodiments, a total of 1 to 10 amino acids have been substituted, inserted and/or deleted in SEQ ID NO: 20. In certain embodiments, a total of 1 to 5 amino acids have been substituted, inserted and/or deleted in SEQ ID NO: 20. In certain embodiments, the substitutions, insertions, or deletions occur in regions outside the HVRs (i.e., in the FRs). In some embodiments, the anti-CD79b immunoconjugate comprises an anti-CD79b antibody that comprises the VL sequence of SEQ ID NO: 20, including post-translational modifications of that sequence. In some embodiments, the VL comprises one, two or three HVRs selected from (a) HVR-L1 comprising an amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the VL comprises one, two or three HVRs selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, the immunoconjugate (e.g., the anti-CD79b immunoconjugate) comprises an anti-CD79b antibody that comprises a VH as in any of the embodiments provided herein, and a VL as in any of the embodiments provided herein. In some embodiments, the immunoconjugate comprises an anti-CD79b antibody that comprises the VH and VL sequences in SEQ ID NO: 19 and SEQ ID NO: 20, respectively, including post-translational modifications of those sequences.

In some embodiments, the immunoconjugate (e.g., anti-CD79b immunoconjugate) comprises an anti-CD79b antibody that binds to the same epitope as an anti-CD79b antibody described herein. For example, in some embodiments, the immunoconjugate (e.g., anti-CD79b immunoconjugate) comprises an anti-CD79b antibody that binds to the same epitope as an anti-CD79b antibody comprising a VH sequence of SEQ ID NO: 19 and a VL sequence of SEQ ID NO: 20.

In some embodiments, the immunoconjugate comprises an anti-CD79b antibody that is a monoclonal antibody, a chimeric antibody, humanized antibody, or human antibody. In some embodiments, immunoconjugate comprises an antigen-binding fragment of an anti-CD79b antibody described herein, e.g., a Fv, Fab, Fab′, scFv, diabody, or F(ab′)₂ fragment. In some embodiments, the immunoconjugate comprises a substantially full length anti-CD79b antibody, e.g., an IgG1 antibody or other antibody class or isotype as described elsewhere herein.

In some embodiments, the immunoconjugate comprises an anti-CD79b antibody comprising a heavy chain comprises the amino acid sequence of SEQ ID NO: 36, and wherein the light chain comprises the amino acid sequence of SEQ ID NO: 35. In some embodiments, the immunoconjugate comprises an anti-CD79 antibody that comprises a heavy chain comprising the amino acid sequence of SEQ ID NO: 37 and a light chain comprising the amino acid sequence of SEQ ID NO: 35. In some embodiments, the immunoconjugate comprises an anti-CD79 antibody that comprises a heavy chain comprising the amino acid sequence of SEQ ID NO: 36 and a light chain comprising the amino acid sequence of SEQ ID NO: 38.

In some embodiments, the immunoconjugate comprises an anti-CD79b antibody comprising a heavy chain comprising the amino acid sequence of SEQ ID NO: 37 and a light chain comprising the amino acid sequence of SEQ ID NO: 35. In some embodiments, the immunoconjugate is iladatuzumab vedotin. In certain embodiments, p is between 2 and 5. In certain embodiments, p is 2. In some embodiments, the immunoconjugate is administered at a dose that is from about 1 mg/kg to about 5 mg/kg. In some embodiments, the immunoconjugate is administered at a dose of about 1.2 mg/kg, about 1.8 mg/kg, about 2.4 mg/kg, about 3.6 mg/kg, or about 4.8 mg/kg. In some embodiments, the immunoconjugate is administered at a dose of about 1.8 mg/kg.

As used herein, the term “iladatuzumab vedotin” refers to an anti-CD79b immunoconjugate having the International Nonproprietary Names for Pharmaceutical Substances (INN) Number 10647, or the CAS Registry Number 1906205-77-3. Iladatuzumab vedotin is also interchangeably referred to as “DCDS0780A” or “R07032005”.

In some embodiments, the immunoconjugate is polatuzumab vedotin-piiq, as described in WHO Drug Information, Vol. 26, No. 4, 2012 (Proposed INN: List 108), which is expressly incorporated by reference herein in its entirety. As shown in WHO Drug Information, Vol. 26, No. 4, 2012, polatuzumab vedotin-piiq has the following structure: immunoglobulin G1-kappa auristatin E conjugate, anti-[Homo sapiens CD79B (immunoglobulin-associated CD79 beta)], humanized monoclonal antibody conjugated to auristatin E; gammal heavy chain (1-447) [humanized VH (Homo sapiens IGHV3-66*01 (79.60%) -(IGHD)-IGHJ4*01) [8.8.13] (1-120) -Homo sapiens IGHG1*03 (CH1 R120>K (214) (121-218), hinge (219-233), CH2 (234-343), CH3 (344-448), CHS (449-450)) (121-450)], (220-218′)-disulfide (if not conjugated) with kappa light chain (1′-218′)[humanized V-KAPPA (Homo sapiens IGKV1-39*01 (80.00%) -IGKJ1*01) [11.3.9] (1′-112′) -Homo sapiens IGKC*01 (113′-218′)]; dimer (226-226″:229-229″)-bisdisulfide; conjugated, on an average of 3 to 4 cysteinyl, to monomethylauristatin E (MMAE), via a cleavable maleimidecaproyl-valyl-citrullinyl-p-aminobenzylcarbamate (mc-val-cit-PABC) linker; the heavy chain of polatuzumab has the following sequence:

| (SEQ ID NO: 56) | |
|---|---|
| EVQLVESGGG LVQPGGSLRL SCAASGYTFS SYWIEWVRQA PGKGLEWIGE | 50 |
| ILPGGGDTNY NEIFKGRATF SADTSKNTAY LQMNSLRAED TAVYYCTRRV | 100 |
| PIRLDYWGQG TLVTVSSAST KGPSVFPLAP SSKSTSGGTA ALGCLVKDYF | 150 |
| PEPVTVSWNS GALTSGVHTF PAVLQSSGLY SLSSVVTVPS SSLGTQTYIC | 200 |
| NVNHKPSNTK VDKKVEPKSC DKTHTCPPCP APELLGGPSV FLFPPKPKDT | 250 |
| LMISRTPEVT CVVVDVSHED PEVKFNWYVD GVEVHNAKTK PREEQYNSTY | 300 |
| RVVSVLTVLH QDWLNGKEYK CKVSNKALPA PIEKTISKAK GQPREPQVYT | 350 |
| LPPSREEMTK NQVSLTCLVK GFYPSDIAVE WESNGQPENN YKTTPPVLDS | 400 |
| DGSFFLYSKL TVDKSRWQQG NVFSCSVMHE ALHNHYTQKS LSLSPGK; | 447 |

the light chain of polatuzumab has the following sequence:

| (SEQ ID NO: 35) | |
|---|---|
| DIQLTQSPSS LSASVGDRVT ITCKASQSVD YEGDSFLNWY QQKPGKAPKL | 50 |
| LIYAASNLES GVPSRFSGSG SGTDFTLTIS SLQPEDFATY YCQQSNEDPL | 100 |
| TFGQGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC; | 218 |

the disulfide bridge locations are:
Intra-H 22-96 144-200 261-321 367-425
   22″-96″ 147-203″ 261″-321″ 367-425″
Intra-L 23′-92′ 138′-198′
   23′-92′″ 138′-198′″
Inter-H-L* 220-218′ 220″-218′″
218 I—H-H* 226-226″ 229-229″
Two or three of the inter-chain disulfide bridges are not present, the antibody being conjugated to an average of 3 to 4 drug linkers each via a thioether bond;
the N-glycosylation sites are H CH2 N84.4: 297, 297″ but lacking carbohydrate;
and other post-translational modifications are: lacking H chain C-terminal lysine.

C. Drugs/Cytotoxic Agents

Anti-CD79 immunoconjugates comprise an anti-CD79b antibody (e.g., an anti-CD79b antibody described herein) conjugated to one or more drugs/cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes (i.e., a radioconjugate). Such immunoconjugates are targeted chemotherapeutic molecules which combine properties of both antibodies and cytotoxic drugs by targeting potent cytotoxic drugs to antigen-expressing cancer cells (such as tumor cells) (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107. That is, the anti-CD79 immunoconjugates selectively deliver an effective dose of a drug to cancerous cells/tissues whereby greater selectivity, i.e. a lower efficacious dose, may be achieved while increasing the therapeutic index (“therapeutic window”) (Polakis P. (2005) Current Opinion in Pharmacology 5:382-387).

Anti-CD79 immunoconjugates used in the methods provided herein include those with anticancer activity. In some embodiments, the anti-CD79 immunoconjugate comprises an anti-CD79b antibody conjugated, i.e. covalently attached, to the drug moiety. In some embodiments, the anti-CD79b antibody is covalently attached to the drug moiety through a linker. The drug moiety (D) of the anti-CD79 immunoconjugate may include any compound, moiety or group that has a cytotoxic or cytostatic effect. Drug moieties may impart their cytotoxic and cytostatic effects by mechanisms including but not limited to tubulin binding, DNA binding or intercalation, and inhibition of RNA polymerase, protein synthesis, and/or topoisomerase. Exemplary drug moieties include, but are not limited to, a maytansinoid, dolastatin, auristatin, calicheamicin, anthracycline, duocarmycin, vinca alkaloid, taxane, trichothecene, CC1065, camptothecin, elinafide, and stereoisomers, isosteres, analogs, and derivatives thereof that have cytotoxic activity.

(i) Maytansine and Maytansinoids

In some embodiments, an anti-CD79b immunoconjugate comprises an anti-CD79b antibody conjugated to one or more maytansinoid molecules. Maytansinoids are derivatives of maytansine, and are mitotic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinoids are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533.

Maytansinoid drug moieties are attractive drug moieties in antibody-drug conjugates because they are: (i) relatively accessible to prepare by fermentation or chemical modification or derivatization of fermentation products, (ii) amenable to derivatization with functional groups suitable for conjugation through non-disulfide linkers to antibodies, (iii) stable in plasma, and (iv) effective against a variety of tumor cell lines.

Certain maytansinoids suitable for use as maytansinoid drug moieties are known in the art and can be isolated from natural sources according to known methods or produced using genetic engineering techniques (see, e.g., Yu et al (2002) PNAS 99:7968-7973). Maytansinoids may also be prepared synthetically according to known methods.

Exemplary maytansinoid drug moieties include, but are not limited to, those having a modified aromatic ring, such as: C-19-dechloro (U.S. Pat. No. 4,256,746) (prepared, for example, by lithium aluminum hydride reduction of ansamitocin P2); C-20-hydroxy (or C-20-demethyl)+/−C-19-dechloro (U.S. Pat. Nos. 4,361,650 and 4,307,016) (prepared, for example, by demethylation using Streptomyces or Actinomyces or dechlorination using LAH); and C-20-demethoxy, C-20-acyloxy (—OCOR), +/−dechloro (U.S. Pat. No. 4,294,757) (prepared, for example, by acylation using acyl chlorides), and those having modifications at other positions of the aromatic ring.

Exemplary maytansinoid drug moieties also include those having modifications such as: C-9-SH (U.S. Pat. No. 4,424,219) (prepared, for example, by the reaction of maytansinol with H₂S or P₂S5); C-14-alkoxymethyl(demethoxy/CH₂OR)(U.S. Pat. No. 4,331,598); C-14-hydroxymethyl or acyloxymethyl (CH₂OH or CH₂OAc) (U.S. Pat. No. 4,450,254) (prepared, for example, from Nocardia); C-15-hydroxy/acyloxy (U.S. Pat. No. 4,364,866) (prepared, for example, by the conversion of maytansinol by Streptomyces); C-15-methoxy (U.S. Pat. Nos. 4,313,946 and 4,315,929) (for example, isolated from Trewia nudlflora); C-18-N-demethyl (U.S. Pat. Nos. 4,362,663 and 4,322,348) (prepared, for example, by the demethylation of maytansinol by Streptomyces); and 4,5-deoxy (U.S. Pat. No. 4,371,533) (prepared, for example, by the titanium trichloride/LAH reduction of maytansinol).

Many positions on maytansinoid compounds are useful as the linkage position. For example, an ester linkage may be formed by reaction with a hydroxyl group using conventional coupling techniques. In some embodiments, the reaction may occur at the C-3 position having a hydroxyl group, the C-14 position modified with hydroxymethyl, the C-15 position modified with a hydroxyl group, and the C-20 position having a hydroxyl group. In some embodiments, the linkage is formed at the C-3 position of maytansinol or a maytansinol analogue.

Maytansinoid drug moieties include those having the structure:

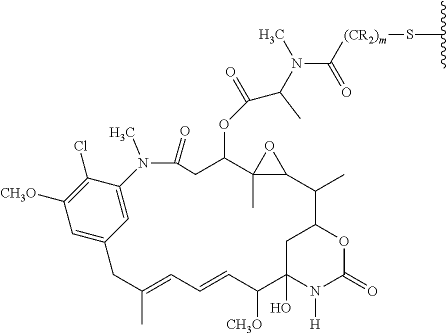

wherein the wavy line indicates the covalent attachment of the sulfur atom of the maytansinoid drug moiety to a linker of an anti-CD79b immunoconjugate. Each R may independently be H or a C₁-C₆ alkyl. The alkylene chain attaching the amide group to the sulfur atom may be methanyl, ethanyl, or propyl, i.e., m is 1, 2, or 3 (US 633410; U.S. Pat. No. 5,208,020; Chari et al (1992) Cancer Res. 52:127-131; Liu et al (1996) Proc. Natl. Acad. Sci USA 93:8618-8623).

All stereoisomers of the maytansinoid drug moiety are contemplated for the anti-CD79b immunoconjugate used in a method provided herein, i.e. any combination of R and S configurations at the chiral carbons (U.S. Pat. Nos. 7,276,497; 6,913,748; 6,441,163; US 633410 (RE39151); U.S. Pat. No. 5,208,020; Widdison et al (2006) J. Med. Chem. 49:4392-4408, which are incorporated by reference in their entirety). In some embodiments, the maytansinoid drug moiety has the following stereochemistry:

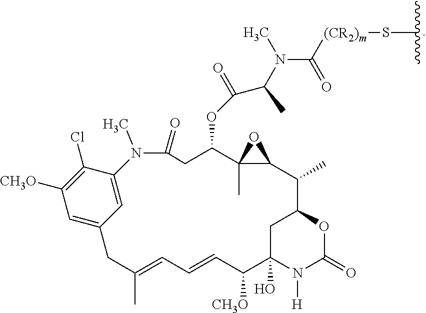

Exemplary embodiments of maytansinoid drug moieties include, but are not limited to, DM1; DM3; and DM4, having the structures:

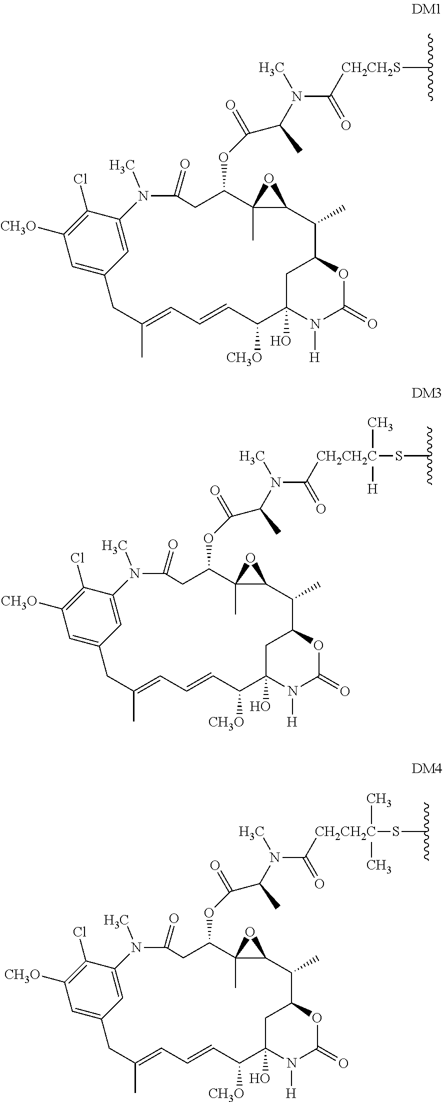

wherein the wavy line indicates the covalent attachment of the sulfur atom of the drug to a linker (L) of an anti-CD79b immunoconjugate.

Other exemplary maytansinoid anti-CD79b immunoconjugates have the following structures and abbreviations (wherein Ab is an anti-CD79b antibody and p is 1 to about 20. In some embodiments, p is 1 to 10, p is 1 to 7, p is 1 to 5, or p is 1 to 4):

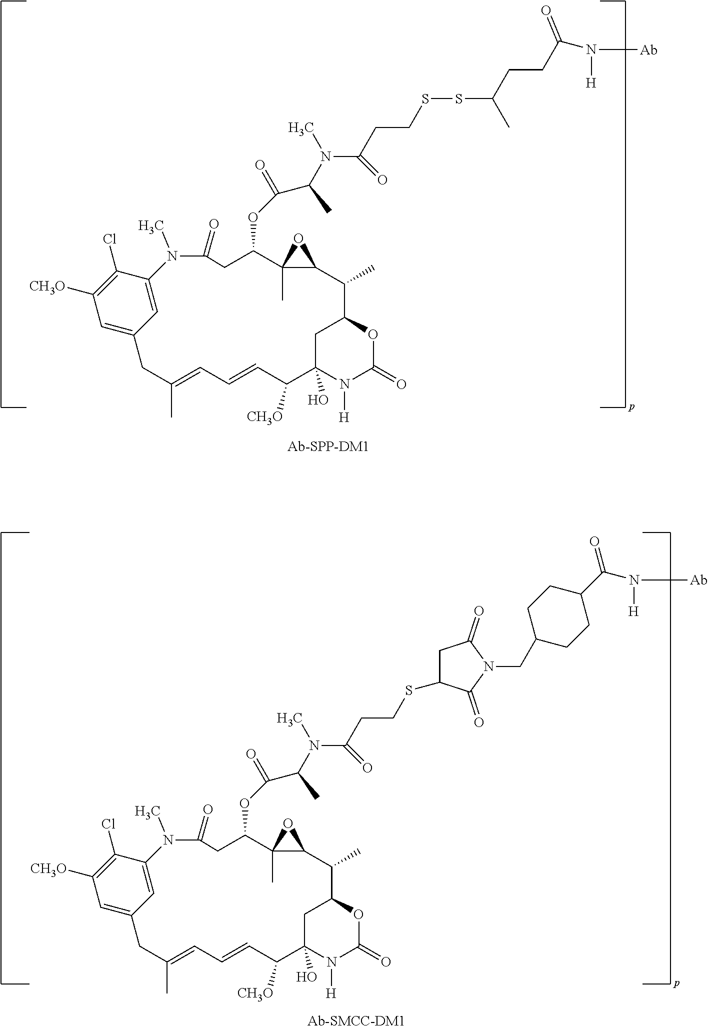

Exemplary antibody-drug conjugates where DM1 is linked through a BMPEO linker to a thiol group of the antibody have the structure and abbreviation:

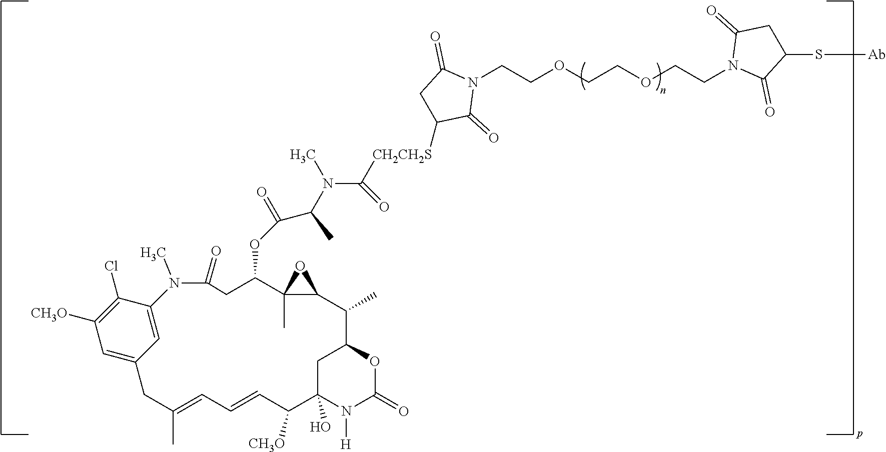

wherein Ab is an anti-CD79b antibody; n is 0, 1, or 2; and p is 1 to about 20. In some embodiments, p is 1 to 10, p is 1 to 7, p is 1 to 5, or p is 1 to 4.

Immunoconjugates containing maytansinoids, methods of making the same, and their therapeutic use are disclosed, for example, in U.S. Pat. Nos. 5,208,020 and 5,416,064; US 2005/0276812 A1; and European Patent EP 0 425 235 B1, the disclosures of which are hereby expressly incorporated by reference. See also Liu et al. Proc. Natl. Acad. Sci. USA 93:8618-8623 (1996); and Chari et al. Cancer Research 52:127-131 (1992).

In some embodiments, anti-CD79b antibody-maytansinoid conjugates may be prepared by chemically linking an anti-CD79b antibody to a maytansinoid molecule without significantly diminishing the biological activity of either the antibody or the maytansinoid molecule. See, e.g., U.S. Pat. No. 5,208,020 (the disclosure of which is hereby expressly incorporated by reference). In some embodiments, an anti-CD79b immunoconjugate with an average of 3-4 maytansinoid molecules conjugated per antibody molecule has shown efficacy in enhancing cytotoxicity of target cells without negatively affecting the function or solubility of the antibody. In some instances, even one molecule of toxin/antibody is expected to enhance cytotoxicity over the use of naked anti-CD79b antibody.

Exemplary linking groups for making antibody-maytansinoid conjugates include, for example, those described herein and those disclosed in U.S. Pat. No. 5,208,020; EP Patent 0 425 235 B1; Chari et al. Cancer Research 52:127-131 (1992); US 2005/0276812 A1; and US 2005/016993 A1, the disclosures of which are hereby expressly incorporated by reference.

(2) Auristatins and Dolastatins

Drug moieties include dolastatins, auristatins, and analogs and derivatives thereof (U.S. Pat. Nos. 5,635,483; 5,780,588; 5,767,237; 6,124,431). Auristatins are derivatives of the marine mollusk compound dolastatin-10. While not intending to be bound by any particular theory, dolastatins and auristatins have been shown to interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). The dolastatin/auristatin drug moiety may be attached to the antibody through the N (amino) terminus or the C (carboxyl) terminus of the peptidic drug moiety (WO 02/088172; Doronina et al (2003) Nature Biotechnology 21(7):778-784; Francisco et al (2003) Blood 102(4):1458-1465).

Exemplary auristatin embodiments include the N-terminus linked monomethylauristatin drug moieties D_{E} and D_{F}, disclosed in U.S. Pat. Nos. 7,498,298 and 7,659,241, the disclosures of which are expressly incorporated by reference in their entirety:

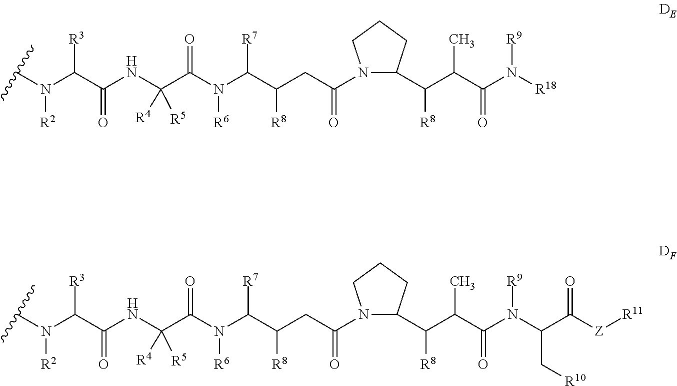

wherein the wavy line of D_{E} and D_{E} indicates the covalent attachment site to an antibody or antibody-linker component, and independently at each location:
R² is selected from H and C₁-C₈ alkyl;
R³ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
R⁴ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
R⁵ is selected from H and methyl;
or R⁴ and R⁵ jointly form a carbocyclic ring and have the formula —(CR^{a}R_{b})ₙ— wherein Rₐ and R_{b} are independently selected from H, C₁-C₈ alkyl and C₃-C₈ carbocycle and n is selected from 2, 3, 4, 5 and 6;
R⁶ is selected from H and C₁-C₈ alkyl;
R⁷ is selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle and C₁-C₈ alkyl-(C₃-C₈ heterocycle);
each R⁸ is independently selected from H, OH, C₁-C₈ alkyl, C₃-C₈ carbocycle and O—(C₁-C₈ alkyl);
R⁹ is selected from H and C₁-C₈ alkyl;
R¹⁰ is selected from aryl or C₃-C₈ heterocycle;
Z is O, S, NH, or NR¹², wherein R¹² is C₁-C₈ alkyl;
R¹¹ is selected from H, C₁-C₂₀ alkyl, aryl, C₃-C₈ heterocycle, —(R¹³O)ₘ—R¹⁴, or —(R¹³O)ₘ—CH(R15)₂;
m is an integer ranging from 1-1000;
R¹³ is C₂-C₈ alkyl;
R¹⁴ is H or C₁-C₈ alkyl;
each occurrence of R¹⁵ is independently H, COOH, —(CH₂)ₙ—N(R¹⁶)₂, —(CH₂)ₙ—SO₃H, or —(CH2)ₙ—SO₃—C₁-C₈ alkyl;
each occurrence of R¹⁶ is independently H, C₁-C₈ alkyl, or —(CH₂)ₙ—COOH;
R¹⁸ is selected from —C(R′)₂—C(R′)₂-aryl, —C(R′)₂—C(R′)₂—(C₃-C₈ heterocycle), and —C(R′)₂—C(R′)₂—(C₃-C₈ carbocycle); and
n is an integer ranging from 0 to 6.

In one embodiment, R³, R⁴ and R⁷ are independently isopropyl or sec-butyl and R⁵ is —H or methyl. In an exemplary embodiment, R³ and R⁴ are each isopropyl, R⁵ is —H, and R⁷ is sec-butyl.

In yet another embodiment, R² and R⁶ are each methyl, and R⁹ is —H.

In still another embodiment, each occurrence of R⁸ is —OCH₃.

In an exemplary embodiment, R³ and R⁴ are each isopropyl, R² and R⁶ are each methyl, R⁵ is —H, R⁷ is sec-butyl, each occurrence of R⁸ is —OCH₃, and R⁹ is —H.

In one embodiment, Z is —O— or —NH—.

In one embodiment, R¹⁰ is aryl.

In an exemplary embodiment, R¹⁰ is -phenyl.

In an exemplary embodiment, when Z is —O—, R¹¹ is —H, methyl or t-butyl.

In one embodiment, when Z is —NH, R¹¹ is —CH(R¹⁵)₂, wherein R¹⁵ is —(CH₂)ₙ—N(R¹⁶)₂, and R¹⁶ is —C₁-C₈ alkyl or —(CH₂)ₙ—COOH.

In another embodiment, when Z is —NH, R¹¹ is —CH(R¹⁵)₂, wherein R¹⁵ is —(CH₂)ₙ—SO₃H.

An exemplary auristatin embodiment of formula D_{E} is MMAE, wherein the wavy line indicates the covalent attachment to a linker (L) of an anti-CD79b immunoconjugate:

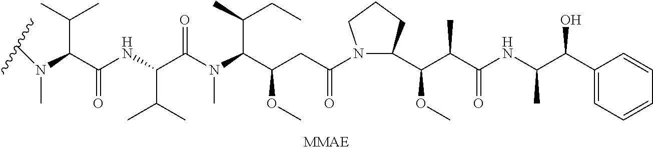 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

An exemplary auristatin embodiment of formula D_{E} is MMAF, wherein the wavy line indicates the covalent attachment to a linker (L) of an anti-CD79b immunoconjugate:

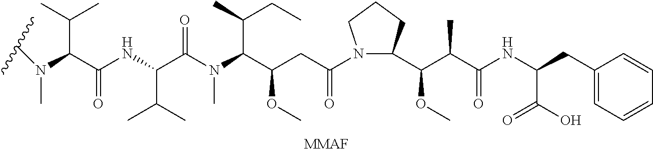

Other exemplary embodiments include monomethylvaline compounds having phenylalanine carboxy modifications at the C-terminus of the pentapeptide auristatin drug moiety (WO 2007/008848) and monomethylvaline compounds having phenylalanine sidechain modifications at the C-terminus of the pentapeptide auristatin drug moiety (WO 2007/008603).

Nonlimiting exemplary embodiments of an anti-CD79b immunoconjugate of Formula I comprising MMAE or MMAF and various linker components have the following structures and abbreviations (wherein “Ab” is an anti-CD79b antibody; p is 1 to about 8, “Val-Cit” is a valine-citrulline dipeptide; and “S” is a sulfur atom:

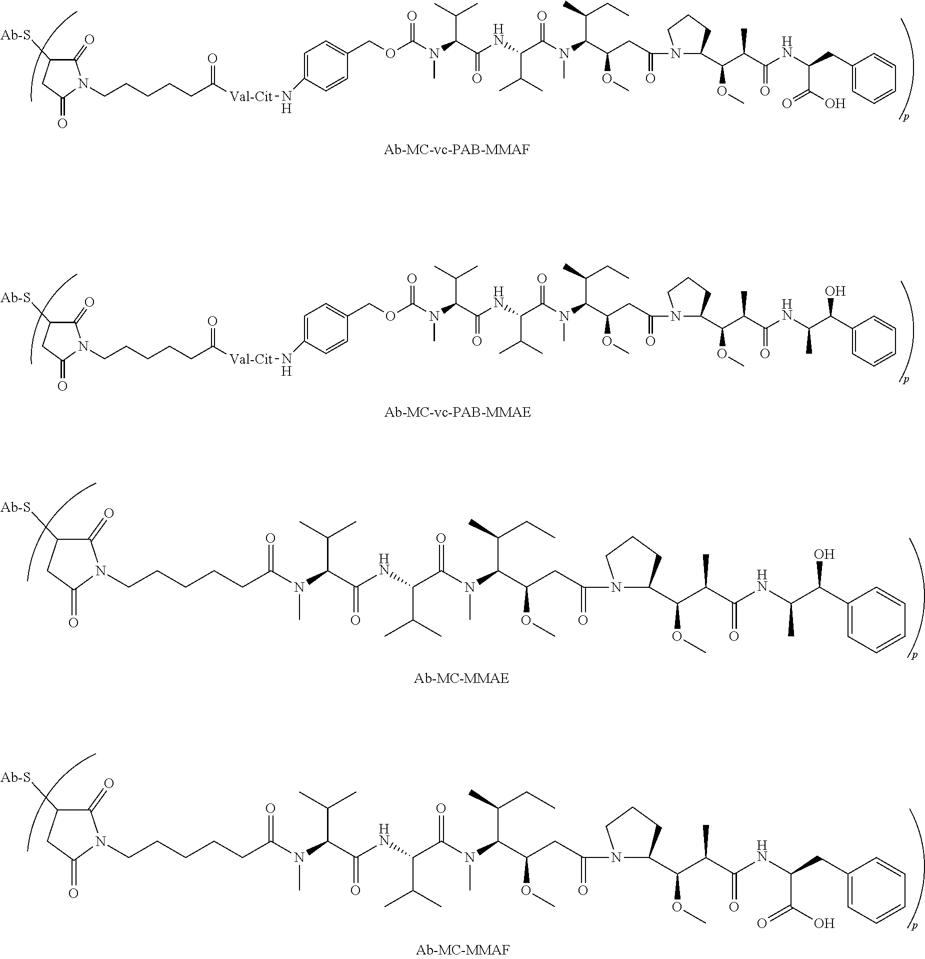

In certain embodiments, the anti-CD79b immunoconjugate comprises the structure of Ab-MC-vc-PAB-MMAE, wherein p is, e.g., about 1 to about 8; about 2 to about 7; about 3 to about 5; about 3 to about 4; or about 3.5. In some embodiments, the anti-CD79b immunoconjugate is huMA79bv28-MC-vc-PAB-MMAE, e.g., an anti-CD79b immunoconjugate comprising the structure of MC-vc-PAB-MMAE, wherein p is, e.g., about 1 to about 8; about 2 to about 7; about 3 to about 5; about 3 to about 4; or about 3.5, wherein the anti-CD79 antibody comprises a heavy chain comprising the amino acid sequence of SEQ ID NO: 36, and wherein the light chain comprises the amino acid sequence of SEQ ID NO: 35. In some embodiments, the anti-CD79b immunoconjugate is polatuzumab vedotin-piiq (CAS Number 1313206-42-6). Polatuzumab vedotin-piiq has the IUPHAR/BPS Number 8404, the KEGG Number D10761, the INN number 9714, and can also be referred to as “DCDS4501A,” or “RG7596.”

Nonlimiting exemplary embodiments of anti-CD79b immunoconjugates of Formula I comprising MMAF and various linker components further include Ab-MC-PAB-MMAF and Ab-PAB-MMAF. Immunoconjugates comprising MMAF attached to an antibody by a linker that is not proteolytically cleavable have been shown to possess activity comparable to immunoconjugates comprising MMAF attached to an antibody by a proteolytically cleavable linker (Doronina et al. (2006) Bioconjugate Chem. 17:114-124). In some such embodiments, drug release is believed to be effected by antibody degradation in the cell.

Typically, peptide-based drug moieties can be prepared by forming a peptide bond between two or more amino acids and/or peptide fragments. Such peptide bonds can be prepared, for example, according to a liquid phase synthesis method (see, e.g., E. Schröder and K. Lübke, “The Peptides”, volume 1, pp 76-136, 1965, Academic Press). Auristatin/dolastatin drug moieties may, in some embodiments, be prepared according to the methods of: U.S. Pat. Nos. 7,498,298; 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 1 5:859-863; and Doronina (2003) Nat. Biotechnol. 21(7):778-784.

In some embodiments, auristatin/dolastatin drug moieties of formulas D_{E} such as MMAE, and D_{F}, such as MMAF, and drug-linker intermediates and derivatives thereof, such as MC-MMAF, MC-MMAE, MC-vc-PAB-MMAF, and MC-vc-PAB-MMAE, may be prepared using methods described in U.S. Pat. No. 7,498,298; Doronina et al. (2006) Bioconjugate Chem. 17:114-124; and Doronina et al. (2003) Nat. Biotech. 21:778-784, and then conjugated to an antibody of interest.

(3) Calicheamicin

In some embodiments, the anti-CD79b immunoconjugate comprises an anti-CD79b antibody conjugated to one or more calicheamicin molecules. The calicheamicin family of antibiotics, and analogues thereof, are capable of producing double-stranded DNA breaks at sub-picomolar concentrations (Hinman et al., (1993) Cancer Research 53:3336-3342; Lode et al., (1998) Cancer Research 58:2925-2928). Calicheamicin has intracellular sites of action but, in certain instances, does not readily cross the plasma membrane. Therefore, cellular uptake of these agents through antibody-mediated internalization may, in some embodiments, greatly enhance their cytotoxic effects. Nonlimiting exemplary methods of preparing anti-CD79b antibody immunoconjugates with a calicheamicin drug moiety are described, for example, in U.S. Pat. Nos. 5,712,374; 5,714,586; 5,739,116; and 5,767,285.

(4) Other Drug Moieties

In some embodiments, an anti-CD79b immunoconjugate comprises geldanamycin (Mandler et al (2000) J. Nat. Cancer Inst. 92(19):1573-1581; Mandler et al (2000) Bioorganic & Med. Chem. Letters 10:1025-1028; Mandler et al (2002) Bioconjugate Chem. 13:786-791); and/or enzymatically active toxins and fragments thereof, including, but not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, e.g., WO 93/21232.

Drug moieties also include compounds with nucleolytic activity (e.g., a ribonuclease or a DNA endonuclease).

In certain embodiments, an anti-CD79b immunoconjugate comprises a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. In some embodiments, when an anti-CD79b immunoconjugate is used for detection, it may comprise a radioactive atom for scintigraphic studies, for example Tc⁹⁹ or I¹²³, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

The radio- or other labels may be incorporated in the anti-CD79b immunoconjugate in known ways. For example, a peptide may be biosynthesized or chemically synthesized using suitable amino acid precursors comprising, for example, one or more fluorine-19 atoms in place of one or more hydrogens. In some embodiments, labels such as Tc⁹⁹, I¹²³, Re¹⁸⁶, Re¹⁸⁸ and In¹¹¹ can be attached via a cysteine residue in the anti-CD79b antibody. In some embodiments, yttrium-90 can be attached via a lysine residue of the anti-CD79b antibody. In some embodiments, the IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes certain other methods.

In certain embodiments, an anti-CD79b immunoconjugate may comprise an anti-CD79b antibody conjugated to a prodrug-activating enzyme. In some such embodiments, a prodrug-activating enzyme converts a prodrug (e.g., a peptidyl chemotherapeutic agent, see WO 81/01145) to an active drug, such as an anti-cancer drug. Such immunoconjugates are useful, in some embodiments, in antibody-dependent enzyme-mediated prodrug therapy (“ADEPT”). Enzymes that may be conjugated to an anti-CD79b antibody include, but are not limited to, alkaline phosphatases, which are useful for converting phosphate-containing prodrugs into free drugs; arylsulfatases, which are useful for converting sulfate-containing prodrugs into free drugs; cytosine deaminase, which is useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibodies by recombinant DNA techniques well known in the art. See, e.g., Neuberger et al., Nature 312:604-608 (1984).

D. Drug Loading

Drug loading is represented by p, the average number of drug moieties per anti-CD79b antibody in a molecule of Formula I. Drug loading may range from 1 to 20 drug moieties (D) per antibody. Anti-CD79b immunoconjugates of Formula I include collections of anti-CD79b antibodies conjugated with a range of drug moieties, from 1 to 20. The average number of drug moieties per anti-CD79b antibody in preparations of anti-CD79b immunoconjugates from conjugation reactions may be characterized by conventional means such as mass spectroscopy, ELISA assay, and HPLC. The quantitative distribution of anti-CD79b immunoconjugates in terms of p may also be determined. In some instances, separation, purification, and characterization of homogeneous anti-CD79b immunoconjugates where p is a certain value from anti-CD79b immunoconjugates with other drug loadings may be achieved by means such as reverse phase HPLC or electrophoresis.

For some anti-CD79b immunoconjugates, p may be limited by the number of attachment sites on the anti-CD79b antibody. For example, where the attachment is a cysteine thiol, as in certain exemplary embodiments above, an anti-CD79b antibody may have only one or several cysteine thiol groups, or may have only one or several sufficiently reactive thiol groups through which a linker may be attached. In certain embodiments, higher drug loading, e.g., p >5, may cause aggregation, insolubility, toxicity, or loss of cellular permeability of certain anti-CD79b immunoconjugates. In certain embodiments, the average drug loading for an anti-CD79b immunoconjugates ranges from 1 to about 8; from about 2 to about 6; from about 3 to about 5; or from about 3 to about 4. Indeed, it has been shown that for certain antibody-drug conjugates, the optimal ratio of drug moieties per antibody may be less than 8, and may be about 2 to about 5 (U.S. Pat. No. 7,498,298). In certain embodiments, the optimal ratio of drug moieties per antibody is about 3 to about 4. In certain embodiments, the optimal ratio of drug moieties per antibody is about 3.5.

In certain embodiments, fewer than the theoretical maximum of drug moieties are conjugated to the anit-CD79b antibody during a conjugation reaction. An antibody may contain, for example, lysine residues that do not react with the drug-linker intermediate or linker reagent, as discussed below. Generally, antibodies do not contain many free and reactive cysteine thiol groups which may be linked to a drug moiety; indeed most cysteine thiol residues in antibodies exist as disulfide bridges. In certain embodiments, an anti-CD79b antibody may be reduced with a reducing agent such as dithiothreitol (DTT) or tricarbonylethylphosphine (TCEP), under partial or total reducing conditions, to generate reactive cysteine thiol groups. In certain embodiments, an anti-CD79b antibody is subjected to denaturing conditions to reveal reactive nucleophilic groups such as lysine or cysteine.

The loading (drug/antibody ratio) of an anti-CD79b immunoconjugate may be controlled in different ways, and for example, by: (i) limiting the molar excess of drug-linker intermediate or linker reagent relative to antibody, (ii) limiting the conjugation reaction time or temperature, and (iii) partial or limiting reductive conditions for cysteine thiol modification.

It is to be understood that where more than one nucleophilic group reacts with a drug-linker intermediate or linker reagent, then the resulting product is a mixture of anti-CD79b immunoconjugate compounds with a distribution of one or more drug moieties attached to an anti-CD79b antibody. The average number of drugs per antibody may be calculated from the mixture by a dual ELISA antibody assay, which is specific for antibody and specific for the drug. Individual anti-CD79b immunoconjugate molecules may be identified in the mixture by mass spectroscopy and separated by HPLC, e.g., hydrophobic interaction chromatography (see, e.g., McDonagh et al (2006) Prot. Engr. Design & Selection 19(7):299-307; Hamblett et al (2004) Clin. Cancer Res. 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al. “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous anti-CD79b immunoconjugate with a single loading value may be isolated from the conjugation mixture by electrophoresis or chromatography.

E. Methods of Preparing Anti-CD79b Immunoconjugates

An anti-CD79b immunoconjugate of Formula I may be prepared by several routes employing organic chemistry reactions, conditions, and reagents known to those skilled in the art, including, but not limited to, e.g., (1) reaction of a nucleophilic group of an anti-CD79b antibody with a bivalent linker reagent to form Ab-L via a covalent bond, followed by reaction with a drug moiety D; and (2) reaction of a nucleophilic group of a drug moiety with a bivalent linker reagent, to form D-L, via a covalent bond, followed by reaction with a nucleophilic group of an anti-CD79b antibody. Exemplary methods for preparing an anti-CD79b immunoconjugate of Formula I via the latter route are described in U.S. Pat. No. 7,498,298, which is expressly incorporated herein by reference.

Nucleophilic groups on antibodies include, but are not limited to: (i) N-terminal amine groups, (ii) side chain amine groups, e.g., lysine, (iii) side chain thiol groups, e.g., cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; and (iii) aldehydes, ketones, carboxyl, and maleimide groups. Certain antibodies have reducible interchain disulfides, i.e. cysteine bridges. Anti-CD79b antibodies may be made reactive for conjugation with linker reagents by treatment with a reducing agent such as DTT (dithiothreitol) or tricarbonylethylphosphine (TCEP), such that the anti-CD79b antibody is fully or partially reduced. Each cysteine bridge will thus form, theoretically, two reactive thiol nucleophiles. Additional nucleophilic groups can be introduced into anti-CD79b antibodies through modification of lysine residues, e.g., by reacting lysine residues with 2-iminothiolane (Traut's reagent), resulting in conversion of an amine into a thiol. Reactive thiol groups may also be introduced into an anti-CD79b antibody by introducing one, two, three, four, or more cysteine residues (e.g., by preparing variant antibodies comprising one or more non-native cysteine amino acid residues).

Anti-CD79b immunoconjugates described herein may also be produced by reaction between an electrophilic group on an anti-CD79b antibody, such as an aldehyde or ketone carbonyl group, with a nucleophilic group on a linker reagent or drug. Useful nucleophilic groups on a linker reagent include, but are not limited to, hydrazide, oxime, amino, hydrazine, thiosemicarbazone, hydrazine carboxylate, and arylhydrazide. In one embodiment, an anti-CD79b antibody is modified to introduce electrophilic moieties that are capable of reacting with nucleophilic substituents on the linker reagent or drug. In another embodiment, the sugars of glycosylated anti-CD79b antibodies may be oxidized, e.g., with periodate oxidizing reagents, to form aldehyde or ketone groups which may react with the amine group of linker reagents or drug moieties. The resulting imine Schiff base groups may form a stable linkage, or may be reduced, e.g., by borohydride reagents to form stable amine linkages. In one embodiment, reaction of the carbohydrate portion of a glycosylated anti-CD79b antibody with either galactose oxidase or sodium meta-periodate may yield carbonyl (aldehyde and ketone) groups in the anti-CD79b antibody that can react with appropriate groups on the drug (Hermanson, Bioconjugate Techniques). In another embodiment, anti-CD79b antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a drug moiety or linker nucleophile.

Exemplary nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Nonlimiting exemplary cross-linker reagents that may be used to prepare anti-CD79b immunoconjugates are described herein in the section titled “Exemplary Linkers.” Methods of using such cross-linker reagents to link two moieties, including a proteinaceous moiety and a chemical moiety, are known in the art. In some embodiments, a fusion protein comprising an anti-CD79b antibody and a cytotoxic agent may be made, e.g., by recombinant techniques or peptide synthesis. A recombinant DNA molecule may comprise regions encoding the antibody and cytotoxic portions of the conjugate either adjacent to one another or separated by a region encoding a linker peptide which does not destroy the desired properties of the conjugate. In yet another embodiment, an anti-CD79b antibody may be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a drug or radionucleotide). Additional details regarding anti-CD79b immunoconjugates are provided in U.S. Pat. No. 8,545,850 and WO/2016/049214, the contents of which are expressly incorporated by reference herein in their entirety.

In some embodiments, provided herein are immunoconjugates comprising the formula

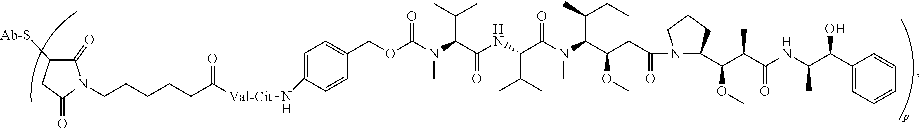

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with a Bcl-2 inhibitor and an anti-CD20 antibody for treating a human in need thereof having diffuse large B-cell lymphoma (DLBCL) according to any method of treatment provided herein.

In some embodiments, provided herein are immunoconjugates comprising the formula

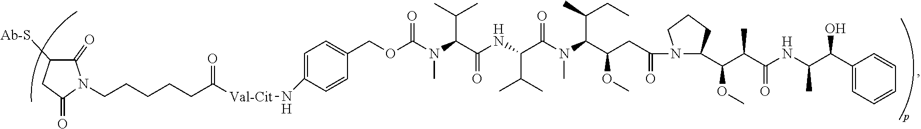

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with venetoclax and rituximab for treating a human in need thereof having diffuse large B-cell lymphoma (DLBCL) according to any method of treatment provided herein.

In some embodiments, p is between 3 and 4 or between 2 and 5. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35.

Also provided herein is polatuzumab vedotin-piiq for use in combination with venetoclax and rituximab for treating a human in need thereof having diffuse large B-cell lymphoma (DLBCL) according to any method of treatment provided herein.

In some embodiments, provided herein is an immunoconjugate comprising the formula

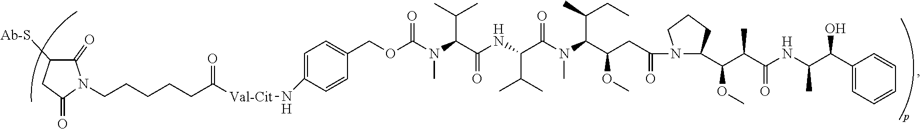

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with a Bcl-2 inhibitor and an anti-CD20 antibody for treating a human in need thereof having follicular lymphoma (FL) according to any method of treatment provided herein.

In some embodiments, provided herein is an immunoconjugate comprising the formula

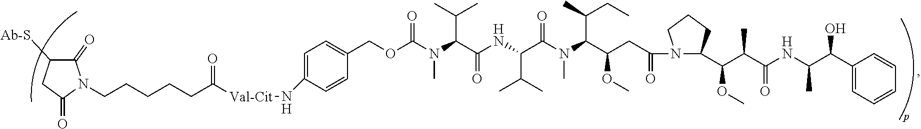

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with venetoclax and obinutuzumab for treating a human in need thereof having follicular lymphoma (FL) according to any method of treatment provided herein.

In some embodiments, p is between 3 and 4 or between 2 and 5. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35.

Also provided herein is polatuzumab vedotin-piiq for use in combination with venetoclax and obinutuzumab for treating a human in need thereof having follicular lymphoma (FL) according to any method of treatment provided herein.

### VI. Bcl-2 Inhibitors

The methods provided herein involve administration of a Bcl-2 inhibitor. Methods of treatment using Bcl-2 inhibitors are disclosed in U.S. Publication No. 2012/0129853, the disclosure of which is hereby incorporated by reference in its entirety. In some embodiments, the combination therapy of the present invention involves the administration of a Bcl-2 inhibitor that selectively inhibits Bcl-2 protein. For example, 4-(4-{[2-(4-chlorophenyl)-4,4-dimethylcyclohex-1-en-1-yl]methyl}piperazin-1-yl)-N-{{3-nitro-4-[(tetrahydro-2H-pyran-4-ylmethyl)amino]phenyl}sulfonyl)-2-(1H-pyrrolo[2,3-b]pyridin-5-yloxy)benzamide (also optionally referred to as, venetoclax, or ABT-199/GDC-0199).

Venetoclax is an orally available, potent and highly selective inhibitor of Bcl-2, a member of the Bcl-2 family of regulator proteins that regulate apoptosis. Venetoclax selectively binds to and elicits a response on Bcl-2 proteins at much lower concentrations than those required to bind to and elicit a response on Bcl-X_{L}. As such, when venetoclax is administered to the patient, the inhibitor is more prone to inhibit Bcl-2, rather than Bcl-X_{L}. Venetoclax tends to have a competitive binding affinity (Ki) for Bcl-2 that is at least about 500, at least about 1000, at least about 2000, at least about 2500, at least about 3000, at least about 3500, and at least about 4000 times less than the binding affinity for Bcl-X_{L}. As such, even at low concentrations (i.e., picomolar concentrations), venetoclax will bind to and inhibit the Bcl-2 protein.

In some embodiments, the Bcl-2 inhibitor comprises 4-(4-{[2-(4-chlorophenyl)-4,4-dimethylcyclohex-1-en-1-yl]methyl}piperazin-1-yl)-N-{{3-nitro-4-[(tetrahydro-2H-pyran-4-ylmethyl)amino]phenyl}sulfonyl)-2-(1H-pyrrolo[2,3-b]pyridin-5-yloxy)benzamide (venetoclax, or ABT-199/GDC-0199) or a pharmaceutically acceptable salt thereof. In some embodiments, the combination therapy of the present disclosure involves administration of a therapeutically effective amount of 4-(4-{[2-(4-chlorophenyl)-4,4-dimethylcyclohex-1-en-1-yl]methyl}piperazin-1-yl)-N-{{3-nitro-4-[(tetrahydro-2H-pyran-4-ylmethyl)amino]phenyl}sulfonyl)-2-(1H-pyrrolo[2,3-b]pyridin-5-yloxy)benzamide (venetoclax, or ABT-199/GDC-0199) or a pharmaceutically acceptable salt thereof to a mammal, e.g., a human patient, in need thereof. Venetoclax has the following structure:

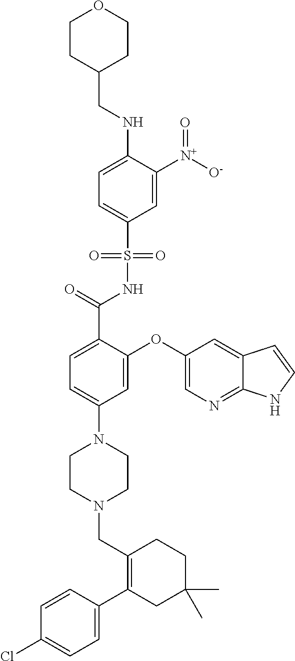 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Venetoclax (or ABT-199/GDC-0199) may be formulated in its parent-compound form (i.e., as a free base), in a pharmaceutically acceptable salt form of the compound, or a combination of the parent-compound form and the pharmaceutically acceptable salt form. Additional suitable forms include the hydrate or solvated forms of ABT-199. In some embodiments, the ABT-199 may be a crystalline polymorph suitable for incorporation into a pharmaceutical composition further comprising a pharmaceutical acceptable excipient.

Salts and crystalline forms of ABT-199 are disclosed in U.S. Publication No. 2012/0157470, the disclosure of which is hereby incorporated by reference in its entirety. The phrase “pharmaceutically acceptable salt(s)”, as used herein, means those salts of ABT-199 that are safe and effective for administration to a patient and that do not adversely affect the therapeutic qualities of the compound.

Pharmaceutically acceptable salts include salts of acidic or basic groups present in compounds of the invention. Salts of ABT-199 can be prepared during isolation or following purification of the compounds.

Acid addition salts are those derived from the reaction of Venetoclax (or ABT-199/GDC-0199) with an acid. For example, salts including the acetate, acid phosphate, adipate, alginate, ascorbate, bicarbonate, citrate, aspartate, benzoate, benzenesulfonate (besylate), bisulfate, bitartrate, butyrate, camphorate, camphorsulfonate, citrate, digluconate, ethane sulfonate, ethanedisulfonate, formate, fumarate, gentisinate, glycerophosphate, gluconate, glucaronate, glutamate, hemisulfate, heptanoate, hexanoate, hydrobromide, hydrochloride, hydroiodide, isonicotinate, 1-hydroxy-2-naphthoate, lactate, lactobionate, malate, maleate, malonate, mesitylenesulfonate, methanesulfonate, naphthalenesulfonate, nicotinate, nitrate, oxalate, P-toluenesulfonate, pamoate (i.e., 1,1′-methylene-bis-(2-hydroxy-3-naphthoate), pantothenate, pectinate, persulfate, phosphate, picrate, propionate, saccharate, salicylate, succinate, sulfate, tartrate, thiocyanate, trichloroacetate, trifluoroacetate, para-toluenesulfonate and undecanoate salts of a compound of ABT-199 can be used in a composition of the invention. Basic addition salts, including those derived from reaction of ABT-199) with the bicarbonate, carbonate, hydroxide or phosphate of cations such as aluminum, lithium, sodium, potassium, calcium, zinc, and magnesium, can likewise be used. (For a review on pharmaceutically acceptable salts see, e.g., Berge et al., 66 J. Pharm. Sci., 1-19(1977), incorporated herein by reference, in its entirety.).

The Bcl-2 family of proteins is a group of proteins that have regulatory effects on many developmental and homeostasis functions, such as apoptosis (programmed cell death). The Bcl-2 family includes other proteins, including Bcl-X_{L}, and Bcl-w. The Bcl-2 inhibitor compounds have shown a higher binding affinity (as evidenced by lower Ki values) for Bcl-2 compared to other Bcl-2 family proteins, such as Bcl-X_{L}, and Bcl-w. Additionally, ABT-199 is a more potent Bcl-2 inhibitor than some Bcl-2 inhibitors known in the art.

The binding affinity for the various proteins is measured as a value of Ki, which represents the amount of the compound required to inhibit a physiologic process or compound (such as a protein) by 50%. See U. S. Publication No. 2012/0129853, the disclosure of which is hereby incorporated by reference in its entirety. The Bcl-2 inhibitors used in the methods provided herein generally have a binding affinity (Ki) of less than about 1 micromolar, less than about 500 nanomolar, less than about 400 nanomolar, less than about 300 nanomolar, less than about 200 nanomolar, less than about 100 nanomolar, less than about 50 nanomolar, less than about 25 nanomolar, less than about 10 nanomolar, less than about 5 nanomolar, less than about 1 nanomolar, less than about 900 picomolar, less than about 800 picomolar, less than about 700 picomolar, less than about 600 picomolar, less than about 500 picomolar, less than about 400 picomolar, less than about 300 picomolar, less than about 200 picomolar, or less than about 100 picomolar to Bcl-2.

In some embodiments, a Bcl-2 inhibitor for use according to methods provided herein is a selective Bcl-2 inhibitor (e.g., venetoclax). In this regard, a Bcl-2 inhibitor is one which selectively binds to a particular protein within the Bcl-2 family (e.g., Bcl-2). In some embodiments, a selective Bcl-2 inhibitor for use according to methods provided herein is venetoclax. In some embodiments, venetoclax selectively binds to a particular protein within the Bcl-2 family, e.g., Bcl-2, e.g., as described above.

### VII. Anti-CD20 Agents

Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1052, see Table M.

TABLE M
Properties of type I and type II anti-CD20 antibodies

| Type I anti-CD20 antibodies | Type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon | Strong cell death induction without |
| cross-linking | cross-linking |

Examples of type I anti-CD20 antibodies include e.g., rituximab, HI47 IgG₃ (ECACC, hybridoma), 2C6 IgG1 (as disclosed in WO 2005/103081), 2F2 IgG1 (as disclosed in WO 2004/035607 and WO 2005/103081), and 2H7 IgG1 (as disclosed in WO 2004/056312).

In some embodiments, the anti-CD20 antibody used in a method of treatment provided herein is rituximab. In some embodiments, the rituximab (reference antibody; example of a type I anti-CD20 antibody) is a genetically engineered chimeric human gamma 1 murine constant domain containing monoclonal antibody directed against the human CD20 antigen. However this antibody is not glycoengineered and not afucosylated and thus has an amount of fucose of at least 85%. This chimeric antibody comprises human gamma 1 constant domains and is identified by the name “C2B8” in U.S. Pat. No. 5,736,137 (Andersen, et al) issued on Apr. 17, 1998, assigned to IDEC Pharmaceuticals Corporation. Rituximab is approved for the treatment of patients with relapsed or refracting low-grade or follicular, CD20 positive, B-cell non-Hodgkin's lymphoma. In vitro mechanism of action studies have shown that rituximab exhibits human complement-dependent cytotoxicity (CDC) (Reff, M. E., et al, Blood 83(2) (1994) 435-445). Additionally, it exhibits activity in assays that measure antibody-dependent cellular cytotoxicity (ADCC).

In some embodiments, the anti-CD20 antibody used in a method of treatment provided herein comprises, according to numbering in Kabat et al., the CDR-H1, CDR-H2, CDR-H3, CDR-L1, CDR-L2, and CDR-L3 of rituximab. In some embodiments, anti-CD20 antibody used in a method of treatment provided herein comprises the VH and the VL of rituximab. In some embodiments, anti-CD20 antibody used in a method of treatment provided herein comprises the heavy chain and the light chain of rituximab. As used herein, the term “rituximab” refers to an anti-CD20 antibody having the CAS Registry Number 174722-31-7.

In some embodiments, the anti-CD20 antibody used in a method of treatment provided herein is an afucosylated anti-CD20 antibody.

Examples of type II anti-CD20 antibodies include e.g., humanized B-Ly1 antibody IgG1 (a chimeric humanized IgG1 antibody as disclosed in WO 2005/044859), 11B8 IgG1 (as disclosed in WO 2004/035607), and AT80 IgG1. Typically type II anti-CD20 antibodies of the IgG1 isotype show characteristic CDC properties. Type II anti-CD20 antibodies have a decreased CDC (if IgG1 isotype) compared to type I antibodies of the IgG1 isotype. In some embodiments, the type II anti-CD20 antibody, e.g., a GA101 antibody, has increased antibody dependent cellular cytotoxicity (ADCC). In some embodiments, the type II anti-CD20 antibodies, more preferably an afucosylated humanized B-Ly1 antibody as described in WO 2005/044859 and WO 2007/031875.

In some embodiments, the anti-CD20 antibody used in a method of treatment provided herein is GA101 antibody. In some embodiments, the GA101 antibody as used herein refers to any one of the following antibodies that bind human CD20: (1) an antibody comprising an HVR-H1 comprising the amino acid sequence of SEQ ID NO:5, an HVR-H2 comprising the amino acid sequence of SEQ ID NO:6, an HVR-H3 comprising the amino acid sequence of SEQ ID NO:7, an HVR-L1 comprising the amino acid sequence of SEQ ID NO:8, an HVR-L2 comprising the amino acid sequence of SEQ ID NO:9, and an HVR-L3 comprising the amino acid sequence of SEQ ID NO:10; (2) an antibody comprising a VH domain comprising the amino acid sequence of SEQ ID NO:11 and a VL domain comprising the amino acid sequence of SEQ ID NO: 12, (3) an antibody comprising an amino acid sequence of SEQ ID NO:13 and an amino acid sequence of SEQ ID NO: 14; (4) an antibody known as obinutuzumab, or (5) an antibody that comprises an amino acid sequence that has at least 95%, 96%, 97%, 98% or 99% sequence identity with amino acid sequence of SEQ ID NO:13 and that comprises an amino acid sequence that has at least 95%, 96%, 97%, 98% or 99% sequence identity with an amino acid sequence of SEQ ID NO: 14. In one embodiment, the GA101 antibody is an IgG1 isotype antibody.

In some embodiments, the anti-CD20 antibody used in a method of treatment provided herein is a humanized B-Ly1 antibody. In some embodiments, the humanized B-Ly1 antibody refers to humanized B-Ly1 antibody as disclosed in WO 2005/044859 and WO 2007/031875, which were obtained from the murine monoclonal anti-CD20 antibody B-Ly1 (variable region of the murine heavy chain (VH): SEQ ID NO: 3; variable region of the murine light chain (VL): SEQ ID NO: 4—see Poppema, S. and Visser, L., Biotest Bulletin 3 (1987) 131-139) by chimerization with a human constant domain from IgG1 and following humanization (see WO 2005/044859 and WO 2007/031875). The humanized B-Ly1 antibodies are disclosed in detail in WO 2005/044859 and WO 2007/031875.

In some embodiments, the humanized B-Ly1 antibody has variable region of the heavy chain (VH) selected from group of SEQ ID NO:15-16 and 40-55 (corresponding to B-HH2 to B-HH9 and B-HL8 to B-HL17 of WO 2005/044859 and WO 2007/031875). In some embodiments, the variable domain is selected from the group consisting of SEQ ID NO: 15, 16, 42, 44, 46, 48 and 50 (corresponding to B-HH2, BHH-3, B-HH6, B-HH8, B-HL8, B-HL11 and B-HL13 of WO 2005/044859 and WO 2007/031875). In some embodiments, the humanized B-Ly1 antibody has variable region of the light chain (VL) of SEQ ID NO:55 (corresponding to B-KV1 of WO 2005/044859 and WO 2007/031875). In some embodiments, the humanized B-Ly1 antibody has a variable region of the heavy chain (VH) of SEQ ID NO:42 (corresponding to B-HH6 of WO 2005/044859 and WO 2007/031875) and a variable region of the light chain (VL) of SEQ ID NO:55 (corresponding to B-KV1 of WO 2005/044859 and WO 2007/031875). In some embodiments, the humanized B-Ly1 antibody is an IgG1 antibody. Such afucosylated humanized B-Ly1 antibodies are glycoengineered (GE) in the Fc region according to the procedures described in WO 2005/044859, WO 2004/065540, WO 2007/031875, Umana, P. et al., Nature Biotechnol. 17 (1999) 176-180 and WO 99/154342. In some embodiments, the afucosylated glyco-engineered humanized B-Ly1 is B-HH6-B-KV1 GE. In some embodiments, the anti-CD20 antibody is obinutuzumab (recommended INN, WHO Drug Information, Vol. 26, No. 4, 2012, p. 453). As used herein, obinutuzumab is synonymous for GA101 or R05072759. It is commercially available for therapeutic use under the trade name GAZYVA®, and is provided as a 1000 mg/40 mL (25 mg/mL) single-dose vial. This replaces all previous versions (e.g., Vol. 25, No. 1, 2011, p. 75-76), and is formerly known as afutuzumab (recommended INN, WHO Drug Information, Vol. 23, No. 2, 2009, p. 176; Vol. 22, No. 2, 2008, p. 124). In some embodiments, the humanized B-Ly1 antibody is an antibody comprising a heavy chain comprising the amino acid sequence of SEQ ID NO:17 and a light chain comprising the amino acid sequence of SEQ ID NO:18, or an antigen-binding fragment thereof such antibody. In some embodiments, the humanized B-Ly1 antibody comprises a heavy chain variable region comprising the three heavy chain CDRs of SEQ ID NO:17 and a light chain variable region comprising the three light chain CDRs of SEQ ID NO:18.

In some embodiments, the humanized B-Ly1 antibody is an afucosylated glyco-engineered humanized B-Ly1. Such glycoengineered humanized B-Ly1 antibodies have an altered pattern of glycosylation in the Fc region, preferably having a reduced level of fucose residues. In some embodiments, the amount of fucose is about 60% or less of the total amount of oligosaccharides at Asn297 (in one embodiment the amount of fucose is between about 40% and about 60%, in another embodiment the amount of fucose is about 50% or less, and in still another embodiment the amount of fucose is about 30% or less). In some embodiments, the oligosaccharides of the Fc region are bisected. These glycoengineered humanized B-Ly1 antibodies have an increased ADCC.

The “ratio of the binding capacities to CD20 on Raji cells (ATCC-No. CCL-86) of an anti-CD20 antibodies compared to rituximab” is determined by direct immunofluorescence measurement (the mean fluorescence intensities (MFI) is measured) using said anti-CD20 antibody conjugated with Cy5 and rituximab conjugated with Cy5 in a FACSArray (Becton Dickinson) with Raji cells (ATCC-No. CCL-86), as described in Example No. 2, and calculated as follows:

$$Ratio of the binding capacities to CD 20 on Raji cells \\ ( ATCC - No . CCL - 86 ) = \frac{M F I ( Cy 5 - anti - CD 20 antibody )}{M F I ( Cy 5 - rituximab )} \times \frac{Cy 5 - labeling ratio ( Cy 5 - rituximab )}{Cy 5 - labeling ratio ( Cy 5 - anti - CD 20 antibody )}$$

MFI is the mean fluorescent intensity. The “Cy5-labeling ratio” as used herein means the number of Cy5-label molecules per molecule antibody.

Typically said type II anti-CD20 antibody has a ratio of the binding capacities to CD20 on Raji cells (ATCC-No. CCL-86) of said second anti-CD20 antibody compared to rituximab of 0.3 to 0.6, and in one embodiment, 0.35 to 0.55, and in yet another embodiment, 0.4 to 0.5.

By “antibody having increased antibody dependent cellular cytotoxicity (ADCC)”, it is meant an antibody, as that term is defined herein, having increased ADCC as determined by any suitable method known to those of ordinary skill in the art.

An exemplary accepted in vitro ADCC assay is described below:
1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
   i) the PBMCs are isolated using standard density centrifugation procedures and are suspended at 5×10⁶ cells/ml in RPMI cell culture medium;
   ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
   iii) 100 microliters of the final target cell suspension above are transferred to each well of a 96-well microtiter plate;
   iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
   v) for the maximum release (MR) controls, 3 additional wells in the plate containing the labeled target cells, receive 50 microliters of a 2% (VN) aqueous solution of non-ionic detergent (Nonidet, Sigma, St. Louis), instead of the antibody solution (point iv above);
   vi) for the spontaneous release (SR) controls, 3 additional wells in the plate containing the labeled target cells, receive 50 microliters of RPMI cell culture medium instead of the antibody solution (point iv above);
   vii) the 96-well microtiter plate is then centrifuged at 50×g for 1 minute and incubated for 1 hour at 4° C.;
   viii) 50 microliters of the PBMC suspension (point i above) are added to each well to yield an effector:target cell ratio of 25:1 and the plates are placed in an incubator under 5% CO₂ atmosphere at 37° C. for 4 hours;
   ix) the cell-free supernatant from each well is harvested and the experimentally released radioactivity (ER) is quantified using a gamma counter;
   x) the percentage of specific lysis is calculated for each antibody concentration according to the formula (ER-MR)/(MR-SR)×100, where ER is the average radioactivity quantified (see point ix above) for that antibody concentration, MR is the average radioactivity quantified (see point ix above) for the MR controls (see point V above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. In one embodiment, the increase in ADCC is relative to the ADCC, measured with the above assay, mediated by the same antibody, produced by the same type of host cells, using the same standard production, purification, formulation and storage methods, which are known to those skilled in the art, except that the comparator antibody (lacking increased ADCC) has not been produced by host cells engineered to overexpress GnTIII and/or engineered to have reduced expression from the fucosyltransferase 8 (FUT8) gene (e.g., including, engineered for FUT8 knock out).

In some embodiments, the “increased ADCC” can be obtained by, for example, mutating and/or glycoengineering of said antibodies. In some embodiments, the anti-CD20 antibody is glycoengineered to have a biantennary oligosaccharide attached to the Fc region of the antibody that is bisected by GlcNAc. In some embodiments, the anti-CD20 antibody is glycoengineered to lack fucose on the carbohydrate attached to the Fc region by expressing the antibody in a host cell that is deficient in protein fucosylation (e.g., Lec13 CHO cells or cells having an alpha-1,6-fucosyltransferase gene (FUT8) deleted or the FUT gene expression knocked down). In some embodiments, the anti-CD20 antibody sequence has been engineered in its Fc region to enhance ADCC. In some embodiments, such engineered anti-CD20 antibody variant comprises an Fc region with one or more amino acid substitutions at positions 298, 333, and/or 334 of the Fc region (EU numbering of residues)).

In some embodiments, the term “complement-dependent cytotoxicity (CDC)” refers to lysis of human cancer target cells by the antibody according to the invention in the presence of complement. CDC can be measured by the treatment of a preparation of CD20 expressing cells with an anti-CD20 antibody according to the invention in the presence of complement. CDC is found if the antibody induces at a concentration of 100 nM the lysis (cell death) of 20% or more of the tumor cells after 4 hours. In some embodiments, the assay is performed with ⁵¹Cr or Eu labeled tumor cells and measurement of released ⁵¹Cr or Eu. Controls include the incubation of the tumor target cells with complement but without the antibody.

In some embodiments, the anti-CD20 antibody is a monoclonal antibody, e.g., a human antibody. In some embodiments, the anti-CD20 antibody is an antibody fragment, e.g., a Fv, Fab, Fab′, scFv, diabody, or F(ab′)₂ fragment. In some embodiments, the anti-CD20 antibody is a substantially full length antibody, e.g., an IgG1 antibody, IgG₂a antibody or other antibody class or isotype as defined herein.

In some embodiments, the anti-CD20 antibody is any of ABP 798 (Amgen, USA), Zytux (AryoGen Pharmed, Iran), AcellBia/Usmal (Biocad, Russia), BI 695500 (Boehringer Ingelheim, Germany), Truxima (Celltrion, South Korea), Blitzima (Celltrion, South Korea), Ritemvia (Celltrion, South Korea), Rituzena/Tuxella (Celltrion, South Korea), CT-P10 (Celltrion, South Korea), Reditux (Dr Reddy's Laboratories, India), Maball (Hetero Group, India), MabTas (Intas Biopharmaceuticals, India), JHL1101 (JHL Biotech, Taiwan), Novex (RTXM83) (mAbxience/Laboratorio Elea, Spain/Argentina), MabionCD20 (Mabion, Poland; Mylan, India), PF-05280586 (Pfizer, USA), Kikuzubam (Probiomed, Mexico), Rituximab (Zenotech Laboratories), RituxiRel (Reliance Life Sciences, India), SAIT101 (Samsung BioLogics, South Korea), Rixathon/Riximyo (GP2013) (Sandoz, Switzerland), HLXO1 (Shanghai Henlius Biotech, China), TLO11 (Teva Pharmaceutical Industries, Israel; Lonza, Switzerland), or Redditux (TRPharma, Turkey).

### VII. Antibodies

In some embodiments, an antibody (e.g., an anti-CD79b antibody or an anti-CD20 antibody) used in a method of treatment provided herein may incorporate any of the features, singly or in combination, as described in below.

A. Antibody Affinity

In certain embodiments, an antibody (e.g., an anti-CD79b antibody or an anti-CD20 antibody) used in a method of treatment provided herein has a dissociation constant (Kd) of ≤1 μM, ≤100 nM, ≤50 nM, ≤10 nM, ≤5 nM, ≤1 nM, ≤0.1 nM, ≤0.01 nM, or ≤0.001 nM, and optionally is ≥10⁻¹³ M. (e.g., 10⁻⁸ M or less, e.g., from 10⁻⁸M to 10⁻¹³ M, e.g., from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER© multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20 ™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE©-2000 or a BIACORE®-3000 (BIAcore® Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

B. Antibody Fragments

In certain embodiments, an antibody (e.g., an anti-CD79b antibody or an anti-CD20 antibody) used in a method of treatment provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

C. Chimeric and Humanized Antibodies

In certain embodiments, an antibody a (e.g., an anti-CD79b antibody or an anti-CD20 antibody) used in a method of treatment provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

D. Human Antibodies

In certain embodiments, an antibody (e.g., an anti-CD79b antibody or an anti-CD20 antibody) used in a method of treatment provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For a review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

E. Library-Derived Antibodies

In some embodiments, an antibody (e.g., an anti-CD79b antibody or an anti-CD20 antibody) used in a method of treatment provided herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

F. Multispecific Antibodies

In certain embodiments, an antibody (e.g., an anti-CD79b antibody or an anti-CD20 antibody) used in a method of treatment provided herein is a multispecific antibody, e.g., a bispecific antibody. Multispecific antibodies are monoclonal antibodies that have binding specificities for at least two different sites. In certain embodiments, one of the binding specificities is for one antigen (e.g., CD79b or CD20) and the other is for any other antigen. In certain embodiments, one of the binding specificities is for one antigen (e.g., CD79b or CD20) and the other is for CD3. See, e.g., U.S. Pat. No. 5,821,337. In certain embodiments, bispecific antibodies may bind to two different epitopes of a single antigen (e.g., CD79b or CD20). Bispecific antibodies may also be used to localize cytotoxic agents to cells which express the antigen (e.g., CD79b or CD20). Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules

(WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to CD79b as well as another, different antigen (see, US 2008/0069820, for example).

G. Antibody Variants

In certain embodiments, amino acid sequence variants of an antibody (e.g., an anti-CD79b antibody or an anti-CD20 antibody) used in a method of treatment provided herein are contemplated. For example, it may be desirable to improve the binding affinity and/or other biological properties of the anti-CD79b antibody or anti-CD20 antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

(i) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table N under the heading of “preferred substitutions.” More substantial changes are provided in Table N under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE N

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies are displayed on phage and screened for a particular biological activity (e.g., binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

(ii) Glycosylation Variants

In certain embodiments, an antibody (e.g., an anti-CD79b antibody or an anti-CD20 antibody) used in a method of treatment provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

(iii) Fc Variants

In certain embodiments, one or more amino acid modifications may be introduced into the Fc region of an antibody (e.g., an anti-CD79b antibody or an anti-CD20 antibody) used in a method of treatment provided herein, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327, and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

(iv) Cysteine Engineered Antibody Variants

In certain embodiments, it may be desirable to create cysteine engineered antibodies, e.g., “thioMAbs,” in which one or more residues of an anti-CD79b antibody or an anti-CD20 antibody used in a method of treatment provided herein are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and 5400 (EU numbering) of the heavy chain Fc region. See, e.g., WO 2009/012268, for exemplary cysteine engineered anti-CD79b antibodies for use in the methods described herein. Cysteine engineered antibodies may be generated as described, e.g., in U.S. Pat. No. 7,521,541.

(v) Antibody Derivatives

In certain embodiments, an antibody (e.g., an anti-CD79b antibody or an anti-CD20 antibody) used in a method of treatment provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

H. Recombinant Methods and Compositions

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

I. Assays

An antibody (e.g., an anti-CD79b antibody or an anti-CD20 antibody) used in a method of treatment provided herein may be identified, screened for, or characterized for their physical/chemical properties and/or biological activities by various assays known in the art.

In one aspect, an antibody (e.g., an anti-CD79b antibody or an anti-CD20 antibody) used in a method of treatment provided herein is tested for its antigen binding activity, e.g., by known methods such as ELISA, BIACore®, FACS, or Western blot.

In another aspect, competition assays may be used to identify an antibody that competes with any of the antibodies described herein for binding to the target antigen. In certain embodiments, such a competing antibody binds to the same epitope (e.g., a linear or a conformational epitope) that is bound by an antibody described herein. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized antigen is incubated in a solution comprising a first labeled antibody that binds to antigen (e.g., any of the antibodies described herein) and a second unlabeled antibody that is being tested for its ability to compete with the first antibody for binding to antigen. The second antibody may be present in a hybridoma supernatant. As a control, immobilized antigen is incubated in a solution comprising the first labeled antibody but not the second unlabeled antibody. After incubation under conditions permissive for binding of the first antibody to antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to antigen. See Harlow and Lane (1988) Antibodies: A Laboratory Manual ch.14 (Cold Spring Harbor Laboratory, Cold Spring Harbor, N.Y.).

### IX. Pharmaceutical Formulations

Pharmaceutical formulations of any of the agents described herein (e.g., anti-CD79b immunoconjugates, anti-CD20 antibodies, and Bcl-2 inhibitors) for use in any of the methods as described herein are prepared by mixing such agent(s) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody or immunoconjugate formulations are described in U.S. Pat. No. 6,267,958. Aqueous antibody or immunoconjugate formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody or immunoconjugate, which matrices are in the form of shaped articles, e.g., films, or microcapsules.

The formulations to be used for in vivo administration are generally sterile. Sterility may be readily accomplished, e.g., by filtration through sterile filtration membranes.

Additional details regarding pharmaceutical formulations comprising an anti-CD79 immunoconjugate are provided in WO 2009/099728 the contents of which are expressly incorporated by reference herein in their entirety.

### X. Kits and Articles of Manufacture

In another embodiment, an article of manufacture or a kit is provided comprising an anti-CD79b immunoconjugate (such as described herein) and at least one additional agent. In some embodiments the at least one additional agent is a Bcl-2 inhibitor (such as venetoclax) and an anti-CD20 antibody (such as obinutuzumab or rituximab). In some embodiments, the article of manufacture or kit further comprises package insert comprising instructions for using the anti-CD79b immunoconjugate in conjunction at least one additional agent, such as a Bcl-2 inhibitor (e.g., venetoclax) and an anti-CD20 antibody (e.g., obinutuzumab or rituximab) to treat or delay progression of a B-cell proliferative disorder (e.g., FL, R/R FL, DLBCL, or R/R DLBCL) in an individual. Any of the anti-CD79b immunoconjugates and anti-cancer agents known in the art may be included in the article of manufacture or kits.

In some embodiments, provided herein is a kit comprising an immunoconjugate comprising the formula

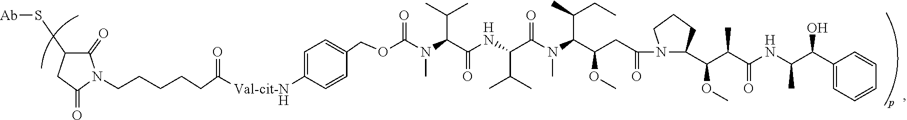

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with a Bcl-2 inhibitor and an anti-CD20 antibody for treating a human in need thereof having diffuse large B-cell lymphoma (DLBCL) according to any method provided herein.

In some embodiments, provided herein is a kit comprising an immunoconjugate comprising the formula

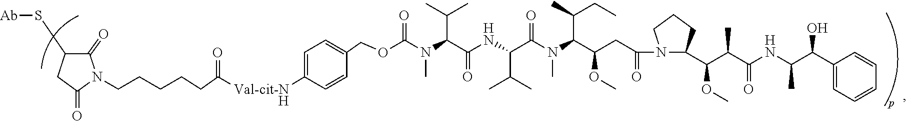

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with venetoclax and rituximab for treating a human in need thereof having diffuse large B-cell lymphoma (DLBCL) according to any method provided herein.

In some embodiments, p is between 3 and 4 or between 2 and 5. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35.

Also provided herein is a kit comprising polatuzumab vedotin-piiq for use in combination with venetoclax and rituximab for treating a human in need thereof having diffuse large B-cell lymphoma (DLBCL) according to any method provided herein.

In some embodiments, provided herein is a kit comprising an immunoconjugate comprising the formula

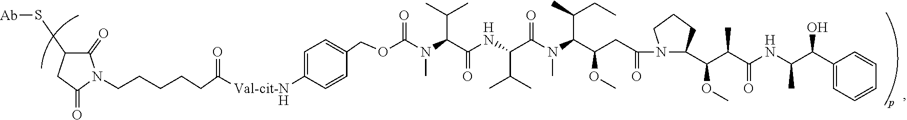

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with a Bcl-2 inhibitor and an anti-CD20 antibody for treating a human in need thereof having follicular lymphoma (FL) according to any method of treatment provided herein.

In some embodiments, provided herein is a kit comprising an immunoconjugate comprising the formula

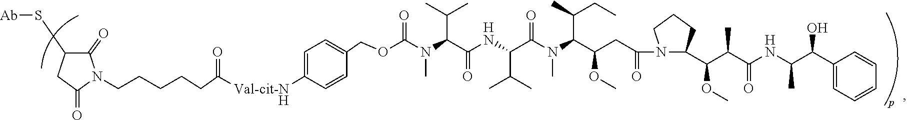

wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and wherein p is between 1 and 8, for use in combination with venetoclax and obinutuzumab for treating a human in need thereof having follicular lymphoma (FL) according to any method of treatment provided herein.

In some embodiments, p is between 3 and 4 or between 2 and 5. In some embodiments, the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20. In some embodiments, the anti-CD79b antibody comprises (i) a heavy chain comprising the amino acid sequence of SEQ ID NO: 36 and (ii) a light chain comprising the amino acid sequence of SEQ ID NO: 35.

Also provided herein is a kit comprising polatuzumab vedotin-piiq for use in combination with venetoclax and obinutuzumab for treating a human in need thereof having follicular lymphoma (FL) according to any method of treatment provided herein.

In some embodiments, the anti-CD79 immunoconjugate, the Bcl-2 inhibitor (such as venetoclax) and the anti-CD20 antibody (such as obinutuzumab or rituximab) are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

TABLE O
Amino Acid Sequences

| NAME | SEQUENCE | SEQ ID NO |
|---|---|---|
| Human CD79b | RFIARKRGFT VKMHCYMNSA SGNVSWLWKQ EMDENPQQLK | 1 |
| precursor; Acc. No. | LEKGRMEESQ NESLATLTIQ GIRFEDNGIY FCQQKCNNTS | |
| NP_000617.1; signal | EVYQGCGTEL RVMGFSTLAQ LKORNTLKDG IIMIQTLLII | |
| sequence = amino | LFIIVPIFLL LDKDDSKAGM EEDHTYEGLD IDQTATYEDI | |
| acids 1 to 28 | VTLRTGEVKW SVGEHPGQE | |
| Human mature CD79b, | AR SEDRYRNPKG SACSRIWQSP RFIARKRGFT VKMHCYMNSA | 2 |
| without signal | SGNVSWLWKQ EMDENPQQLK LEKGRMEESQ NESLATLTIQ | |
| sequence; amino | GIRFEDNGIY FCQQKCNNTS EVYQGCGTEL RVMGFSTLAQ | |
| acids 29 to 229 | LKQRNTLKDG IIMIQTLLII LFIIVPIFLL LDKDDSKAGM | |
| | EEDHTYEGLD IDQTATYEDI VTLRTGEVKW SVGEHPGQE | |
| VH of mMAb anti- | Gly Pro Glu Leu Val Lys Pro Gly Ala Ser Val | 3 |
| CD20 antibody B-Ly1 | Lys Ile Ser Cys Lys Ala Ser Gly Tyr Ala Phe | |
| | Ser Tyr Ser Trp Met Asn Trp Val Lys Leu Arg | |
| | Pro Gly Gln Gly Leu Glu Trp Ile Gly Arg Ile | |
| | Phe Pro Gly Asp Gly Asp Thr Asp Tyr Asn Gly | |
| | Lys Phe Lys Gly Lys Ala Thr Leu Thr Ala Asp | |
| | Lys Ser Ser Asn Thr Ala Tyr Met Gln Leu Thr | |
| | Ser Leu Thr Ser Val Asp Ser Ala Val Tyr Leu | |
| | Cys Ala Arg Asn Val Phe Asp Gly Tyr Trp Leu | |
| | Val Tyr Trp Gly Gln Gly Thr Leu Val Thr Val | |
| | Ser Ala | |
| VL of mMAb anti- | Asn Pro Val Thr Leu Gly Thr Ser Ala Ser Ile | 4 |
| CD20 antibody B-Ly1 | Ser Cys Arg Ser Ser Lys Ser Leu Leu His Ser | |
| | Asn Gly Ile Thr Tyr Leu Tyr Trp Tyr Leu Gln | |
| | Lys Pro Gly Gln Ser Pro Gln Leu Leu Ile Tyr | |
| | Gln Met Ser Asn Leu Val Ser Gly Val Pro Asp | |
| | Arg Phe Ser Ser Ser Gly Ser Gly Thr Asp Phe | |
| | Thr Leu Arg Ile Ser Arg Val Glu Ala Glu Asp | |
| | Val Gly Val Tyr Tyr Cys Ala Gln Asn Leu Glu | |
| | Leu Pro Tyr Thr Phe Gly Gly Gly Thr Lys Leu | |
| | Glu Ile Lys Arg | |
| GA101 HVR-H1 | Gly Tyr Ala Phe Ser Tyr | 5 |
| GA101 HVR-H2 | Phe Pro Gly Asp Gly Asp Thr Asp | 6 |
| GA101 HVR-H3 | Asn Val Phe Asp Gly Tyr Trp Leu Val Tyr | 7 |
| GA101 HVR-L1 | Arg Ser Ser Lys Ser Leu Leu His Ser Asn Gly | 8 |
| | Ile Thr Tyr Leu Tyr | |
| GA101 HVR-L2 | Gln Met Ser Asn Leu Val Ser | 9 |
| GA101 HVR-L3 | Ala Gln Asn Leu Glu Leu Pro Tyr Thr | 10 |
| GA101 VH | Gln Val Gln Leu Val Gln Ser Gly Ala Glu Val | 11 |
| | Lys Lys Pro Gly Ser Ser Val Lys Val Ser Cys | |
| | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| GA101 VL | Asp Ile Val Met Thr Gln Thr Pro Leu Ser Leu | 12 |
| | Pro Val Thr Pro Gly Glu Pro Ala Ser Ile Ser | |
| | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys Arg Thr Val | |
| GA101 Heavy Chain | Gln Val Gln Leu Val Gln Ser Gly Ala Glu Val | 13 |
| | Lys Lys Pro Gly Ser Ser Val Lys Val Ser Cys | |
| | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser Ala Ser | |
| | Thr Lys Gly Pro Ser Val Phe Pro Leu Ala Pro | |
| | Ser Ser Lys Ser Thr Ser Gly Gly Thr Ala Ala | |
| | Leu Gly Cys Leu Val Lys Asp Tyr Phe Pro Glu | |
| | Pro Val Thr Val Ser Trp Asn Ser Gly Ala Leu | |
| | Thr Ser Gly Val His Thr Phe Pro Ala Val Leu | |
| | Gln Ser Ser Gly Leu Tyr Ser Leu Ser Ser Val | |
| | Val Thr Val Pro Ser Ser Ser Leu Gly Thr Gln | |
| | Thr Tyr Ile Cys Asn Val Asn His Lys Pro Ser | |
| | Asn Thr Lys Val Asp Lys Lys Val Glu Pro Lys | |
| | Ser Cys Asp Lys Thr His Thr Cys Pro Pro Cys | |
| | Pro Ala Pro Glu Leu Leu Gly Gly Pro Ser Val | |
| | Phe Leu Phe Pro Pro Lys Pro Lys Asp Thr Leu | |
| | Met Ile Ser Arg Thr Pro Glu Val Thr Cys Val | |
| | Val Val Asp Val Ser His Glu Asp Pro Glu Val | |
| | Lys Phe Asn Trp Tyr Val Asp Gly Val Glu Val | |
| | His Asn Ala Lys Thr Lys Pro Arg Glu Glu Gln | |
| | Tyr Asn Ser Thr Tyr Arg Val Val Ser Val Leu | |
| | Thr Val Leu His Gln Asp Trp Leu Asn Gly Lys | |
| | Glu Tyr Lys Cys Lys Val Ser Asn Lys Ala Leu | |
| | Pro Ala Pro Ile Glu Lys Thr Ile Ser Lys Ala | |
| | Lys Gly Gln Pro Arg Glu Pro Gln Val Tyr Thr | |
| | Leu Pro Pro Ser Arg Asp Glu Leu Thr Lys Asn | |
| | Gln Val Ser Leu Thr Cys Leu Val Lys Gly Phe | |
| | Tyr Pro Ser Asp Ile Ala Val Glu Trp Glu Ser | |
| | Asn Gly Gln Pro Glu Asn Asn Tyr Lys Thr Thr | |
| | Pro Pro Val Leu Asp Ser Asp Gly Ser Phe Phe | |
| | Leu Tyr Ser Lys Leu Thr Val Asp Lys Ser Arg | |
| | Trp Gln Gln Gly Asn Val Phe Ser Cys Ser Val | |
| | Met His Glu Ala Leu His Asn His Tyr Thr Gln | |
| | Lys Ser Leu Ser Leu Ser Pro Gly | |
| GA101 Light Chain | Asp Ile Val Met Thr Gln Thr Pro Leu Ser Leu | 14 |
| | Pro Val Thr Pro Gly Glu Pro Ala Ser Ile Ser | |
| | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe Gly Gly Gly Thr Lys Val Glu | |
| | Ile Lys Arg Thr Val Ala Ala Pro Ser Val Phe | |
| | Ile Phe Pro Pro Ser Asp Glu Gln Leu Lys Ser | |
| | Gly Thr Ala Ser Val Val Cys Leu Leu Asn Asn | |
| | Phe Tyr Pro Arg Glu Ala Lys Val Gln Trp Lys | |
| | Val Asp Asn Ala Leu Gln Ser Gly Asn Ser Gln | |
| | Glu Ser Val Thr Glu Gln Asp Ser Lys Asp Ser | |
| | Thr Tyr Ser Leu Ser Ser Thr Leu Thr Leu Ser | |
| | Lys Ala Asp Tyr Glu Lys His Lys Val Tyr Ala | |
| | Cys Glu Val Thr His Gln Gly Leu Ser Ser Pro | |
| | Val Thr Lys Ser Phe Asn Arg Gly Glu Cys | |
| VH of humanized B- | Gln Val Gln Leu Val Gln Ser Gly Ala Glu Val | 15 |
| Ly1 antibody (B- | Lys Lys Pro Gly Ser Ser Val Lys Val Ser Cys | |
| HH2) | Lys Ala Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 16 |
| Ly1 antibody (B- | Lys Lys Pro Gly Ser Ser Val Lys Val Ser Cys | |
| HH3) | Lys Ala Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Leu Cys Ala Arg Asn | |
| | Val Phe Asp Gly Tyr Trp Leu Val Tyr Trp Gly | |
| | Gln Gly Thr Leu Val Thr Val Ser Ser | |
| humanized B-Ly1 | QVQLVQSGAE VKKPGSSVKV SCKASGYAFS YSWINWVRQA | 17 |
| Heavy Chain | PGQGLEWMGR IFPGDGDTDY NGKFKGRVTI TADKSTSTAY | |
| | MELSSLRSED TAVYYCARNV FDGYWLVYWG QGTLVTVSSA | |
| | STKGPSVFPL APSSKSTSGG TAALGCLVKD YFPEPVTVSW | |
| | NSGALTSGVH TFPAVLQSSG LYSLSSVVTV PSSSLGTQTY | |
| | ICNVNHKPSN TKVDKKVEPK SCDKTHTCPP CPAPELLGGP | |
| | SVFLFPPKPK DTLMISRTPE VTCVVVDVSH EDPEVKENWY | |
| | VDGVEVHNAK TKPREEQYNS TYRVVSVLTV LHQDWLNGKE | |
| | YKCKVSNKAL PAPIEKTISK AKGQPREPQV YTLPPSRDEL | |
| | TKNQVSLTCL VKGFYPSDIA VEWESNGQPE NNYKTTPPVL | |
| | DSDGSFFLYS KLTVDKSRWQ QGNVFSCSVM HEALHNHYTQ | |
| | KSLSLSPG | |
| humanized B-Ly1 | DIVMTQTPLS LPVTPGEPAS ISCRSSKSLL HSNGITYLYW | 18 |
| Light Chain | YLQKPGQSPQ LLIYQMSNLV SGVPDRFSGS GSGTDFTLKI | |
| | SRVEAEDVGV YYCAQNLELP YTFGGGTKVE IKRTVAAPSV | |
| | FIFPPSDEQL KSGTASVVCL LNNFYPREAK VQWKVDNALQ | |
| | SGNSQESVTE QDSKDSTYSL SSTLTLSKAD YEKHKVYACE | |
| | VTHQGLSSPV TKSFNRGEC | |
| huMA 79bv28 heavy | EVQLVESGGG LVQPGGSLRL SCAASGYTFS SYWIEWVRQA | 19 |
| chain variable | PGKGLEWIGE ILPGGGDTNY NEIFKGRATF SADTSKNTAY | |
| region | LQMNSLRAED TAVYYCTRRV PIRLDYWGQG TLVTVSS | |
| huMA79bv28 light | DIQLTQSPSS LSASVGDRVT ITCKASQSVD YEGDSFLNWY | 20 |
| chain variable | QQKPGKAPKL LIYAASNLES GVPSRFSGSG SGTDFTLTIS | |
| region | SLQPEDFATY YCQQSNEDPL TFGQGTKVEI KR | |
| huMA79bv28 HVR H1 | GYTFSSYWIE | 21 |
| huMA79bv28 HVR H2 | GEILPGGGDTNYNEIFKG | 22 |
| huMA79bv28 HVR H3 | TRRVPIRLDY | 23 |
| huMA79bv28 HVR L1 | KASQSVDYEGDSFLN | 24 |
| huMA79bv28 HVR L2 | AASNLES | 25 |
| huMA79bv28 HVR L3 | QQSNEDPLT | 26 |
| huMA79bv28 heavy | EVOLVESGGGLVQPGGSLRLSCAAS | 27 |
| chain (HC) | | |
| framework region | | |
| (FR) 1 | | |
| huMA79bv28 HC FR2 | WVRQAPGKGLEWI | 28 |
| huMA79bv28 HC FR3 | RATF SADTSKNTAYLQMNSLRAEDTAVYYC | 29 |
| huMA79bv28 HC FR4 | WGQGTLVTVSS | 30 |
| huMA79bv28 light | DIQLTQSPSSLSASVGDRVTITC | 31 |
| chain (LC) FR1 | | |
| huMA79bv28 LC FR2 | WYQQKPGKAPKLLIY | 32 |
| huMA79bv28 LC FR3 | GVPSRFSGSGSGTDFTLTISSLQPEDFATYYC | 33 |
| huMA79bv28 LC FR4 | FGQGTKVEIKR | 34 |
| huMA79bv28 light | DIQLTQSPSS LSASVGDRVT ITCKASQSVD YEGDSFLNWY | 35 |
| chain (Igκ) | QQKPGKAPKL LIYAASNLES GVPSRFSGSG SGTDFTLTIS | |
| | SLQPEDFATY YCQQSNEDPL TFGQGTKVEI KRTVAAPSVF | |
| | IFPPSDEQLK SGTASVVCLL NNFYPREAKV QWKVDNALQS | |
| | GNSQESVTEQ DSKDSTYSLS STLTLSKADY EKHKVYACEV | |
| | THQGLSSPVT KSFNRGEC | |
| huMA79bv28 heavy | EVQLVESGGG LVQPGGSLRL SCAASGYTFS SYWIEWVRQA | 36 |
| chain (IgG1) | PGKGLEWIGE ILPGGGDTNY NEIFKGRATF SADTSKNTAY | |
| | LQMNSLRAED TAVYYCTRRV PIRLDYWGQG TLVTVSSAST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKFNWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDS | |
| | DGSFFLYSKL TVDKSRWQQG NVFSCSVMHE ALHNHYTQKS | |
| | LSLSPG | |
| huMA79bv28 A118C | EVQLVESGGG LVQPGGSLRL SCAASGYTFS SYWIEWVRQA | 37 |
| cysteine engineered | PGKGLEWIGE ILPGGGDTNY NEIFKGRATF SADTSKNTAY | |
| heavy chain (IgG1) | LQMNSLRAED TAVYYCTRRV PIRLDYWGQG TLVTVSSCST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKFNWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDS | |
| | DGSFFLYSKL TVDKSRWQQG NVFSCSVMHE ALHNHYTQKS | |
| | LSLSPG | |
| huMA79bv28 V205C | DIQLTQSPSS LSASVGDRVT ITCKASQSVD YEGDSFLNWY | 38 |
| cysteine engineered | QQKPGKAPKL LIYAASNLES GVPSRFSGSG SGTDFTLTIS | |
| light chain (Igκ) | SLQPEDFATY YCQQSNEDPL TFGQGTKVEI KRTVAAPSVF | |
| | IFPPSDEQLK SGTASVVCLL NNFYPREAKV QWKVDNALQS | |
| | GNSQESVTEQ DSKDSTYSLS STLTLSKADY EKHKVYACEV | |
| | THQGLSSPCT KSFNRGEC | |
| huMA79bv28 S400C | EVQLVESGGG LVQPGGSLRL SCAASGYTFS SYWIEWVRQA | 39 |
| cysteine engineered | PGKGLEWIGE ILPGGGDTNY NEIFKGRATF SADTSKNTAY | |
| heavy chain (IgG1) | LQMNSLRAED TAVYYCTRRV PIRLDYWGQG TLVTVSSAST | |
| | KGPSVFPLAP SSKSTSGGTA ALGCLVKDYF PEPVTVSWNS | |
| | GALTSGVHTF PAVLQSSGLY SLSSVVTVPS SSLGTQTYIC | |
| | NVNHKPSNTK VDKKVEPKSC DKTHTCPPCP APELLGGPSV | |
| | FLFPPKPKDT LMISRTPEVT CVVVDVSHED PEVKFNWYVD | |
| | GVEVHNAKTK PREEQYNSTY RVVSVLTVLH QDWLNGKEYK | |
| | CKVSNKALPA PIEKTISKAK GQPREPQVYT LPPSREEMTK | |
| | NQVSLTCLVK GFYPSDIAVE WESNGQPENN YKTTPPVLDC | |
| | DGSFFLYSKL TVDKSRWQQG NVFSCSVMHE ALHNHYTQKS | |
| | LSLSPGK | |
| VH of humanized B- | Gln Val Gln Leu Val Gln Ser Gly Ala Glu Val | 40 |
| Ly1 antibody (B- | Lys Lys Pro Gly Ala Ser Val Lys Val Ser Cys | |
| HH4) | Lys Val Ser Gly Tyr Ala Phe Ser Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 41 |
| Ly1 antibody (B- | Lys Lys Pro Gly Ser Ser Val Lys Val Ser Cys | |
| HH5) | Lys Ala Ser Gly Tyr Ala Phe Ser Tyr Ser Trp | |
| | Met Ser Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 42 |
| Ly1 antibody (B- | Lys Lys Pro Gly Ser Ser Val Lys Val Ser Cys | |
| HH6) | Lys Ala Ser Gly Tyr Ala Phe Ser Tyr Ser Trp | |
| | Ile Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 43 |
| Ly1 antibody (B- | Lys Lys Pro Gly Ser Ser Val Lys Val Ser Cys | |
| HH7) | Lys Ala Ser Gly Tyr Ala Phe Ser Tyr Ser Trp | |
| | Ile Ser Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 44 |
| Ly1 antibody (B- | Lys Lys Pro Gly Ala Ser Val Lys Val Ser Cys | |
| HH8) | Lys Ala Ser Gly Tyr Thr Phe Thr Tyr Ser Trp | |
| | Met Asn Trp Val Arg Gln Ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Gln Ser Gly Ala Glu Val | 45 |
| Ly1 antibody (B- | Lys Lys Pro Gly Ala Ser Val Lys Val Ser Cys | |
| HH9) | Lys Ala Ser Gly Tyr Thr Phe Ser Tyr Ser Trp | |
| | Met Asn Trp Val Arg Gln ala Pro Gly Gln Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 46 |
| Ly1 antibody (B- | Val Lys Pro Gly Gly Ser Leu Arg Leu Ser Cys | |
| HL8) | Ala Ala Ser Gly Phe Thr Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 47 |
| Ly1 antibody (B- | Val Lys Pro Gly Gly Ser Leu Arg Leu Ser Cys | |
| HL10) | Ala Ala Ser Gly Phe Ala Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Gln Val Gln Leu Val Glu Ser Gly Gly Gly Leu | 48 |
| Ly1 antibody (B- | Val Lys Pro Gly Gly Ser Leu Arg Leu Ser Cys | |
| HL11) | Ala Ala Ser Gly Phe Thr Phe Ser Tyr Ser Trp | |
| | Met Asn Trp Val Arg Gln Ala Pro Gly Lys Gly | |
| | Leu Glu Trp Val Gly Arg Ile Phe Pro Gly Asp | |
| | Gly Asp Thr Asp Tyr Asn Gly Lys Phe Lys Gly | |
| | Arg Val Thr Ile thr Ala Asp Lys Ser Thr Ser | |
| | Thr Ala Tyr Met Glu Leu Ser Ser Leu Arg Ser | |
| | Glu Asp Thr Ala Val Tyr Tyr cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Ala Gly Leu | 49 |
| Ly1 antibody (B- | Val Lys Pro Gly Gly Ser Leu Arg Leu Ser Cys | |
| HL12) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Ser Ser Val | |
| VH of humanized B- | Glu Val Gln Leu Val Glu Ser Gly Gly Gly Val | 50 |
| Ly1 antibody (B- | Val Lys Pro Gly Gly Ser Leu Arg Leu Ser Cys | |
| HL13) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 51 |
| Ly1 antibody (B- | Lys Lys Pro Gly Gly Ser Leu Arg Leu Ser Cys | |
| HL14) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 52 |
| Ly1 antibody (B- | Val Lys Pro Gly Ser Ser Leu Arg Leu Ser Cys | |
| HL15) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly Thr Leu Val Thr Val Ser Ser | |
| VH of humanized B- | Glu Val Gln Leu Val Glu Ser Gly Gly Gly Leu | 53 |
| Ly1 antibody (B- | Val Lys Pro Gly Gly Ser Leu Arg Val Ser Cys | |
| HL16) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile Thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly thr Leu Val Thr Val Ser Ser | |
| VH of humanized B- | Glu Val Gln Leu Val Glu Ser Gly Gly Gly Leu | 54 |
| Ly1 antibody (B- | Val Lys Pro Gly Gly Ser Leu Arg Leu Ser Cys | |
| HL17) | Ala Ala Ser Gly Phe Thr Phe Ser Tyr Ser Trp | |
| | Met Asn Trp Val Arg Gln Ala Pro Gly Lys Gly | |
| | Leu Glu Trp Met Gly Arg Ile Phe Pro Gly Asp | |
| | Gly Asp Thr Asp Tyr Asn Gly Lys Phe Lys Gly | |
| | Arg Val Thr Ile thr Ala Asp Lys Ser Thr Ser | |
| | Thr Ala Tyr Met Glu Leu Ser Ser Leu Arg Ser | |
| | Glu Asp Thr Ala Val Tyr Tyr Cys Ala Arg Asn | |
| | Val Phe Asp Gly Tyr Trp Leu Val Tyr Trp Gly | |
| | Gln Gly thr Leu Val Thr Val Ser Ser | |
| VL of humanized B- | Asp Ile Val Met Thr Gln Thr Pro Leu Ser Leu | 55 |
| Ly1 antibody (B- | Pro Val Thr Pro Gly Glu Pro Ala Ser Ile Ser | |
| KVI) | Cys Arg Ser Ser Lys Ser Leu Leu His Ser Asn | |
| | Gly Ile Thr Tyr Leu Tyr Trp Tyr Leu Gln Lys | |
| | Pro Gly Gln Ser Pro Gln Leu Leu Ile Tyr Gln | |
| | Met Ser Asn Leu Val Ser Gly Val Pro Asp Arg | |
| | Phe Ser Gly Ser Gly Ser Gly Thr Asp Phe Thr | |
| | Leu Lys Ile Ser Arg Val Glu Ala Glu Asp Val | |
| | Gly Val Tyr Tyr Cys Ala Gln Asn Leu Glu Leu | |
| | Pro Tyr Thr Phe gly Gly Gly Thr Lys Val Glu | |
| | Ile Lys Arg Thr Val | |

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The following are examples of methods and compositions of the disclosure. It is understood that various other embodiments may be practiced, given the general description provided above.

### Example 1: An Anti-CD79b Immunoconjugate (Polatuzumab Vedotin) in Combination with an Anti-CD20 Antibody (Obinutuzumab or Rituximab) and a Bcl-2 Inhibitor (Venetoclax) in Relapsed or Refractory Follicular Lymphoma (FL) or Relapsed or Refractory Diffuse Large B-Cell Lymphoma (DLBCL)

This Example describes a Phase Ib/II study evaluating the safety and efficacy of obinutuzumab (G) in combination with polatuzumab vedotin (Pola) and venetoclax (V) in patients with relapsed or refractory follicular lymphoma (R/R FL) and rituximab (R) in combination with polatuzumab vedotin and venetoclax in patients with relapsed or refractory diffuse large B-cell lymphoma (R/R DLBCL).

### I. Study Objectives

The safety objectives of this study include determining the recommended Phase II dose (RP2D) for polatuzumab vedotin and venetoclax when given in combination with a fixed dose of obinutuzumab and the RP2D of venetoclax when given in combination with a fixed dose of polatuzumab vedotin based on the incidence of dose-limiting toxicities (DLTs) during the first cycle of study treatment. The safety objectives also include evaluating the safety and tolerability of G+Pola+V and R+Pola+V on the basis of the nature, frequency, severity, and timing of adverse events, including DLTs, and changes in vital signs, ECGs, and clinical laboratory results during and following study treatment administration.

Responses are determined on the basis of positron emission tomography and computed tomography (PET-CT) scans or CT scans alone, using Revised Lugano Response Criteria for Malignant Lymphoma, hereinafter referred to as Lugano 2014 criteria. Responses are determined by an Independent Review Committee (IRC) and by the investigator. The primary efficacy objective for this study was to evaluate the efficacy of G+Pola+V in patients with R/R FL and R+Pola+V in patients with R/R DLBCL on the basis of CR at end of induction (EOI), as determined by the IRC on the basis of PET-CT scans (by Modified Lugano 2014 criteria).

The secondary efficacy objectives for this study include evaluating the efficacy of G+Pola+V and maintenance treatment with G+V in patients with R/R FL and R+Pola+V and consolidation treatment with R+V in patients with R/R DLBCL on the basis of the following endpoints:
CR at EOI, as determined by the investigator on the basis of PET-CT scans.
CR at EOI, as determined by the IRC and by the investigator on the basis of CT scans alone.
Objective response (defined as a CR or PR) at EOI, as determined by the IRC and by the investigator on the basis of PET-CT scans.
Objective response (defined as a CR or PR) at EOI, as determined by the IRC and by the investigator on the basis of CT scans alone.
Best response of CR or PR during the study, as determined by the investigator on the basis of CT scans alone.

Exploratory efficacy objectives for this study include evaluating the long-term efficacy of G+Pola+V and R+Pola+V on the basis of the following endpoints:
For patients who have positive PET scans at EOI: CR at 12 months, as determined by the IRC and by the investigator on the basis of PET-CT scans in FL patients; CR at end of consolidation (EOC) as determined by the IRC and by the investigator on the basis of PET-CT scans in DLBCL patients.
Progression-free survival, defined as the time from initiation of study treatment to first occurrence of disease progression or relapse, as determined by investigator, or death from any cause.
Event-free survival, defined as the time from initiation of study treatment to any treatment failure, including disease progression or relapse, as determined by investigator, initiation of new anti-lymphoma therapy, or death from any cause, whichever occurs first.
Disease-free survival, defined, among patients who achieve a CR, as the time from the first occurrence of a documented CR to relapse, as determined by the investigator, or death from any cause, whichever occurs first.
Overall survival, defined as the time from initiation of study treatment to death from any cause.

Exploratory objectives of this study include characterization of biomarker profiles, pharmacokinetics, and immunogenicity of the triplet combination.

The pharmacokinetic (PK) objective for this study is to characterize the PK profiles of obinutuzumab, rituximab, polatuzumab vedotin, and venetoclax when given in combination, on the basis of the following endpoints:
Observed serum concentration of obinutuzumab at specified timepoints.
Observed serum concentration of rituximab at specified timepoints.
Observed serum and plasma concentrations of polatuzumab vedotin and relevant analytes (total antibody, antibody-conjugated mono-methyl auristatin E and unconjugated mono-methyl auristatin) at specified timepoints.
Observed plasma concentration of venetoclax at specified timepoints.

The immunogenicity objective for this study is to evaluate the immune response to obinutuzumab and to polatuzumab vedotin on the basis of the following endpoints:
Incidence of human anti-human antibodies (HAHAs) to obinutuzumab during the study relative to the prevalence of HAHAs at baseline.
Incidence of anti-therapeutic antibody (ATAs) to polatuzumab vedotin during the study relative to the prevalence of ATAs at baseline.

The exploratory immunogenicity objective for this study is to evaluate potential relationships between HAHAs or ATAs and other endpoints on the basis of the following endpoint:
Correlation between HAHA or ATA status and efficacy, safety, or PK endpoints.

The exploratory biomarker objective for this study is to identify non-inherited biomarkers that are predictive of response to study treatment (i.e., predictive biomarkers), are associated with progression to a more severe disease state (i.e., prognostic biomarkers), are associated with acquired resistance to study treatment, are associated with susceptibility to developing adverse events, can provide evidence of study treatment activity, can increase the knowledge and understanding of lymphoma biology or study treatment mechanism of action, or can contribute to improvement of diagnostic assays, on the basis of the following endpoint: Association between non-inherited biomarkers and efficacy, safety, PK, or immunogenicity endpoints.

### II. Study Design

This Phase Ib/II, open-label, multicenter, non-randomized study evaluated the safety, efficacy, and pharmacokinetics of obinutuzumab (G)+Polatuzumab vedotin (Pola)+venetoclax (V) in patients with R/R FL and rituximab (R)+Pola+V in patients with R/R DLBCL.

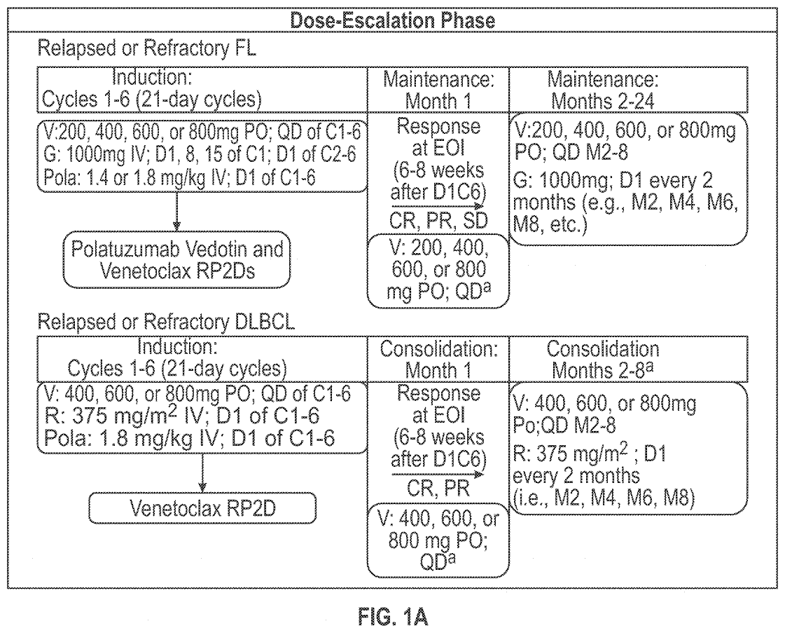

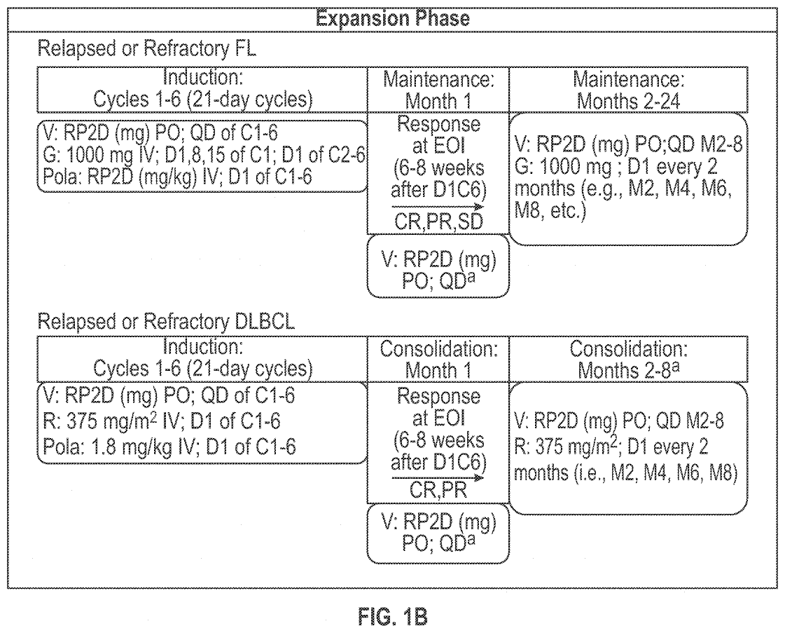

This study included an initial dose-escalation phase followed by an expansion phase during which polatuzumab vedotin and venetoclax will be given at their recommended Phase II doses (RP2Ds). Patients received induction treatment with obinutuzumab or rituximab, polatuzumab vedotin, and venetoclax. Patients with FL who achieve a CR, PR, or stable disease at end of induction (EOI) received post-induction treatment with obinutuzumab and venetoclax, and patients with DLBCL who achieve a CR or PR at EOI receive post-induction treatment with rituximab and venetoclax. The design of this study is shown in FIGS. 1A-1B.

### A. Patient Inclusion Criteria

This study enrolled patients with R/R FL or R/R DLBCL who met the following inclusion criteria:
Eastern Cooperative Oncology Group (ECOG) Performance Status of 0, 1, or 2.
For the G+Pola+V group: relapsed or refractory (R/R) FL after treatment with at least 1 prior chemoimmunotherapy regimen that included an anti-CD20 monoclonal antibody and for which no other more appropriate treatment option exists.
For the R+Pola+V group: R/R DLBCL after treatment with at least 1 prior chemoimmunotherapy regimen that included an anti-CD20 monoclonal antibody, with no curative option.
Histologically documented CD20-positive Non-Hodgkin lymphoma.
Fluorodeoxyglucose (FDG)-avid lymphoma (i.e., PET-positive lymphoma).
At least one bi-dimensionally measurable lesion (>1.5 cm in its largest dimension by computed tomography [CT] scan or magnetic resonance imaging [MRI]).
Availability of a representative tumor specimen and the corresponding pathology report for retrospective central confirmation of the diagnosis of FL or DLBCL. If the archival tissue is unavailable or unacceptable, a pretreatment core, excisional, or incisional tumor biopsy is required. If the patient received anti-lymphoma treatment between the time of the most recent available biopsy and initiation of study treatment, a core-needle biopsy was strongly recommended.

### B. Patient Exclusion Criteria

Patients meeting any of the following criteria were excluded from this study:
Known CD20-negative status at relapse or progression.
Prior allogeneic stem cell transplant (SCT).
Completion of autologous SCT within 100 days prior to Day 1 of Cycle 1.
Prior standard or investigational anti-cancer therapy as specified below:
   Radioimmunoconjugate within 12 weeks prior to Day 1 of Cycle 1.
   Monoclonal antibody or antibody-drug conjugate (ADC) therapy within 5 half-lives or 4 weeks prior to Day 1 of Cycle 1, whichever was longer.
   Radiotherapy, chemotherapy, hormonal therapy, or targeted small-molecule therapy within 2 weeks prior to Day 1 of Cycle 1.
Clinically significant toxicity (other than alopecia) from prior therapy that did not resolved to Grade ≤2 (per National Cancer Institute Common Terminology Criteria for Adverse Events [NCI CTCAE], Version 4.0) prior to Day 1 of Cycle 1.
Grade 3b FL.
History of transformation of indolent disease to DLBCL.
Current Grade >1 peripheral neuropathy.
Central nervous system (CNS) lymphoma or leptomeningeal infiltration.
Treatment with systemic corticosteroids >20 mg/day prednisone or equivalent.
   Patients who were receiving corticosteroids ≤20 mg/day, prednisone or equivalent, for non-lymphoma treatment reasons were documented to be on a stable dose for at least 4 weeks prior to Day 1 of Cycle 1.
   If corticosteroid treatment was urgently required for lymphoma symptom control prior to the start of study treatment, up to 100 mg/day of prednisone or equivalent were given for a maximum of 5 days, but all tumor assessments were completed prior to start of corticosteroid treatment.
History of severe allergic or anaphylactic reaction to humanized or murine monoclonal antibodies.
Known sensitivity or allergy to murine products or any component of the obinutuzumab, rituximab, polatuzumab vedotin, or venetoclax formulations.
Active bacterial, viral, fungal, or other infection.
Requirement for warfarin treatment.
Treatment with the following agents within 7 days prior to the first dose of venetoclax:
   Strong and moderate CYP3A inhibitors such as fluconazole, ketoconazole, and clarithromycin.
   Strong and moderate CYP3A inducers such as rifampin and carbamazepine.
Consumption of grapefruit, grapefruit products, Seville oranges (including marmalade that contains Seville oranges), or star fruit within 3 days prior to the first dose of venetoclax.
Clinically significant history of liver disease, including viral or other hepatitis, current alcohol abuse, or cirrhosis.
Positive for hepatitis B surface antigen (HBsAg), total hepatitis B core antibody (HBcAb), or hepatitis C virus (HCV) antibody at screening.
Known history of HIV positive status.
History of progression multifocal leukoencephalopathy (PML).
Vaccination with a live virus vaccine within 28 days prior to Day 1 of Cycle 1.
Significant cardiovascular or liver disease.
Inadequate renal or liver function, unless due to underlying disease.
Inadequate hematologic function (unless due to underlying lymphoma), defined as follows:
   Hemoglobin<9 g/dL.
   ANC<1.5×10⁹/L.
   Platelet count<75×10⁹/L.
Any of the following abnormal laboratory values (unless due to underlying lymphoma):
   Calculated creatinine clearance <50 mL/min with the use of 24-hour creatinine clearance or modified Cockcroft-Gault equation (eCCr; with use of the ideal body mass [IBM] instead of mass): eCCR=((140−Age)·IBM (kg)·[0.85 if female])/(72·serum creatinine (mg/dL)); or
   If serum creatinine is in mol/L: eCCR=((140−Age)·IBM (kg)·[1.23 if male, 1.04 if female])/(serum creatinine (μmol/L)).
   AST or ALT>2.5×by ULN.
   Serum total bilirubin>1.5×ULN (or >3×ULN for patients with Gilbert syndrome).
   INR or PT>1.5×ULN in the absence of therapeutic anticoagulation.
   PTT or aPTT>1.5×ULN in the absence of a lupus anticoagulant.

### C. Study Treatment

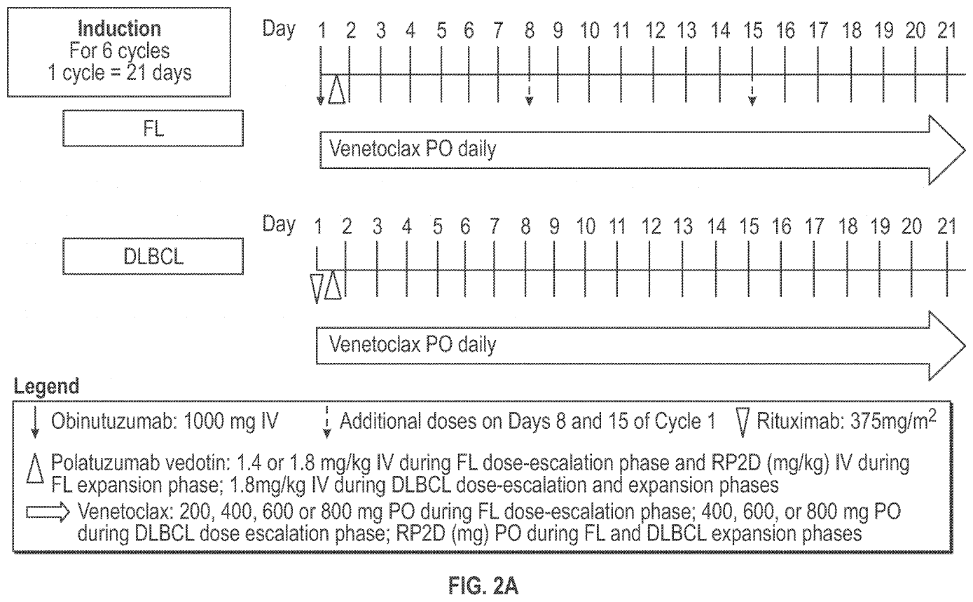

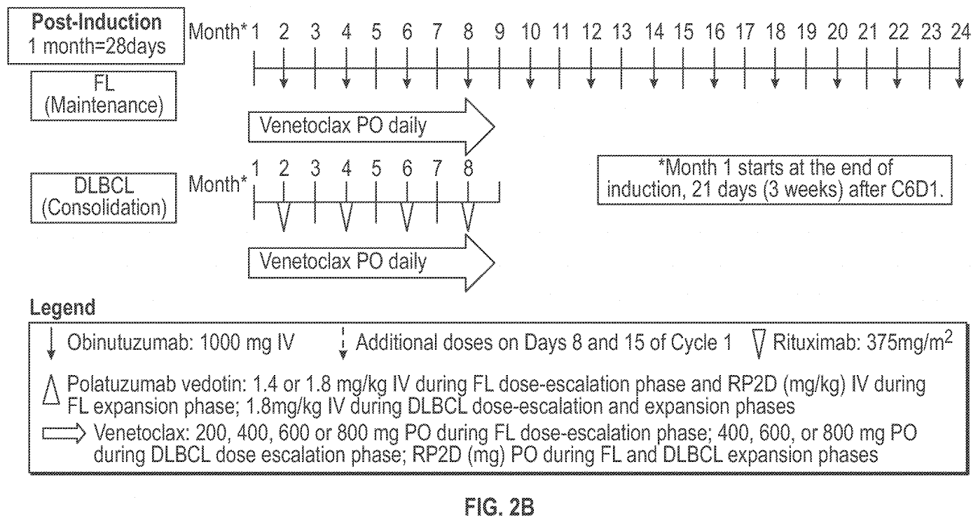

An overview of the study treatment regimens for the induction and post induction phases of this study is provided in FIGS. 2A-2B.

(i) FL Dose Escalation Phase

The purpose of the FL dose-escalation phase was to identify the RP2D for polatuzumab vedotin and the RP2D for venetoclax when combined with a fixed dose of obinutuzumab as induction treatment. This dose-escalation phase included FL patients only. The RP2D was based on the maximum tolerated doses (MTDs) and the totality of data for polatuzumab vedotin and venetoclax.

All patients enrolled in the dose-escalation phase received induction treatment, administered in 21-day cycles as shown in Table 1.

TABLE 1
Induction Treatment for the Follicular
Lymphoma Dose-Escalation Phase.

| | G + Pola + V (21-Day Cycles) |
|---|---|
| Cycle 1 | Venetoclax 200, 400, 600, or 800 mg PO once daily on |
| | Days 1-21. |
| | Obinutuzumab 1000 mg IV on Days 1, 8, and 15. |
| | Polatuzumab vedotin 1.4 or 1.8 mg/kg IV on Day 1. |
| Cycles 2-6 | Venetoclax 200, 400, 600, or 800 mg PO once daily |
| | on Days 1-21. |
| | Obinutuzumab 1000 mg IV on Day 1. |
| | Polatuzumab vedotin 1.4 or 1.8 mg/kg IV on Day 1. |

G = obinutuzumab; IV = intravenous; PO = by mouth; Pola = polatuzumab vedotin; V = venetoclax.
Note:
When study treatments were given on the same day, they were administered sequentially in the following order: venetoclax, obinutuzumab, and polatuzumab vedotin.

After completion of induction treatment, patients continued to receive daily venetoclax treatment (during Month 1) until response was assessed at EOI. Patients who achieved a CR, PR, or stable disease at EOI received maintenance treatment with obinutuzumab and venetoclax. During maintenance treatment, venetoclax was administered at a dose of 200, 400, 600, or 800 mg PO once daily for 8 months (Months 1-8) and obinutuzumab was administered at a dose of about 1000 mg IV on Day 1 of every other month (i.e., every 2 months) for 24 months, starting with Month 2 (e.g., Months 2, 4, 6, 8, etc.). Maintenance treatment continued until disease progression or unacceptable toxicity for up to 24 months. When study treatments were given on the same day, venetoclax was administered prior to obinutuzumab. A month was defined as 28 days.

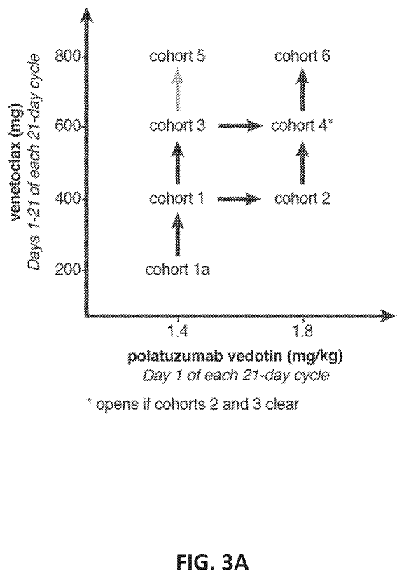

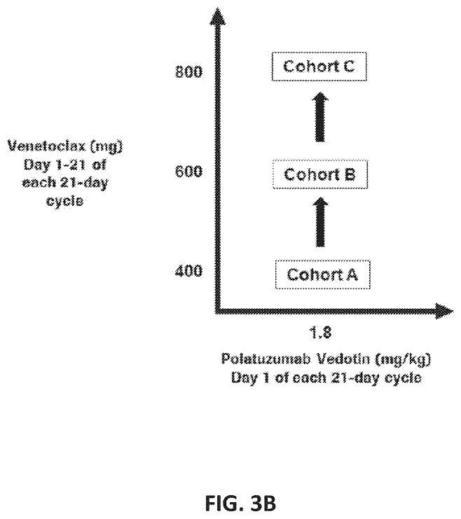

As shown in FIG. 3A, both polatuzumab vedotin and venetoclax were escalated in a 3+3 design, with ≥3 patients enrolled in each arm per cohort. The obinutuzumab dose remained fixed at 1000 mg during the dose-escalation phase. The starting doses in Cohort 1a were 1.4 mg/kg for polatuzumab vedotin and 200 mg for venetoclax. For polatuzumab vedotin, there were 2 possible dose levels: 1.4 mg/kg or 1.8 mg/kg. For venetoclax, there were 4 dose levels: 200, 400, 600, or 800 mg. The doses for each cohort are summarized in Table 2.

TABLE 2
Follicular Lymphoma Dose-Escalation Cohorts.

| Cohort | Obinutuzumab | Polatuzumab Vedotin | Venetoclax |
|---|---|---|---|
| 1a | 1000 mg | 1.4 mg/kg | 200 mg |
| 1 | 1000 mg | 1.4 mg/kg | 400 mg |
| 2 | 1000 mg | 1.8 mg/kg | 400 mg |
| 3 | 1000 mg | 1.4 mg/kg | 600 mg |
| 4 | 1000 mg | 1.8 mg/kg | 600 mg |
| 5 | 1000 mg | 1.4 mg/kg | 800 mg |
| 6 | 1000 mg | 1.8 mg/kg | 800 mg |

The observation period was based on evaluation of dose-limiting toxicities (DLTs) in cycle 1. If none of the first three evaluable patients experienced a DLT, the next dose cohort was opened. If a DLT was observed in one patient, additional patients were enrolled at that dose level until ≥6 evaluable patients had completed the DLT observation window or a second DLT occurred. If no additional DLTs were reported, the next dose could be evaluated. The MTD was defined as the highest dose resulting in DLTs in less than one-third of patients in a cohort of six or more patients. If the MTD was reached, this would be the RP2D. If the MTD was not exceeded in any cohort, then the highest dose combination administered would be confirmed as the RP2D, validated in six or more patients.

(ii) FL Expansion Phase

The expansion phase was designed to further assess the safety and efficacy of polatuzumab vedotin and venetoclax at their respective RP2Ds when combined with a fixed dose of obinutuzumab in FL patients.

All patients enrolled in the expansion phase received induction treatment, administered in 21-day cycles as outlined in Table 3.

TABLE 3
Induction Treatment for the Follicular
Lymphoma Expansion Phase.

| | G+ Pola + V (21-Day Cycles) |
|---|---|
| Cycle 1 | Venetoclax RP2D (mg) PO once daily on Days 1-21. |
| | Obinutuzumab 1000 mg IV on Days 1, 8, and 15. |
| | Polatuzumab vedotin RP2D (mg/kg) IV on Day 1. |
| Cycles 2-6 | Venetoclax RP2D (mg) PO once daily on Days 1-21. |
| | Obinutuzumab 1000 mg IV on Day 1. |
| | Polatuzumab vedotin RP2D (mg/kg) IV on Day 1. |

G = obinutuzumab; IV = intravenous; PO = by mouth; Pola = polatuzumab vedotin; V = venetoclax.
Note:
When study treatments were given on the same day, they were administered sequentially in the following order: venetoclax, obinutuzumab, and polatuzumab vedotin.

After completion of induction treatment, patients continued to receive daily venetoclax treatment (during Month 1) until response was assessed at EOI. Patients with FL who achieved a CR, PR, or stable disease at EOI received post-induction treatment (referred to as maintenance) with obinutuzumab and venetoclax. Post-induction treatment continued until disease progression or unacceptable toxicity for up to 24 months for maintenance treatment. During the maintenance phase, venetoclax was administered at the RP2D (mg) PO once daily for 8 months (Months 1-8) and obinutuzumab was administered at the dose of 1000 mg IV on Day 1 of every other month (i.e., every 2 months) for 24 months, starting with Month 2 (e.g., Months 2, 4, 6, 8, etc.). When study treatments were given on the same day, they were administered sequentially in the following order: venetoclax, obinutuzumab. A month was defined as 28 days.

(iii) DLBCL Dose Escalation Phase

The purpose of the DLBCL dose-escalation phase is to identify the RP2D for venetoclax when combined with polatuzumab vedotin at 1.8 mg/kg and rituximab at 375 mg/m² as induction treatment in patients with R/R DLBCL.

Patients enrolled in the DLBCL dose-escalation phase receive induction treatment, administered in 21-day cycles for up to six cycles. In cycles 1-6, venetoclax is administered at a dose of 400, 600, or 800 mg PO once daily on Days 1-21; rituximab is administered a the dose of 375 mg/m²IV on Day 1; and polatuzumab vedotin is administered at the dose of 1.8 mg/kg IV on Day 1. When study treatments are given on the same day, they are administered sequentially in the following order: venetoclax, rituximab, and polatuzumab vedotin.

After completion of induction treatment, patients continue to receive daily venetoclax treatment (during Month 1) until response is assessed at EOI. Patients who achieve a CR or PR at EOI receive consolidation treatment with rituximab and venetoclax. Consolidation treatment continues until disease progression or unacceptable toxicity for up to 8 months. When study treatments are given on the same day, venetoclax is administered prior to rituximab. During consolidation treatment, venetoclax is administered at a dose of 400, 600, or 800 mg PO once daily for 8 months (Months 1-8), and rituximab is administered at the dose of 375 mg/m²IV on Day 1 of every other month (i.e., every 2 months) starting with Month 2 (i.e., Months 2, 4, 6, and 8) for 8 months. Treatments are administered sequentially in the following order: venetoclax followed by rituximab.

A standard 3+3 dose-escalation schema is used. The rituximab and polatuzumab dose levels remain fixed during the dose-escalation phase and only the venetoclax is dose escalated. The polatuzumab dose of 1.8 mg/kg. The dose escalation plan is shown in FIG. 3B.

The study treatment doses for each cohort are shown in Table 4. If Cohort A doses are deemed safe and tolerable, escalation continues with enrollment of Cohort B. If Cohort B doses are deemed safe and tolerable, escalation continues with enrollment of Cohort C.

TABLE 4
DLBCL Dose-Escalation Cohorts.

| | | Polatuzumab | |
|---|---|---|---|
| Cohort | Rituximab | vedotin | Venetoclax |
| A | 375 mg/m² | 1.8 mg/kg | 400 mg |
| B | 375 mg/m² | 1.8 mg/kg | 600 mg |
| C | 375 mg/m² | 1.8 mg/kg | 800 mg |

Dose escalation occurs as follows: A minimum of 3 patients are initially enrolled in each cohort. The first 3 patients in each cohort are sequentially enrolled and dosed at least 48 hours apart. If none of the first 3 DLT-evaluable patients experience a DLT, the doses in that cohort are deemed safe and tolerable and escalation continues. If 1 of the first 3 DLT-evaluable patients experiences a DLT, the cohort is expanded to 6 patients. If there are no further DLTs in the first 6 DLT-evaluable patients, the doses in that cohort is deemed safe and tolerable and escalation continues. If a DLT is observed in ≥33% of patients (e.g., 2 or more of up to 6 DLT-evaluable patients), the dose combination at which this occurs is considered intolerable and the MTD is exceeded for venetoclax in the R+Pola+V treatment combination. If the MTD is exceeded in any cohort, the highest dose combination at which <33% of patients (e.g., 2 of 6 DLT-evaluable patients) experience a DLT is declared the combination MTD (i.e., the MTD venetoclax in the R+Pola+V treatment combination). If the MTD is not exceeded at any dose level, the highest dose combination administered in this study is declared the maximum administered dose for polatuzumab vedotin and venetoclax in the R+Pola+V treatment combination. If the MTD is exceeded in any cohort, de-escalation of the venetoclax dose and/or polatuzumab vedotin dose, and/or adjustment of treatment schedules (e.g., venetoclax treatment on Days 1-10) occurs. Additional patients are enrolled in a given cohort in the absence of DLTs to acquire additional safety data for the appropriate dose levels in the expansion phase of the study.

(iv) DLBCL Expansion Phase

The expansion phase is designed to further assess the safety and efficacy of venetoclax when combined with a fixed dose of rituximab and polatuzumab vedotin in DLBCL patients.

All patients enrolled in the expansion phase receive induction treatment in 21-day cycles as follows: venetoclax is administered at the RP2D (mg) PO once daily on Days 1-21 of cycles 1-6; rituximab is administered at the dose of 375 mg/m²IV on Day 1 of cycles 1-6; and polatuzumab vedotin is administered at the dose of 1.8 mg/kg IV on Day 1 of cycles 1-6. When study treatments are given on the same day, they are administered sequentially in the following order: venetoclax, rituximab, and polatuzumab vedotin.

After completion of induction treatment, patients continue to receive daily venetoclax treatment (during Month 1) until response is assessed at EOI. Patients with DLBCL who achieve a CR or PR at EOI receive post-induction treatment (referred to as consolidation) with rituximab and venetoclax. Consolidation treatment is administered as follows for 8 months: venetoclax is administered at the RP2D (mg) PO once daily for 8 months (Months 1-8) and rituximab is administered at the dose of 375 mg/m²IV on Day 1 of every other month (i.e., every 2 months) starting with Month 2 (i.e., Months 2, 4, 6, and 8) for 8 months. Post-induction treatment continues until disease progression or unacceptable toxicity for up to 8 months for consolidation treatment. When study treatments are given on the same day, venetoclax is administered prior to rituximab.

(v) Post-Treatment and Follow-Up

Patients who complete treatment or discontinue treatment for reasons other than disease progression undergo assessments every 3 months during the post-treatment follow-up period, which continues until disease progression, the start of new anti-lymphoma treatment, or the end of the study, whichever occurs first. Patients who experience disease progression are evaluated for survival status and initiation of new anti-lymphoma treatment every 3 months until the end of the study.

(vi) Study Drugs

Obinutuzumab is supplied as a single-dose, sterile liquid formulation in a 50-mL glass vial that contains 1000 mg of obinutuzumab.

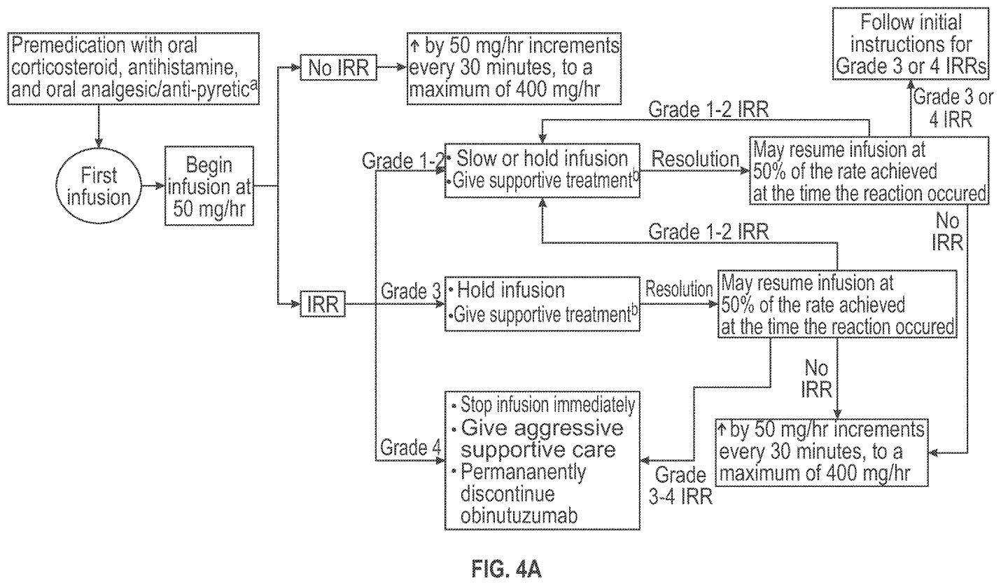

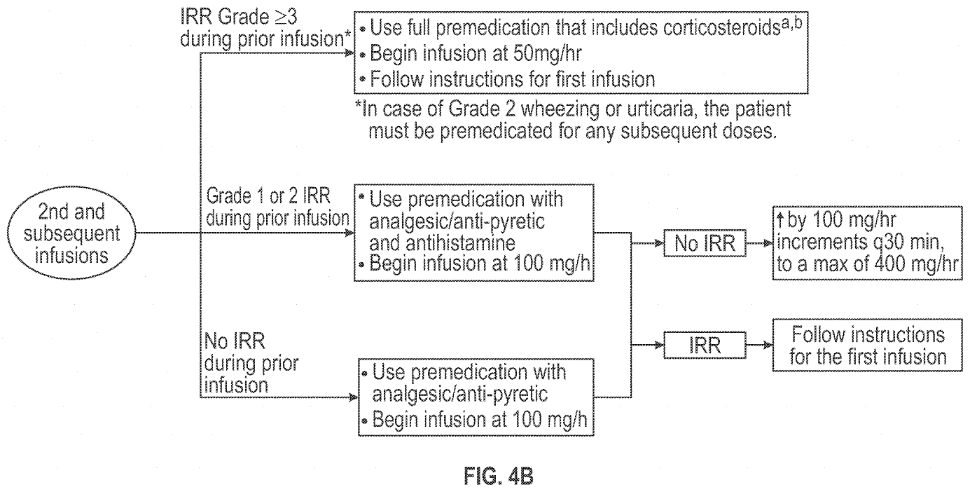

Obinutuzumab is administered as an IV infusion through a dedicated line. Obinutuzumab infusions are administered as shown in FIGS. 4A-4B. For patients with bulky lymphadenopathy, the infusion may be given slowly over a longer period of time, or the dose may be split and given over more than 1 day. No dose modifications for obinutuzumab are allowed. Premedication with a corticosteroid, antihistamine, and analgesic/antipyretic is required to reduce the incidence and severity of infusion related reactions (IRRs).

Rituximab is supplied packaged in 10-mL (100-mg) and 50-mL (500-mg) single-dose, pharmaceutical-grade glass vials at a concentration of 10 mg/mL of protein. The antibody is formulated for IV injection as a sterile product in a solution of sodium chloride (pH 6.5) containing polysorbate 80 and sodium citrate. Body surface area (BSA) is determined at screening and is used to calculate the dose of rituximab throughout the study unless the patient's weight increases or decreases by >10% from screening, in which case BSA is recalculated and used for subsequent dosing. In obese patients (defined as body mass index ≥30 kg/m²), there is no BSA cap and actual body weight, not adjusted weight, is recommended. Empiric dose adjustment for obese patients may be implemented per institutional guidelines. The infusion of rituximab may be split over 2 days if the patient is at increased risk for an IRR (high tumor burden or high peripheral lymphocyte count). Administration of rituximab may be continued on the following day, if needed, for patients who experience an adverse event during the rituximab infusion. If a dose of rituximab is split over 2 days, both infusions occur with premedication and at the first infusion rate. If a patient tolerates the first cycle of study treatment without significant infusion reactions, rituximab may be administered as a rapid infusion (over 60-90 minutes). Rituximab is administered as a slow IV infusion through a dedicated line. No dose modification for rituximab is allowed. Premedication with a corticosteroid, analgesic/antipyretic, and antihistamine is required to reduce the incidence and severity of IRRs.

The first infusion of rituximab (Day 1 of Cycle 1) begins at an initial rate of 50 mg/hr. If no infusion-related or hypersensitivity reaction occurs, the infusion rate is increased in 50-mg/hr increments every 30 minutes to a maximum of 400 mg/hr. If a reaction develops, the infusion.is stopped or slowed and medications and supportive care are administered. If the reaction has resolved, infusion is resumed at a 50% reduction in rate (i.e., 50% of rate being used at the time when the reaction occurred).

Subsequent rituximab infusions occur as follows: If the patient experienced an infusion related or hypersensitivity reaction during the prior infusion, full premedication is used, including 100 mg of prednisone/prednisolone or 80 mg of methylprednisolone or equivalent (until no further IRR occurs); the infusion begins at an initial rate of 50 mg/hr; and instructions for first infusion are followed. If the patient tolerated the prior infusion well (defined by an absence of Grade 2 reactions during a final infusion rate of ≥100 mg/hr), infusion begins at a rate of 100 mg/hr. If no reaction occurs, the infusion rate is increased in 100-mg/hr increments every 30 minutes to a maximum of 400 mg/hr. If a reaction develops, the infusion is stopped and slowed, and medications and supportive care are administered. If the reaction has resolved, the infusion resumes at a 50% reduction in rate (i.e., 50% of rate being used at the time when the reaction occurred).

Polatuzumab vedotin is supplied as a sterile, white to off-white, preservative-free lyophilisate in single-use vials. The patient's weight obtained during screening (Day −28 to Day −1) is used for dose determination for all treatment cycles. If the patient's weight within 96 hours prior to Day 1 of a given treatment cycle is >10% from the weight obtained during screening, the new weight is used to calculate the dose. The weight that triggered a dose adjustment is the new reference weight for future dose adjustments. After reconstitution with Sterile Water for Injection (SWFI) and dilution into IV bags that contain isotonic sodium chloride solution (0.9% NaCl), polatuzumab vedotin is administered by IV infusion using a dedicated standard administration set with 0.2-μm or 0.22-μm in-line filters at a final polatuzumab vedotin concentration determined by the patient-specific dose. The initial dose is administered to patients who are well hydrated over 90 (±10) minutes. Premedication (e.g., 500-1000 mg of oral acetaminophen or paracetamol and 50-100 mg diphenhydramine) may be administered to an individual patient before administration of polatuzumab vedotin. Administration of corticosteroids is permitted at the discretion of the treating physician. If IRRs are observed with the first infusion in the absence of premedication, premedication is administered before subsequent doses. The polatuzumab vedotin infusion may be slowed or interrupted for patients who experience infusion-associated symptoms. Following the initial dose, patients are observed for 90 minutes for fever, chills, rigors, hypotension, nausea, or other infusion-associated symptoms. If prior infusions are well tolerated, subsequent doses of polatuzumab vedotin are administered over 30 (±10) minutes, followed by a 30-minute observation period after the infusion. There are no dose reductions of polatuzumab vedotin for any toxicity except neurotoxicity.

Venetoclax is supplied as oral film-coated tablets of 100-mg strength in high-density polyethylene plastic bottles. The dose of venetoclax may be reduced according to the dose reduction steps shown in Table 5 based on the starting dose.

TABLE 5
Venetoclax dose reduction steps.
Venetoclax Dose Reduction Steps during Induction

Dose Reduction

| | | |
|---|---|---|
| Starting Dose | Step 1 | Step 2 |

Venetoclax Dose Reduction Steps during Induction

| | | |
|---|---|---|
| 800 mg | 600 mg | 400 mg |
| 600 mg | 400 mg | 200 mg |
| 400 mg | 200 mg | 100 mg |
| 200 mg | 100 mg | None |

Venetoclax Dose Reduction Steps during Maintenance

Dose Reduction

| Starting Dose | Step 1 | Step 2 | Step 3 | Step 4 |
|---|---|---|---|---|
| 800 mg | 600 mg | 400 mg | 200 mg | 100 mg |
| 600 mg | 400 mg | 200 mg | 100 mg | None |
| 400 mg | 200 mg | 100 mg | None | None |
| 200 mg | 100 mg | None | None | None |

All patients who receive venetoclax receive prophylaxis for tumor lysis syndrome (TLS) before the initiation of venetoclax in the G+Pola+V and R+Pola+V combination treatment. Patients who receive venetoclax who are at high risk for TLS or with compromised renal function are hospitalized on the first day of Cycle 1. Patients self-administer venetoclax tablets by mouth once daily. Each dose of venetoclax is taken orally once daily with approximately 240 mL of water within approximately 30 minutes after the completion of breakfast or the subject's first meal of the day. A meal containing approximately 30% of the total caloric content from fat is recommended to ensure adequate absorption of venetoclax.

(vii) Premedication

Premedication is administered as set forth in Table 6.

TABLE 6
Premedication.

| | Patients who Require | | |
|---|---|---|---|
| Timepoint | Premedication | Premedication | Administration |
| Cycle 1 Day 1 | All patients | Oral corticosteroid^{a} | Complete ≥1 hour |
| | | | prior to obinutuzumab |
| | | | or rituximab infusion. |
| | All patients | Antihistamine drug^{b} | Administer ≥30 |
| | | Oral analgesic/ | minutes prior to |
| | | anti-pyretic^{c} | obinutuzumab or |
| | | | rituximab Infusion. |
| | All patients | Allopurinol or suitable | Administer prior to |
| | | alternative such as | venetoclax. |
| | | rasburicase, along with | |
| | | adequate hydration | |
| Cycle 1, | Patients with no IRR | Oral analgesic/ | Administer ≥30 |
| Days 8 and 15 | during the previous | anti-pyretic^{c} | minutes prior to |
| Cycles 2 and | infusion | | obinutuzumab infusion. |
| beyond, Day 1 | | | For patients receiving |
| | | | rituximab, |
| | | | premedication may be |
| | | | omitted. |
| | Patients with Grade | Antihistamine drug^{b} | Administer ≥30 |
| | 1 or 2 IRR during | Oral analgesic/ | minutes prior to |
| | the previous infusion | anti-pyretic^{c} | obinutuzumab or |
| | | | rituximab infusion. |
| | Patients with Grade 3 | Oral corticosteroid^{a} | Complete ≥1 hour |
| | IRR, wheezing, | | prior to obinutuzumab |
| | urticaria, or other | | or rituximab infusion. |
| | symptoms of | Antihistamine drug^{b} | Administer ≥30 |
| | anaphylaxis during the | Oral analgesic/ | minutes prior to |
| | previous infusion | anti-pyretic^{c} | obinutuzumab or |
| | Patients with bulky | | rituximab infusion. |
| | disease | Allopurinol or suitable | Administer prior to |
| | Patients still at risk | alternative such as | obinutuzumab or |
| | for tumor lysis | rasburicase, along with | rituximab infusion. |
| | syndrome | adequate hydration | |

IRR = infusion-related reaction.
^{a}Treat with 100 mg of prednisone or prednisolone, 20 mg of dexamethasone, or 80 mg of methylprednisolone. Hydrocortisone should not be used, as it has not been effective in reducing rates of IRR.
^{b}For example, 50 mg of diphenhydramine.
^{c}For example, 1000 mg of acetaminophen/paracetamol.

(viii) Concomitant Therapy

Patients who use oral contraceptives, hormone-replacement therapy, or other maintenance therapy continue their use. Prior vitamin K antagonist therapy is replaced with low-molecular-weight heparin (LMWH) prior to Day 1 of Cycle 1. Hematopoietic growth factors are allowed. G-CSF may be administered in each cycle of therapy as primary prophylaxis for neutropenia, per American Society of Clinical Oncology (ASCO), EORTC, and European Society for Medical Oncology (ESMO) guidelines (Smith et al. 2006) or in the event of grade 3-4 neutropenia. Prophylactic treatment with antibiotics, e.g., for viral, fungal, bacterial, or pneumocystis infections is permitted. A brief (≤5 days) course of steroids (up to 100 mg of prednisone or equivalent per day) is allowed prior to initiation of study treatment for control of lymphoma-related symptoms.

(ix) Overlapping Toxicities

The overlapping toxicities from the combined administration of obinutuzumab or rituximab, polatuzumab vedotin, and venetoclax are anticipated in this clinical trial and are closely monitored and managed throughout the study.

Rituximab was safely combined with polatuzumab vedotin in patients with R/R FL or DLBCL. Grade 3 or 4 neutropenia (21%) appeared to be the most important hematologic adverse event associated with this combination.

When given as monotherapy for the treatment of patients with R/R NHL, obinutuzumab was associated with a 5% incidence of Grade 3-4 neutropenia. Because obinutuzumab is to have an incidence of neutropenia that is higher than that with rituximab monotherapy, there is a risk of increase incidence of neutropenia.

Venetoclax has also been associated with hematologic adverse events, including neutropenia. Therefore, the combination of obinutuzumab or rituximab, polatuzumab vedotin, and venetoclax is anticipated to have overlapping hematologic toxicity and is closely monitored.

There is the identified risk of TLS for treatment with obinutuzumab or rituximab or venetoclax and a theoretical risk for polatuzumab vedotin since these agents can result in the rapid destruction of a large number of tumor cells. Therefore, overlapping toxicity in regard to TLS cannot be excluded.

### III. Study Assessments

### A. Patient Characteristics

Medical history includes clinically significant diseases, surgeries, reproductive status, smoking history, and alcohol and drug abuse. In addition, all medications (e.g., prescription drugs, over-the-counter drugs, herbal or homeopathic remedies, nutritional supplements) used by the patient within 7 days prior to the screening period are recorded. Demographic data includes age, sex, and self-reported race/ethnicity. The following clinical parameters relative to disease history, diagnosis, and prognostic indices are recorded at screening: Date of initial diagnosis; ECOG Performance Status; B symptoms (unexplained fever >38° C., night sweats, unexplained weight loss >10% of body weight over 6 months); Ann Arbor staging; for patients with FL, Follicular Lymphoma International Prognostic Index (FLIPI) and FLIPI2; for patients with DLBCL, IPI; previous lines of anti-lymphoma treatment and response to prior therapy, date of disease progression in relation to start date of prior therapy, and date of last dose of prior therapy. Complete physical examinations are performed and vital signs are assessed. Electrocardiograms are performed. Multiple acquisition scan/echocardiograms are performed.

### B. Tumor Response Evaluations

All evaluable or measurable disease is documented at screening and re-assessed at each subsequent tumor evaluation. Response is assessed by the IRC and the investigator on the basis of physical examinations and PET and CT scans using the Lugano 2014 criteria, taking into account results of bone marrow examinations for patients with bone marrow involvement at screening.

In this study, the Lugano 2014 criteria for a PET-CT-based CR have been slightly modified to require normal bone marrow by morphology for patients with bone marrow involvement at screening. If indeterminate by morphology, immunohistochemistry should be negative. Additionally, designation of PET-CT-based PR requires that CT-based response criteria for a CR or PR be met in addition to the PET-CT-based response criteria for a PR. The modified Lugano 2014 criteria (Cheson et al, 2014) are provided in Table 7.

TABLE 7
Modified Lugano Response Criteria for Malignant Lymphoma
Revised Criteria for Response Assessment

| Response and Site | PET-CT-Based Response | CT-Based Response |
|---|---|---|
| Complete | Complete metabolic response | Complete radiologic response |
| | | (all of the following) |
| Lymph nodes and | Score 1, 2, or 3^{a} with or without | Target nodes/nodal masses must |
| extralymphatic sites | a residual mass on 5PS.^{b} | regress to ≤1.5 cm in LDi |
| | It is recognized that in | No extralymphatic sites of |
| | Waldeyer's ring or extranodal | disease |
| | sites with high physiologic | |
| | uptake or with activation within | |
| | spleen or marrow (e.g., with | |
| | chemotherapy or myeloid | |
| | colony-stimulating factors), | |
| | uptake may be greater than | |
| | normal mediastinum and/or | |
| | liver. In this circumstance, | |
| | complete metabolic response | |
| | may be inferred if uptake at sites | |
| | of initial involvement is no | |
| | greater than surrounding normal | |
| | tissue even if the tissue has high | |
| | physiologic uptake. | |
| Non-measured lesion | Not applicable | Absent |
| Organ enlargement | Not applicable | Regress to normal |
| New lesions | None | None |
| Bone marrow | No evidence of FDG-avid | Normal by morphology; if |
| | disease in marrow | indeterminate, IHC negative |
| Partial | Partial metabolic response | Partial remission (all of the |
| | | following) |
| Lymph nodes and | Score 4 or 5^{b} with reduced | ≥50% decrease in SPD of up to |
| extralymphatic sites | uptake compared with baseline | 6 target measurable nodes and |
| | and residual mass(es) of any | extranodal sites. |
| | size. | |
| | At interim, these findings | When a lesion is too small to |
| | suggest responding disease. | measure on CT, assign 5 mm × |
| | | 5 mm as the default value. |
| | At end of treatment, these | When no longer visible, 0 × 0 |
| | findings indicate residual | mm. |
| | disease. | For a node >5 mm × 5 mm, but |
| | | smaller than normal, use actual |
| | | measurement for calculation. |
| Non-measured lesion | Not applicable | Absent/normal, regressed, but |
| | | no increase |
| Organ enlargement | Not applicable | Spleen must have regressed by >50% |
| | | in length beyond normal. |
| Bone marrow | None | None |
| New lesions | Residual uptake higher than | Not applicable |
| | uptake in normal marrow but | |
| | reduced compared with baseline | |
| | (diffuse uptake compatible with | |
| | reactive changes from | |
| | chemotherapy | |
| | allowed). If there are persistent | |
| | focal changes in the marrow in | |
| | the context of a nodal response, | |
| | consideration should be given to | |
| | further evaluation with MRI or | |
| | biopsy or an interval scan. | |
| No response or stable disease | No metabolic response | Stable disease |
| Target nodes/nodal masses, | Score 4 or 5^{b} with no significant | <50% decrease from baseline in |
| extranodal lesions | change in FDG uptake | SPD of up to 6 dominant, |
| | from baseline at interim or end | measurable nodes and |
| | of treatment. | extranodal sites; no criteria for |
| | | progressive disease are met |
| Non-measured lesion | Not applicable | No increase consistent with |
| | | progression |
| Organ enlargement | Not applicable | No increase consistent with |
| | | progression |
| New lesions | None | None |
| Bone marrow | No change from baseline | Not applicable |
| Progressive disease | Progressive metabolic disease | Progressive disease requires at |
| | | least 1 of the following |
| Individual target nodes/nodal | Score 4 or 5^{b} with an increase in | PPD progression: |
| masses | intensity of uptake from | |
| | baseline and/or | |
| Extranodal lesions | New FDG-avid foci consistent | An individual node/lesion must |
| | with lymphoma at interim or | be abnormal with: LDi >1.5 cm |
| | end-of-treatment assessment | and |
| | | Increase by ≥50% from PPD |
| | | nadir and |
| | | An increase in LDi or SDi from |
| | | nadir 0.5 cm for lesions ≤2 cm |
| | | 1.0 cm for lesions >2 cm. |
| | | In the setting of splenomegaly, |
| | | the splenic length must |
| | | increase by >50% of the extent |
| | | of its prior increase beyond |
| | | baseline (e.g., a 15-cm spleen |
| | | must increase |
| | | to >16 cm). If no prior |
| | | splenomegaly, must increase by |
| | | at least 2 cm from baseline. |
| | | New or recurrent splenomegaly |
| | | New or clear progression of |
| | | preexisting non-measured |
| | | lesions |
| New lesions | New FDG-avid foci consistent | Regrowth of previously resolved |
| | with lymphoma rather than | lesions. |
| | another etiology (e.g., infection, | A new node >1.5 cm in any |
| | inflammation); if uncertain | axis. |
| | regarding etiology of new | A new extranodal site >1.0 cm |
| | lesions, biopsy or interval scan | in any axis; if <1.0 cm in any |
| | may be considered. | axis, its presence must be |
| | | unequivocal and must be |
| | | attributable to lymphoma. |
| | | Assessable disease of any size |
| | | unequivocally attributable |
| | | to lymphoma. |
| Bone marrow | New or recurrent FDG-avid foci | New or recurrent involvement |

5PS = 5-point scale; CT = computed tomography; FDG = fluorodeoxyglucose; GI = gastrointestinal; IHC = immunohistochemistry; LDi = longest transverse diameter of a lesion; MRI = magnetic resonance imaging; PET = positron emission tomography; PPD = cross product of the LDi and perpendicular diameter; SDi = shortest axis perpendicular to the LDi; SPD = sum of the product of the perpendicular diameters for multiple lesions.
^{a}A score of 3 in many patients indicates a good prognosis with standard treatmant, especially if at the time of an interim scan. However, in trials involving PET where de-escalation is investigated, it may be preferable to consider a score of 3 as inadequate response (to avoid undertreatment).
Measured dominant lesions: Up to six of the largest dominant nodes, nodal masses, and extranodal lesions selected to be clearly measurable in two diameters. Nodes should preferably be from disparate regions of the body and should include, where applicable, mediastinal and retroperitoneal areas.
Non-nodal lesions include those in solid organs (e.g., liver, spleen, kidneys, lungs), GI involvement, cutaneous lesions, or those noted on palpation.
Non-measured lesions: Any disease not selected as measured; dominant disease and truly assessable disease should be considered not measured.
These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
^{b}PET 5PS: 1 = no uptake above background; 2 = uptake ≤ mediastinum; 3 = uptake > mediastinum but ≤ liver; 4 = uptake moderately > liver; 5 = uptake markedly higher than liver and/or new lesions; X = new areas of uptake unlikely to be related to lymphoma.

### C. Radiographic Assessments

PET scans include the base of the skull to mid-thigh region. Full body PET scans are performed when clinically appropriate. CT with oral and IV contrast include chest, abdomen, and pelvic scans. CT scans of the neck are included if clinically indicated (i.e., evidence of disease upon physical examination) and are repeated throughout the study if there is disease involvement at baseline. If contrast is medically contraindicated (e.g., patients with contrast allergy or impaired renal clearance), MRI scans of the chest, abdomen, and pelvis (and neck, if clinically indicated) and a non-contrast CT scan of the chest are performed. If MRI scans cannot be obtained, CT scans without contrast are permitted as long as this allows consistent and precise measurement of the targeted lesions during the study treatment period. The same radiographic assessment modality is used for all response evaluations to ensure consistency across different timepoints (including unscheduled assessments). A full tumor assessment, including radiographic assessment, is performed any time disease progression or relapse is suspected.

### D. Bone Marrow Assessments

Bone marrow examinations are required at screening for staging purposes in all patients and are performed within approximately 3 months prior to Day 1 of Cycle 1. If bone marrow infiltration is present at screening, a bone marrow biopsy is required at the EOI response assessment for all patients who may have achieved a CR. In patients with a PR and continued bone marrow involvement, a subsequent bone marrow examination may be required to confirm a CR at a later timepoint.

### E. Biomarker Assessments

Exploratory biomarkers, but are not limited to, the biomarkers listed in Table 8.

TABLE 8
Biomarkers.

| Sample Type | Timing | Proposed Non-Inherited Biomarkers |
|---|---|---|
| Archival or fresh pre-treatment, | Prior to study | For DLBCL patients only: DLBCL cell-of- |
| and progression tumor tissue | (archival) or | origin subtype (ABC vs. GCB), BCL2, MYC. |
| | baseline (fresh); | For both DLBCL and FL patients: Target |
| | at disease | expression BCL2 and CD79b. |
| | progression | Lymphoma-related genetic changes (DNA) |
| | | and gene expression (mRNA) or protein |
| | | expression (IHC) associated with response or |
| | | potential resistance. |
| | | Lymphoma index clone in MRD. |
| Peripheral blood mononuclear | Baseline, | Circulating lymphoma cells and/or cell-free |
| cells and plasma isolated from | subsequent | circulating tumor DNA (detection of minimal |
| whole blood | timepoints during | residual disease). |
| | treatment | |
| Whole blood | Baseline and | Lymphocyte immunophenotyping, including |
| | subsequent | B-cell counts (CD19), T-cell counts (CD3, |
| | timepoints during | CD4, and CD8), and NK-cell counts (CD16 |
| | and after | and CD56). |
| | treatment | |

ABC = activated B cell-like; DLBCL = diffuse large B-cell lymphoma; FL = follicular lymphoma; GCB = germinal-center B cell-like; IHC = immunohistochemistry; MRD = minimum residual disease; mRNA = messenger RNA; NK = natural killer.

Exploratory analyses of biomarkers related to tumor biology and study treatment mechanisms of action are conducted. Analyses assess the prognostic and/or predictive value of candidate biomarker for each histological subtype with respect to both IRC- and investigator-assessed outcomes. Specifically, the association between candidate biomarkers and PET-CT-defined CR rate and OR rate, and potentially other measures of efficacy and safety, are explored to assess potential prognostic or predictive value.

### F. Safety Assessments

Adverse events are assessed according to the adverse event severity grading scale for the NCI CTCAE (v4.0). Adverse events of special interest include cases of potential drug-induced liver injury that include an elevated ALT or AST in combination with either an elevated bilirubin or clinical jaundice, as defined by Hy's law; tumor lysis syndrome of any grade; Grade 4 thrombocytopenia; Grade ≥3 infection; and second malignancies. Selected adverse events for which additional analyses are performed include thrombocytopenia, hepatitis B reactivation, cardiac events, tumor lysis syndrome, infusion related reactions, all infections, PML, neutropenia, peripheral neuropathy, and gastrointestinal perforation. Any findings of treatment-emergent ALT or AST >3×baseline value in combination with total bilirubin >2×ULN (of which ≥35% is direct bilirubin), or treatment-emergent ALT or AST >3×baseline value in combination with clinical jaundice are considered adverse events.

In this study, a dose limiting toxicity (DLT) was defined as any one of the following events that occurred during the first cycle of treatment and is assessed by the investigator as related to study treatment that is not attributed to disease progression or another clearly identified cause:
Any adverse event of any grade that leads to a delay of more than 14 days in the start of the next treatment cycle.
Any Grade 3 or 4 non-hematologic adverse event, with the following exceptions:
   Grade 3 or 4 infusion-related reactions (IRRs).
   Grade 3 diarrhea that responds to therapy within 72 hours.
   Grade 3 nausea or vomiting that occurs in the absence of premedication and responds to adequate therapy within 72 hours.
   Grade 3 fatigue that resolves to Grade ≤2 within 7 days.
   Grade 3 laboratory tumor lysis syndrome without manifestations of clinical tumor lysis syndrome (i.e., creatinine ≥1.5×upper limit of normal [ULN] and/or renal dysfunction, cardiac arrhythmias, seizures, or sudden death) that resolves within 7 days.
   Grade 3 laboratory abnormality that is asymptomatic and deemed by the investigator not to be clinically significant.
   Grade 3 elevation in ALT or AST, provided the following criteria are met:
      ALT or AST level is no greater than 8×ULN.
      ALT or AST elevation resolves to Grade <2 (<5×ULN) within 7 days.
      Total and direct bilirubin and other laboratory parameters of liver synthetic function (e.g., prothrombin time) are normal.
      No clinical signs or symptoms of hepatic injury.
Any increase in hepatic transaminase >3×baseline AND an increase in direct bilirubin >2×ULN, WITHOUT any findings of cholestasis or jaundice or signs of hepatic dysfunction AND in the absence of other contributory factors (e.g., worsening of metastatic disease or concomitant exposure to known hepatotoxic agent or of a documented infectious etiology) is suggestive of potential drug-induced liver injury (according to Hy's Law) and will be considered a DLT.
In patients with Grade 1 ALT or AST elevation at baseline as a result of liver metastases, only a Grade ≥3 elevation that is also ≥3×baseline lasting >7 days will be considered a DLT.
Hematologic adverse event that meets any of the following criteria:
   Grade 3 or 4 neutropenia in the presence of sustained fever of >38° C. (lasting >5 days) or a documented infection.
   Grade 4 neutropenia lasting >7 days.
   Grade 3 or 4 thrombocytopenia that results in significant bleeding.
   Grade 4 thrombocytopenia lasting >7 days.

Other toxicities occurring during the first cycle that are considered clinically relevant and related to study treatment may also be considered DLTs.

### G. Statistical Analyses

Data from the dose-escalation phase are summarized by cohort (assigned dose level). Data from the expansion phase are summarized by histological subtype (i.e., FL or DLBCL).

The number of doses, treatment cycles, average dose received, and relative dose intensity for each study treatment are summarized using descriptive statistics (mean, standard deviation, median, and range).

The primary safety and efficacy populations include patients who receive at least one dose of any component of the combination.

The intent-to-treat population includes all patients enrolled in the study.

Demographic and baseline characteristics, such as age, sex, race, and duration of malignancy are summarized using descriptive statistics (mean, standard deviation, median, and range) for continuous variables and frequencies and percentages for categorical variables.

Safety analyses include all treated patients (i.e., patients who received any amount of study treatment). Patients in the dose-escalation phase are summarized by cohort and histology type, and patients in the expansion phase are summarized by histology type (FL or DLBCL). Safety is assessed through summaries of adverse events and changes from baseline laboratory test results, shift-tables of ECGs findings, and vital signs. All adverse events occurring on or after first study treatment are summarized by mapped term, appropriate thesaurus levels, and NCI CTCAE v4.0 grade. All serious adverse events, adverse events of special interest, and selected adverse events are summarized. Deaths reported during the treatment period and during post-treatment follow-up are summarized. Relevant laboratory results are analyzed by time, with Grade 3 and 4 values identified.

### H. Efficacy Assessments

The primary and secondary efficacy analyses include the primary efficacy population and the intent-to-treat population for patients enrolled in the expansion phase, with patients grouped according to histologic subtype, and are performed by treatment group. In addition, patients with FL and DLBCL who received polatuzumab vedotin and venetoclax at the RP2D during the dose-escalation phases are pooled for analysis by histology with patients treated in the expansion phase at the same dose levels. Response is determined on the basis of PET-CT scans or CT scans alone, using the Lugano 2014 criteria.

The primary efficacy endpoint is the proportion of patients achieving a CR at EOI, as determined by the IRC on the basis of PET-CT scans according to Lugano 2014. Point estimates are presented, along with the corresponding 90% Clopper-Pearson exact CIs. Patients without a post-baseline tumor assessment are considered non-responders.

The secondary efficacy analyses include estimation of the proportion of patients who achieve each of the following endpoints:
CR at EOI, as determined by the investigator on the basis of PET-CT scans.
CR at EOI, as determined by the IRC and by the investigator on the basis of CT scans alone.
Objective response (defined as a CR or PR) at EOI, as determined by the IRC and by the investigator on the basis of PET-CT scans.
Objective response (defined as a CR or PR) at EOI, as determined by the IRC and by the investigator on the basis of CT scans alone.
Best response of CR or PR during the study, as determined by the investigator on the basis of CT scans alone.

Point estimates are presented, along with the corresponding two-sided 90% Clopper-Pearson exact CIs. Patients without a post-baseline tumor assessment are considered non-responders.

Exploratory efficacy analyses include estimation of the proportion of patients achieving each of the following endpoints:
For patients who have positive PET scans at EOI:
   CR at 12 months, as determined by the IRC and by the investigator on the basis of PET-CT scans, in FL patients.
   CR at end of consolidation (EOC), as determined by the IRC and by the investigator on the basis of PET-CT scans, in DLBCL patients.

Point estimates are presented, along with the corresponding two-sided 90% Clopper-Pearson exact CIs. Patients without a post-baseline tumor assessment are considered non-responders.

Exploratory efficacy analyses are performed on the following endpoints: progression-free survival (PFS), event-free survival (EFS), disease-free survival (DFS), and overall survival (OS). PFS, EFS, DFS, and OS are summarized descriptively using the Kaplan-Meier method (Kaplan and Meier 1958). For the PFS, EFS, and DFS analyses, data for patients without an event of interest are censored at the date of the last tumor assessment. For patients without post-baseline tumor assessments, data are censored at the date of initiation of study treatment plus 1. For the OS analysis, data for patients who have not died are censored at the date the patient was last known to be alive. Where medians are reached, the corresponding estimated median are provided, along with the 95% CI using the method of Brookmeyer and Crowley, 1982. In addition, landmark estimates of the proportion of patients who are event free at 6 months, 9 months, 1 year, and 2 years are provided, along with 95% asymptotic Cis using Greenwood's formula for standard errors.

### I. Pharmacokinetics Assessments

The pharmacokinetics (PK) population for analysis includes all patients who have at least one evaluable PK sample post dose for at least one analyte. Serum or plasma concentrations of obinutuzumab, rituximab, polatuzumab vedotin and relevant analytes, and venetoclax are tabulated and plotted over time after appropriate grouping. Summary statistics of concentration data are computed for each scheduled sampling time for each analyte after appropriate grouping. Interpatient variability and drug accumulation after multiple doses are evaluated as appropriate. Compartmental, non-compartmental, and/or population approaches are considered as appropriate. Potential correlations between PK variability and pharmacodynamic, efficacy, and safety endpoints are explored by exploratory graphical analysis and PK-pharmacodynamic modeling.

### J. Immunogenicity

The immunogenicity analyses include patients with at least one pre-dose and one post-dose human anti-human antibody (HAHA) or anti-therapeutic antibody (ATA) assessment, with patients grouped according to histology. The numbers and proportions of HAHA- or ATA-positive patients and HAHA- or ATA-negative patients during both the treatment and follow-up periods are summarized by histology group. Patients are considered to be HAHA- or ATA-positive if they are HAHA- or ATA-negative at baseline but develop an HAHA or ATA response following study drug administration (treatment-induced HAHA or ATA response), or if they are HAHA- or ATA-positive at baseline and the titer of 1 or more post-baseline samples is at least 4-fold greater (i.e., ≥0.60 titer units) than the titer of the baseline sample (treatment-enhanced HAHA or ATA response). Patients are considered to be HAHA- or ATA-negative if they are HAHA- or ATA-negative at baseline and all post-baseline samples are negative, or if they are HAHA- or ATA-positive at baseline but do not have any post-baseline samples with a titer that is at least 4-fold greater than the titer of the baseline sample (treatment unaffected). The relationship between HAHA or ATA status and safety, efficacy, PK, and biomarker endpoints is analyzed and reported descriptively via subgroup analyses.

### K. Toxicities

(i) Toxicities During Induction

Hematologic toxicity is defined as neutropenia, anemia, or thrombocytopenia. Lymphopenia is not considered hematologic toxicity but an outcome of therapy. Table 9 provides guidelines for management of hematologic toxicities that occur during induction treatment, with the exception of Days 8 and 15 of Cycle 1. Table 10 provides guidelines for management of hematologic toxicities that occur at Days 8 and 15 of Cycle 1, when patients are to receive treatment with obinutuzumab only.

TABLE 9
Guidelines for Management of Hematologic Toxicities That
Occur during Induction Treatment (Except Days 8 and
15 of Cycle 1 for Patients Receiving Obinutuzumab).

| Event | Action to be Taken |
|---|---|
| Grade 3 or 4 | For patients who have had one or no prior |
| hematologic | venetoclax dose reductions: |
| toxicity^{a,b} | Withhold study treatment.^{a} |
| | Administer RBCs or platelets as required. |
| | If patient has not already initiated G-CSF, |
| | initiate prophylactic G-CSF for current and |
| | subsequent cycles. |
| | For patients who develop platelet count |
| | of <20,000/μL while receiving LMWH, reduce the |
| | dose of LMWH. For patients who develop platelet |
| | count of <20,000/μL while receiving platelet |
| | inhibitors, consider temporarily withholding |
| | platelet inhibitors. |
| | If improvement to Grade ≤2 or baseline occurs |
| | within 7 days after the scheduled date for the next |
| | cycle, resume obinutuzumab or rituximab and |
| | polatuzumab vedotin at full dose and resume |
| | venetoclax at current dose. |
| | If improvement to Grade ≤2 or baseline occurs |
| | within 8-14 days after the scheduled date for the |
| | next cycle, resume obinutuzumab or rituximab |
| | and polatuzumab vedotin at full dose and resume |
| | venetoclax at a reduced dose^{a,b} for current and |
| | subsequent cycles. No more than 2 dose level |
| | reductions from the original dose of venetoclax |
| | are allowed during induction. |
| | If study treatment is withheld for >21 days, |
| | permanently discontinue study treatment. |
| | For patients who have had two prior |
| | venetoclax dose reductions: |
| | Permanently discontinue study treatment. |

G-CSF = granulocyte colony-stimulating factor; LMWH = low molecular weight heparin.
^{a}Treatment delays apply to all toxicities; dose modifications apply only to toxicities that are considered to be related to any of the study treatment components. Toxicities that occur during the cycle and subside prior to the next cycle should not trigger the suggested dose modifications.
^{b}If cytopenia is thought to be caused mainly by B-cell lymphoma infiltration of the bone marrow, the investigator may decide not to reduce the venetoclax dose.

TABLE 10
Guidelines for Management of Hematologic Toxicities That Occur on
Days 8 and 15 of Cycle 1 for Patients Receiving Obinutuzumab.

| Event | Action to be Taken |
|---|---|
| Febrile neutropenia | Withhold obinutuzumab and venetoclax until |
| or neutropenia | resolution of fever and infection (as applicable). |
| with infection | If the event is ongoing at Day 1 Cycle 2, follow |
| | instructions in Table 11. Note: Obinutuzumab and |
| | venetoclax are not withheld for asymptomatic |
| | neutropenia. |
| Severe | Withhold obinutuzumab and venetoclax until |
| thrombocytopenia^{a} | platelet count ≥50,000/μL and resolution of |
| or bleeding | bleeding. |
| | If receiving LMWH, reduce the dose. |
| | If receiving platelet inhibitors, consider |
| | temporarily withholding platelet inhibitors. |
| | If the event is ongoing at Day 1 Cycle 2, follow |
| | instructions in Table 9. |

LMWH = low-molecular-weight heparin.
^{a}Severe thrombocytopenia is defined as a platelet count <10,000/μL for patients who are not receiving concomitant anticoagulants or platelet inhibitors and <20,000/μL for patients who are receiving concomitant anticoagulants or platelet inhibitors.

Table 11 provides guidelines for management of non-hematologic toxicities that occur during induction treatment.

TABLE 11
Guidelines for Management of Non-Hematologic
Toxicities That Occur During Induction.

| Event | | Action to be Taken |
|---|---|---|
| General guidance for treatment | | If study treatment is withheld for >21 days |
| delays and discontinuation | | because of a toxicity that is attributable to study |
| | | treatment, permanently discontinue study |
| | | treatment. |
| | | When a treatment cycle is delayed because of |
| | | toxicity resulting from any component of the |
| | | regimen, all study treatment should be held and |
| | | resumed together to remain synchronized. |
| IRRs and anaphylaxis | | Guidelines for the management of IRRs are as |
| | | described above for obinutuzumab, polatuzumab |
| | | vedotin, and rituximab. Anaphylaxis procedures |
| | | are described below. |
| Tumor lysis syndrome | | Withhold study treatment.a |
| | | Correct electrolyte abnormalities, monitor renal |
| | | function and fluid balance, and administer |
| | | supportive care, including dialysis as indicated. |
| | | If symptoms have resolved completely, resume |
| | | obinutuzumab or rituximab and polatuzumab |
| | | vedotin at full dose and resume venetoclax at |
| | | current dose. |
| New-onset neurologic | | Withhold study treatment.^{a} |
| manifestations suggestive of PML | | Consult with a neurologist if PML is suspected. |
| | | If PML is ruled out, resume obinutuzumab or |
| | | rituximab and polatuzumab vedotin at full dose |
| | | and resume venetoclax at current dose. |
| | | If PML is confirmed, permanently discontinue |
| | | study treatment. |
| AST, ALT, or bilirubin increase: | | Withhold study treatment.^{a} |
| Grade ≥3 (or ≥10× ULN for | | If improvement to Grade ≤1, resume |
| patients with liver | | obinutuzumab or rituximab and polatuzumab |
| involvement) | | vedotin at full dose and resume venetoclax at next |
| | | lower dose^{a} for current and subsequent cycles. No |
| | | more than 2 dose level reductions from original |
| | | dose of venetoclax are allowed during induction. |
| | | Patients who have had two prior dose reductions |
| | | should be permanently discontinued. |
| | | Permanently discontinue study treatment for |
| | | life-threatening liver toxicity. |
| Neurotoxicity | Grade 4 | Permanently discontinue polatuzumab vedotin |
| | | and all other study treatment. |
| | Grade 2 or | Withhold study treatment.^{a} |
| | Grade 3 | If improvement to Grade ≤1 within 21 days, |
| | | resume study treatment for current and subsequent |
| | | cycles as follows: |
| | | Resume obinutuzumab or rituximab at full dose |
| | | Resume venetoclax at current dose^{a} |
| | | For patients who started at 1.8 mg/kg, resume |
| | | polatuzumab vedotin at the permanently reduced |
| | | dose of 1.4 mg/kg; for patients who started at |
| | | 1.4 mg/kg, permanently discontinue polatuzumab |
| | | vedotina |
| Other non-hematologic | Grade 3 or | For patients who have had no prior dose |
| toxicities (i.e., not | Grade 4 | reductions: |
| described above), | | Withhold study treatment.a |
| excluding alopecia, | | If improvement to Grade ≤1 or baseline, resume |
| nausea, and vomiting | | obinutuzumab or rituximab and polatuzumab |
| | | vedotin at full dose and resume venetoclax at next |
| | | lower dose^{a} for current and subsequent cycles. |
| | | For patients who have had one prior dose |
| | | reduction: |
| | | Grade 4 events: |
| | | Permanently discontinue study treatment. |
| | | Grade 3 events: |
| | | Withhold study treatment.^{a} |
| | | If improvement to Grade ≤1 or baseline, resume |
| | | obinutuzumab or rituximab and polatuzumab |
| | | vedotin at full dose and resume venetoclax at next |
| | | lower dose^{a} for subsequent cycles. No more than |
| | | two dose level reductions from the original dose |
| | | of venetoclax are allowed during induction. |
| | | For patients who have had two prior dose |
| | | reductions: |
| | | Permanently discontinue study treatment. |
| | Grade 2 | Withhold study treatment.a |
| | | If improvement to Grade ≤1 or baseline, resume |
| | | obinutuzumab or rituximab and polatuzumab |
| | | vedotin at full dose and resume venetoclax at |
| | | current dose. |

IRR = infusion-related reaction; PML = progressive multifocal leukoencephalopathy; TLS = tumor lysis syndrome; ULN = upper limit normal.
^{a}Treatment delays apply to all events; dose modifications apply only to events that are considered to be related to any of the study treatment components. Toxicities that occur during the cycle and subside prior to the next cycle should not trigger the suggested dose modifications.

(ii) Toxicities During Consolidation or Maintenance Treatment

Table 12 provides guidelines for management of toxicities that occur during consolidation or maintenance treatment.

TABLE 12
Guidelines for Management of Toxicities That Occur
during Consolidation or Maintenance Treatment.

| Event | Action to be Taken |
|---|---|
| Hematologic toxicity: | Withhold obinutuzumab or rituximab and |
| Grade 3 or 4 | venetoclax. |
| | Administer G-CSF for neutropenia. |
| | Administer red blood cells or platelets as |
| | required. |
| | If improvement to Grade ≤2, resume |
| | obinutuzumab or rituximab at full dose and |
| | resume venetoclax at next lower dose for |
| | subsequent cycles. Patients who are not eligible |
| | for further venetoclax dose reductions should be |
| | permanently discontinued. |
| | If study treatment is withheld for >42 days, |
| | permanently discontinue study treatment. |
| Non-hematologic | Withhold obinutuzumab or rituximab and |
| toxicity: | venetoclax. |
| Grade ≥ 2 | If improvement to Grade ≤1 or baseline, resume |
| | obinutuzumab or rituximab at full dose and |
| | resume venetoclax at next lower dose for |
| | subsequent cycles. Patients who are not eligible |
| | for further venetoclax dose reductions should be |
| | permanently discontinued. |
| | If study treatment is withheld for >42 days, |
| | permanently discontinue study treatment. |

### L. Anaphylaxis

In the event of a suspected anaphylactic reaction during study treatment infusion, the following procedures are performed:
Stop the study treatment infusion.
Apply a tourniquet proximal to the injection site to slow systemic absorption of study treatment. Do not obstruct arterial flow in the limb.
Maintain an adequate airway.
Administer glucocorticoids, antihistamines, epinephrine, or other medications as required by patient status and directed by the physician in charge.
Continue to observe the patient and document observations.

### IV. Results of the FL Dose Escalation Phase

The sample size estimate for the FL dose-escalation phase was based on a 3+3 escalation rule with six possible dose levels, and, therefore, needed to include 21-36 patients in order to establish the RP2D. Safety evaluations were summarized descriptively by cohort. The primary safety and efficacy populations included all patients who received at least one dose of any component of the combination.

### A. Patient Characteristics

Thirty-three patients with FL were enrolled in this study. All patients were included in both the safety and efficacy evaluable populations.

Patient demographics and baseline characteristics are shown in Table 13. Patients mostly had Ann Arbor stage 3-4 disease (69.7%), and 9, 11, and 13 patients had low-, intermediate-, and high-risk disease, respectively, per the Follicular Lymphoma International Prognostic Index score (Solal-Céligny et al, 2004). Eight patients (24.2%) had experienced progression of disease within 24 months of their initial lymphoma treatment. In patients with FL, the progression of disease within 24 months of initial therapy is a well-established prognostic factor associated with inferior survival (Casulo et al, 2015). The median number of prior anti-lymphoma treatments was three (range, one to seven). Overall, 60.6% of patients had disease that was refractory to their last lymphoma therapy, and 48.5% were refractory to rituximab.

TABLE 13
Patient demographics and baseline characteristics.

| Characteristic | Total (N = 33) | |
|---|---|---|
| Mean age (range), years | 60.2 | (36-76) |
| Sex, n (%) | | |
| Male | 21 | (63.6) |
| Female | 12 | (36.4) |
| ECOG PS, n (%) | | |
| 0 | 21 | (63.6) |
| 1 | 12 | (36.4) |
| Histologic grade, n (%) | | |
| 1 | 7 | (21.2) |
| 2 | 21 | (63.6) |
| 3a | 5 | (15.2) |
| Bone marrow involvement, n (%) | 12 | (36.4) |
| Ann Arbor stage, n (%) | | |
| 1-2 | 10 | (30.3) |
| 3-4 | 23 | (69.7) |
| FLIPI score*, n (%) | | |
| 0-1 | 9 | (27.3) |
| 2 | 11 | (33.3) |
| 3-5 | 13 | (39.4) |
| Median no. of prior treatments (range) | 3 | (1-7) |
| Bulky disease (>7 cm), n (%) | 6 | (18.2) |
| Refractory to any line anti-CD20, n (%)^{†} | 18 | (54.5) |
| Refractory to last prior regimen, n (%)^{‡} | 16 | (48.5) |
| POD24, n (%) | 8 | (24.2) |

*FLIPI score indicates low (0 or 1), intermediate (2), or high (3 to 5) risk on the basis of a scoring system that gives one point for each of the following risk factors: age older than 60 years old, Ann Arbor stage III or IV disease, a haemoglobin level of less than 12 g/dL, more than four involved nodal areas, and a lactate dehydrogenase level greater than the upper limit of normal.
^{†}Defined as no response or progression or relapse within 6 months of therapy with an anti-CD20 agent at any prior line of treatment.
^{‡}Defined as no response or progression or relapse within 6 months of last anti-lymphoma therapy end date.
ECOG PS = Eastern Cooperative Oncology Group performance status; FLIPI = Follicular Lymphoma International Prognostic Index; POD24 = progression of disease within 24 months of completing initial lymphoma treatment.

### B. Safety

Laboratory assessments and adverse events (AEs) were assessed throughout the study, with complete blood counts performed on days 1, 8, and 15 of cycle 1, on day 1 of cycles 2-6, and monthly during maintenance treatment. AEs were coded using the most recent version of the Medical Dictionary for Regulatory Activities and graded according to the National Cancer Institute Common Terminology Criteria for Adverse Events, version 4.0.

Premedication was mandatory before the first obinutuzumab infusion and included a corticosteroid, antihistamine, and analgesic/antipyretic. All patients received prophylaxis for tumor lysis syndrome (TLS), including hydration and administration of a uric-acid reducing agent (e.g., allopurinol) orally, beginning 72 hours prior to the first venetoclax dose. The use of recombinant urate oxidase (rasburicase) was also allowed. Patients considered to be at high risk for TLS (high tumor burden or circulating lymphoma cells) required hospitalization for more intensive prophylaxis and monitoring during the initial dose of venetoclax. Supportive measures were permitted.

The DLT window was defined as the first cycle and included events assessed by the investigator as related to study treatment that was not attributed to disease progression or another clearly identified cause. DLTs included any AE of any grade that led to a delay of >14 days in the start of the next treatment. Hematologic DLTs were defined as grade 3-4 neutropenia in the presence of a sustained fever of >38° C. (lasting >5 days) or a documented infection, grade 3/4 thrombocytopenia that resulted in significant bleeding, or grade 4 neutropenia or thrombocytopenia lasting >7 days. Non-hematologic DLTs were defined as any grade ≥3 non-hematologic AEs attributed to study treatment with the following exceptions: grade 3/4 infusion-related reactions; grade 3 diarrhea that responded to therapy within 72 hours; grade 3 nausea or vomiting that occurred in the absence of premedication and responded to adequate therapy within 72 hours; grade 3 fatigue that resolved to grade ≤2 within 7 days; grade 3 laboratory TLS without manifestations of clinical TLS; grade 3 laboratory abnormality that was asymptomatic and deemed not to be clinically significant; grade 3 elevation in alanine or aspartate transaminases that resolved within 7 days and did not include clinical signs or symptoms of hepatic injury. Drug-induced liver injury according to Hy's Law was also considered a DLT. Dose delays or dose reductions were mandated for grade 3-4 cytopenias.

Two patients in cohort 1 (polatuzumab vedotin 1.4 mg/kg and venetoclax 400 mg) experienced DLTs: one grade 3 laboratory tumor lysis syndrome (TLS) and one grade 3 aspartate transaminase/alanine transaminase elevation. Neither case resulted in clinical sequelae, and both patients recovered with supportive care and temporary interruption of all study drugs. Additionally, both patients were able to restart all treatment and complete induction therapy. Based on the predictability and reversibility of these events, the DLT criteria were amended to allow asymptomatic laboratory TLS and increased liver function tests up to eight times the upper limit of normal, resolving within 7 days. Cohort 1a was added to include polatuzumab vedotin at 1.4 mg/kg and a lower dose of venetoclax at 200 mg.

Following cohort 1a clearing, an additional three patients were enrolled into cohort 1; no DLTs were reported in this cohort. Subsequently, one patient in cohort 4 (polatuzumab vedotin 1.8 mg/kg and venetoclax 600 mg) experienced a DLT of neutropenic sepsis; however, this patient had recently undergone an unusual transhepatic line placement for vascular access and chemotherapy administration prior to the start of the study, which confounded the clinical presentation.

Cohort 4 was expanded to include an additional three patients, none of whom experienced DLTs. Therefore, cohort 4 cleared, and cohort 6 was opened. An additional three patients were enrolled in cohort 6 to confirm tolerability at this dose level. The maximum tolerated dose (MTD) of polatuzumab vedotin in combination with venetoclax was not reached and the RP2D for this combination was identified as polatuzumab vedotin 1.8 mg/kg and venetoclax 800 mg, with a fixed dose of obinutuzumab 1,000 mg.

Diarrhea, nausea, and cytopenias (including neutropenia and thrombocytopenia) were reported at a higher frequency than would be with single agent or doublet regimens, which was anticipated due to overlapping toxicities (see Table 14). These adverse events were adequately managed with medical intervention and dose interruptions.

TABLE 14
Grade 3-4 adverse events occurring in ≥2 patients (n (%)).

| Cohort | Cohort 1a | Cohort 1 | Cohort 2 | Cohort 3 | Cohort 4 | Cohort 6 | Total |
|---|---|---|---|---|---|---|---|
| Pola dose | 1.4 mg/kg | 1.4 mg/kg | 1.8 mg/kg | 1.4 mg/kg | 1.8 mg/kg | 1.8 mg/kg | |
| Venetoclax dose | 200 mg | 400 mg | 400 mg | 600 mg | 600 mg | 800 mg | |
| Obinutuzumab dose | 1000 mg | 1000 mg | 1000 mg | 1000 mg | 1000 mg | 1000 mg | |
| N | 3 | 7 | 3 | 3 | 9 | 8 | 33 |
| Neutropenia | 1 (33.3) | 2 (28.6) | 1 (33.3) | 1 (33.3) | 4 (44.4) | 5 (62.5) | 14 (42.4) |
| Thrombocytopenia | 0 | 1 (14.3) | 2 (66.7) | 0 | 0 | 4 (50.0) | 7 (21.2) |
| Infections (SOC)* | 1 (14.3) | 1 (33.3) | 1 (33.3) | 0 | 1 (11.1) | 3 (37.5) | 7 (21.2) |
| Anemia | 0 | 1 (14.3) | 1 (33.3) | 0 | 0 | 1 (12.5) | 3 (9.1) |
| AST increased | 0 | 2 (28.6) | 0 | 0 | 0 | 0 | 2 (6.1) |
| Febrile neutropenia | 0 | 0 | 0 | 0 | 0 | 2 (25.0) | 2 (6.1) |
| IRR | 0 | 1 (14.3) | 1 (33.3) | 0 | 0 | 0 | 2 (6.1) |

*Infections were reported as System Organ Class and included cellulitis, Clostridium difficile colitis, infective exacerbation of chronic obstructive airways disease, lung infection, neutropenic sepsis, pneumonia, Pseudomonas infection, rhinovirus infection, sinusitis, and urinary tract infection.
AST = aspartate aminotransferase; IRR = infusion-related reaction; Pola = polatuzumab vedotin.

As shown Table 15, all 33 patients (100%) experienced at least one adverse event (AE). The median duration of treatment across all cohorts was 11.37 months (range 0.2-26.0 months). The most common all-grade AEs were diarrhea (63.6%), fatigue (45.5%), and neutropenia (45.5%). Grade 3-4 AEs were reported in 21 (63.6%) patients and were primarily cytopenias including neutropenia (42.4%), thrombocytopenia (21.2%), anemia (9.1%), and febrile neutropenia (6.1%). Seven patients experienced grade 3-4 infections, including two cases each of Clostridium difficile colitis and pneumonia. A total of 11 patients (33.3%) experienced a serious AE (SAE). The most common type of SAEs reported were infections that required hospitalization for treatment (six patients, 18.2%).

No deaths due to AEs were reported.

TABLE 15
All-grade adverse events occurring in ≥10% patients (n (%)).

| Cohort | Cohort 1a | Cohort 1 | Cohort 2 | Cohort 3 | Cohort 4 | Cohort 6 | Total |
|---|---|---|---|---|---|---|---|
| Pola dose | 1.4 mg/kg | 1.4 mg/kg | 1.8 mg/kg | 1.4 mg/kg | 1.8 mg/kg | 1.8 mg/kg | |
| Venetoclax dose | 200 mg | 400 mg | 400 mg | 600 mg | 600 mg | 800 mg | |
| Obinutuzumab dose | 1000 mg | 1000 mg | 1000 mg | 1000 mg | 1000 mg | 1000 mg | |
| N | 3 | 7 | 3 | 3 | 9 | 8 | 33 |
| Patients with ≥1 AE | 3 (100) | 7 (100) | 3 (100) | 3 (100) | 9 (100) | 8 (100) | 33 (100) |
| Diarrhea | 2 (66.7) | 3 (42.9) | 3 (100) | 2 (66.7) | 5 (55.6) | 6 (75.0) | 21 (63.6) |
| Fatigue | 2 (66.7) | 5 (71.4) | 2 (66.7) | 1 (33.3) | 2 (22.2) | 3 (37.5) | 15 (45.5) |
| Neutropenia | 1 (33.3) | 2 (28.6) | 2 (66.7) | 1 (33.3) | 4 (44.4) | 5 (62.5) | 15 (45.5) |
| Nausea | 1 (33.3) | 2 (28.6) | 2 (66.7) | 1 (33.3) | 2 (22.2) | 4 (50.0) | 12 (36.4) |
| Cough | 1 (33.3) | 3 (42.9) | 1 (33.3) | 1 (33.3) | 1 (11.1) | 4 (50.0) | 11 (33.3) |
| IRR | 0 | 3 (42.9) | 2 (66.7) | 0 | 4 (44.4) | 1 (12.5) | 10 (30.3) |
| Thrombocytopenia | 0 | 2 (28.6) | 2 (66.7) | 0 | 2 (22.2) | 4 (50.0) | 10 (30.3) |
| Vomiting | 0 | 2 (28.6) | 2 (66.7) | 1 (33.3) | 1 (11.1) | 3 (37.5) | 9 (27.3) |
| ALT increased | 1 (33.3) | 3 (42.9) | 0 | 2 (66.7) | 1 (11.1) | 1 (12.5) | 8 (24.2) |
| URI | 0 | 3 (42.9) | 2 (66.7) | 2 (66.7) | 0 | 1 (12.5) | 8 (24.2) |
| Pyrexia | 0 | 1 (14.3) | 0 | 0 | 1 (11.1) | 4 (50.0) | 6 (18.2) |
| Arthralgia | 0 | 2 (28.6) | 1 (33.3) | 0 | 1 (11.1) | 1 (12.5) | 5 (15.2) |
| AST increased | 0 | 2 (28.6) | 0 | 2 (66.7) | 0 | 1 (12.5) | 5 (15.2) |
| Hypokalemia | 0 | 2 (28.6) | 1 (33.3) | 0 | 0 | 2 (25) | 5 (15.2) |
| Insomnia | 1 (33.3) | 2 (28.6) | 1 (33.3) | 0 | 0 | 1 (12.5) | 5 (15.2) |
| LDH increased | 2 (66.7) | 1 (14.3) | 0 | 1 (33.3) | 0 | 1 (12.5) | 5 (15.2) |
| UTI | 0 | 1 (14.3) | 1 (33.3) | 0 | 2 (22.2) | 1 (12.5) | 5 (15.2) |
| Abdominal pain | 0 | 1 (14.3) | 1 (33.3) | 0 | 0 | 2 (25.0) | 4 (12.1) |
| Abdominal pain upper | 0 | 0 | 0 | 1 (33.3) | 3 (33.3) | 0 | 4 (12.1) |
| Alopecia | 1 (33.3) | 1 (14.3) | 0 | 0 | 1 (11.1) | 1 (12.5) | 4 (12.1) |
| Anemia | 0 | 1 (14.3) | 1 (33.3) | 0 | 0 | 2 (25.0) | 4 (12.1) |
| Chills | 1 (33.3) | 2 (28.6) | 1 (33.3) | 0 | 0 | 0 | 4 (12.1) |
| Constipation | 0 | 2 (28.6) | 0 | 0 | 0 | 2 (25.0) | 4 (12.1) |
| Dry mouth | 0 | 0 | 0 | 0 | 1 (11.1) | 3 (37.5) | 4 (12.1) |
| GERD | 0 | 0 | 1 (33.3) | 1 (33.3) | 1 (11.1) | 1 (12.5) | 4 (12.1) |
| Musculoskeletal pain | 1 (33.3) | 1 (14.3) | 1 (33.3) | 0 | 0 | 1 (12.5) | 4 (12.1) |
| Rash | 1 (33.3) | 0 | 0 | 0 | 2 (22.2) | 1 (12.5) | 4 (12.1) |
| Sinusitis | 0 | 0 | 1 (33.3) | 1 (33.3) | 1 (11.1) | 1 (12.5) | 4 (12.1) |

ALT = alanine aminotransferase; GERD = gastroesophageal reflux disease; IRR = infusion-related reactions; LDH = lactate dehydrogenase; Pola = polatuzumab vedotin; URI = upper respiratory tract infection; UTI = urinary tract infection.

As shown in Table 16, one patient in cohort 1 developed laboratory TLS without clinical sequelae in cycle 1 of day 1, after the first dose of venetoclax. No additional cases of TLS were reported. Eleven patients (33%) experienced grade 1 or 2 peripheral neuropathy. No peripheral neuropathy higher than grade 2 in severity was observed for any patient. The incidence was similar across all cohorts and polatuzumab vedotin dose levels. One patient in cohort 6 required a dose reduction from polatuzumab vedotin 1.8 mg/kg to 1.4 mg/kg after five cycles of induction due to grade 2 peripheral neuropathy.

TABLE 16
Selected adverse events (n(%)).

| Cohort | Cohort 1a | Cohort 1 | Cohort 2 | Cohort 3 | Cohort 4 | Cohort 6 | Total |
|---|---|---|---|---|---|---|---|
| Pola dose | 1.4 mg/kg | 1.4 mg/kg | 1.8 mg/kg | 1.4 mg/kg | 1.8 mg/kg | 1.8 mg/kg | |
| Venetoclax dose | 200 mg | 400 mg | 400 mg | 600 mg | 600 mg | 800 mg | |
| Obinutuzumab dose | 1000 mg | 1000 mg | 1000 mg | 1000 mg | 1000 mg | 1000 mg | |
| N | 3 | 7 | 3 | 3 | 9 | 8 | 33 |
| Peripheral neuropathy | 0 | 3 (42.9) | 2 (66.7) | 1 (33.3) | 3 (33.3) | 2 (25) | 11 (33.3) |
| SMQ-w | | | | | | | |
| Tumor lysis syndrome | 1 (14.3) | 0 | 0 | 0 | 0 | 0 | 1 (3.0) |

SMQ-w = Standardized Medical Dictionary for Regulatory Activities Query-w; Pola = polatuzumab vedotin.

Overall, these results showed that the combination of polatuzumab vedotin, obinutuzumab, and venetoclax had an acceptable safety profile. The combination treatment was well-tolerated by most patients, and the overall grade 3-4 AE rate was 64%. Cytopenias were the most common Grade 304 AE and were manageable with dose modifications, delays, and supportive care.

### C. Efficacy

Positon emission tomography/computed tomography (PET/CT) scans were assessed by the investigators and an independent review committee. Responses to treatment were determined using Modified Lugano 2014 criteria. The designation of a CR using PET/CT-based response required normal bone marrow by morphology for patients with bone marrow involvement at baseline. If indeterminate by morphology, immunohistochemistry was negative. Additionally, designation of PET/CT-based PR required that CT-based response criteria for CR or PR were met in addition to the PET/CT-based response criteria for a PR.

Response assessment by clinical evaluation and imaging occurred prior to cycle 3 and at EOI. PET/CT scans were mandatory at screening and EOI, but diagnostic CT could be used at the cycle 3 assessment. During maintenance treatment, clinical assessment occurred every 2 months, and diagnostic CT imaging studies were repeated at months 12, 18, and 24. If a patient had a positive PET scan at EOI, then a repeat PET was required at month 12, otherwise diagnostic CT scans were accepted for the month 12, 18, and 24 response assessments. After the end of treatment, clinical assessment occurred every 3 months until progressive disease or end of study, with CT scans performed every 6 months for 2 years and as clinically indicated.

Thirty-three patients were included in the efficacy-evaluable population. Responses were assessed using Modified Lugano criteria, based on metabolic response using positron emission tomography/computed tomography (PET/CT) scans.

The median duration of follow-up was 17.74 months (range 5.7-39 months).

As shown in Table 17, the overall response rate for the efficacy evaluable population was 75.8%, with 57.6% of patients achieving a complete response (CR). The overall response rate in the eight patients in cohort 6 treated at the identified RP2D dose combination was 100%, and all achieved CR on PET/CT scans at EOI.

TABLE 17
Investigator-assessed clinical response rates at EOI (after 6 cycles of treatment) by Modified Lugano criteria.

| Cohort | Cohort 1a | Cohort 1 | Cohort 2 | Cohort 3 | Cohort 4 | Cohort 6 | Total |
|---|---|---|---|---|---|---|---|
| Pola dose | 1.4 mg/kg | 1.4 mg/kg | 1.8 mg/kg | 1.4 mg/kg | 1.8 mg/kg | 1.8 mg/kg | |
| Venetoclax dose | 200 mg | 400 mg | 400 mg | 600 mg | 600 mg | 800 mg | |
| Obinutuzumab dose | 1000 mg | 1000 mg | 1000 mg | 1000 mg | 1000 mg | 1000 mg | |
| N | 3 | 7 | 3 | 3 | 9 | 8 | 33 |
| ORR | 3 (100.0) | 4 (57.1) | 2 (66.7) | 2 (66.7) | 6 (66.7) | 8 (100.0) | 25 (75.8) |
| CR | 2 (66.7) | 2 (28.6) | 1 (33.3) | 1 (33.3) | 5 (55.6) | 8 (100.0) | 19 (57.6) |
| PR | 1 (33.3) | 2 (28.6) | 1 (33.3) | 1 (33.3) | 1 (11.1) | 0 | 6 (18.2) |
| SD | 0 | 2 (28.6) | 0 | 1 (33.3)* | 2 (22.2)* | 0 | 5 (15.2) |
| PD | 0 | 1 (14.3) | 0 | 0 | 0 | 0 | 1 (3.0) |
| NE/missing | 0 | 0 | 1 (33.3) | 0 | 1 (11.1) | 0 | 2 (6.1) |

*One patient in cohort 3 and one patient in cohort 4 had downgrades from PMR to NMR due to CT responses of SD per Modified Lugano definition.
MRI = magnetic resonance imaging; NE = not evaluable; NMR = no metabolic response; ORR = objective response rate; PD = progressive disease; Pola = polatuzumab vedotin; PMR = partial metabolic response; PR = partial response; SD = stable disease.

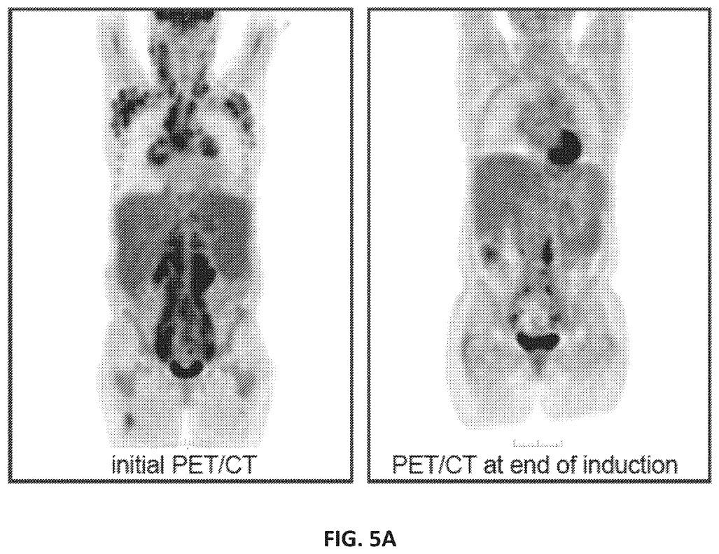

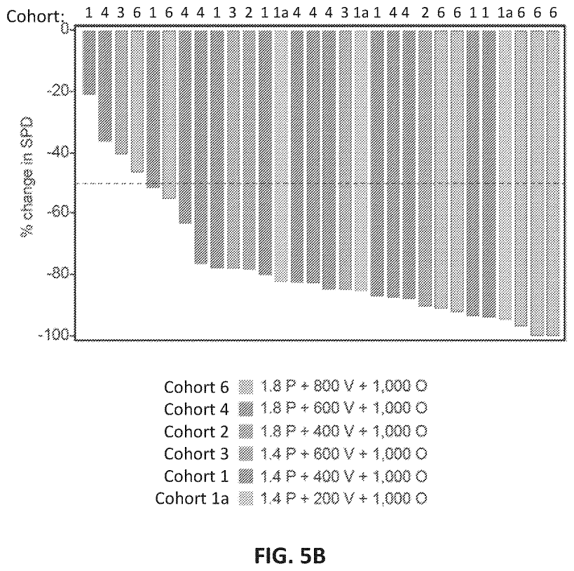

Representative PET/CT images of the patient in cohort 1 who achieved a CR at EOI are shown in FIG. 5A, and they reveal remarkably reduced disease burden after induction treatment. Specifically, at the end of induction treatment (right panel), there was a significant signal decrease in cervical, axillary, mediastinal, and abdominal lymph nodes compared to initial screening (left panel).

FIG. 51B shows responses, determined by computed tomography (CT) scans, as percent change in the sum of perpendicular diameters (SPD) from baseline and at end of induction in patients with follicular lymphoma, separated by cohort. These results showed that a majority of patients achieved greater than 50% decrease in tumor size from baseline.

### D. Discussion

The study described in this Example is the first clinical study to combine polatuzumab vedotin and venetoclax with the anti-CD20 antibody obinutuzumab in patients with relapsed/refractory FL. The phase 1b dose finding study identified the RP2D for polatuzumab vedotin as 1.8 mg/kg in combination with venetoclax 800 mg and obinutuzumab 1,000 mg. This triplet combination was well tolerated by most patients and had an acceptable safety profile. Due to the overlapping toxicities of the single agents, the most significant safety finding was cytopenias that trended toward higher rates of neutropenia and thrombocytopenia as the venetoclax dose increased. However, the components in this triple combination (Pola-G-Ven) have minimally overlapping dose-limiting toxicities with each other and with common chemotherapy drugs used in NHL. The myelo-suppressive effects of polatuzumab vedotin combined with venetoclax and obinutuzumab were manageable with granulocyte colony-stimulating factor (G-CSF) prophylaxis, supportive measures, and dose modifications or delays.

Excellent response rates for polatuzumab vedotin+venetoclax and obinutuzumab triplet combination were observed in heavily pretreated patients with relapsed/refractory FL, where the majority of patients were refractory to their last line of treatment. These results compared favorably with historical response rates seen in studies evaluating doublet combinations such as ROMULUS (polatuzumab vedotin+obinutuzumab/rituximab) or CONTRALTO (venetoclax+rituximab) (Morschhauser et al. Polatuzumab vedotin or pinatuzumab vedotin plus rituximab in patients with relapsed or refractory non-Hodgkin lymphoma: final results from a phase 2 randomised study (ROMULUS). Lancet Haematol. 2019 May; 6(5):e254-e265; Zinzani et al. Efficacy and safety of venetoclax (Ven)+rituximab (R) or Ven+bendamustine (B)+R randomized versus B+R in patients (pts) with relapsed/refractory (R/R) follicular lymphoma (FL): final analysis of Phase II CONTRALTO study. Blood 2018; 132(Suppl 1):1614; Phillips et al. Polatuzumab vedotin combined with obinutuzumab for patients with relapsed or refractory non-Hodgkin lymphoma: preliminary safety and clinical activity of a phase Ib/II study. Blood 2016; 128:1) that had CR rates of 33% and 17%, respectively, in relapsed/refractory FL patients.

In particular, the trend towards early CR/partial response (PR) rates (87.5%) after only two treatment cycles in patients receiving polatuzumab 1.8 mg/kg and venetoclax 800 mg (cohort 6) was notable and distinct from response kinetics observed at lower dose cohorts. This translated into a 100% CR rate for patients in cohort 6 at the end of induction.

### V. Results of the FL Expansion Phase

An interim analysis of the FL expansion phase was performed. The analysis included 71 patients included in the FL dose escalation phase (Phase Ib) and the expansion phase (Phase II)

### A. Patient Characteristics

The median patient age was 63 years (range 36-78) years. 55% of patients were male. 49% of patients had Follicular Lymphoma International Prognostic Index (FLIPI) of 3-5. 73% of patients had received ≥2 prior therapy lines. 52% of patients were refractory to their last treatment. 16% of patients had bulky disease (≥7 cm).

### B. Safety

Following the initial dose escalation phase, the combination of polatuzumab vedotin 1.8 mg/kg+venetoclax 800 mg was selected as the RP2D for expansion in combination with 1000 mg obinutuzumab.

The most common all grade non-hematologic adverse events (AEs) in patients in the dose escalation and expansion phases were infections, diarrhea, nausea, and fatigue. Grade 3/4 AEs were reported in 59% of patients, most commonly: neutropenia (31%), thrombocytopenia (18%), infections (13%), and anemia (6%). Peripheral neuropathy (PN) was reported in 41% of patients, all were Grade 1 or 2. Four patients required a dose reduction of polatuzumab vedotin and 2 patients discontinued polatuzumab vedotin due to peripheral neuropathy. AEs leading to venetoclax dose reduction or interruption occurred in 30% and 47% of patients, respectively, with the most common cause being cytopenias.

These results showed that the combination of polatuzumab vedotin, obinutuzumab, and venetoclax had an acceptable safety profile.

### C. Efficacy

Currently available data for 15 efficacy evaluable patients from the FL expansion phase showed an independent review committee-assessed modified Lugano 2014 criteria response rate of 87% and a CR rate of 60%. Fourteen patients continue on maintenance treatment. With a median follow-up duration of 7.4 months in the efficacy-evaluable population, median progression-free survival has not been reached. A summary of the responses at the end of induction (EOI) for the efficacy evaluable population treated at the RP2D is provided in Table 18.

TABLE 18
Responses at the end of induction (efficacy-evaluable
population; recommended phase II dose; N = 15).

Modified Lugano Criteria

| End of induction response, n(%) | INV | IRC |
|---|---|---|
| Objective response rate | 14 (93) | 13 (87) |
| CR | 9 (60) | 9 (60) |
| PR | 5 (33) | 4 (27) |
| SD | 1 (7) | 2 (13) |
| PD | 0 | 0 |
| Missing/unevaluable | 0 | 0 |

Modified Lugano criteria required a negative bone marrow biopsy to confirm PET-CR, and PET-PR was required to meet CT-PR criteria.

These results showed that response rates at end of induction with Pola-G-Ven are promising, with high CR rates compared with available R/R FL treatments

### Example 2: An Interim Analysis of a Phase Ib/II Study of an Anti-CD79b Immunoconjugate (Polatuzumab Vedotin) in Combination with an Anti-CD20 Antibody (Obinutuzumab) and a Bcl-2 Inhibitor (Venetoclax) in Relapsed or Refractory Follicular Lymphoma (FL)

This Example describes an interim analysis of safety and efficacy results of the Phase Ib/II study described in Example 1, which evaluated the safety and efficacy of obinutuzumab (G) in combination with polatuzumab vedotin (Pola) and venetoclax (V) in patients with relapsed or refractory follicular lymphoma (R/R FL).

### A. Patient Characteristics

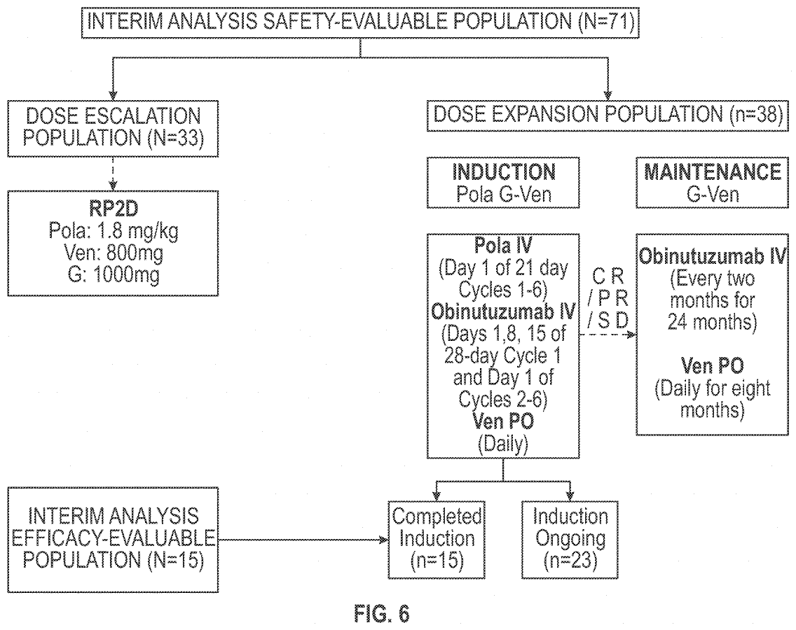

A total of 71 patients with R/R FL were included in the interim analysis described in this Example. As shown in FIG. 6, all 71 patients were included in the safety evaluable population, 33 of whom were enrolled in the dose escalation phase of this study, and 38 of whom were enrolled in the expansion phase of this study. Fifteen patients from the dose expansion phase of this study who had completed induction treatment were included in the efficacy evaluable population.

A summary of baseline patient demographics and disease characteristics for patients in the safety evaluable population is provided in Table 19.

TABLE 19
Baseline characteristics.

| Characteristic at study entry, n (%) | N = 71 | |
|---|---|---|
| Median age, years (range) | 63 | (36-78) |
| Male | 39 | (55) |
| ECOG PS 0-1 | 69 | (97) |
| Ann Arbor Stage III/IV | 58 | (82) |
| Bulky disease (≥7 cm) | 11 | (16) |
| Bone marrow involvement | 28 | (39) |
| FLIPI high ≥3 | 35 | (49) |
| No. of prior lines of treatment* | | |
| 1 | 19 | (27) |
| 2 | 21 | (30) |
| ≥3 | 31 | (44) |
| Median no. of prior lines (range) | 2 | (1-8) |
| Refractory to last prior therapy^{†} | 37 | (52) |
| Refractory to any line of anti-CD20^{‡} | 35 | (50) |
| POD24 on first-line treatment^{¶} | 19 | (27) |

FLIPI = follicular lymphoma international prognostic index; ECOG PS = Eastern Cooperative Oncology Group performance status; POD24 = progression of FL within 24 months of diagnosis;
*Prior lines of chemotherapy;
^{†}Defined as no response or progression or relapse within six months of last anti-lymphoma
therapy end date;
^{‡}Defined as no response or progression or relapse within six months of therapy with an anti-CD20 agent at any prior line of treatment;
^{¶}Defined as progression of disease within 24 months of initiation of first anti-lymphoma treatment with chemoimmunotherapy.

### B. Safety

Seventy-one patients were included in the safety evaluable population, 33 of whom were enrolled in the dose escalation phase of this study, and 38 of whom were enrolled in the expansion phase of this study (FIG. 6). The median duration of follow-up for the safety evaluable population was 8.11 months (range: 0.3-42.2). All 71 patients in the safety evaluable population experienced at least one adverse event. The most common AEs were diarrhea (49%), nausea (39%), and fatigue (35%). Grade 3 or Grade 4 AEs were experienced by 42 patients (59%), and 17 patients (24%) experienced a serious AE. The most common Grade 3 or Grade 4 AEs were neutropenia (35%), thrombocytopenia (30%), and infections (13%). Febrile neutropenia was reported in two patients (3%). Peripheral neuropathy occurred in 29 patients (41%); all cases were Grade 1 or Grade 2. Adverse events led to treatment discontinuation in 10 patients (14%), delay or interruption of any study drug in 38 patients (54%), and dose reductions of any study drug in 23 patients (32%). No grade 5 AEs were reported. A summary of the most common AEs occurring in ≥20% of patients is presented in Table 20.

TABLE 20
Adverse events occurring in ≥20% of patients.

| | Safety-evaluable population (N = 71) | |
|---|---|---|
| Adverse event by preferred term | All Grade, n (%) | Grade 3 or 4, n (%) |

Blood and lymphatic system disorders

| | | |
|---|---|---|
| Neutropenia | 25 (35) | 22 (31) |
| Thrombocytopenia | 21 (30) | 13 (18) |
| Anemia | 10 (14) | 4 (6) |

Non-hematologic events

| | | |
|---|---|---|
| Infections (by Systems | 39 (55) | 9 (13) |
| Organ Class) | | |
| Diarrhea | 35 (49) | 1 (1) |
| Nausea | 28 (39) | 3 (4) |
| Fatigue | 25 (35) | 1 (1) |
| Infusion related reaction | 20 (28) | 2 (3) |
| Cough | 18 (25) | 0 |
| Vomiting | 17 (24) | 2 (3) |
| Neuropathy peripheral* | 29 (41) | 0 |
| Headache | 15 (21) | 0 |

*Peripheral neuropathy standard MedDRA query included: neuropathy peripheral, paraesthesia, peripheral motor neuropathy, and peripheral sensory neuropathy.

### C. Efficacy

The efficacy evaluable population included 15 patients from the dose expansion phase of this study who had completed induction treatment (FIG. 6). The median duration of follow-up for the efficacy evaluable population was 7.43 months (range: 5.6-8.3). As shown in Table 21, 13 patients (87%) achieved a complete response or a partial response (IRC-assessed using the Modified Lugano 2014 criteria) at the end of induction, with 9 patients (60%) achieving a complete response.

TABLE 21
Summary of Efficacy Results.

| | Efficacy-evaluable | | | | |
|---|---|---|---|---|---|
| | population (N = 15) | | | | |
| | Modified | | | | |
| | Lugano 2014* | | | Lugano 2014 | |
| EOI response, n (%) | INV | IRC | | INV | IRC |
| Objective response | 14 (93) | 13 | (87) | 14 (93) | 14 (93) |
| Complete response (CR) | 9 (60) | 9 | (60)^{†} | 9 (60) | 11 (73) |
| Partial response (PR) | 5 (33) | 4 | (27) | 5 (33) | 3 (20) |
| Stable disease (SD) | 1 (7) | 2 | (13)^{‡} | 1 (7) | 1 (7) |

| | | | | |
|---|---|---|---|---|
| Disease progression (PD) | 0 | 0 | 0 | 0 |

*Modified Lugano 2014 criteria require a negative bone marrow biopsy to confirm PET-CR and PET-PR must also meet CT-PR criteria.
^{†}CR downgraded to PR due to missing bone marrow biopsy in 2 patients by IRC.
^{‡}PR downgraded to SD due to CT-SD + PET-PMR by IRC in 1 patient by IRC.
Lugano 2014 criteria according to Cheson et al, 2014. INV = investigator; IRC = independent review committee; PMR = partial metabolic response; EOI = end of induction.

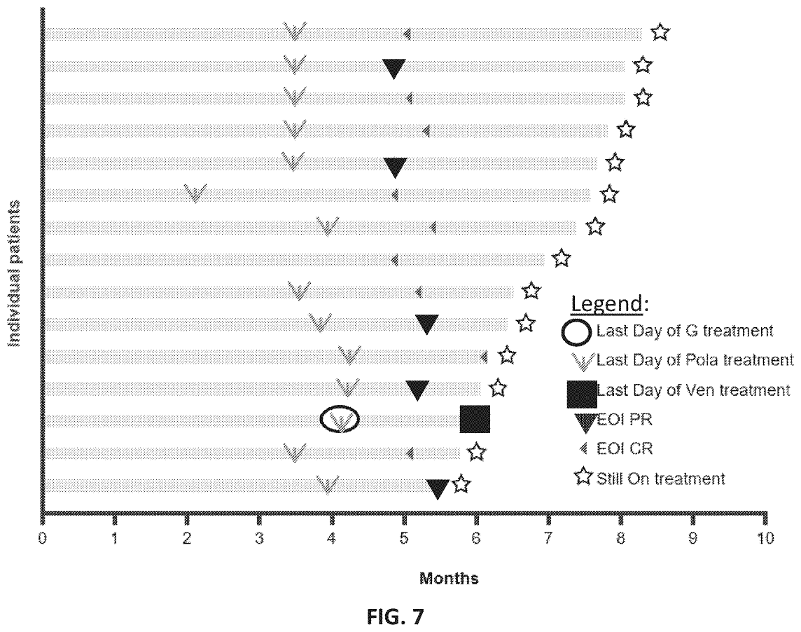

The time to response and the duration of responses (assessed by the investigator) in the efficacy evaluable population are provided in FIG. 7. Fourteen patients in the efficacy evaluable population remain on treatment.

### D. Discussion

The safety results described in this Example showed that the Pola-G-Ven combination was tolerable and had a safety profile consistent with known profiles of the individual study drugs in patients with R/R FL. In addition, adverse events were manageable with supportive care. The efficacy results described in this Example showed that the Pola-G-Ven combination resulted in 87% of patients responding at the end of induction, and 60% of patients achieving a complete response.

### Example 3: Primary Analysis of a Phase Ib/II Study of an Anti-CD79b Immunoconjugate (Polatuzumab Vedotin) in Combination with an Anti-CD20 Antibody (Rituximab) and a Bcl-2 Inhibitor (Venetoclax) in Relapsed or Refractory Diffuse Large B-Cell Lymphoma (DLBCL)

This Example describes a primary analysis of the Phase Ib/II study described in Example 1, which evaluated the safety and efficacy of rituximab (R) in combination with polatuzumab vedotin (Pola) and venetoclax (V; Ven) in patients with relapsed or refractory diffuse large B-cell lymphoma (R/R DLBCL).

### I. Overview of Study

As described in detail in Example 1, this study was an open-label, multicenter study of patients with R/R DLBCL who had received ≥1 prior anti-CD20 chemoimmunotherapy regimen, and had histologically documented CD20+ cells and at least one bi-dimensionally measurable lesion ≥1.5 cm in its longest dimension.

The recommended Phase II dose (RP2D) for the Pola-R-Ven combination was initially defined in a 3+3 dose escalation phase, and was then expanded into Phase II. Patients in the expansion cohort received induction therapy with six 21-day cycles of: intravenous (IV) Pola at a dose of 1.8 mg/kg administered on Day 1 of Cycles 1-6; venetoclax administered by mouth daily at a dose of 800 mg; and rituximab administered IV at a dose of 375 mg/m² on Day 1 of Cycles 1-6. Responders received consolidation therapy for 8 months (venetoclax at a dose of 800 mg daily and rituximab at a dose of 375 mg/m² on Day 1 of every 2 months).

The primary safety objectives were to determine the RP2D for Pola and Ven when given in combination with rituximab and to evaluate the safety and tolerability ofthe Pola-R-Ven combination.

The primary efficacy endpoint was complete response (CR) at end of induction (EOI), as determined by the Independent Review Committee (IRC) based on positron emission tomography-computed tomography (PET-CT) scans using the modified Lugano 2014 response criteria. Secondary objectives included the CR-rate at EOI and the best overall response (BOR) determined by the investigator (INV). Exploratory objectives included INV-assessed progression-free survival (PFS), overall survival (OS), and biomarker analyses.

### II. Primary Efficacy and Safety Analysis

A. Patient Characteristics

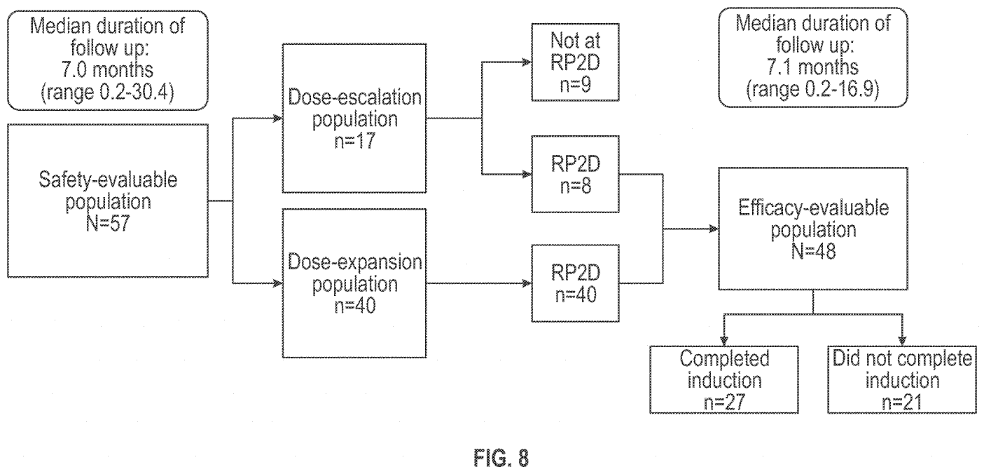

An overview of the study populations at the time of the primary analysis described in this Example is provided in FIG. 8. Fifty-seven patients from the Phase Tb/II populations were enrolled and received at least one study drug. The median duration of follow-up was 7.0 months (range 0.2-30.4). The median age of patients in the safety evaluable population was 65 years. 49% of patients were male, and 84% of patients had disease with Ann Arbor Stage III-IV. 54% of patients had an International Prognostic Index score of 3. The median number of prior lines of therapy was 3, and 83% of patients were refractory to the last line of therapy. 65% of patients had primary refractory disease. A summary of the baseline patient characteristics is provided in Table 22.

TABLE 22
Baseline Patient Characteristics.

| | Pola-R-Ven | | Pola-R-Ven | |
|---|---|---|---|---|
| | Safety Evaluable | | Efficacy Evaluable | |
| | Population | | Population | |
| Characteristic | (N = 57) | | (N = 48) | |
| Age in years, median (range) | 65 | (31-88) | 65 | (43-88) |
| Male sex, n (%) | 28 | (49) | 25 | (52) |
| ECOG PS, n (%) | | | | |
| 0-1 | 51 | (89) | N/A | |
| 2 | 6 | (11) | 4 | (8) |
| Ann Arbor Stage III/IV disease, | 48 | (84) | 42 | (88) |
| n (%) | | | | |
| IPI 3-5 at enrollment, n (%) | 31 | (54) | 27 | (56) |
| Bone marrow involvement, n(%) | 4 | (7) | N/A | |
| Bulky disease (27 cm), n (%) | 18 | (32) | N/A | |
| Extranodal disease, n (%) | 36 | (63) | N/A | |
| Number of prior therapies, | 3 | (1-7) | 3 | (1-7) |
| median (range) | | | | |
| 1 | 13 | (23%) | 10 | (21) |
| 2 | 15 | (26%) | 14 | (29) |
| ≥3 | 29 | (51%) | 24 | (50) |
| Refractory, n (%) | | | | |
| Last prior therapy* | 47 | (83) | 32 | (67) |
| Primary refractory** | 37 | (65) | 39 | (81) |
| Prior autologous bone marrow | 12 | (21) | 10 | (21) |
| transplant, n (%) | | | | |
| Prior CAR-T therapy | 2 | (4) | 2 | (4) |

*Defined as no response or progression or relapse within 6 months of the last prior anti-lymphoma therapy end date.
** Defined as no response or progression or relapse within 6 months of the first anti-lymphoma therapy end date.
ECOG PS = Eastern Cooperative Oncology Group performance status; IPI = International Prognostic Index.
N/A = not available.

Overall, baseline patient characteristics showed that patients included in this study were highly pre-treated and refractory at baseline.

### B. Safety

Dose-limiting toxicities were not observed in patients in Phase I. Therefore, the maximum dose level was chosen as the recommended Phase II dose (RP2D).

All except for 2 patients experienced at least one adverse event (AE). 21 patients (37%) had a serious AE, and 45 patients (79%) had a Grade 3-4 AE. The most common Grade 3-4 AEs were neutropenia (30 patients, 53%), infections (9 patients, 16%), and anemia (6 patients, 11%).

AEs leading to dose reduction or interruption of any study drug occurred in 10 patients (18%) and 35 patients (61%), respectively. The majority of dose modifications were changes to venetoclax dosing. Seven patients (12%) had an AE that led to the discontinuation of any study drug (Pola [n=5]; Ven [n=7]; R [n=6]).

One Grade 5 AE was reported (pneumonia). However, it was not considered related to study treatment because the patient had received a new anti-lymphoma therapy following disease progression.

An overview of adverse events that occurred in ≥10% of patients, Grade 3-4 adverse events, and serious adverse events is provided in Table 23.

TABLE 23
Adverse Events that Occurred in ≥10%
of Patients, Grade 3-4 AEs, and SAEs.

Safety Evaluable N = 57

| | All Grade | | Grade 3-4 | SAE |
|---|---|---|---|---|
| Total | 55 | (97)¹ | 45 (79) | 21 (37) |

Hematologic AEs

| | | | | |
|---|---|---|---|---|
| Neutropenia | 30 | (53) | 30 (53) | 3 (5) |
| Anemia | 13 | (23) | 6 (11) | 1 (2) |
| Thrombocytopenia | 7 | (12) | 4 (7) | 0 |

Non-hematologic AEs

| | | | | |
|---|---|---|---|---|
| Infections and infestations² | 26 | (46) | 9 (16) | 6 (11) |
| Diarrhea | 26 | (46) | 3 (5) | 2 (4) |
| Nausea | 21 | (37) | 0 | 0 |
| Vomiting | 17 | (30) | 0 | 0 |
| Fatigue | 15 | (26) | 0 | 0 |
| Peripheral neuropathy³ | 13 | (23) | 0 | 0 |
| Pyrexia | 13 | (23) | 2 (4) | 2 (4) |
| Decreased appetite | 13 | (23) | 0 | 0 |
| Cough | 12 | (21) | 0 | 0 |
| Hypokalemia | 11 | (19) | 3 (5) | 0 |
| Constipation | 10 | (18) | 1 (2) | 1 (2) |
| Weight decreased | 7 | (12) | 0 | 0 |
| Hypomagnesemia | 6 | (11) | 0 | 0 |
| Dyspnea | 6 | (11) | 2 (4) | 1 (2) |
| Pruritus | 6 | (11) | 0 | 0 |
| Infusion related reaction | 6 | (11) | 0 | 0 |

¹Grade 5 AE: one Grade 5 AE of pneumonia after progressive disease and new anti-lymphoma treatment (CAR-T cell therapy) considered unrelated to study drugs occurred.
²Infections presented as Systems Organ Class terms - all other AEs are reported by ‘preferred terms.’
³Peripheral neuropathy standard MedDRA query included: peripheral motor neuropathy, peripheral
sensory neuropathy, neuropathy peripheral, paresthesia, hypoesthesia, and neuralgia.
SAE = serious adverse event.

As shown in Table 23, the majority of adverse events were gastrointestinal-related or blood and lymphatic system-related adverse events. Toxicities were mainly hematologic, gastrointestinal, and infections, with infections being mostly low-grade.

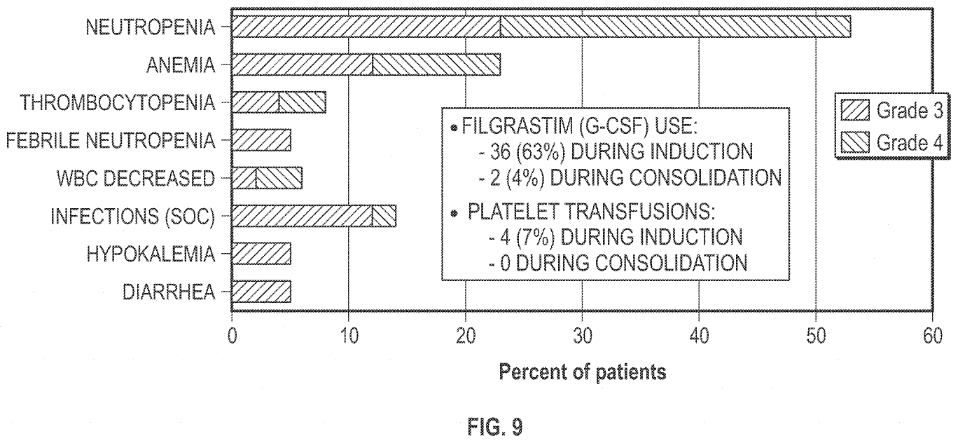

Grade 3-4 adverse events included neutropenia, anemia, thrombocytopenia, febrile neutropenia, white blood cell decreased, infections, hypokalemia, and diarrhea. An overview of Grade 3-4 adverse events that occurred in ≥5% of patients, reported by preferred term, is provided in FIG. 9. G-CSF (filgrastim) was administered to 36 patients (63%) during the induction phase and to 2 patients (4%) during consolidation. Platelet transfusions were administered to 4 patients (7%) during the induction phase. No patients received platelet transfusions during consolidation.

A summary of adverse events to monitor and adverse events of special interest (AESIs) is provided in Table 23a. Observed toxicities were manageable with supportive care and dose interruptions or reductions. The incidence of peripheral neuropathy was low, and events that occurred were low grade. There were few cases of febrile neutropenia and Grade 3-4 infections.

TABLE 23a
Adverse events to monitor and adverse
events of special interest (AESIs).

| | |
|---|---|
| Events to monitor, n (%) | Safety-evaluable (N = 57) |

| | | |
|---|---|---|
| Neutropenia | All grade | 30 (53) |
| | Grade 3 | 13 (23) |
| | Grade 4 | 17 (30) |
| | Led to dose reduction | 4 (7) |
| Febrile neutropenia | Grade 3 | 2 (5) |
| Thrombocytopenia | All grade | 7 (12) |
| | Grade 3 | 2 (4) |
| | Grade 4 | 2 (4) |
| | Led to dose reduction | 0 |
| Peripheral neuropathy1 | All grade | 13 (23) |
| | Grade 2 | 3 (5) |
| | Grade 3-4 | 0 |
| | Led to dose reduction | 2 (4) |

| AESIs, n (%) | Safety-evaluable N = 57 |
|---|---|
| Tumor lysis syndrome | 1 (2) |
| Second malignancies | 0 |

¹Peripheral neuropathy standard MedDRA query.

### C. Efficacy

The median duration of follow up was 7.1 months (0.2-16.9). In the primary efficacy-evaluable population (n=48), the IRC-assessed modified Lugano CR rate at EOI was 29% and the INV-assessed CR rate at EOI was 31%. A summary of the primary efficacy results at EOI is provided in Table 24.

TABLE 24
Primary Efficacy Results at EOI (PET/CT responses).

| | Pola-R-Ven | |
|---|---|---|
| Outcome | (N = 48) | |
| Complete Response (IRC)*, n (%) | 14 | (29) |
| Complete Response (INV), n (%) | 15 | (31) |
| Objective Response (IRC), n (%) | 14 | (29) |
| Objective Response (INV), n (%) | 20 | (42) |
| Partial Response (IRC), n (%) | 0 | |
| Partial Response (INV), n (%) | 5 | (10) |
| Stable Disease (IRC), n (%) | 5 | (10) |
| Stable Disease (INV), n (%) | 0 | |
| Disease Progression (IRC), n (%) | 12 | (25) |
| Disease Progression (INV), n (%) | 23 | (48) |
| Missing/not evaluable (IRC), n (%) | 17 | (35) |
| Missing/not evaluable (INV), n (%) | 5 | (10) |

*Primary efficacy endpoint. INV = investigator; IRC = independent review committee. Responses reported according to modified Lugano criteria.

The INV-assessed best overall response (BOR) rate was 65%. The median INV-assessed overall survival was 11.0 months (95% CI: 6.7, not evaluable). A summary of INV-assessed efficacy results is provided in Table 25.

TABLE 25
Investigator-Assessed Efficacy Results Summary.

| | Pola-R-Ven | |
|---|---|---|
| Outcome | (N = 48) | |
| BOR** , n (%) | 31 | (65) |
| Complete Response | 18 | (38) |
| Partial Response | 13 | (27) |
| Median DOR, months (95% CI) | 5.8 | (3.4, 6.7) |
| Median PFS, months (95% CI) | 4.4 | (3.0, 7.1) |
| Median OS, months (95% CI) | 11.0 | (6.7, NE) |

**Defined as best response of CR or partial response during the study; based on composite result: modified Lugano > Lugano PET-CT > Lugano CT only.
BOR = best overall response; CI = confidence interval; DOR = duration of response; OS = overall survival; PFS = progression-free survival; NE = not evaluable.

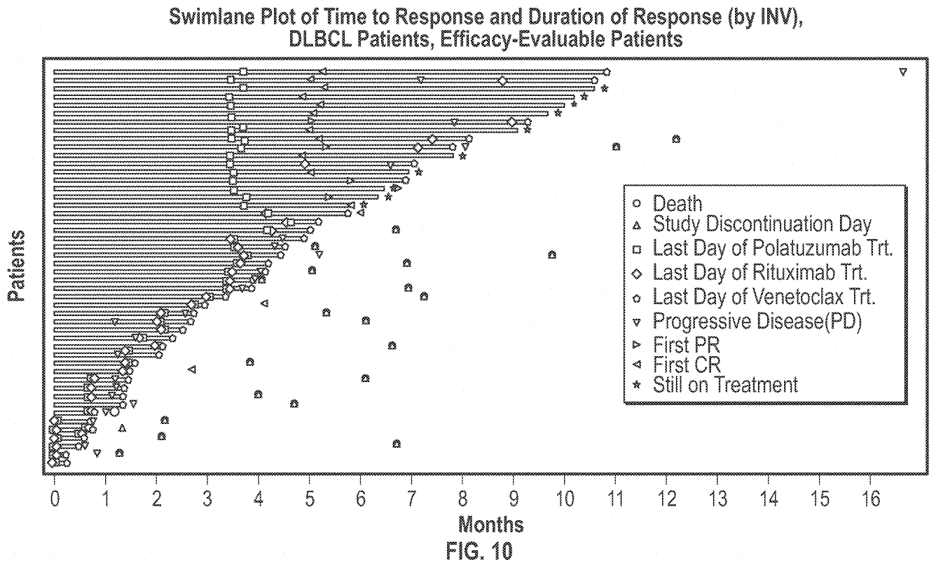

FIG. 10 provides a Swimlane plot showing the time to response and the duration of INV-assessed responses observed in the efficacy-evaluable population. The median duration of INV-assessed responses was 5.8 months (95% confidence interval [CI]: 3.4, 6.7).

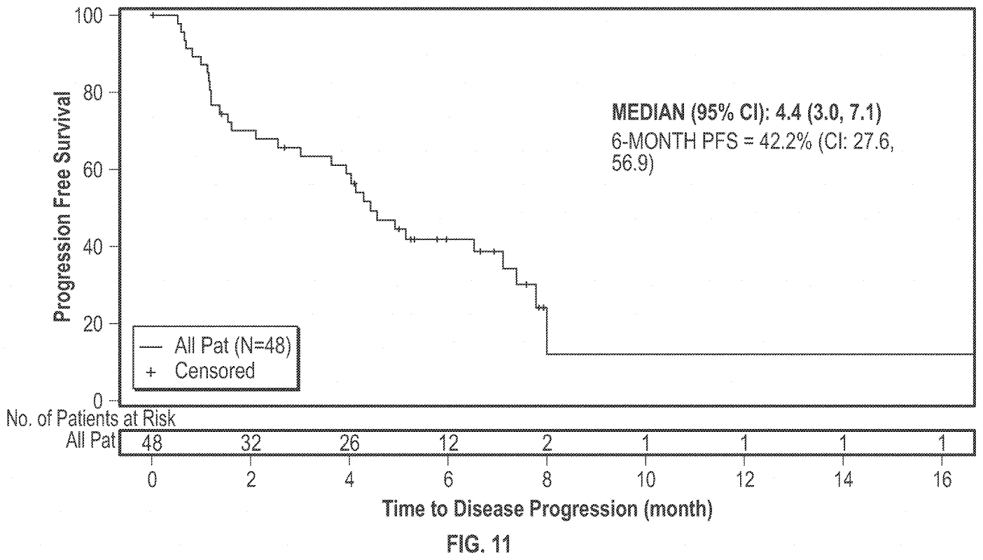

As shown in FIG. 11, the median INV-assessed PFS was 4.4 months (95% CI: 3.0, 7.1) and the 6-month PFS was 42.2% (CI: 27.6, 56.9).

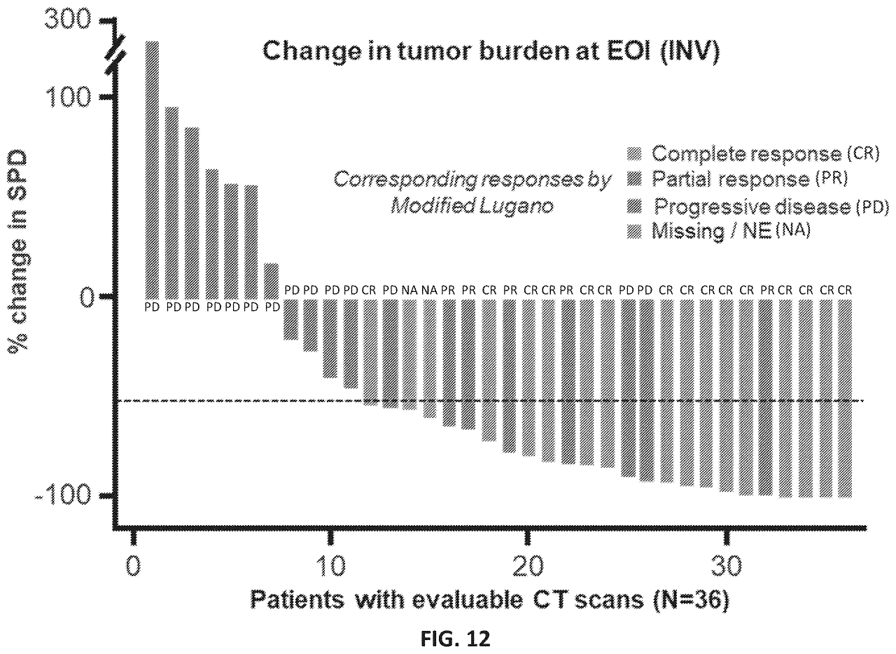

As shown in FIG. 12, most patients achieved a greater than 50% reduction in the sum of the product of diameters (SPD) at EOI, assessed by the investigator.

Efficacy results were analyzed based on cell of origin (COO) and other biomarker subgroups, including Bcl-2 and Myc expression. COO (activated B cell/germinal center B cell) was determined using a NanoString assay. Bcl-2 protein expression was assessed by immunohistochemistry (IHC) using the anti-Bcl-2 (124) mouse monoclonal antibody. Bcl-2 IHC scoring incorporated the percentage of positively stained tumor cells (≥50% of tumor cells as previously defined; Morschhauser F, et al., Blood 2020) and the intensity of tumor cell staining. Myc expression was assessed by IHC using the clone Y69 Epitomics antibody. Tumors were classified as Myc IHC-positive if ≥40% of cells showed Myc nuclear staining above background intensity.

As shown in Table 26, efficacy was consistent across COO subgroups. In addition, the study population was enriched for double expresser (DEL+) patients (BCL2+, MYC+), which had higher CR rates than DEL-patients.

TABLE 26
Biomarker analyses.

| | | Cell of Origin (COO) | | BCL2 and MYC protein | | |
|---|---|---|---|---|---|---|
| | | by NanoString | | expression by IHC | | |
| | Total | GCB | ABC | BCL2− | DEL | Non-DEL |
| Response (INV) | (N = 48) | (N = 14) | (N = 12) | (N = 6) | (N = 20) | (N = 10) |
| Best ORR, n (%)* | 31 (65) | 5 (36) | 6 (50) | 2 (33) | 8 (40) | 4 (40) |
| Complete response | 18 (38) | 3 (21) | 6 (50) | 1 (17) | 7 (35) | 1 (10) |
| Partial response | 13 (27) | 2 (14) | 0 (0) | 1 (17) | 1 (5) | 3 (30) |
| Median PFS, months | 4.4 | 4.4 | 5.7 | 1.2 | 4.6 | 2.1 |
| (95% CI) | (3.0, 7.1) | (4.0, NE) | (1.5, NE) | (0.8, NE) | (4.0, NE) | (1.0, NE) |
| Median OS, months | 11.0 | 11.0 | 6.7 | 6.7 | 9.8 | 7.2 |
| (95% CI) | (6.7, NE) | (6.9, NE) | (5.1, NE) | (6.1, NE) | (6.6, NE) | (6.1, NE) |

*Based on composite result: Modified Lugano > Lugano PET-CT > Lugano CT only.
ABC = activated B cell; BCL2 = B cell lymphoma 2; DEL = double expresser lymphoma; GCB = germinal center B cell; IHC = immunohistochemistry; ORR = objective response rate.

### D. Conclusions

The safety results described in this Example showed that the novel combination of Pola-R-Ven was well tolerated and had a safety profile consistent with the known profiles of the individual drugs. In particular, hematological and gastrointestinal toxicities were consistent with overlapping toxicity profiles of the individual drugs and were manageable with supportive care and dose interruptions/reductions. Cytopenias were mostly driven by neutropenia.

In addition, the triplet combination exhibited promising efficacy results in a heavily pre-treated and refractory population of patients with R/R DLBCL.

### Example 4: Primary Analysis of a Phase Ib/II Study of an Anti-CD79b Immunoconjugate (Polatuzumab Vedotin) in Combination with an Anti-CD20 Antibody (Obinutuzumab) and a Bcl-2 Inhibitor (Venetoclax) in Relapsed or Refractory Follicular Lymphoma (FL)

This Example describes a primary analysis of safety and efficacy results of the Phase Ib/II study described in Examples 1 and 2, which evaluated the safety and efficacy of obinutuzumab (G) in combination with polatuzumab vedotin (Pola) and venetoclax (V) in patients with relapsed or refractory follicular lymphoma (R/R FL).

### I. Methods

As described in detail in Example 1, patients, including patients with Grade 1, 2 or 3a R/R FL, received induction treatment every 21 days for 6 cycles as follows:
Polatuzumab vedotin administered intravenously on day 1 at a dose of 1.4-1.8 mg/kg during the dose escalation phase, or at the recommended Phase 2 dose (RP2D).
Obinutuzumab administered intravenously at a dose of 1000 mg on days 1, 8, and 15 of Cycle 1, and on day 1 of cycles 2-6.
Venetoclax administered orally on days 1-21 at a dose of 200-800 mg during the dose escalation phase, or at the RP2D.

Patients who achieved a complete response, partial response or stable disease (CR/PR/SD) at end of induction (EOI) received 24 months of maintenance treatment with obinutuzumab (1000 mg on day 1 of every 2 months) and venetoclax (200-800 mg daily) for 8 months.

Primary endpoints of the study were safety, tolerability and positron emission tomography-computed tomography (PET-CT)-CR rate at EOI, assessed by an independent review committee (IRC) using the modified Lugano 2014 criteria.

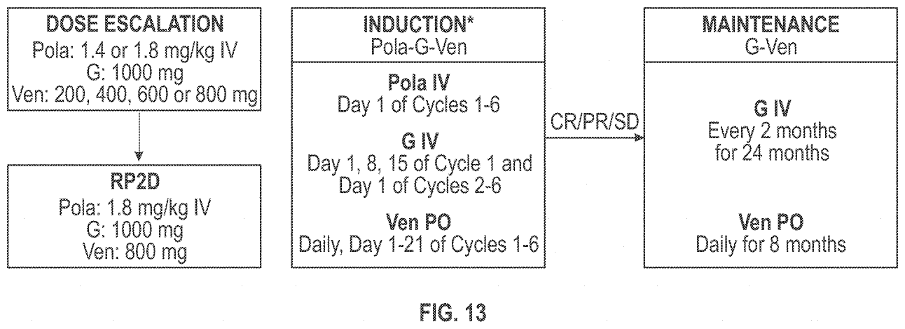

An overview of the study design is provided in FIG. 13.

### II. Results

A. Patient Characteristics

At primary analysis, 74 patients were enrolled in the study. A summary of patient characteristics is provided in Table 27.

TABLE 27
Patient characteristics.

| Characteristic | Value (N = 74) | |
|---|---|---|
| Median age (range) | 64 years | (range 36-78) |
| Male, n(%) | 42 | (57%) |
| Ann Arbor Stage III-IV, n(%) | 64 | (86%) |
| FL International Prognostic Index high | 41 | (55%) |
| risk ≥3, n(%) | | |
| Bulky disease ≥7 cm, % | 16% | |
| Number of prior therapies, median (range) | 2 | (1-9) |
| 1, n(%) | 19 | (26%) |
| 2, n(%) | 20 | (27%) |
| ≥3, n(%) | 35 | (47%) |
| Prior lines of therapy ≥2, % | 74% | |
| Refractory to last prior therapy*, n(%) | 38 | (51%) |
| Refractory to any prior anti-CD20 | 41 | (55%) |
| therapy†, n(%) | | |
| Baseline ECOG PS 2, n(%) | 3 | (4%) |
| Bone marrow involvement, n(%) | 29 | (39%) |
| POD24 with first-line treatment | 19 | (26%) |

*Defined as no response or progression or relapse within 6 months of last anti-lymphoma therapy end date.
†Defined as no response or progression or relapse within 6 months of therapy with an anti-CD20 agent at any prior line of treatment.
POD24, progression of FL within 24 months of initiation of first anti-lymphoma treatment with chemoimmunotherapy.

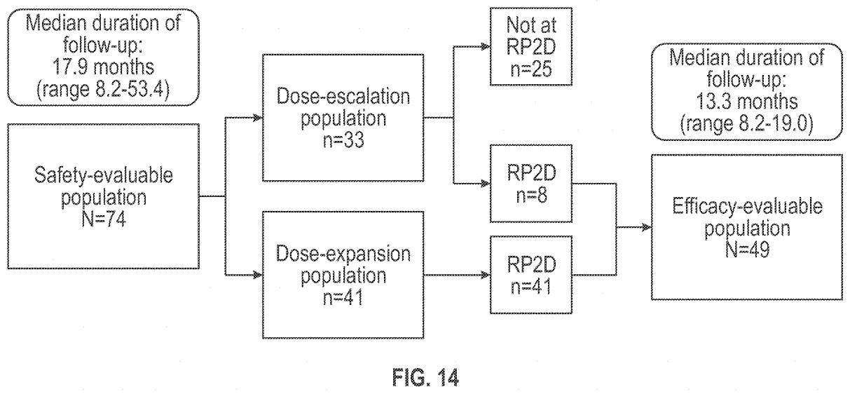

An overview of the primary analysis populations is provided in FIG. 14.

B. Safety

All patients had ≥1 adverse event (AE). 23 patients (31%) had a serious AE, and 54 patients (73%) had a Grade 3-4 AE. The most common Grade 3-4 AEs were neutropenia (39%), thrombocytopenia (19%), and infections (16%), mainly pneumonia. AEs that led to treatment discontinuation, delay/interruption or dose reduction of any drug occurred in 14 (19%), 50 (68%) and 28 (38%) of patients, respectively. Most dose reductions or interruptions were modifications to venetoclax dosing. One fatal AE of pneumonia was reported. Serious AEs occurred in 23 (31%) patients and were mainly infections (16%; pneumonia [7%]), infusion related reactions [5%], and febrile neutropenia [4%]). One treatment-related Grade 5 AE was reported (pneumonia), which occurred after new anti-lymphoma treatment (cyclophosphamide, doxorubicin and prednisone). Peripheral neuropathy was reported in 33 (45%) patients.

Table 28 provides a summary of AEs that occurred in ≥20% of patients.

TABLE 28
AEs that occurred in ≥20% of patients.

Safety evaluable
(N = 74)

| | | |
|---|---|---|
| AE, n (%) | All grade | Grade 3-4 |

Hematologic AEs

| | | |
|---|---|---|
| Neutropenia | 31 (42) | 29 (39) |
| Thrombocytopenia | 23 (31) | 14 (19) |

Non-hematologic AEs

| | | |
|---|---|---|
| Infections* | 48 (65) | 12 (16) |
| Diarrhea | 41 (55) | 4 (5) |
| Nausea | 35 (47) | 3 (4) |
| Peripheral neuropathy† | 33 (45) | 0 |
| Fatigue | 28 (38) | 1 (1) |
| Infusion-related reaction | 25 (34) | 3 (4) |
| Cough | 22 (30) | 0 |
| Vomiting | 21 (28) | 2 (3) |
| Headache | 17 (23) | 0 |
| Constipation | 16 (22) | 0 |

*Reported by systems organ class.
†Reported per Standardised MedDRA Query and includes peripheral neuropathy (23%), peripheral sensory neuropathy (10%), paraesthesia (10%), muscular weakness (3%), neuralgia (1%), and peripheral motor neuropathy (1%).

Table 29 provides a summary of AEs to monitor and AEs of special interest (AESIs).

TABLE 29
AEs to monitor and AESIs.

Safety evaluable
(N = 74)

AEs to monitor, n (%)

| | | |
|---|---|---|
| Peripheral neuropathy^{†} | All grade | 33 (45) |
| | Grade 2 | 13 (18) |
| | Grade 3 | 0 |
| | Led to dose reduction | 4 (5) |

AESIs, n (%)

| | |
|---|---|
| Tumor lysis syndrome | 2 (3) |
| Neoplasms, benign, malignant and unspecific | 6 (8) |
| Squamous cell carcinoma | 3 (4) |
| Intraocular melanoma | 1 (1) |
| Myelodysplastic syndrome | 1 (1) |
| Skin cancer | 1 (1) |

*Reported by systems organ class.
^{†}Reported per Standardised MedDRA Query and includes peripheral neuropathy (23%), peripheral sensory neuropathy (10%), paraesthesia (10%), muscular weakness (3%), neuralgia (1%), and peripheral motor neuropathy (1%).

C. Efficacy

49 patients were treated at the RP2D (Pola 1.8 mg/kg+Ven 800 mg+Obinutuzumab 1000 mg) and were evaluable for efficacy. The PET-CR rate at EOI assessed by an IRC was 57%. Three responses were downgraded due to missing bone marrow biopsies (n=2) and due to bone marrow status being assessed >28 days after EOI (n=1). Two cases of PET-PR were downgraded to SD by IRC as they failed to meet CT-PR requirements per Modified Lugano criteria. With a median follow-up time of 13.3 months (range 8.2-19.0), the median progression-free survival (PFS) was not reached. The IRC-assessed overall response rates at EOI were largely consistent among patients with high-risk disease characteristics.

A summary of efficacy results is provided in Table 30.

TABLE 30
Responses at EOI (RP2D; N = 49).

| | Efficacy evaluable | | | |
|---|---|---|---|---|
| | (N = 49) | | | |
| | Modified | | | |
| | Lugano 2014* | | Lugano 2014 | |
| Response, n (%) | IRC | INV | IRC | INV |
| Objective response | 35 (71) | 38 (78) | 37 (76) | 38 (78) |
| Complete response | 28 (57) | 28 (57) | 31 (63) | 28 (57) |
| Partial response | 7 (14) | 10 (20) | 6 (10) | 10 (20) |
| Stable disease | 8 (16) | 6 (12) | 6 (12) | 6 (12) |
| Disease progression | 4 (8) | 3 (6) | 4 (8) | 3 (6) |
| Missing/Not evaluable | 2 (4) | 2 (4) | 2 (4) | 2 (4) |

*Modified Lugano requires a negative bone marrow biopsy to confirm PET-CR and PET-PR must also meet CT-PR criteria.
CR, complete response; CT, computed tomography; EOI, end of induction; INV, investigator-assessed; IRC, independent review committee-assessed; PET, positron emission tomography; PR, partial response.

D. Conclusions

The safety profile of the Pola-G-Ven combination is consistent with known profiles of the individual drugs. AEs were manageable with supportive care. Most infections and events of peripheral neuropathy were low grade Response rates at EOI with Pola-G-Ven are encouraging in this R/R FL patient population.

1. A method for treating follicular lymphoma (FL) in a human in need thereof comprising administering to the human an effective amount of:
(a) an immunoconjugate comprising the formula 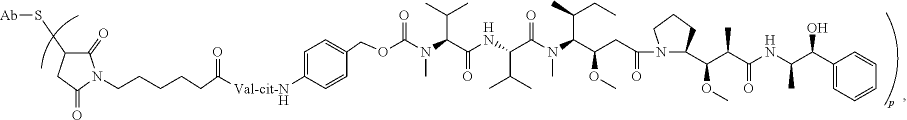
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) a selective Bcl-2 inhibitor, or a pharmaceutically acceptable salt thereof, and
(c) an anti-CD20 antibody,
wherein the human achieves a complete response (CR) during or after administration of the immunoconjugate, the selective Bcl-2 inhibitor, or a pharmaceutically acceptable salt thereof, and the anti-CD20 antibody.

2. (canceled)

3. The method of claim 1, wherein:
(a) the anti-CD79b antibody comprises (i) a heavy chain variable domain (VH) comprising the amino acid sequence of SEQ ID NO: 19 and (ii) a light chain variable domain (VL) comprising the amino acid sequence of SEQ ID NO: 20;
(b) the anti-CD79b antibody comprises (i) a heavy chain comprising the amino acid sequence of SEQ ID NO: 36 and (ii) a light chain comprising the amino acid sequence of SEQ ID NO: 35; and/or
(c) the immunoconjugate is polatuzumab vedotin-piiq.

4-5. (canceled)

6. The method of claim 1, wherein:
(a) the selective Bcl-2 inhibitor is venetoclax, or a pharmaceutically acceptable salt thereof; and/or
(b) the anti-CD20 antibody is rituximab or obinutuzumab.

7-9. (canceled)

10. The method of claim 6, wherein the immunoconjugate is polatuzumab vedotin-piiq administered at a dose of about 1.8 mg/kg, the venetoclax or a pharmaceutically acceptable salt thereof is administered at a dose of about 800 mg, and the obinutuzumab is administered at a dose of about 1000 mg.

11. The method of claim 10, wherein:
(a) administration of the polatuzumab vedotin-piiq, the venetoclax or a pharmaceutically acceptable salt thereof, and the obinutuzumab to a plurality of humans results in a complete response in at least about 55%, at least about 57%, at least about 60%, at least about 65%, at least about 70%, at least about 75%, at least about 80%, at least about 85%, at least about 90%, at least about 95%, or 100% of the humans during or after administration of the polatuzumab vedotin-piiq, the venetoclax or a pharmaceutically acceptable salt thereof, and the obinutuzumab;
(b) administration of the polatuzumab vedotin-piiq, the venetoclax or a pharmaceutically acceptable salt thereof, and the obinutuzumab to a plurality of humans results in an objective response in at least about 87%, at least about 90%, at least about 95%, or 100% of the humans during or after administration of the polatuzumab vedotin-piiq, the venetoclax or a pharmaceutically acceptable salt thereof, and the obinutuzumab: or administration of the polatuzumab vedotin-piiq, the venetoclax or a pharmaceutically acceptable salt thereof, and the obinutuzumab to a plurality of humans results in an objective response in at least about 70%, at least about 75%, at least about 78%, at least about 80%, at least about 85%, at least about 90%, at least about 95%, or 100% of the humans during or after administration of the polatuzumab vedotin-piiq, the venetoclax or a pharmaceutically acceptable salt thereof, and the obinutuzumab;
(c) administration of the polatuzumab vedotin-piiq, the venetoclax or a pharmaceutically acceptable salt thereof, and the obinutuzumab does not result in peripheral neuropathy of Grade 3 or greater in the human;
(d) administration of the polatuzumab vedotin-piiq, the venetoclax or a pharmaceutically acceptable salt thereof, and the obinutuzumab does not result in tumor lysis syndrome in the human; and/or
(e) administration of the polatuzumab vedotin-piiq, the venetoclax or a pharmaceutically acceptable salt thereof, and the obinutuzumab to a plurality of humans results in a Grade 3 or Grade 4 adverse event in about 64% or less of the humans: or administration of the polatuzumab vedotin-piiq, the venetoclax or a pharmaceutically acceptable salt thereof, and the obinutuzumab to a plurality of humans results in a Grade 3 or Grade 4 adverse event in about 59% or less of the humans: or administration of the polatuzumab vedotin-piiq, the venetoclax or a pharmaceutically acceptable salt thereof, and the obinutuzumab to a plurality of humans results in a Grade 3 or Grade 4 adverse event in about 73% or less of the humans.

12. The method of claim 10, wherein the duration of the complete response is at least about 1 month, at least about 2 months, at least about 3 months, or more.

13-19. (canceled)

20. The method of claim 10, wherein the polatuzumab vedotin-piiq, the venetoclax or a pharmaceutically acceptable salt thereof, and the obinutuzumab are administered during an induction phase, optionally, wherein the induction phase comprises at least six 21-day cycles.

21. The method of claim 20, wherein
(i) the polatuzumab vedotin-piiq is administered intravenously at a dose of about 1.8 mg/kg on Day 1 of the first 21-day cycle, the venetoclax or a pharmaceutically acceptable salt thereof is administered orally at a dose of about 800 mg on each of Days 1-21 of the first 21-day cycle, and the obinutuzumab is administered intravenously at a dose of about 1000 mg on each of Days 1, 8, and 15 of the first 21-day cycle, and
(ii) the polatuzumab vedotin-piiq is administered intravenously at a dose of about 1.8 mg/kg on Day 1 of each of the second, third, fourth, fifth, and sixth 21-day cycles, the venetoclax or a pharmaceutically acceptable salt thereof is administered orally at a dose of about 800 mg on each of Days 1-21 of each of the second, third, fourth, fifth, and sixth 21-day cycles, and the obinutuzumab is administered intravenously at a dose of about 1000 mg on Day 1 of each of the second, third, fourth, fifth, and sixth 21-day cycles.

22. The method of claim 20, wherein the polatuzumab vedotin-piiq, the venetoclax or a pharmaceutically acceptable salt thereof, and the obinutuzumab are administered sequentially during the induction phase.

23. The method of claim 22, wherein
(i) the venetoclax or a pharmaceutically acceptable salt thereof is administered prior to the obinutuzumab and the obinutuzumab is administered prior to the polatuzumab vedotin-piiq on Day 1 of the first 21-day cycle, and the venetoclax or a pharmaceutically acceptable salt thereof is administered prior to the obinutuzumab on days 8 and 15 of the first 21-day cycle; and
(ii) the venetoclax or a pharmaceutically acceptable salt thereof is administered prior to the obinutuzumab and the obinutuzumab is administered prior to the polatuzumab vedotin-piiq on Day 1 of each of the second, third, fourth, fifth, and sixth 21-day cycles.

24. The method of claim 20, wherein:
(a) administration of the polatuzumab vedotin-piiq, the venetoclax or a pharmaceutically acceptable salt thereof, and the obinutuzumab results in a complete response in the human after the six 21-day cycles;
(b) administration of the polatuzumab vedotin-piiq, the venetoclax or a pharmaceutically acceptable salt thereof, and the obinutuzumab to a plurality of humans results in a complete response in at least about 55%, at least about 57%, at least about 60%, at least about 65%, at least about 70%, at least about 75%, at least about 80%, at least about 85%, at least about 90%, at least about 95%, or 100% of the humans after the six 21-day cycles; and/or
(c) administration of the polatuzumab vedotin-piiq, the venetoclax or a pharmaceutically acceptable salt thereof, and the obinutuzumab to a plurality of humans results in an objective response in at least about 87%, at least about 90%, at least about 95%, or 100% of the humans after the six 21-day cycles: or administration of the polatuzumab vedotin-piiq, the venetoclax or a pharmaceutically acceptable salt thereof, and the obinutuzumab to a plurality of humans results in an objective response in at least about 70%, at least about 75%, at least about 78%, at least about 80%, at least about 85%, at least about 90%, at least about 95%, or 100% of the humans after the six 21-day cycles.

25. (canceled)

26. The method of claim 24, wherein the duration of the complete response is at least about 1 month, at least about 2 months, at least about 3 months, or more.

27-28. (canceled)

29. The method of claim 20, wherein the venetoclax or a pharmaceutically acceptable salt thereof and the obinutuzumab are further administered during a maintenance phase after the sixth 21-day cycle of the induction phase, wherein the venetoclax or a pharmaceutically acceptable salt thereof is administered orally at a dose of about 800 mg once per day during the maintenance phase, and wherein the obinutuzumab is administered intravenously at a dose of about 1000 mg once every two months during the maintenance phase.

30. The method of claim 29, wherein the venetoclax or a pharmaceutically acceptable salt thereof is administered for a maximum of 8 months during the maintenance phase and/or the obinutuzumab is administered for a maximum of 24 months during the maintenance phase.

31. The method of claim 29, wherein the obinutuzumab is administered during the maintenance phase starting on Day 1 of the second month after the sixth 21-day cycle of the induction phase.

32. (canceled)

33. The method of claim 29, wherein the venetoclax or a pharmaceutically acceptable salt thereof and the obinutuzumab are administered sequentially during the maintenance phase.

34. The method of claim 33, wherein the venetoclax or a pharmaceutically acceptable salt thereof is administered prior to the obinutuzumab on Day 1 of each of months 2, 4, 6, 8, 10, 12, 14, 16, 18, 20, 22, and 24 during the maintenance phase.

35. The method of claim 1, wherein:
(a) the anti-CD79b antibody comprises a heavy chain comprising the amino acid sequence of SEQ ID NO: 37 and a light chain comprising the amino acid sequence of SEQ ID NO: 35;
(b) the anti-CD79b antibody comprises a heavy chain comprising the amino acid sequence of SEQ ID NO: 36 and a light chain comprising the amino acid sequence of SEQ ID NO: 38: or
(c) the immunoconjugate is iladatuzumab vedotin.

36-37. (canceled)

38. A method for treating follicular lymphoma (FL) in a human in need thereof comprising administering to the human an effective amount of:
(a) an immunoconjugate at a dose of about 1.8 mg/kg, wherein the immunoconjugate comprises the formula 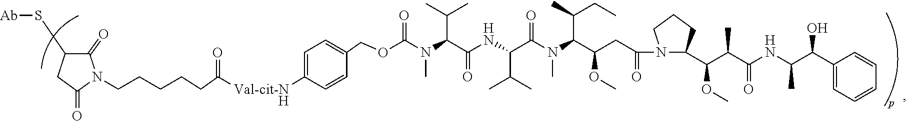
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) venetoclax or a pharmaceutically acceptable salt thereof at a dose of about 800 mg, and
(c) obinutuzumab at a dose of about 1000 mg.

39-69. (canceled)

70. A method for treating follicular lymphoma (FL) in a human in need thereof comprising administering to the human an effective amount of:
(a) an immunoconjugate at dose of about 1.8 mg/kg, wherein the immunoconjugate comprises the formula 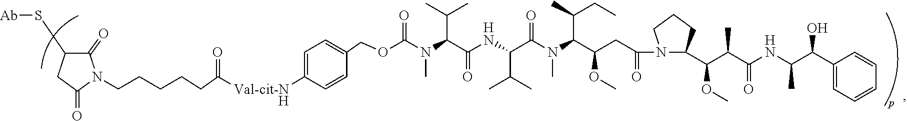
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) venetoclax or a pharmaceutically acceptable salt thereof at a dose of about 800 mg, and
(c) obinutuzumab at a dose of about 1000 mg,
wherein the human achieves a complete response (CR) during or after administration of the immunoconjugate, the venetoclax, and the obinutuzumab.

71-101. (canceled)

102. A method of treating follicular lymphoma (FL) in a human in need thereof, comprising administering to the human, during an induction phase, an effective amount of:
(a) polatuzumab vedotin-piiq at a dose of about 1.8 mg/kg,
(b) venetoclax or a pharmaceutically acceptable salt thereof at a dose of about 800 mg, and
(c) obinutuzumab at a dose of about 1000 mg,
wherein the human achieves a complete response during or after the induction phase.

103-121. (canceled)

122. The method of claim 1, further comprising: (a) administering a prophylactic treatment for tumor lysis syndrome (TLS), wherein the prophylactic treatment for TLS comprises a uric acid-reducing agent and/or a hydration regimen prior to the start of treatment; and/or (b) administering granulocyte colony stimulating factor (G-CSF) if a Grade 3 or Grade 4 adverse event of neutropenia occurs.

123-127. (canceled)

128. The method of claim 1, wherein the human:
(a) has an Eastern Cooperative Oncology Group (ECOG) Performance Status score of 0, 1, or 2 prior to the start of treatment;
(b) has at least one bi-dimensionally measurable lesion prior to treatment, wherein the lesion is at least 1.5 centimeters in its largest dimension as measured by computed tomography (CT) scan or magnetic resonance imaging (MRI);
(c) does not have peripheral neuropathy of grade greater than 1 prior to treatment;
(d) has FL with bone marrow involvement prior to treatment;
(e) has FL with an Ann Arbor stage of 1, 2, 3, or 4 prior to treatment;
(f) has FL with a Follicular Lymphoma International Prognostic Index (FLIPI) score of 0, 1, 2, 3, 4, or 5 prior to treatment;
(g) has received at least one prior treatment for FL; and/or
(h) has bulky disease of greater than 7 centimeters prior to treatment.

129. The method of claim 1, wherein the FL:
(a is relapsed or refractory to a prior treatment for FL;
(b) is histologically documented as being CD20-positive;
(c) is fluorodeoxyglucose (FDG)-avid FL;
(d) is positron emission tomography (PET)-positive FL;
(e) is not grade 3b FL;
(f) has a histologic grade of 1, 2, or 3a prior to treatment;
(g) is refractory to a prior treatment comprising an anti-CD20 agent; and/or
(h) progressed within 24 months of completing a first treatment for FL.

130-144. (canceled)

145. A kit comprising an immunoconjugate comprising the formula 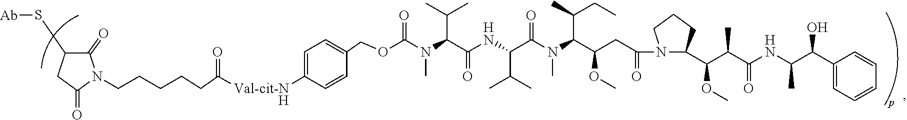
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and
wherein p is between 1 and 8,
for use in combination with a selective Bcl-2 inhibitor or a pharmaceutically acceptable salt thereof and anti-CD20 antibody for treating a human in need thereof having follicular lymphoma (FL) according to the method of claim 1.

146. A kit comprising an immunoconjugate comprising the formula 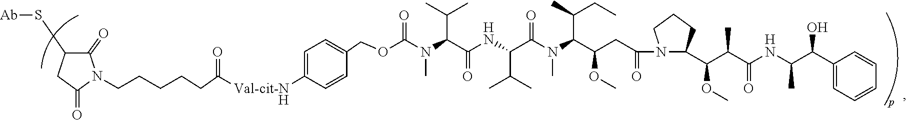
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and
wherein p is between 1 and 8,
for use in combination with venetoclax or a pharmaceutically acceptable salt thereof and obinutuzumab for treating a human in need thereof having follicular lymphoma (FL) according to the method of claim 1.

147-149. (canceled)

150. A kit comprising polatuzumab vedotin-piiq for use in combination with venetoclax or a pharmaceutically acceptable salt thereof and obinutuzumab for treating a human in need thereof having follicular lymphoma (FL) according to the method of claim 1.

151. A method for treating diffuse large B-cell lymphoma (DLBCL) in a human in need thereof comprising administering to the human an effective amount of:
(a) an immunoconjugate comprising the formula 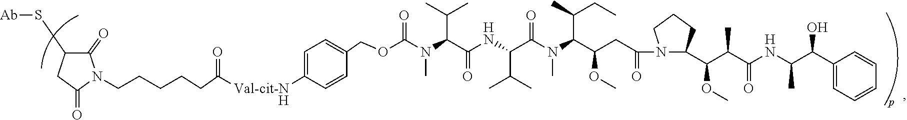
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) a selective Bcl-2 inhibitor, or a pharmaceutically acceptable salt thereof, and
(c) an anti-CD20 antibody,
wherein the human achieves a complete response (CR) during or after administration of the immunoconjugate, the selective Bcl-2 inhibitor, or a pharmaceutically acceptable salt thereof, and the anti-CD20 antibody.

152-192. (canceled)

193. A method for treating diffuse large B-cell lymphoma (DLBCL) in a human in need thereof comprising administering to the human an effective amount of:
(a) an immunoconjugate at a dose of about 1.8 mg/kg, wherein the immunoconjugate comprises the formula 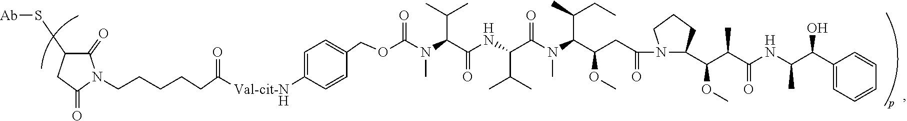
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) venetoclax or a pharmaceutically acceptable salt thereof at a dose of about 800 mg, and
(c) rituximab at a dose of about 375 mg/m².

194-230. (canceled)

231. A method for treating diffuse large B-cell lymphoma (DLBCL) in a human in need thereof comprising administering to the human an effective amount of:
(a) an immunoconjugate at a dose of about 1.8 mg/kg, wherein the immunoconjugate comprises the formula 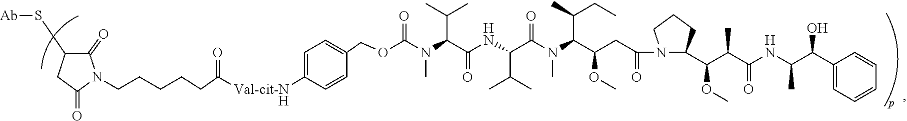
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO: 26, and
wherein p is between 1 and 8,
(b) venetoclax or a pharmaceutically acceptable salt thereof at a dose of about 800 mg, and
(c) rituximab at a dose of about 375 mg/m²,
wherein the human achieves a complete response (CR) during or after administration of the immunoconjugate, the venetoclax, and the rituximab.

232-268. (canceled)

269. A method of treating diffuse large B-cell lymphoma (DLBCL) in a human in need thereof, comprising administering to the human, during an induction phase, an effective amount of:
(a) polatuzumab vedotin-piiq at a dose of about 1.8 mg/kg,
(b) venetoclax or a pharmaceutically acceptable salt thereof at a dose of about 800 mg, and
(c) rituximab at a dose of about 375 mg/m²,
wherein the human achieves a complete response during or after the induction phase.

270-319. (canceled)

320. A kit comprising an immunoconjugate comprising the formula 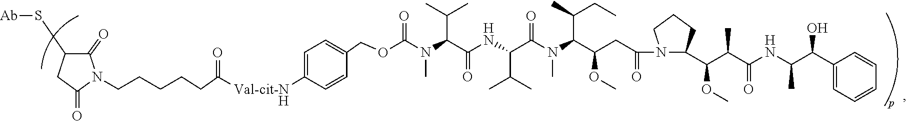
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and
wherein p is between 1 and 8,
for use in combination with a selective Bcl-2 inhibitor or a pharmaceutically acceptable salt thereof and an anti-CD20 antibody for treating a human in need thereof having diffuse large B-cell lymphoma (DLBCL) according to the method of claim 151.

321. A kit comprising an immunoconjugate comprising the formula 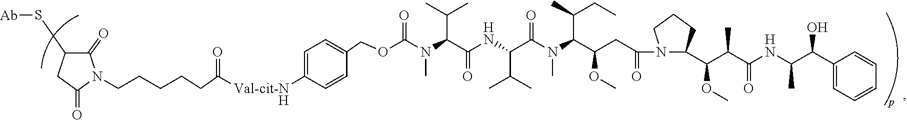
wherein Ab is an anti-CD79b antibody comprising (i) a hypervariable region-H1 (HVR-H1) that comprises the amino acid sequence of SEQ ID NO: 21; (ii) an HVR-H2 comprising the amino acid sequence of SEQ ID NO: 22; (iii) an HVR-H3 comprising the amino acid sequence of SEQ ID NO: 23; (iv) an HVR-L1 comprising the amino acid sequence of SEQ ID NO: 24; (v) an HVR-L2 comprising the amino acid sequence of SEQ ID NO: 25; and (vi) an HVR-L3 comprising the amino acid sequence of SEQ ID NO:26, and
wherein p is between 1 and 8,
for use in combination with venetoclax or a pharmaceutically acceptable salt thereof and rituximab for treating a human in need thereof having diffuse large B-cell lymphoma (DLBCL) according to the method of claim 151.

322-324. (canceled)

325. A kit comprising polatuzumab vedotin-piiq for use in combination with venetoclax or a pharmaceutically acceptable salt thereof and rituximab for treating a human in need thereof having diffuse large B-cell lymphoma (DLBCL) according to the method of claim 151.


SEQUENCE LISTING (56 sequences)

SEQ ID NO: 1 - protein, 179 aa, Homo sapiens
RFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 2 - protein, 201 aa, Homo sapiens
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 3 - protein, 112 aa, synthetic construct
  REGION 1..112: note = Synthetic Construct
GPELVKPGASVKISCKASGYAFSYSWMNWVKLRPGQGLEWIGRIFPGDGDTDYNGKFKGKATLTADKSSNTAYMQLTSLTSVDSAVYLCARNVFDGYWLVYWGQGTLVTVSA

SEQ ID NO: 4 - protein, 103 aa, synthetic construct
  REGION 1..103: note = Synthetic Construct
NPVTLGTSASISCRSSKSLLHSNGITYLYWYLQKPGQSPQLLIYQMSNLVSGVPDRFSSSGSGTDFTLRISRVEAEDVGVYYCAQNLELPYTFGGGTKLEIKR

SEQ ID NO: 5 - protein, 6 aa, synthetic construct
  REGION 1..6: note = Synthetic Construct
GYAFSY

SEQ ID NO: 6 - protein, 8 aa, synthetic construct
  REGION 1..8: note = Synthetic Construct
FPGDGDTD

SEQ ID NO: 7 - protein, 10 aa, synthetic construct
  REGION 1..10: note = Synthetic Construct
NVFDGYWLVY

SEQ ID NO: 8 - protein, 16 aa, synthetic construct
  REGION 1..16: note = Synthetic Construct
RSSKSLLHSNGITYLY

SEQ ID NO: 9 - protein, 7 aa, synthetic construct
  REGION 1..7: note = Synthetic Construct
QMSNLVS

SEQ ID NO: 10 - protein, 9 aa, synthetic construct
  REGION 1..9: note = Synthetic Construct
AQNLELPYT

SEQ ID NO: 11 - protein, 119 aa, synthetic construct
  REGION 1..119: note = Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 12 - protein, 115 aa, synthetic construct
  REGION 1..115: note = Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 13 - protein, 448 aa, synthetic construct
  REGION 1..448: note = Synthetic Construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 219 aa, synthetic construct
  REGION 1..219: note = Synthetic Construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 119 aa, synthetic construct
  REGION 1..119: note = Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 119 aa, synthetic construct
  REGION 1..119: note = Synthetic Construct
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYLCARNVFDGYWLVYWGQGTLVTVSS

SEQ ID NO: 17 - protein, 448 aa, synthetic construct; identical to SEQ ID NO: 13
  REGION 1..448: note = Synthetic Construct

SEQ ID NO: 18 - protein, 219 aa, synthetic construct; identical to SEQ ID NO: 14
  REGION 1..219: note = Synthetic Construct

SEQ ID NO: 19 - protein, 117 aa, synthetic construct [sequence omitted]

SEQ ID NO: 20 - protein, 112 aa, synthetic construct
  REGION 1..112: note = Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 21 - protein, 10 aa, synthetic construct
  REGION 1..10: note = Synthetic Construct
GYTFSSYWIE

SEQ ID NO: 22 - protein, 18 aa, synthetic construct
  REGION 1..18: note = Synthetic Construct
GEILPGGGDTNYNEIFKG

SEQ ID NO: 23 - protein, 10 aa, synthetic construct
  REGION 1..10: note = Synthetic Construct
TRRVPIRLDY

SEQ ID NO: 24 - protein, 15 aa, synthetic construct
  REGION 1..15: note = Synthetic Construct
KASQSVDYEGDSFLN

SEQ ID NO: 25 - protein, 7 aa, synthetic construct
  REGION 1..7: note = Synthetic Construct
AASNLES

SEQ ID NO: 26 - protein, 9 aa, synthetic construct
  REGION 1..9: note = Synthetic Construct
QQSNEDPLT

SEQ ID NO: 27 - protein, 25 aa, synthetic construct [sequence omitted]

SEQ ID NO: 28 - protein, 13 aa, synthetic construct
  REGION 1..13: note = Synthetic Construct
WVRQAPGKGLEWI

SEQ ID NO: 29 - protein, 30 aa, synthetic construct
  REGION 1..30: note = Synthetic Construct
RATFSADTSKNTAYLQMNSLRAEDTAVYYC

SEQ ID NO: 30 - protein, 11 aa, synthetic construct
  REGION 1..11: note = Synthetic Construct
WGQGTLVTVSS

SEQ ID NO: 31 - protein, 23 aa, synthetic construct
  REGION 1..23: note = Synthetic Construct
DIQLTQSPSSLSASVGDRVTITC

SEQ ID NO: 32 - protein, 15 aa, synthetic construct
  REGION 1..15: note = Synthetic Construct
WYQQKPGKAPKLLIY

SEQ ID NO: 33 - protein, 32 aa, synthetic construct
  REGION 1..32: note = Synthetic Construct
GVPSRFSGSGSGTDFTLTISSLQPEDFATYYC

SEQ ID NO: 34 - protein, 11 aa, synthetic construct
  REGION 1..11: note = Synthetic Construct
FGQGTKVEIKR

SEQ ID NO: 35 - protein, 218 aa, synthetic construct
  REGION 1..218: note = Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 446 aa, synthetic construct
  REGION 1..446: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 446 aa, synthetic construct
  REGION 1..446: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 218 aa, synthetic construct
  REGION 1..218: note = Synthetic Construct
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 39 - protein, 447 aa, synthetic construct
  REGION 1..447: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDCDGSFFLYSKLTVDKSRWQQGNVFSCSVMHEALHNHYTQKSLSLSPGK

SEQ ID NO: 40 - protein, 119 aa, synthetic construct
  REGION 1..119: note = Synthetic Construct
QVQLVQSGAEVKKPGASVKVSCKVSGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 41 - protein, 119 aa, synthetic construct
  REGION 1..119: note = Synthetic Construct
QVQLVQSGAEVKKPGSSVKVSCKASGYAFSYSWMSWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 42 - protein, 119 aa, synthetic construct; identical to SEQ ID NO: 11
  REGION 1..119: note = Synthetic Construct

SEQ ID NO: 43 - protein, 119 aa, synthetic construct
  REGION 1..119: note = Synthetic Construct
QVQLVQSGAEVKKPGSSVKVSCKASGYAFSYSWISWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 44 - protein, 119 aa, synthetic construct
  REGION 1..119: note = Synthetic Construct
QVQLVQSGAEVKKPGASVKVSCKASGYTFTYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 45 - protein, 119 aa, synthetic construct
  REGION 1..119: note = Synthetic Construct
QVQLVQSGAEVKKPGASVKVSCKASGYTFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 46 - protein, 119 aa, synthetic construct
  REGION 1..119: note = Synthetic Construct
EVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 47 - protein, 119 aa, synthetic construct
  REGION 1..119: note = Synthetic Construct
EVQLVESGGGLVKPGGSLRLSCAASGFAFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 48 - protein, 119 aa, synthetic construct
  REGION 1..119: note = Synthetic Construct
QVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 49 - protein, 119 aa, synthetic construct
  REGION 1..119: note = Synthetic Construct
EVQLVESGAGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 50 - protein, 119 aa, synthetic construct
  REGION 1..119: note = Synthetic Construct
EVQLVESGGGVVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 51 - protein, 119 aa, synthetic construct
  REGION 1..119: note = Synthetic Construct
EVQLVESGGGLKKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 52 - protein, 119 aa, synthetic construct
  REGION 1..119: note = Synthetic Construct
EVQLVESGGGLVKPGSSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 53 - protein, 119 aa, synthetic construct
  REGION 1..119: note = Synthetic Construct
EVQLVESGGGLVKPGGSLRVSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 54 - protein, 119 aa, synthetic construct
  REGION 1..119: note = Synthetic Construct
EVQLVESGGGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 55 - protein, 115 aa, synthetic construct; identical to SEQ ID NO: 12
  REGION 1..115: note = Synthetic Construct

SEQ ID NO: 56 - protein, 447 aa, synthetic construct
  REGION 1..447: note = Synthetic Construct
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK